SEQUENCE LISTING (449 sequences)

SEQ ID NO: 1 - DNA, 19 bp, synthetic construct
  misc_feature 1..19: note = Synthetic construct
  misc_feature 1..19: note = gRNA DDTI3_a spacer DNA sequence
  source 1..19: mol_type = other DNA
ccctagcgagagggagcga

SEQ ID NO: 2 - DNA, 19 bp, synthetic construct
  misc_feature 1..19: note = Synthetic construct
  misc_feature 1..19: note = gRNA HES2_a spacer DNA sequence
  source 1..19: mol_type = other DNA
ccgtcggggatctgcgggc

SEQ ID NO: 3 - DNA, 19 bp, synthetic construct
  misc_feature 1..19: note = Synthetic construct
  misc_feature 1..19: note = gRNA HKR1_a spacer DNA sequence
  source 1..19: mol_type = other DNA
cagtcccgcgatgccttcg

SEQ ID NO: 4 - DNA, 19 bp, synthetic construct
  misc_feature 1..19: note = Synthetic construct
  misc_feature 1..19: note = gRNA PLAG1_a spacer DNA sequence
  source 1..19: mol_type = other DNA
aacacttcggcttgacggg

SEQ ID NO: 5 - DNA, 19 bp, synthetic construct
  misc_feature 1..19: note = Synthetic construct
  misc_feature 1..19: note = gRNA PRDM4_a spacer DNA sequence
  source 1..19: mol_type = other DNA
tggccgcgagaggctcggc

SEQ ID NO: 6 - DNA, 19 bp, synthetic construct
  misc_feature 1..19: note = Synthetic construct
  misc_feature 1..19: note = gRNA TEF_a spacer DNA sequence
  source 1..19: mol_type = other DNA
aggctgggagccataactg

SEQ ID NO: 7 - DNA, 19 bp, synthetic construct
  misc_feature 1..19: note = Synthetic construct
  misc_feature 1..19: note = gRNA TSHZ1_a spacer DNA sequence
  source 1..19: mol_type = other DNA
gacctatttgtaattgtca

SEQ ID NO: 8 - DNA, 19 bp, synthetic construct
  misc_feature 1..19: note = Synthetic construct
  misc_feature 1..19: note = gRNA ZNF141_a spacer DNA sequence
  source 1..19: mol_type = other DNA
gagagctgtgcacccaact

SEQ ID NO: 9 - DNA, 19 bp, synthetic construct
  misc_feature 1..19: note = Synthetic construct
  misc_feature 1..19: note = gRNA ZNF562_a spacer DNA sequence
  source 1..19: mol_type = other DNA
tggacggaaccgtgggcga

SEQ ID NO: 10 - DNA, 19 bp, synthetic construct
  misc_feature 1..19: note = Synthetic construct
  misc_feature 1..19: note = gRNA ZNF691_a spacer DNA sequence
  source 1..19: mol_type = other DNA
cgccctactactccaggat

SEQ ID NO: 11 - DNA, 19 bp, synthetic construct
  misc_feature 1..19: note = Synthetic construct
  misc_feature 1..19: note = gRNA ZNF692_a spacer DNA sequence
  source 1..19: mol_type = other DNA
ccccgcggcagttgtggtt

SEQ ID NO: 12 - DNA, 19 bp, synthetic construct
  misc_feature 1..19: note = Synthetic construct
  misc_feature 1..19: note = gRNA ZSCAN5A_a spacer DNA sequence
  source 1..19: mol_type = other DNA
acgtaatcggcgttgatta

SEQ ID NO: 13 - DNA, 19 bp, synthetic construct
  misc_feature 1..19: note = Synthetic construct
  misc_feature 1..19: note = gRNA EBF4_a spacer DNA sequence
  source 1..19: mol_type = other DNA
aagacgccggggcttcgtc

SEQ ID NO: 14 - DNA, 19 bp, synthetic construct
  misc_feature 1..19: note = Synthetic construct
  misc_feature 1..19: note = gRNA ETS1_a spacer DNA sequence
  source 1..19: mol_type = other DNA
agaaacgccctaaagagga

SEQ ID NO: 15 - DNA, 19 bp, synthetic construct
  misc_feature 1..19: note = Synthetic construct
  misc_feature 1..19: note = gRNA FOXD3_a spacer DNA sequence
  source 1..19: mol_type = other DNA
gctgcaagggcggtgttgg

SEQ ID NO: 16 - DNA, 19 bp, synthetic construct
  misc_feature 1..19: note = Synthetic construct
  misc_feature 1..19: note = gRNA HELT_a spacer DNA sequence
  source 1..19: mol_type = other DNA
caagcgcctgcagcagtcg

SEQ ID NO: 17 - DNA, 19 bp, synthetic construct
  misc_feature 1..19: note = Synthetic construct
  misc_feature 1..19: note = gRNA HMGN3_a spacer DNA sequence
  source 1..19: mol_type = other DNA
gcgtaccgcgcgcggaggt

SEQ ID NO: 18 - DNA, 19 bp, synthetic construct
  misc_feature 1..19: note = Synthetic construct
  misc_feature 1..19: note = gRNA PATZ1_a spacer DNA sequence
  source 1..19: mol_type = other DNA
gaaacggcgagctcagagt

SEQ ID NO: 19 - DNA, 19 bp, synthetic construct
  misc_feature 1..19: note = Synthetic construct
  misc_feature 1..19: note = gRNA PRDM8_a spacer DNA sequence
  source 1..19: mol_type = other DNA
cccagctctccatccccgg

SEQ ID NO: 20 - DNA, 19 bp, synthetic construct
  misc_feature 1..19: note = Synthetic construct
  misc_feature 1..19: note = gRNA SOX21_a spacer DNA sequence
  source 1..19: mol_type = other DNA
cgggagaggaagactcgag

SEQ ID NO: 21 - DNA, 19 bp, synthetic construct
  misc_feature 1..19: note = Synthetic construct
  misc_feature 1..19: note = gRNA TBX15_a spacer DNA sequence
  source 1..19: mol_type = other DNA
gatcacagggaggacggga

SEQ ID NO: 22 - DNA, 19 bp, synthetic construct
  misc_feature 1..19: note = Synthetic construct
  misc_feature 1..19: note = gRNA ZBED5_a spacer DNA sequence
  source 1..19: mol_type = other DNA
aaaggaccaagctcggttg

SEQ ID NO: 23 - DNA, 19 bp, synthetic construct
  misc_feature 1..19: note = Synthetic construct
  misc_feature 1..19: note = gRNA ZBTB7A_a spacer DNA sequence
  source 1..19: mol_type = other DNA
tccctccgccgtttgcagc

SEQ ID NO: 24 - DNA, 19 bp, synthetic construct
  misc_feature 1..19: note = Synthetic construct
  misc_feature 1..19: note = gRNA ZNF219_a spacer DNA sequence
  source 1..19: mol_type = other DNA
cctaaacccggacatacca

SEQ ID NO: 25 - DNA, 19 bp, synthetic construct
  misc_feature 1..19: note = Synthetic construct
  misc_feature 1..19: note = gRNA ZNF319_a spacer DNA sequence
  source 1..19: mol_type = other DNA
taccactctaagccttctc

SEQ ID NO: 26 - DNA, 19 bp, synthetic construct
  misc_feature 1..19: note = Synthetic construct
  misc_feature 1..19: note = gRNA ZNF470_a spacer DNA sequence
  source 1..19: mol_type = other DNA
ttcccaggggctattgcag

SEQ ID NO: 27 - DNA, 19 bp, synthetic construct
  misc_feature 1..19: note = Synthetic construct
  misc_feature 1..19: note = gRNA ZNF816_a spacer DNA sequence
  source 1..19: mol_type = other DNA
agcaggaatgcgggcgcgg

SEQ ID NO: 28 - DNA, 19 bp, synthetic construct
  misc_feature 1..19: note = Synthetic construct
  misc_feature 1..19: note = gRNA ZSCAN23_a spacer DNA sequence
  source 1..19: mol_type = other DNA
ctccgcgaaacagcggttc

SEQ ID NO: 29 - DNA, 19 bp, synthetic construct
  misc_feature 1..19: note = Synthetic construct
  misc_feature 1..19: note = gRNA EMX1_a spacer DNA sequence
  source 1..19: mol_type = other DNA
agcgccgggagccgtggtt

SEQ ID NO: 30 - DNA, 19 bp, synthetic construct
  misc_feature 1..19: note = Synthetic construct
  misc_feature 1..19: note = gRNA FOXO3_a spacer DNA sequence
  source 1..19: mol_type = other DNA
gccgcgggaggcactagag

SEQ ID NO: 31 - DNA, 19 bp, synthetic construct
  misc_feature 1..19: note = Synthetic construct
  misc_feature 1..19: note = gRNA HIC2_a spacer DNA sequence
  source 1..19: mol_type = other DNA
tgggctcctggggaagccc

SEQ ID NO: 32 - DNA, 19 bp, synthetic construct
  misc_feature 1..19: note = Synthetic construct
  misc_feature 1..19: note = gRNA SOX30_a spacer DNA sequence
  source 1..19: mol_type = other DNA
gccgcggaaagcagatctg

SEQ ID NO: 33 - DNA, 19 bp, synthetic construct
  misc_feature 1..19: note = Synthetic construct
  misc_feature 1..19: note = gRNA STAT1_a spacer DNA sequence
  source 1..19: mol_type = other DNA
cagcgtggcaacacgggac

SEQ ID NO: 34 - DNA, 19 bp, synthetic construct
  misc_feature 1..19: note = Synthetic construct
  misc_feature 1..19: note = gRNA ZBTB46_a spacer DNA sequence
  source 1..19: mol_type = other DNA
ccgcgccgcggggtcgcag

SEQ ID NO: 35 - DNA, 19 bp, synthetic construct
  misc_feature 1..19: note = Synthetic construct
  misc_feature 1..19: note = gRNA ZNF331_a spacer DNA sequence
  source 1..19: mol_type = other DNA
caggtgagattggcgccca

SEQ ID NO: 36 - DNA, 19 bp, synthetic construct
  misc_feature 1..19: note = Synthetic construct
  misc_feature 1..19: note = gRNA ZNF541_a spacer DNA sequence
  source 1..19: mol_type = other DNA
caagtcatgtgtcagataa

SEQ ID NO: 37 - DNA, 19 bp, synthetic construct
  misc_feature 1..19: note = Synthetic construct
  misc_feature 1..19: note = gRNA ZNF619_a spacer DNA sequence
  source 1..19: mol_type = other DNA
cgcagagcggttgtggaga

SEQ ID NO: 38 - DNA, 19 bp, synthetic construct
  misc_feature 1..19: note = Synthetic construct
  misc_feature 1..19: note = gRNA AEBP1_a spacer DNA sequence
  source 1..19: mol_type = other DNA
cgggaccccgagtccaccg

SEQ ID NO: 39 - DNA, 19 bp, synthetic construct
  misc_feature 1..19: note = Synthetic construct
  misc_feature 1..19: note = gRNA ARID3A_a spacer DNA sequence
  source 1..19: mol_type = other DNA
cccgcccccggcctaggga

SEQ ID NO: 40 - DNA, 19 bp, synthetic construct
  misc_feature 1..19: note = Synthetic construct
  misc_feature 1..19: note = gRNA ARNTL2_a spacer DNA sequence
  source 1..19: mol_type = other DNA
accctcggggcaaaggtaa

SEQ ID NO: 41 - DNA, 19 bp, synthetic construct
  misc_feature 1..19: note = Synthetic construct
  misc_feature 1..19: note = gRNA BACH1_a spacer DNA sequence
  source 1..19: mol_type = other DNA
tcgctgacagaggcttcga

SEQ ID NO: 42 - DNA, 19 bp, synthetic construct
  misc_feature 1..19: note = Synthetic construct
  misc_feature 1..19: note = gRNA BATF2_a spacer DNA sequence
  source 1..19: mol_type = other DNA
caaaggccaggtatgagga

SEQ ID NO: 43 - DNA, 19 bp, synthetic construct
  misc_feature 1..19: note = Synthetic construct
  misc_feature 1..19: note = gRNA BRD4_a spacer DNA sequence
  source 1..19: mol_type = other DNA
tgctgaagtcatgaggtgt

SEQ ID NO: 44 - DNA, 19 bp, synthetic construct
  misc_feature 1..19: note = Synthetic construct
  misc_feature 1..19: note = gRNA CDX4_a spacer DNA sequence
  source 1..19: mol_type = other DNA
gggaagatcggtgaaaggg

SEQ ID NO: 45 - DNA, 19 bp, synthetic construct
  misc_feature 1..19: note = Synthetic construct
  misc_feature 1..19: note = gRNA CENPB_a spacer DNA sequence
  source 1..19: mol_type = other DNA
gcccccccgcggtatgtcc

SEQ ID NO: 46 - DNA, 19 bp, synthetic construct
  misc_feature 1..19: note = Synthetic construct
  misc_feature 1..19: note = gRNA DMRT1_a spacer DNA sequence
  source 1..19: mol_type = other DNA
agagcggtggctggagtcg

SEQ ID NO: 47 - DNA, 19 bp, synthetic construct
  misc_feature 1..19: note = Synthetic construct
  misc_feature 1..19: note = gRNA E2F2_a spacer DNA sequence
  source 1..19: mol_type = other DNA
cggaaagaacgcgtcctcg

SEQ ID NO: 48 - DNA, 19 bp, synthetic construct
  misc_feature 1..19: note = Synthetic construct
  misc_feature 1..19: note = gRNA ESRRB_a spacer DNA sequence
  source 1..19: mol_type = other DNA
gaggggcaacccccgactc

SEQ ID NO: 49 - DNA, 19 bp, synthetic construct
  misc_feature 1..19: note = Synthetic construct
  misc_feature 1..19: note = gRNA FERD3L_a spacer DNA sequence
  source 1..19: mol_type = other DNA
tgagtaggaggtaaaggta

SEQ ID NO: 50 - DNA, 19 bp, synthetic construct
  misc_feature 1..19: note = Synthetic construct
  misc_feature 1..19: note = gRNA FEV_a spacer DNA sequence
  source 1..19: mol_type = other DNA
cgggagccgactggaagaa

SEQ ID NO: 51 - DNA, 19 bp, synthetic construct
  misc_feature 1..19: note = Synthetic construct
  misc_feature 1..19: note = gRNA FLYWCH1_a spacer DNA sequence
  source 1..19: mol_type = other DNA
gggacgtaactcacggaaa

SEQ ID NO: 52 - DNA, 19 bp, synthetic construct
  misc_feature 1..19: note = Synthetic construct
  misc_feature 1..19: note = gRNA FOXJ3_a spacer DNA sequence
  source 1..19: mol_type = other DNA
cagagcctcgggagtctca

SEQ ID NO: 53 - DNA, 19 bp, synthetic construct
  misc_feature 1..19: note = Synthetic construct
  misc_feature 1..19: note = gRNA GLIS3_a spacer DNA sequence
  source 1..19: mol_type = other DNA
ggctcgcgggtcactaggc

SEQ ID NO: 54 - DNA, 19 bp, synthetic construct
  misc_feature 1..19: note = Synthetic construct
  misc_feature 1..19: note = gRNA HDAC9_a spacer DNA sequence
  source 1..19: mol_type = other DNA
gtaaagcaagcttcgagag

SEQ ID NO: 55 - DNA, 19 bp, synthetic construct
  misc_feature 1..19: note = Synthetic construct
  misc_feature 1..19: note = gRNA HIVEP1_2 spacer DNA sequence
  source 1..19: mol_type = other DNA
cagtcacgcggccccggga

SEQ ID NO: 56 - DNA, 19 bp, synthetic construct
  misc_feature 1..19: note = Synthetic construct
  misc_feature 1..19: note = gRNA HOXA2_4 spacer DNA sequence
  source 1..19: mol_type = other DNA
ggaaggaacgcctcattgg

SEQ ID NO: 57 - DNA, 19 bp, synthetic construct
  misc_feature 1..19: note = Synthetic construct
  misc_feature 1..19: note = gRNA HOXC9_1 spacer DNA sequence
  source 1..19: mol_type = other DNA
gagctaggagccgtcctga

SEQ ID NO: 58 - DNA, 19 bp, synthetic construct
  misc_feature 1..19: note = Synthetic construct
  misc_feature 1..19: note = gRNA HSFY1_5 spacer DNA sequence
  source 1..19: mol_type = other DNA
ggtaggttacgggttgtca

SEQ ID NO: 59 - DNA, 19 bp, synthetic construct
  misc_feature 1..19: note = Synthetic construct
  misc_feature 1..19: note = gRNA ISL2_2 spacer DNA sequence
  source 1..19: mol_type = other DNA
aacggccgcggagccagct

SEQ ID NO: 60 - DNA, 19 bp, synthetic construct
  misc_feature 1..19: note = Synthetic construct
  misc_feature 1..19: note = gRNA KAT2A_5 spacer DNA sequence
  source 1..19: mol_type = other DNA
tccggagccgcgtggggca

SEQ ID NO: 61 - DNA, 19 bp, synthetic construct
  misc_feature 1..19: note = Synthetic construct
  misc_feature 1..19: note = gRNA KDM5D_5 spacer DNA sequence
  source 1..19: mol_type = other DNA
gcaagagcgattttaccag

SEQ ID NO: 62 - DNA, 19 bp, synthetic construct
  misc_feature 1..19: note = Synthetic construct
  misc_feature 1..19: note = gRNA KLF13_a spacer DNA sequence
  source 1..19: mol_type = other DNA
accgggcacgcccctctaa

SEQ ID NO: 63 - DNA, 19 bp, synthetic construct
  misc_feature 1..19: note = Synthetic construct
  misc_feature 1..19: note = gRNA KLF6_a spacer DNA sequence
  source 1..19: mol_type = other DNA
tgaccgagactctccatca

SEQ ID NO: 64 - DNA, 19 bp, synthetic construct
  misc_feature 1..19: note = Synthetic construct
  misc_feature 1..19: note = gRNA MEOX2_a spacer DNA sequence
  source 1..19: mol_type = other DNA
aatcagggtgctaggttgg

SEQ ID NO: 65 - DNA, 19 bp, synthetic construct
  misc_feature 1..19: note = Synthetic construct
  misc_feature 1..19: note = gRNA MLXIPL_a spacer DNA sequence
  source 1..19: mol_type = other DNA
cgccagagctccagagcac

SEQ ID NO: 66 - DNA, 19 bp, synthetic construct
  misc_feature 1..19: note = Synthetic construct
  misc_feature 1..19: note = gRNA MNT_a spacer DNA sequence
  source 1..19: mol_type = other DNA
gggccgcggcgtctcgcga

SEQ ID NO: 67 - DNA, 19 bp, synthetic construct
  misc_feature 1..19: note = Synthetic construct
  misc_feature 1..19: note = gRNA MYCN_a spacer DNA sequence
  source 1..19: mol_type = other DNA
caagcccgggggtccaaaa

SEQ ID NO: 68 - DNA, 19 bp, synthetic construct
  misc_feature 1..19: note = Synthetic construct
  misc_feature 1..19: note = gRNA MYT1L_a spacer DNA sequence
  source 1..19: mol_type = other DNA
gcctcatagataatgatga

SEQ ID NO: 69 - DNA, 19 bp, synthetic construct
  misc_feature 1..19: note = Synthetic construct
  misc_feature 1..19: note = gRNA NEUROG3_a spacer DNA sequence
  source 1..19: mol_type = other DNA
ctcgcgtggcgcggcccca

SEQ ID NO: 70 - DNA, 19 bp, synthetic construct
  misc_feature 1..19: note = Synthetic construct
  misc_feature 1..19: note = gRNA NFE2_a spacer DNA sequence
  source 1..19: mol_type = other DNA
aggagagccacggggaaag

SEQ ID NO: 71 - DNA, 19 bp, synthetic construct
  misc_feature 1..19: note = Synthetic construct
  misc_feature 1..19: note = gRNA NR2F1_a spacer DNA sequence
  source 1..19: mol_type = other DNA
tttctctccccccgattca

SEQ ID NO: 72 - DNA, 19 bp, synthetic construct
  misc_feature 1..19: note = Synthetic construct
  misc_feature 1..19: note = gRNA NR2F2_a spacer DNA sequence
  source 1..19: mol_type = other DNA
ccccctcgccgtcattggc

SEQ ID NO: 73 - DNA, 19 bp, synthetic construct
  misc_feature 1..19: note = Synthetic construct
  misc_feature 1..19: note = gRNA NR3C1_a spacer DNA sequence
  source 1..19: mol_type = other DNA
gggcccgcgcggagggcgt

SEQ ID NO: 74 - DNA, 19 bp, synthetic construct
  misc_feature 1..19: note = Synthetic construct
  misc_feature 1..19: note = gRNA PHF20_a spacer DNA sequence
  source 1..19: mol_type = other DNA
aggccgttgccacagtgat

SEQ ID NO: 75 - DNA, 19 bp, synthetic construct
  misc_feature 1..19: note = Synthetic construct
  misc_feature 1..19: note = gRNA PITX2_a spacer DNA sequence
  source 1..19: mol_type = other DNA
caggacagtccctccaccg

SEQ ID NO: 76 - DNA, 19 bp, synthetic construct
  misc_feature 1..19: note = Synthetic construct
  misc_feature 1..19: note = gRNA POU2AF1_a spacer DNA sequence
  source 1..19: mol_type = other DNA
gccgctggggcgcgcacag

SEQ ID NO: 77 - DNA, 19 bp, synthetic construct
  misc_feature 1..19: note = Synthetic construct
  misc_feature 1..19: note = gRNA POU3F2_a spacer DNA sequence
  source 1..19: mol_type = other DNA
gcggggcccatccgagaga

SEQ ID NO: 78 - DNA, 19 bp, synthetic construct
  misc_feature 1..19: note = Synthetic construct
  misc_feature 1..19: note = gRNA POU3F3_a spacer DNA sequence
  source 1..19: mol_type = other DNA
ggccacgaccccccctgaa

SEQ ID NO: 79 - DNA, 19 bp, synthetic construct
  misc_feature 1..19: note = Synthetic construct
  misc_feature 1..19: note = gRNA PRDM11_a spacer DNA sequence
  source 1..19: mol_type = other DNA
tgggtgtggaggggttcca

SEQ ID NO: 80 - DNA, 19 bp, synthetic construct
  misc_feature 1..19: note = Synthetic construct
  misc_feature 1..19: note = gRNA PRMT3_a spacer DNA sequence
  source 1..19: mol_type = other DNA
tgaagaggcgcgctgatga

SEQ ID NO: 81 - DNA, 19 bp, synthetic construct
  misc_feature 1..19: note = Synthetic construct
  misc_feature 1..19: note = gRNA PROP1_a spacer DNA sequence
  source 1..19: mol_type = other DNA
tcggagggtccagggactg

SEQ ID NO: 82 - DNA, 19 bp, synthetic construct
  misc_feature 1..19: note = Synthetic construct
  misc_feature 1..19: note = gRNA PURA_a spacer DNA sequence
  source 1..19: mol_type = other DNA
ctcacgcgcgagaaggggg

SEQ ID NO: 83 - DNA, 19 bp, synthetic construct
  misc_feature 1..19: note = Synthetic construct
  misc_feature 1..19: note = gRNA RELB_a spacer DNA sequence
  source 1..19: mol_type = other DNA
tgggcgctcaggaggggtt

SEQ ID NO: 84 - DNA, 19 bp, synthetic construct
  misc_feature 1..19: note = Synthetic construct
  misc_feature 1..19: note = gRNA RFX3_a spacer DNA sequence
  source 1..19: mol_type = other DNA
actgacggcccccaaaccc

SEQ ID NO: 85 - DNA, 19 bp, synthetic construct
  misc_feature 1..19: note = Synthetic construct
  misc_feature 1..19: note = gRNA SAFB_a spacer DNA sequence
  source 1..19: mol_type = other DNA
ctgctaagtacgctagtgg

SEQ ID NO: 86 - DNA, 19 bp, synthetic construct
  misc_feature 1..19: note = Synthetic construct
  misc_feature 1..19: note = gRNA SETBP1_a spacer DNA sequence
  source 1..19: mol_type = other DNA
tatggagagagatatcaag

SEQ ID NO: 87 - DNA, 19 bp, synthetic construct
  misc_feature 1..19: note = Synthetic construct
  misc_feature 1..19: note = gRNA SMAD2_a spacer DNA sequence
  source 1..19: mol_type = other DNA
aagccgcgcggggcgggta

SEQ ID NO: 88 - DNA, 19 bp, synthetic construct
  misc_feature 1..19: note = Synthetic construct
  misc_feature 1..19: note = gRNA SMAD6_a spacer DNA sequence
  source 1..19: mol_type = other DNA
cccggggcgggccgagctc

SEQ ID NO: 89 - DNA, 19 bp, synthetic construct
  misc_feature 1..19: note = Synthetic construct
  misc_feature 1..19: note = gRNA SNAPC2_a spacer DNA sequence
  source 1..19: mol_type = other DNA
agggagggcatgggatcca

SEQ ID NO: 90 - DNA, 19 bp, synthetic construct
  misc_feature 1..19: note = Synthetic construct
  misc_feature 1..19: note = gRNA SNAPC4_a spacer DNA sequence
  source 1..19: mol_type = other DNA
tctcagagcccaccctcga

SEQ ID NO: 91 - DNA, 19 bp, synthetic construct
  misc_feature 1..19: note = Synthetic construct
  misc_feature 1..19: note = gRNA SON_a spacer DNA sequence
  source 1..19: mol_type = other DNA
ctgtacttggcctgtacta

SEQ ID NO: 92 - DNA, 19 bp, synthetic construct
  misc_feature 1..19: note = Synthetic construct
  misc_feature 1..19: note = gRNA SOX11_a spacer DNA sequence
  source 1..19: mol_type = other DNA
gggctgctttgcaaagatg

SEQ ID NO: 93 - DNA, 19 bp, synthetic construct
  misc_feature 1..19: note = Synthetic construct
  misc_feature 1..19: note = gRNA SOX12_a spacer DNA sequence
  source 1..19: mol_type = other DNA
ggcccggccgcagcccaag

SEQ ID NO: 94 - DNA, 19 bp, synthetic construct
  misc_feature 1..19: note = Synthetic construct
  misc_feature 1..19: note = gRNA SOX14_a spacer DNA sequence
  source 1..19: mol_type = other DNA
caaaagcggcgaagttcaa

SEQ ID NO: 95 - DNA, 19 bp, synthetic construct
  misc_feature 1..19: note = Synthetic construct
  misc_feature 1..19: note = gRNA SPIC_a spacer DNA sequence
  source 1..19: mol_type = other DNA
tgcaacctaagttgggcgg

SEQ ID NO: 96 - DNA, 19 bp, synthetic construct
  misc_feature 1..19: note = Synthetic construct
  misc_feature 1..19: note = gRNA THAP7_a spacer DNA sequence
  source 1..19: mol_type = other DNA
aatcggctgggacggggtt

SEQ ID NO: 97 - DNA, 19 bp, synthetic construct
  misc_feature 1..19: note = Synthetic construct
  misc_feature 1..19: note = gRNA TOX2_a spacer DNA sequence
  source 1..19: mol_type = other DNA
gctcccgcgggcagtgcct

SEQ ID NO: 98 - DNA, 19 bp, synthetic construct
  misc_feature 1..19: note = Synthetic construct
  misc_feature 1..19: note = gRNA TRERF1_a spacer DNA sequence
  source 1..19: mol_type = other DNA
gggtaccggggagcggata

SEQ ID NO: 99 - DNA, 19 bp, synthetic construct
  misc_feature 1..19: note = Synthetic construct
  misc_feature 1..19: note = gRNA TRIM3_a spacer DNA sequence
  source 1..19: mol_type = other DNA
gggcgtgcgggtgcggtag

SEQ ID NO: 100 - DNA, 19 bp, synthetic construct
  misc_feature 1..19: note = Synthetic construct
  misc_feature 1..19: note = gRNA VDR_a spacer DNA sequence
  source 1..19: mol_type = other DNA
tacagcgtggcctataggg

SEQ ID NO: 101 - DNA, 19 bp, synthetic construct
  misc_feature 1..19: note = Synthetic construct
  misc_feature 1..19: note = gRNA YBX2_a spacer DNA sequence
  source 1..19: mol_type = other DNA
gggcccaggtgtacttact

SEQ ID NO: 102 - DNA, 19 bp, synthetic construct
  misc_feature 1..19: note = Synthetic construct
  misc_feature 1..19: note = gRNA ZBTB14_a spacer DNA sequence
  source 1..19: mol_type = other DNA
cgcgcgcgtgcgcaggcca

SEQ ID NO: 103 - DNA, 19 bp, synthetic construct
  misc_feature 1..19: note = Synthetic construct
  misc_feature 1..19: note = gRNA ZBTB4_a spacer DNA sequence
  source 1..19: mol_type = other DNA
ccgggacggttggagaaga

SEQ ID NO: 104 - DNA, 19 bp, synthetic construct
  misc_feature 1..19: note = Synthetic construct
  misc_feature 1..19: note = gRNA ZFHX2_a spacer DNA sequence
  source 1..19: mol_type = other DNA
ggtacaggtctcatgcctg

SEQ ID NO: 105 - DNA, 19 bp, synthetic construct
  misc_feature 1..19: note = Synthetic construct
  misc_feature 1..19: note = gRNA ZFP57_a spacer DNA sequence
  source 1..19: mol_type = other DNA
gcagctatcagttggtcgg

SEQ ID NO: 106 - DNA, 19 bp, synthetic construct
  misc_feature 1..19: note = Synthetic construct
  misc_feature 1..19: note = gRNA ZFP62_a spacer DNA sequence
  source 1..19: mol_type = other DNA
gagggcagagagacctcta

SEQ ID NO: 107 - DNA, 19 bp, synthetic construct
  misc_feature 1..19: note = Synthetic construct
  misc_feature 1..19: note = gRNA ZFP69B_a spacer DNA sequence
  source 1..19: mol_type = other DNA
tgatgatgctcgcagggat

SEQ ID NO: 108 - DNA, 19 bp, synthetic construct
  misc_feature 1..19: note = Synthetic construct
  misc_feature 1..19: note = gRNA ZIC1_a spacer DNA sequence
  source 1..19: mol_type = other DNA
gggaatgcgggcgcaccaa

SEQ ID NO: 109 - DNA, 19 bp, synthetic construct
  misc_feature 1..19: note = Synthetic construct
  misc_feature 1..19: note = gRNA ZIC4_a spacer DNA sequence
  source 1..19: mol_type = other DNA
caacggggacgttgacaag

SEQ ID NO: 110 - DNA, 19 bp, synthetic construct
  misc_feature 1..19: note = Synthetic construct
  misc_feature 1..19: note = gRNA ZNF136_a spacer DNA sequence
  source 1..19: mol_type = other DNA
ccccgacggcgcgcctgat

SEQ ID NO: 111 - DNA, 19 bp, synthetic construct
  misc_feature 1..19: note = Synthetic construct
  misc_feature 1..19: note = gRNA ZNF16_a spacer DNA sequence
  source 1..19: mol_type = other DNA
gctagtgggaccaggtctg

SEQ ID NO: 112 - DNA, 19 bp, synthetic construct
  misc_feature 1..19: note = Synthetic construct
  misc_feature 1..19: note = gRNA ZNF174_a spacer DNA sequence
  source 1..19: mol_type = other DNA
caggggcgtgcgtaatggg

SEQ ID NO: 113 - DNA, 19 bp, synthetic construct
  misc_feature 1..19: note = Synthetic construct
  misc_feature 1..19: note = gRNA ZNF253_a spacer DNA sequence
  source 1..19: mol_type = other DNA
tgtcactcagcggctgggg

SEQ ID NO: 114 - DNA, 19 bp, synthetic construct
  misc_feature 1..19: note = Synthetic construct
  misc_feature 1..19: note = gRNA ZNF26_a spacer DNA sequence
  source 1..19: mol_type = other DNA
acagcctgacgggaaagga

SEQ ID NO: 115 - DNA, 19 bp, synthetic construct
  misc_feature 1..19: note = Synthetic construct
  misc_feature 1..19: note = gRNA ZNF263_a spacer DNA sequence
  source 1..19: mol_type = other DNA
gggcgcgccctgaaattct

SEQ ID NO: 116 - DNA, 19 bp, synthetic construct
  misc_feature 1..19: note = Synthetic construct
  misc_feature 1..19: note = gRNA ZNF285_a spacer DNA sequence
  source 1..19: mol_type = other DNA
aaagccccgcgcgcagcag

SEQ ID NO: 117 - DNA, 19 bp, synthetic construct
  misc_feature 1..19: note = Synthetic construct
  misc_feature 1..19: note = gRNA ZNF317_a spacer DNA sequence
  source 1..19: mol_type = other DNA
tcccgcgagaattggaagg

SEQ ID NO: 118 - DNA, 19 bp, synthetic construct
  misc_feature 1..19: note = Synthetic construct
  misc_feature 1..19: note = gRNA ZNF319_b spacer DNA sequence
  source 1..19: mol_type = other DNA
catcttccggaagggaggg

SEQ ID NO: 119 - DNA, 19 bp, synthetic construct
  misc_feature 1..19: note = Synthetic construct
  misc_feature 1..19: note = gRNA ZNF345_a spacer DNA sequence
  source 1..19: mol_type = other DNA
cgcgccaggggaatcggga

SEQ ID NO: 120 - DNA, 19 bp, synthetic construct
  misc_feature 1..19: note = Synthetic construct
  misc_feature 1..19: note = gRNA ZNF420_a spacer DNA sequence
  source 1..19: mol_type = other DNA
gagcccacgagcccctcga

SEQ ID NO: 121 - DNA, 19 bp, synthetic construct
  misc_feature 1..19: note = Synthetic construct
  misc_feature 1..19: note = gRNA ZNF423_a spacer DNA sequence
  source 1..19: mol_type = other DNA
caggggatcagccatccag

SEQ ID NO: 122 - DNA, 19 bp, synthetic construct
  misc_feature 1..19: note = Synthetic construct
  misc_feature 1..19: note = gRNA ZNF470_b spacer DNA sequence
  source 1..19: mol_type = other DNA
gaccaggagaggcccaaaa

SEQ ID NO: 123 - DNA, 19 bp, synthetic construct
  misc_feature 1..19: note = Synthetic construct
  misc_feature 1..19: note = gRNA ZNF48_a spacer DNA sequence
  source 1..19: mol_type = other DNA
gaggccggcggcccgaact

SEQ ID NO: 124 - DNA, 19 bp, synthetic construct
  misc_feature 1..19: note = Synthetic construct
  misc_feature 1..19: note = gRNA ZNF496_a spacer DNA sequence
  source 1..19: mol_type = other DNA
ggagcaggaagtgtcccag

SEQ ID NO: 125 - DNA, 19 bp, synthetic construct
  misc_feature 1..19: note = Synthetic construct
  misc_feature 1..19: note = gRNA ZNF501_a spacer DNA sequence
  source 1..19: mol_type = other DNA
aaacgggatggaagtcaga

SEQ ID NO: 126 - DNA, 19 bp, synthetic construct
  misc_feature 1..19: note = Synthetic construct
  misc_feature 1..19: note = gRNA ZNF524_a spacer DNA sequence
  source 1..19: mol_type = other DNA
gggtcctaacgaccggtcg

SEQ ID NO: 127 - DNA, 19 bp, synthetic construct
  misc_feature 1..19: note = Synthetic construct
  misc_feature 1..19: note = gRNA ZNF557_a spacer DNA sequence
  source 1..19: mol_type = other DNA
ctggcgcgggtctgtggca

SEQ ID NO: 128 - DNA, 19 bp, synthetic construct
  misc_feature 1..19: note = Synthetic construct
  misc_feature 1..19: note = gRNA ZNF735_a spacer DNA sequence
  source 1..19: mol_type = other DNA
aaaaggcccggtaaaatta

SEQ ID NO: 129 - DNA, 19 bp, synthetic construct
  misc_feature 1..19: note = Synthetic construct
  misc_feature 1..19: note = gRNA ZNF747_a spacer DNA sequence
  source 1..19: mol_type = other DNA
ggaacaaagcagcgccctg

SEQ ID NO: 130 - DNA, 19 bp, synthetic construct
  misc_feature 1..19: note = Synthetic construct
  misc_feature 1..19: note = gRNA ZNF783_a spacer DNA sequence
  source 1..19: mol_type = other DNA
aggcgctgcaacagagtct

SEQ ID NO: 131 - DNA, 19 bp, synthetic construct
  misc_feature 1..19: note = Synthetic construct
  misc_feature 1..19: note = gRNA ZNF805_a spacer DNA sequence
  source 1..19: mol_type = other DNA
ctggggccaatgatggtcc

SEQ ID NO: 132 - DNA, 19 bp, synthetic construct
  misc_feature 1..19: note = Synthetic construct
  misc_feature 1..19: note = gRNA ZSCAN10_a spacer DNA sequence
  source 1..19: mol_type = other DNA
ttacactctcacacaggct

SEQ ID NO: 133 - RNA, 19 nt, synthetic construct
  misc_feature 1..19: note = Synthetic construct
  misc_feature 1..19: note = gRNA DDTI3_a spacer RNA sequence
  source 1..19: mol_type = other RNA
ccctagcgagagggagcga

SEQ ID NO: 134 - RNA, 19 nt, synthetic construct
  misc_feature 1..19: note = Synthetic construct
  misc_feature 1..19: note = gRNA HES2_a spacer RNA sequence
  source 1..19: mol_type = other RNA
ccgtcggggatctgcgggc

SEQ ID NO: 135 - RNA, 19 nt, synthetic construct
  misc_feature 1..19: note = Synthetic construct
  misc_feature 1..19: note = gRNA HKR1_a spacer RNA sequence
  source 1..19: mol_type = other RNA
cagtcccgcgatgccttcg

SEQ ID NO: 136 - RNA, 19 nt, synthetic construct
  misc_feature 1..19: note = Synthetic construct
  misc_feature 1..19: note = gRNA PLAG1_a spacer RNA sequence
  source 1..19: mol_type = other RNA
aacacttcggcttgacggg

SEQ ID NO: 137 - RNA, 19 nt, synthetic construct
  misc_feature 1..19: note = Synthetic construct
  misc_feature 1..19: note = gRNA PRDM4_a spacer RNA sequence
  source 1..19: mol_type = other RNA
tggccgcgagaggctcggc

SEQ ID NO: 138 - RNA, 19 nt, synthetic construct
  misc_feature 1..19: note = Synthetic construct
  misc_feature 1..19: note = gRNA TEF_a spacer RNA sequence
  source 1..19: mol_type = other RNA
aggctgggagccataactg

SEQ ID NO: 139 - RNA, 19 nt, synthetic construct
  misc_feature 1..19: note = Synthetic construct
  misc_feature 1..19: note = gRNA TSHZ1_a spacer RNA sequence
  source 1..19: mol_type = other RNA
gacctatttgtaattgtca

SEQ ID NO: 140 - RNA, 19 nt, synthetic construct
  misc_feature 1..19: note = Synthetic construct
  misc_feature 1..19: note = gRNA ZNF141_a spacer RNA sequence
  source 1..19: mol_type = other RNA
gagagctgtgcacccaact

SEQ ID NO: 141 - RNA, 19 nt, synthetic construct
  misc_feature 1..19: note = Synthetic construct
  misc_feature 1..19: note = gRNA ZNF562_a spacer RNA sequence
  source 1..19: mol_type = other RNA
tggacggaaccgtgggcga

SEQ ID NO: 142 - RNA, 19 nt, synthetic construct
  misc_feature 1..19: note = Synthetic construct
  misc_feature 1..19: note = gRNA ZNF691_a spacer RNA sequence
  source 1..19: mol_type = other RNA
cgccctactactccaggat

SEQ ID NO: 143 - RNA, 19 nt, synthetic construct
  misc_feature 1..19: note = Synthetic construct
  misc_feature 1..19: note = gRNA ZNF692_a spacer RNA sequence
  source 1..19: mol_type = other RNA
ccccgcggcagttgtggtt

SEQ ID NO: 144 - RNA, 19 nt, synthetic construct
  misc_feature 1..19: note = Synthetic construct
  misc_feature 1..19: note = gRNA ZSCAN5A_a spacer RNA sequence
  source 1..19: mol_type = other RNA
acgtaatcggcgttgatta

SEQ ID NO: 145 - RNA, 19 nt, synthetic construct
  misc_feature 1..19: note = Synthetic construct
  misc_feature 1..19: note = gRNA EBF4_a spacer RNA sequence
  source 1..19: mol_type = other RNA
aagacgccggggcttcgtc

SEQ ID NO: 146 - RNA, 19 nt, synthetic construct
  misc_feature 1..19: note = Synthetic construct
  misc_feature 1..19: note = gRNA ETS1_a spacer RNA sequence
  source 1..19: mol_type = other RNA
agaaacgccctaaagagga

SEQ ID NO: 147 - RNA, 19 nt, synthetic construct
  misc_feature 1..19: note = Synthetic construct
  misc_feature 1..19: note = gRNA FOXD3_a spacer RNA sequence
  source 1..19: mol_type = other RNA
gctgcaagggcggtgttgg

SEQ ID NO: 148 - RNA, 19 nt, synthetic construct
  misc_feature 1..19: note = Synthetic construct
  misc_feature 1..19: note = gRNA HELT_a spacer RNA sequence
  source 1..19: mol_type = other RNA
caagcgcctgcagcagtcg

SEQ ID NO: 149 - RNA, 19 nt, synthetic construct
  misc_feature 1..19: note = Synthetic construct
  misc_feature 1..19: note = gRNA HMGN3_a spacer RNA sequence
  source 1..19: mol_type = other RNA
gcgtaccgcgcgcggaggt

SEQ ID NO: 150 - RNA, 19 nt, synthetic construct
  misc_feature 1..19: note = Synthetic construct
  misc_feature 1..19: note = gRNA PATZ1_a spacer RNA sequence
  source 1..19: mol_type = other RNA
gaaacggcgagctcagagt

SEQ ID NO: 151 - RNA, 19 nt, synthetic construct
  misc_feature 1..19: note = Synthetic construct
  misc_feature 1..19: note = gRNA PRDM8_a spacer RNA sequence
  source 1..19: mol_type = other RNA
cccagctctccatccccgg

SEQ ID NO: 152 - RNA, 19 nt, synthetic construct
  misc_feature 1..19: note = Synthetic construct
  misc_feature 1..19: note = gRNA SOX21_a spacer RNA sequence
  source 1..19: mol_type = other RNA
cgggagaggaagactcgag

SEQ ID NO: 153 - RNA, 19 nt, synthetic construct
  misc_feature 1..19: note = Synthetic construct
  misc_feature 1..19: note = gRNA TBX15_a spacer RNA sequence
  source 1..19: mol_type = other RNA
gatcacagggaggacggga

SEQ ID NO: 154 - RNA, 19 nt, synthetic construct
  misc_feature 1..19: note = Synthetic construct
  misc_feature 1..19: note = gRNA ZBED5_a spacer RNA sequence
  source 1..19: mol_type = other RNA
aaaggaccaagctcggttg

SEQ ID NO: 155 - RNA, 19 nt, synthetic construct
  misc_feature 1..19: note = Synthetic construct
  misc_feature 1..19: note = gRNA ZBTB7A_a spacer RNA sequence
  source 1..19: mol_type = other RNA
tccctccgccgtttgcagc

SEQ ID NO: 156 - RNA, 19 nt, synthetic construct
  misc_feature 1..19: note = Synthetic construct
  misc_feature 1..19: note = gRNA ZNF219_a spacer RNA sequence
  source 1..19: mol_type = other RNA
cctaaacccggacatacca

SEQ ID NO: 157 - RNA, 19 nt, synthetic construct
  misc_feature 1..19: note = Synthetic construct
  misc_feature 1..19: note = gRNA ZNF319_a spacer RNA sequence
  source 1..19: mol_type = other RNA
taccactctaagccttctc

SEQ ID NO: 158 - RNA, 19 nt, synthetic construct
  misc_feature 1..19: note = Synthetic construct
  misc_feature 1..19: note = gRNA ZNF470_a spacer RNA sequence
  source 1..19: mol_type = other RNA
ttcccaggggctattgcag

SEQ ID NO: 159 - RNA, 19 nt, synthetic construct
  misc_feature 1..19: note = Synthetic construct
  misc_feature 1..19: note = gRNA ZNF816_a spacer RNA sequence
  source 1..19: mol_type = other RNA
agcaggaatgcgggcgcgg

SEQ ID NO: 160 - RNA, 19 nt, synthetic construct
  misc_feature 1..19: note = Synthetic construct
  misc_feature 1..19: note = gRNA ZSCAN23_a spacer RNA sequence
  source 1..19: mol_type = other RNA
ctccgcgaaacagcggttc

SEQ ID NO: 161 - RNA, 19 nt, synthetic construct
  misc_feature 1..19: note = Synthetic construct
  misc_feature 1..19: note = gRNA EMX1_a spacer RNA sequence
  source 1..19: mol_type = other RNA
agcgccgggagccgtggtt

SEQ ID NO: 162 - RNA, 19 nt, synthetic construct
  misc_feature 1..19: note = Synthetic construct
  misc_feature 1..19: note = gRNA FOXO3_a spacer RNA sequence
  source 1..19: mol_type = other RNA
gccgcgggaggcactagag

SEQ ID NO: 163 - RNA, 19 nt, synthetic construct
  misc_feature 1..19: note = Synthetic construct
  misc_feature 1..19: note = gRNA HIC2_a spacer RNA sequence
  source 1..19: mol_type = other RNA
tgggctcctggggaagccc

SEQ ID NO: 164 - RNA, 19 nt, synthetic construct
  misc_feature 1..19: note = Synthetic construct
  misc_feature 1..19: note = gRNA SOX30_a spacer RNA sequence
  source 1..19: mol_type = other RNA
gccgcggaaagcagatctg

SEQ ID NO: 165 - RNA, 19 nt, synthetic construct
  misc_feature 1..19: note = Synthetic construct
  misc_feature 1..19: note = gRNA STAT1_a spacer RNA sequence
  source 1..19: mol_type = other RNA
cagcgtggcaacacgggac

SEQ ID NO: 166 - RNA, 19 nt, synthetic construct
  misc_feature 1..19: note = Synthetic construct
  misc_feature 1..19: note = gRNA ZBTB46_a spacer RNA sequence
  source 1..19: mol_type = other RNA
ccgcgccgcggggtcgcag

SEQ ID NO: 167 - RNA, 19 nt, synthetic construct
  misc_feature 1..19: note = Synthetic construct
  misc_feature 1..19: note = gRNA ZNF331_a spacer RNA sequence
  source 1..19: mol_type = other RNA
caggtgagattggcgccca

SEQ ID NO: 168 - RNA, 19 nt, synthetic construct
  misc_feature 1..19: note = Synthetic construct
  misc_feature 1..19: note = gRNA ZNF541_a spacer RNA sequence
  source 1..19: mol_type = other RNA
caagtcatgtgtcagataa

SEQ ID NO: 169 - RNA, 19 nt, synthetic construct
  misc_feature 1..19: note = Synthetic construct
  misc_feature 1..19: note = gRNA ZNF619_a spacer RNA sequence
  source 1..19: mol_type = other RNA
cgcagagcggttgtggaga

SEQ ID NO: 170 - RNA, 19 nt, synthetic construct
  misc_feature 1..19: note = Synthetic construct
  misc_feature 1..19: note = gRNA AEBP1_a spacer RNA sequence
  source 1..19: mol_type = other RNA
cgggaccccgagtccaccg

SEQ ID NO: 171 - RNA, 19 nt, synthetic construct
  misc_feature 1..19: note = Synthetic construct
  misc_feature 1..19: note = gRNA ARID3A_a spacer RNA sequence
  source 1..19: mol_type = other RNA
cccgcccccggcctaggga

SEQ ID NO: 172 - RNA, 19 nt, synthetic construct
  misc_feature 1..19: note = Synthetic construct
  misc_feature 1..19: note = gRNA ARNTL2_a spacer RNA sequence
  source 1..19: mol_type = other RNA
accctcggggcaaaggtaa

SEQ ID NO: 173 - RNA, 19 nt, synthetic construct
  misc_feature 1..19: note = Synthetic construct
  misc_feature 1..19: note = gRNA BACH1_a spacer RNA sequence
  source 1..19: mol_type = other RNA
tcgctgacagaggcttcga

SEQ ID NO: 174 - RNA, 19 nt, synthetic construct
  misc_feature 1..19: note = Synthetic construct
  misc_feature 1..19: note = gRNA BATF2_a spacer RNA sequence
  source 1..19: mol_type = other RNA
caaaggccaggtatgagga

SEQ ID NO: 175 - RNA, 19 nt, synthetic construct
  misc_feature 1..19: note = Synthetic construct
  misc_feature 1..19: note = gRNA BRD4_a spacer RNA sequence
  source 1..19: mol_type = other RNA
tgctgaagtcatgaggtgt

SEQ ID NO: 176 - RNA, 19 nt, synthetic construct
  misc_feature 1..19: note = Synthetic construct
  misc_feature 1..19: note = gRNA CDX4_a spacer RNA sequence
  source 1..19: mol_type = other RNA
gggaagatcggtgaaaggg

SEQ ID NO: 177 - RNA, 19 nt, synthetic construct
  misc_feature 1..19: note = Synthetic construct
  misc_feature 1..19: note = gRNA CENPB_a spacer RNA sequence
  source 1..19: mol_type = other RNA
gcccccccgcggtatgtcc

SEQ ID NO: 178 - RNA, 19 nt, synthetic construct
  misc_feature 1..19: note = Synthetic construct
  misc_feature 1..19: note = gRNA DMRT1_a spacer RNA sequence
  source 1..19: mol_type = other RNA
agagcggtggctggagtcg

SEQ ID NO: 179 - RNA, 19 nt, synthetic construct
  misc_feature 1..19: note = Synthetic construct
  misc_feature 1..19: note = gRNA E2F2_a spacer RNA sequence
  source 1..19: mol_type = other RNA
cggaaagaacgcgtcctcg

SEQ ID NO: 180 - RNA, 19 nt, synthetic construct
  misc_feature 1..19: note = Synthetic construct
  misc_feature 1..19: note = gRNA ESRRB_a spacer RNA sequence
  source 1..19: mol_type = other RNA
gaggggcaacccccgactc

SEQ ID NO: 181 - RNA, 19 nt, synthetic construct
  misc_feature 1..19: note = Synthetic construct
  misc_feature 1..19: note = gRNA FERD3L_a spacer RNA sequence
  source 1..19: mol_type = other RNA
tgagtaggaggtaaaggta

SEQ ID NO: 182 - RNA, 19 nt, synthetic construct
  misc_feature 1..19: note = Synthetic construct
  misc_feature 1..19: note = gRNA FEV_a spacer RNA sequence
  source 1..19: mol_type = other RNA
cgggagccgactggaagaa

SEQ ID NO: 183 - RNA, 19 nt, synthetic construct
  misc_feature 1..19: note = Synthetic construct
  misc_feature 1..19: note = gRNA FLYWCH1_a spacer RNA sequence
  source 1..19: mol_type = other RNA
gggacgtaactcacggaaa

SEQ ID NO: 184 - RNA, 19 nt, synthetic construct
  misc_feature 1..19: note = Synthetic construct
  misc_feature 1..19: note = gRNA FOXJ3_a spacer RNA sequence
  source 1..19: mol_type = other RNA
cagagcctcgggagtctca

SEQ ID NO: 185 - RNA, 19 nt, synthetic construct
  misc_feature 1..19: note = Synthetic construct
  misc_feature 1..19: note = gRNA GLIS3_a spacer RNA sequence
  source 1..19: mol_type = other RNA
ggctcgcgggtcactaggc

SEQ ID NO: 186 - RNA, 19 nt, synthetic construct
  misc_feature 1..19: note = Synthetic construct
  misc_feature 1..19: note = gRNA HDAC9_a spacer RNA sequence
  source 1..19: mol_type = other RNA
gtaaagcaagcttcgagag

SEQ ID NO: 187 - RNA, 19 nt, synthetic construct
  misc_feature 1..19: note = Synthetic construct
  misc_feature 1..19: note = gRNA HIVEP1_2 spacer RNA sequence
  source 1..19: mol_type = other RNA
cagtcacgcggccccggga

SEQ ID NO: 188 - RNA, 19 nt, synthetic construct
  misc_feature 1..19: note = Synthetic construct
  misc_feature 1..19: note = gRNA HOXA2_4 spacer RNA sequence
  source 1..19: mol_type = other RNA
ggaaggaacgcctcattgg

SEQ ID NO: 189 - RNA, 19 nt, synthetic construct
  misc_feature 1..19: note = Synthetic construct
  misc_feature 1..19: note = gRNA HOXC9_1 spacer RNA sequence
  source 1..19: mol_type = other RNA
gagctaggagccgtcctga

SEQ ID NO: 190 - RNA, 19 nt, synthetic construct
  misc_feature 1..19: note = Synthetic construct
  misc_feature 1..19: note = gRNA HSFY1_5 spacer RNA sequence
  source 1..19: mol_type = other RNA
ggtaggttacgggttgtca

SEQ ID NO: 191 - RNA, 19 nt, synthetic construct
  misc_feature 1..19: note = Synthetic construct
  misc_feature 1..19: note = gRNA ISL2_2 spacer RNA sequence
  source 1..19: mol_type = other RNA
aacggccgcggagccagct

SEQ ID NO: 192 - RNA, 19 nt, synthetic construct
  misc_feature 1..19: note = Synthetic construct
  misc_feature 1..19: note = gRNA KAT2A_5 spacer RNA sequence
  source 1..19: mol_type = other RNA
tccggagccgcgtggggca

SEQ ID NO: 193 - RNA, 19 nt, synthetic construct
  misc_feature 1..19: note = Synthetic construct
  misc_feature 1..19: note = gRNA KDM5D_5 spacer RNA sequence
  source 1..19: mol_type = other RNA
gcaagagcgattttaccag

SEQ ID NO: 194 - RNA, 19 nt, synthetic construct
  misc_feature 1..19: note = Synthetic construct
  misc_feature 1..19: note = gRNA KLF13_a spacer RNA sequence
  source 1..19: mol_type = other RNA
accgggcacgcccctctaa

SEQ ID NO: 195 - RNA, 19 nt, synthetic construct
  misc_feature 1..19: note = Synthetic construct
  misc_feature 1..19: note = gRNA KLF6_a spacer RNA sequence
  source 1..19: mol_type = other RNA
tgaccgagactctccatca

SEQ ID NO: 196 - RNA, 19 nt, synthetic construct
  misc_feature 1..19: note = Synthetic construct
  misc_feature 1..19: note = gRNA MEOX2_a spacer RNA sequence
  source 1..19: mol_type = other RNA
aatcagggtgctaggttgg

SEQ ID NO: 197 - RNA, 19 nt, synthetic construct
  misc_feature 1..19: note = Synthetic construct
  misc_feature 1..19: note = gRNA MLXIPL_a spacer RNA sequence
  source 1..19: mol_type = other RNA
cgccagagctccagagcac

SEQ ID NO: 198 - RNA, 19 nt, synthetic construct
  misc_feature 1..19: note = Synthetic construct
  misc_feature 1..19: note = gRNA MNT_a spacer RNA sequence
  source 1..19: mol_type = other RNA
gggccgcggcgtctcgcga

SEQ ID NO: 199 - RNA, 19 nt, synthetic construct
  misc_feature 1..19: note = Synthetic construct
  misc_feature 1..19: note = gRNA MYCN_a spacer RNA sequence
  source 1..19: mol_type = other RNA
caagcccgggggtccaaaa

SEQ ID NO: 200 - RNA, 19 nt, synthetic construct
  misc_feature 1..19: note = Synthetic construct
  misc_feature 1..19: note = gRNA MYT1L_a spacer RNA sequence
  source 1..19: mol_type = other RNA
gcctcatagataatgatga

SEQ ID NO: 201 - RNA, 19 nt, synthetic construct
  misc_feature 1..19: note = Synthetic construct
  misc_feature 1..19: note = gRNA NEUROG3_a spacer RNA sequence
  source 1..19: mol_type = other RNA
ctcgcgtggcgcggcccca

SEQ ID NO: 202 - RNA, 19 nt, synthetic construct
  misc_feature 1..19: note = Synthetic construct
  misc_feature 1..19: note = gRNA NFE2_a spacer RNA sequence
  source 1..19: mol_type = other RNA
aggagagccacggggaaag

SEQ ID NO: 203 - RNA, 19 nt, synthetic construct
  misc_feature 1..19: note = Synthetic construct
  misc_feature 1..19: note = gRNA NR2F1_a spacer RNA sequence
  source 1..19: mol_type = other RNA
tttctctccccccgattca

SEQ ID NO: 204 - RNA, 19 nt, synthetic construct
  misc_feature 1..19: note = Synthetic construct
  misc_feature 1..19: note = gRNA NR2F2_a spacer RNA sequence
  source 1..19: mol_type = other RNA
ccccctcgccgtcattggc

SEQ ID NO: 205 - RNA, 19 nt, synthetic construct
  misc_feature 1..19: note = Synthetic construct
  misc_feature 1..19: note = gRNA NR3C1_a spacer RNA sequence
  source 1..19: mol_type = other RNA
gggcccgcgcggagggcgt

SEQ ID NO: 206 - RNA, 19 nt, synthetic construct
  misc_feature 1..19: note = Synthetic construct
  misc_feature 1..19: note = gRNA PHF20_a spacer RNA sequence
  source 1..19: mol_type = other RNA
aggccgttgccacagtgat

SEQ ID NO: 207 - RNA, 19 nt, synthetic construct
  misc_feature 1..19: note = Synthetic construct
  misc_feature 1..19: note = gRNA PITX2_a spacer RNA sequence
  source 1..19: mol_type = other RNA
caggacagtccctccaccg

SEQ ID NO: 208 - RNA, 19 nt, synthetic construct
  misc_feature 1..19: note = Synthetic construct
  misc_feature 1..19: note = gRNA POU2AF1_a spacer RNA sequence
  source 1..19: mol_type = other RNA
gccgctggggcgcgcacag

SEQ ID NO: 209 - RNA, 19 nt, synthetic construct
  misc_feature 1..19: note = Synthetic construct
  misc_feature 1..19: note = gRNA POU3F2_a spacer RNA sequence
  source 1..19: mol_type = other RNA
gcggggcccatccgagaga

SEQ ID NO: 210 - RNA, 19 nt, synthetic construct
  misc_feature 1..19: note = Synthetic construct
  misc_feature 1..19: note = gRNA POU3F3_a spacer RNA sequence
  source 1..19: mol_type = other RNA
ggccacgaccccccctgaa

SEQ ID NO: 211 - RNA, 19 nt, synthetic construct
  misc_feature 1..19: note = Synthetic construct
  misc_feature 1..19: note = gRNA PRDM11_a spacer RNA sequence
  source 1..19: mol_type = other RNA
tgggtgtggaggggttcca

SEQ ID NO: 212 - RNA, 19 nt, synthetic construct
  misc_feature 1..19: note = Synthetic construct
  misc_feature 1..19: note = gRNA PRMT3_a spacer RNA sequence
  source 1..19: mol_type = other RNA
tgaagaggcgcgctgatga

SEQ ID NO: 213 - RNA, 19 nt, synthetic construct
  misc_feature 1..19: note = Synthetic construct
  misc_feature 1..19: note = gRNA PROP1_a spacer RNA sequence
  source 1..19: mol_type = other RNA
tcggagggtccagggactg

SEQ ID NO: 214 - RNA, 19 nt, synthetic construct
  misc_feature 1..19: note = Synthetic construct
  misc_feature 1..19: note = gRNA PURA_a spacer RNA sequence
  source 1..19: mol_type = other RNA
ctcacgcgcgagaaggggg

SEQ ID NO: 215 - RNA, 19 nt, synthetic construct
  misc_feature 1..19: note = Synthetic construct
  misc_feature 1..19: note = gRNA RELB_a spacer RNA sequence
  source 1..19: mol_type = other RNA
tgggcgctcaggaggggtt

SEQ ID NO: 216 - RNA, 19 nt, synthetic construct
  misc_feature 1..19: note = Synthetic construct
  misc_feature 1..19: note = gRNA RFX3_a spacer RNA sequence
  source 1..19: mol_type = other RNA
actgacggcccccaaaccc

SEQ ID NO: 217 - RNA, 19 nt, synthetic construct
  misc_feature 1..19: note = Synthetic construct
  misc_feature 1..19: note = gRNA SAFB_a spacer RNA sequence
  source 1..19: mol_type = other RNA
ctgctaagtacgctagtgg

SEQ ID NO: 218 - RNA, 19 nt, synthetic construct
  misc_feature 1..19: note = Synthetic construct
  misc_feature 1..19: note = gRNA SETBP1_a spacer RNA sequence
  source 1..19: mol_type = other RNA
tatggagagagatatcaag

SEQ ID NO: 219 - RNA, 19 nt, synthetic construct
  misc_feature 1..19: note = Synthetic construct
  misc_feature 1..19: note = gRNA SMAD2_a spacer RNA sequence
  source 1..19: mol_type = other RNA
aagccgcgcggggcgggta

SEQ ID NO: 220 - RNA, 19 nt, synthetic construct
  misc_feature 1..19: note = Synthetic construct
  misc_feature 1..19: note = gRNA SMAD6_a spacer RNA sequence
  source 1..19: mol_type = other RNA
cccggggcgggccgagctc

SEQ ID NO: 221 - RNA, 19 nt, synthetic construct
  misc_feature 1..19: note = Synthetic construct
  misc_feature 1..19: note = gRNA SNAPC2_a spacer RNA sequence
  source 1..19: mol_type = other RNA
agggagggcatgggatcca

SEQ ID NO: 222 - RNA, 19 nt, synthetic construct
  misc_feature 1..19: note = Synthetic construct
  misc_feature 1..19: note = gRNA SNAPC4_a spacer RNA sequence
  source 1..19: mol_type = other RNA
tctcagagcccaccctcga

SEQ ID NO: 223 - RNA, 19 nt, synthetic construct
  misc_feature 1..19: note = Synthetic construct
  misc_feature 1..19: note = gRNA SON_a spacer RNA sequence
  source 1..19: mol_type = other RNA
ctgtacttggcctgtacta

SEQ ID NO: 224 - RNA, 19 nt, synthetic construct
  misc_feature 1..19: note = Synthetic construct
  misc_feature 1..19: note = gRNA SOX11_a spacer RNA sequence
  source 1..19: mol_type = other RNA
gggctgctttgcaaagatg

SEQ ID NO: 225 - RNA, 19 nt, synthetic construct
  misc_feature 1..19: note = Synthetic construct
  misc_feature 1..19: note = gRNA SOX12_a spacer RNA sequence
  source 1..19: mol_type = other RNA
ggcccggccgcagcccaag

SEQ ID NO: 226 - RNA, 19 nt, synthetic construct
  misc_feature 1..19: note = Synthetic construct
  misc_feature 1..19: note = gRNA SOX14_a spacer RNA sequence
  source 1..19: mol_type = other RNA
caaaagcggcgaagttcaa

SEQ ID NO: 227 - RNA, 19 nt, synthetic construct
  misc_feature 1..19: note = Synthetic construct
  misc_feature 1..19: note = gRNA SPIC_a spacer RNA sequence
  source 1..19: mol_type = other RNA
tgcaacctaagttgggcgg

SEQ ID NO: 228 - RNA, 19 nt, synthetic construct
  misc_feature 1..19: note = Synthetic construct
  misc_feature 1..19: note = gRNA THAP7_a spacer RNA sequence
  source 1..19: mol_type = other RNA
aatcggctgggacggggtt

SEQ ID NO: 229 - RNA, 19 nt, synthetic construct
  misc_feature 1..19: note = Synthetic construct
  misc_feature 1..19: note = gRNA TOX2_a spacer RNA sequence
  source 1..19: mol_type = other RNA
gctcccgcgggcagtgcct

SEQ ID NO: 230 - RNA, 19 nt, synthetic construct
  misc_feature 1..19: note = Synthetic construct
  misc_feature 1..19: note = gRNA TRERF1_a spacer RNA sequence
  source 1..19: mol_type = other RNA
gggtaccggggagcggata

SEQ ID NO: 231 - RNA, 19 nt, synthetic construct
  misc_feature 1..19: note = Synthetic construct
  misc_feature 1..19: note = gRNA TRIM3_a spacer RNA sequence
  source 1..19: mol_type = other RNA
gggcgtgcgggtgcggtag

SEQ ID NO: 232 - RNA, 19 nt, synthetic construct
  misc_feature 1..19: note = Synthetic construct
  misc_feature 1..19: note = gRNA VDR_a spacer RNA sequence
  source 1..19: mol_type = other RNA
tacagcgtggcctataggg

SEQ ID NO: 233 - RNA, 19 nt, synthetic construct
  misc_feature 1..19: note = Synthetic construct
  misc_feature 1..19: note = gRNA YBX2_a spacer RNA sequence
  source 1..19: mol_type = other RNA
gggcccaggtgtacttact

SEQ ID NO: 234 - RNA, 19 nt, synthetic construct
  misc_feature 1..19: note = Synthetic construct
  misc_feature 1..19: note = gRNA ZBTB14_a spacer RNA sequence
  source 1..19: mol_type = other RNA
cgcgcgcgtgcgcaggcca

SEQ ID NO: 235 - RNA, 19 nt, synthetic construct
  misc_feature 1..19: note = Synthetic construct
  misc_feature 1..19: note = gRNA ZBTB4_a spacer RNA sequence
  source 1..19: mol_type = other RNA
ccgggacggttggagaaga

SEQ ID NO: 236 - RNA, 19 nt, synthetic construct
  misc_feature 1..19: note = Synthetic construct
  misc_feature 1..19: note = gRNA ZFHX2_a spacer RNA sequence
  source 1..19: mol_type = other RNA
ggtacaggtctcatgcctg

SEQ ID NO: 237 - RNA, 19 nt, synthetic construct
  misc_feature 1..19: note = Synthetic construct
  misc_feature 1..19: note = gRNA ZFP57_a spacer RNA sequence
  source 1..19: mol_type = other RNA
gcagctatcagttggtcgg

SEQ ID NO: 238 - RNA, 19 nt, synthetic construct
  misc_feature 1..19: note = Synthetic construct
  misc_feature 1..19: note = gRNA ZFP62_a spacer RNA sequence
  source 1..19: mol_type = other RNA
gagggcagagagacctcta

SEQ ID NO: 239 - RNA, 19 nt, synthetic construct
  misc_feature 1..19: note = Synthetic construct
  misc_feature 1..19: note = gRNA ZFP69B_a spacer RNA sequence
  source 1..19: mol_type = other RNA
tgatgatgctcgcagggat

SEQ ID NO: 240 - RNA, 19 nt, synthetic construct
  misc_feature 1..19: note = Synthetic construct
  misc_feature 1..19: note = gRNA ZIC1_a spacer RNA sequence
  source 1..19: mol_type = other RNA
gggaatgcgggcgcaccaa

SEQ ID NO: 241 - RNA, 19 nt, synthetic construct
  misc_feature 1..19: note = Synthetic construct
  misc_feature 1..19: note = gRNA ZIC4_a spacer RNA sequence
  source 1..19: mol_type = other RNA
caacggggacgttgacaag

SEQ ID NO: 242 - RNA, 19 nt, synthetic construct
  misc_feature 1..19: note = Synthetic construct
  misc_feature 1..19: note = gRNA ZNF136_a spacer RNA sequence
  source 1..19: mol_type = other RNA
ccccgacggcgcgcctgat

SEQ ID NO: 243 - RNA, 19 nt, synthetic construct
  misc_feature 1..19: note = Synthetic construct
  misc_feature 1..19: note = gRNA ZNF16_a spacer RNA sequence
  source 1..19: mol_type = other RNA
gctagtgggaccaggtctg

SEQ ID NO: 244 - RNA, 19 nt, synthetic construct
  misc_feature 1..19: note = Synthetic construct
  misc_feature 1..19: note = gRNA ZNF174_a spacer RNA sequence
  source 1..19: mol_type = other RNA
caggggcgtgcgtaatggg

SEQ ID NO: 245 - RNA, 19 nt, synthetic construct
  misc_feature 1..19: note = Synthetic construct
  misc_feature 1..19: note = gRNA ZNF253_a spacer RNA sequence
  source 1..19: mol_type = other RNA
tgtcactcagcggctgggg

SEQ ID NO: 246 - RNA, 19 nt, synthetic construct
  misc_feature 1..19: note = Synthetic construct
  misc_feature 1..19: note = gRNA ZNF26_a spacer RNA sequence
  source 1..19: mol_type = other RNA
acagcctgacgggaaagga

SEQ ID NO: 247 - RNA, 19 nt, synthetic construct
  misc_feature 1..19: note = Synthetic construct
  misc_feature 1..19: note = gRNA ZNF263_a spacer RNA sequence
  source 1..19: mol_type = other RNA
gggcgcgccctgaaattct

SEQ ID NO: 248 - RNA, 19 nt, synthetic construct
  misc_feature 1..19: note = Synthetic construct
  misc_feature 1..19: note = gRNA ZNF285_a spacer RNA sequence
  source 1..19: mol_type = other RNA
aaagccccgcgcgcagcag

SEQ ID NO: 249 - RNA, 19 nt, synthetic construct
  misc_feature 1..19: note = Synthetic construct
  misc_feature 1..19: note = gRNA ZNF317_a spacer RNA sequence
  source 1..19: mol_type = other RNA
tcccgcgagaattggaagg

SEQ ID NO: 250 - RNA, 19 nt, synthetic construct
  misc_feature 1..19: note = Synthetic construct
  misc_feature 1..19: note = gRNA ZNF319_b spacer RNA sequence
  source 1..19: mol_type = other RNA
catcttccggaagggaggg

SEQ ID NO: 251 - RNA, 19 nt, synthetic construct
  misc_feature 1..19: note = Synthetic construct
  misc_feature 1..19: note = gRNA ZNF345_a spacer RNA sequence
  source 1..19: mol_type = other RNA
cgcgccaggggaatcggga

SEQ ID NO: 252 - RNA, 19 nt, synthetic construct
  misc_feature 1..19: note = Synthetic construct
  misc_feature 1..19: note = gRNA ZNF420_a spacer RNA sequence
  source 1..19: mol_type = other RNA
gagcccacgagcccctcga

SEQ ID NO: 253 - RNA, 19 nt, synthetic construct
  misc_feature 1..19: note = Synthetic construct
  misc_feature 1..19: note = gRNA ZNF423_a spacer RNA sequence
  source 1..19: mol_type = other RNA
caggggatcagccatccag

SEQ ID NO: 254 - RNA, 19 nt, synthetic construct
  misc_feature 1..19: note = Synthetic construct
  misc_feature 1..19: note = gRNA ZNF470_b spacer RNA sequence
  source 1..19: mol_type = other RNA
gaccaggagaggcccaaaa

SEQ ID NO: 255 - RNA, 19 nt, synthetic construct
  misc_feature 1..19: note = Synthetic construct
  misc_feature 1..19: note = gRNA ZNF48_a spacer RNA sequence
  source 1..19: mol_type = other RNA
gaggccggcggcccgaact

SEQ ID NO: 256 - RNA, 19 nt, synthetic construct
  misc_feature 1..19: note = Synthetic construct
  misc_feature 1..19: note = gRNA ZNF496_a spacer RNA sequence
  source 1..19: mol_type = other RNA
ggagcaggaagtgtcccag

SEQ ID NO: 257 - RNA, 19 nt, synthetic construct
  misc_feature 1..19: note = Synthetic construct
  misc_feature 1..19: note = gRNA ZNF501_a spacer RNA sequence
  source 1..19: mol_type = other RNA
aaacgggatggaagtcaga

SEQ ID NO: 258 - RNA, 19 nt, synthetic construct
  misc_feature 1..19: note = Synthetic construct
  misc_feature 1..19: note = gRNA ZNF524_a spacer RNA sequence
  source 1..19: mol_type = other RNA
gggtcctaacgaccggtcg

SEQ ID NO: 259 - RNA, 19 nt, synthetic construct
  misc_feature 1..19: note = Synthetic construct
  misc_feature 1..19: note = gRNA ZNF557_a spacer RNA sequence
  source 1..19: mol_type = other RNA
ctggcgcgggtctgtggca

SEQ ID NO: 260 - RNA, 19 nt, synthetic construct
  misc_feature 1..19: note = Synthetic construct
  misc_feature 1..19: note = gRNA ZNF735_a spacer RNA sequence
  source 1..19: mol_type = other RNA
aaaaggcccggtaaaatta

SEQ ID NO: 261 - RNA, 19 nt, synthetic construct
  misc_feature 1..19: note = Synthetic construct
  misc_feature 1..19: note = gRNA ZNF747_a spacer RNA sequence
  source 1..19: mol_type = other RNA
ggaacaaagcagcgccctg

SEQ ID NO: 262 - RNA, 19 nt, synthetic construct
  misc_feature 1..19: note = Synthetic construct
  misc_feature 1..19: note = gRNA ZNF783_a spacer RNA sequence
  source 1..19: mol_type = other RNA
aggcgctgcaacagagtct

SEQ ID NO: 263 - RNA, 19 nt, synthetic construct
  misc_feature 1..19: note = Synthetic construct
  misc_feature 1..19: note = gRNA ZNF805_a spacer RNA sequence
  source 1..19: mol_type = other RNA
ctggggccaatgatggtcc

SEQ ID NO: 264 - RNA, 19 nt, synthetic construct
  misc_feature 1..19: note = Synthetic construct
  misc_feature 1..19: note = gRNA ZSCAN10_a spacer RNA sequence
  source 1..19: mol_type = other RNA
ttacactctcacacaggct

SEQ ID NO: 265 - RNA, 105 nt, synthetic construct
  misc_feature 1..105: note = Synthetic construct
  misc_feature 1..105: note = gRNA DDIT3_a
  source 1..105: mol_type = other RNA
ccctagcgagagggagcgagtttaagagctatgctggaaacagcatagcaagtttaaataaggctagtccgttatcaacttgaaaaagtggcaccgagtcggtgc

SEQ ID NO: 266 - RNA, 105 nt, synthetic construct
  misc_feature 1..105: note = Synthetic construct
  misc_feature 1..105: note = gRNA HES2_a
  source 1..105: mol_type = other RNA
ccgtcggggatctgcgggcgtttaagagctatgctggaaacagcatagcaagtttaaataaggctagtccgttatcaacttgaaaaagtggcaccgagtcggtgc

SEQ ID NO: 267 - RNA, 105 nt, synthetic construct
  misc_feature 1..105: note = Synthetic construct
  misc_feature 1..105: note = gRNA HKR1_a
  source 1..105: mol_type = other RNA
cagtcccgcgatgccttcggtttaagagctatgctggaaacagcatagcaagtttaaataaggctagtccgttatcaacttgaaaaagtggcaccgagtcggtgc

SEQ ID NO: 268 - RNA, 105 nt, synthetic construct
  misc_feature 1..105: note = Synthetic construct
  misc_feature 1..105: note = gRNA PLAG1_a
  source 1..105: mol_type = other RNA
aacacttcggcttgacggggtttaagagctatgctggaaacagcatagcaagtttaaataaggctagtccgttatcaacttgaaaaagtggcaccgagtcggtgc

SEQ ID NO: 269 - RNA, 105 nt, synthetic construct
  misc_feature 1..105: note = Synthetic construct
  misc_feature 1..105: note = gRNA PRDM4_a
  source 1..105: mol_type = other RNA
tggccgcgagaggctcggcgtttaagagctatgctggaaacagcatagcaagtttaaataaggctagtccgttatcaacttgaaaaagtggcaccgagtcggtgc

SEQ ID NO: 270 - RNA, 105 nt, synthetic construct
  misc_feature 1..105: note = Synthetic construct
  misc_feature 1..105: note = gRNA TEF_a
  source 1..105: mol_type = other RNA
aggctgggagccataactggtttaagagctatgctggaaacagcatagcaagtttaaataaggctagtccgttatcaacttgaaaaagtggcaccgagtcggtgc

SEQ ID NO: 271 - RNA, 105 nt, synthetic construct
  misc_feature 1..105: note = Synthetic construct
  misc_feature 1..105: note = gRNA TSHZ1_a
  source 1..105: mol_type = other RNA
gacctatttgtaattgtcagtttaagagctatgctggaaacagcatagcaagtttaaataaggctagtccgttatcaacttgaaaaagtggcaccgagtcggtgc

SEQ ID NO: 272 - RNA, 105 nt, synthetic construct
  misc_feature 1..105: note = Synthetic construct
  misc_feature 1..105: note = gRNA ZNF141_a
  source 1..105: mol_type = other RNA
gagagctgtgcacccaactgtttaagagctatgctggaaacagcatagcaagtttaaataaggctagtccgttatcaacttgaaaaagtggcaccgagtcggtgc

SEQ ID NO: 273 - RNA, 105 nt, synthetic construct
  misc_feature 1..105: note = Synthetic construct
  misc_feature 1..105: note = gRNA ZNF562_a
  source 1..105: mol_type = other RNA
tggacggaaccgtgggcgagtttaagagctatgctggaaacagcatagcaagtttaaataaggctagtccgttatcaacttgaaaaagtggcaccgagtcggtgc

SEQ ID NO: 274 - RNA, 105 nt, synthetic construct
  misc_feature 1..105: note = Synthetic construct
  misc_feature 1..105: note = gRNA ZNF691_a
  source 1..105: mol_type = other RNA
cgccctactactccaggatgtttaagagctatgctggaaacagcatagcaagtttaaataaggctagtccgttatcaacttgaaaaagtggcaccgagtcggtgc

SEQ ID NO: 275 - RNA, 105 nt, synthetic construct
  misc_feature 1..105: note = Synthetic construct
  misc_feature 1..105: note = gRNA ZNF692_a
  source 1..105: mol_type = other RNA
ccccgcggcagttgtggttgtttaagagctatgctggaaacagcatagcaagtttaaataaggctagtccgttatcaacttgaaaaagtggcaccgagtcggtgc

SEQ ID NO: 276 - RNA, 105 nt, synthetic construct
  misc_feature 1..105: note = Synthetic construct
  misc_feature 1..105: note = gRNA ZSCAN5A_a
  source 1..105: mol_type = other RNA
acgtaatcggcgttgattagtttaagagctatgctggaaacagcatagcaagtttaaataaggctagtccgttatcaacttgaaaaagtggcaccgagtcggtgc

SEQ ID NO: 277 - RNA, 105 nt, synthetic construct
  misc_feature 1..105: note = Synthetic construct
  misc_feature 1..105: note = gRNA EBF4_a
  source 1..105: mol_type = other RNA
aagacgccggggcttcgtcgtttaagagctatgctggaaacagcatagcaagtttaaataaggctagtccgttatcaacttgaaaaagtggcaccgagtcggtgc

SEQ ID NO: 278 - RNA, 105 nt, synthetic construct
  misc_feature 1..105: note = Synthetic construct
  misc_feature 1..105: note = gRNA ETS1_a
  source 1..105: mol_type = other RNA
agaaacgccctaaagaggagtttaagagctatgctggaaacagcatagcaagtttaaataaggctagtccgttatcaacttgaaaaagtggcaccgagtcggtgc

SEQ ID NO: 279 - RNA, 105 nt, synthetic construct
  misc_feature 1..105: note = Synthetic construct
  misc_feature 1..105: note = gRNA FOXD3_a
  source 1..105: mol_type = other RNA
gctgcaagggcggtgttgggtttaagagctatgctggaaacagcatagcaagtttaaataaggctagtccgttatcaacttgaaaaagtggcaccgagtcggtgc

SEQ ID NO: 280 - RNA, 105 nt, synthetic construct
  misc_feature 1..105: note = Synthetic construct
  misc_feature 1..105: note = gRNA HELT_a
  source 1..105: mol_type = other RNA
caagcgcctgcagcagtcggtttaagagctatgctggaaacagcatagcaagtttaaataaggctagtccgttatcaacttgaaaaagtggcaccgagtcggtgc

SEQ ID NO: 281 - RNA, 105 nt, synthetic construct
  misc_feature 1..105: note = Synthetic construct
  misc_feature 1..105: note = gRNA HMGN3_a
  source 1..105: mol_type = other RNA
gcgtaccgcgcgcggaggtgtttaagagctatgctggaaacagcatagcaagtttaaataaggctagtccgttatcaacttgaaaaagtggcaccgagtcggtgc

SEQ ID NO: 282 - RNA, 105 nt, synthetic construct
  misc_feature 1..105: note = Synthetic construct
  misc_feature 1..105: note = gRNA PATZ1_a
  source 1..105: mol_type = other RNA
gaaacggcgagctcagagtgtttaagagctatgctggaaacagcatagcaagtttaaataaggctagtccgttatcaacttgaaaaagtggcaccgagtcggtgc

SEQ ID NO: 283 - RNA, 105 nt, synthetic construct
  misc_feature 1..105: note = Synthetic construct
  misc_feature 1..105: note = gRNA PRDM8_a
  source 1..105: mol_type = other RNA
cccagctctccatccccgggtttaagagctatgctggaaacagcatagcaagtttaaataaggctagtccgttatcaacttgaaaaagtggcaccgagtcggtgc

SEQ ID NO: 284 - RNA, 105 nt, synthetic construct
  misc_feature 1..105: note = Synthetic construct
  misc_feature 1..105: note = gRNA SOX21_a
  source 1..105: mol_type = other RNA
cgggagaggaagactcgaggtttaagagctatgctggaaacagcatagcaagtttaaataaggctagtccgttatcaacttgaaaaagtggcaccgagtcggtgc

SEQ ID NO: 285 - RNA, 105 nt, synthetic construct
  misc_feature 1..105: note = Synthetic construct
  misc_feature 1..105: note = gRNA TBX15_a
  source 1..105: mol_type = other RNA
gatcacagggaggacgggagtttaagagctatgctggaaacagcatagcaagtttaaataaggctagtccgttatcaacttgaaaaagtggcaccgagtcggtgc

SEQ ID NO: 286 - RNA, 105 nt, synthetic construct
  misc_feature 1..105: note = Synthetic construct
  misc_feature 1..105: note = gRNA ZBED5_a
  source 1..105: mol_type = other RNA
aaaggaccaagctcggttggtttaagagctatgctggaaacagcatagcaagtttaaataaggctagtccgttatcaacttgaaaaagtggcaccgagtcggtgc

SEQ ID NO: 287 - RNA, 105 nt, synthetic construct
  misc_feature 1..105: note = Synthetic construct
  misc_feature 1..105: note = gRNA ZBTB7A_a
  source 1..105: mol_type = other RNA
tccctccgccgtttgcagcgtttaagagctatgctggaaacagcatagcaagtttaaataaggctagtccgttatcaacttgaaaaagtggcaccgagtcggtgc

SEQ ID NO: 288 - RNA, 105 nt, synthetic construct
  misc_feature 1..105: note = Synthetic construct
  misc_feature 1..105: note = gRNA ZNF219_a
  source 1..105: mol_type = other RNA
cctaaacccggacataccagtttaagagctatgctggaaacagcatagcaagtttaaataaggctagtccgttatcaacttgaaaaagtggcaccgagtcggtgc

SEQ ID NO: 289 - RNA, 105 nt, synthetic construct
  misc_feature 1..105: note = Synthetic construct
  misc_feature 1..105: note = gRNA ZNF319_a
  source 1..105: mol_type = other RNA
taccactctaagccttctcgtttaagagctatgctggaaacagcatagcaagtttaaataaggctagtccgttatcaacttgaaaaagtggcaccgagtcggtgc

SEQ ID NO: 290 - RNA, 105 nt, synthetic construct
  misc_feature 1..105: note = Synthetic construct
  misc_feature 1..105: note = gRNA ZNF470_a
  source 1..105: mol_type = other RNA
ttcccaggggctattgcaggtttaagagctatgctggaaacagcatagcaagtttaaataaggctagtccgttatcaacttgaaaaagtggcaccgagtcggtgc

SEQ ID NO: 291 - RNA, 105 nt, synthetic construct
  misc_feature 1..105: note = Synthetic construct
  misc_feature 1..105: note = gRNA ZNF816_a
  source 1..105: mol_type = other RNA
agcaggaatgcgggcgcgggtttaagagctatgctggaaacagcatagcaagtttaaataaggctagtccgttatcaacttgaaaaagtggcaccgagtcggtgc

SEQ ID NO: 292 - RNA, 105 nt, synthetic construct
  misc_feature 1..105: note = Synthetic construct
  misc_feature 1..105: note = gRNA ZSCAN23_a
  source 1..105: mol_type = other RNA
ctccgcgaaacagcggttcgtttaagagctatgctggaaacagcatagcaagtttaaataaggctagtccgttatcaacttgaaaaagtggcaccgagtcggtgc

SEQ ID NO: 293 - RNA, 105 nt, synthetic construct
  misc_feature 1..105: note = Synthetic construct
  misc_feature 1..105: note = gRNA EMX1_a
  source 1..105: mol_type = other RNA
agcgccgggagccgtggttgtttaagagctatgctggaaacagcatagcaagtttaaataaggctagtccgttatcaacttgaaaaagtggcaccgagtcggtgc

SEQ ID NO: 294 - RNA, 105 nt, synthetic construct
  misc_feature 1..105: note = Synthetic construct
  misc_feature 1..105: note = gRNA FOXO3_a
  source 1..105: mol_type = other RNA
gccgcgggaggcactagaggtttaagagctatgctggaaacagcatagcaagtttaaataaggctagtccgttatcaacttgaaaaagtggcaccgagtcggtgc

SEQ ID NO: 295 - RNA, 105 nt, synthetic construct
  misc_feature 1..105: note = Synthetic construct
  misc_feature 1..105: note = gRNA HIC2_a
  source 1..105: mol_type = other RNA
tgggctcctggggaagcccgtttaagagctatgctggaaacagcatagcaagtttaaataaggctagtccgttatcaacttgaaaaagtggcaccgagtcggtgc

SEQ ID NO: 296 - RNA, 105 nt, synthetic construct
  misc_feature 1..105: note = Synthetic construct
  misc_feature 1..105: note = gRNA SOX30_a
  source 1..105: mol_type = other RNA
gccgcggaaagcagatctggtttaagagctatgctggaaacagcatagcaagtttaaataaggctagtccgttatcaacttgaaaaagtggcaccgagtcggtgc

SEQ ID NO: 297 - RNA, 105 nt, synthetic construct
  misc_feature 1..105: note = Synthetic construct
  misc_feature 1..105: note = gRNA STAT1_a
  source 1..105: mol_type = other RNA
cagcgtggcaacacgggacgtttaagagctatgctggaaacagcatagcaagtttaaataaggctagtccgttatcaacttgaaaaagtggcaccgagtcggtgc

SEQ ID NO: 298 - RNA, 105 nt, synthetic construct
  misc_feature 1..105: note = Synthetic construct
  misc_feature 1..105: note = gRNA ZBTB46_a
  source 1..105: mol_type = other RNA
ccgcgccgcggggtcgcaggtttaagagctatgctggaaacagcatagcaagtttaaataaggctagtccgttatcaacttgaaaaagtggcaccgagtcggtgc

SEQ ID NO: 299 - RNA, 105 nt, synthetic construct
  misc_feature 1..105: note = Synthetic construct
  misc_feature 1..105: note = gRNA ZNF331_a
  source 1..105: mol_type = other RNA
caggtgagattggcgcccagtttaagagctatgctggaaacagcatagcaagtttaaataaggctagtccgttatcaacttgaaaaagtggcaccgagtcggtgc

SEQ ID NO: 300 - RNA, 105 nt, synthetic construct
  misc_feature 1..105: note = Synthetic construct
  misc_feature 1..105: note = gRNA ZNF541_a
  source 1..105: mol_type = other RNA
caagtcatgtgtcagataagtttaagagctatgctggaaacagcatagcaagtttaaataaggctagtccgttatcaacttgaaaaagtggcaccgagtcggtgc

SEQ ID NO: 301 - RNA, 105 nt, synthetic construct
  misc_feature 1..105: note = Synthetic construct
  misc_feature 1..105: note = gRNA ZNF619_a
  source 1..105: mol_type = other RNA
cgcagagcggttgtggagagtttaagagctatgctggaaacagcatagcaagtttaaataaggctagtccgttatcaacttgaaaaagtggcaccgagtcggtgc

SEQ ID NO: 302 - RNA, 105 nt, synthetic construct
  misc_feature 1..105: note = Synthetic construct
  misc_feature 1..105: note = gRNA AEBP1_a
  source 1..105: mol_type = other RNA
cgggaccccgagtccaccggtttaagagctatgctggaaacagcatagcaagtttaaataaggctagtccgttatcaacttgaaaaagtggcaccgagtcggtgc

SEQ ID NO: 303 - RNA, 105 nt, synthetic construct
  misc_feature 1..105: note = Synthetic construct
  misc_feature 1..105: note = gRNA ARID3A_a
  source 1..105: mol_type = other RNA
cccgcccccggcctagggagtttaagagctatgctggaaacagcatagcaagtttaaataaggctagtccgttatcaacttgaaaaagtggcaccgagtcggtgc

SEQ ID NO: 304 - RNA, 105 nt, synthetic construct
  misc_feature 1..105: note = Synthetic construct
  misc_feature 1..105: note = gRNA ARNTL2_a
  source 1..105: mol_type = other RNA
accctcggggcaaaggtaagtttaagagctatgctggaaacagcatagcaagtttaaataaggctagtccgttatcaacttgaaaaagtggcaccgagtcggtgc

SEQ ID NO: 305 - RNA, 105 nt, synthetic construct
  misc_feature 1..105: note = Synthetic construct
  misc_feature 1..105: note = gRNA BACH1_a
  source 1..105: mol_type = other RNA
tcgctgacagaggcttcgagtttaagagctatgctggaaacagcatagcaagtttaaataaggctagtccgttatcaacttgaaaaagtggcaccgagtcggtgc

SEQ ID NO: 306 - RNA, 105 nt, synthetic construct
  misc_feature 1..105: note = Synthetic construct
  misc_feature 1..105: note = gRNA BATF2_a
  source 1..105: mol_type = other RNA
caaaggccaggtatgaggagtttaagagctatgctggaaacagcatagcaagtttaaataaggctagtccgttatcaacttgaaaaagtggcaccgagtcggtgc

SEQ ID NO: 307 - RNA, 105 nt, synthetic construct
  misc_feature 1..105: note = Synthetic construct
  misc_feature 1..105: note = gRNA BRD4_a
  source 1..105: mol_type = other RNA
tgctgaagtcatgaggtgtgtttaagagctatgctggaaacagcatagcaagtttaaataaggctagtccgttatcaacttgaaaaagtggcaccgagtcggtgc

SEQ ID NO: 308 - RNA, 105 nt, synthetic construct
  misc_feature 1..105: note = Synthetic construct
  misc_feature 1..105: note = gRNA CDX4_a
  source 1..105: mol_type = other RNA
gggaagatcggtgaaaggggtttaagagctatgctggaaacagcatagcaagtttaaataaggctagtccgttatcaacttgaaaaagtggcaccgagtcggtgc

SEQ ID NO: 309 - RNA, 105 nt, synthetic construct
  misc_feature 1..105: note = Synthetic construct
  misc_feature 1..105: note = gRNA CENPB_a
  source 1..105: mol_type = other RNA
gcccccccgcggtatgtccgtttaagagctatgctggaaacagcatagcaagtttaaataaggctagtccgttatcaacttgaaaaagtggcaccgagtcggtgc

SEQ ID NO: 310 - RNA, 105 nt, synthetic construct
  misc_feature 1..105: note = Synthetic construct
  misc_feature 1..105: note = gRNA DMRT1_a
  source 1..105: mol_type = other RNA
agagcggtggctggagtcggtttaagagctatgctggaaacagcatagcaagtttaaataaggctagtccgttatcaacttgaaaaagtggcaccgagtcggtgc

SEQ ID NO: 311 - RNA, 105 nt, synthetic construct
  misc_feature 1..105: note = Synthetic construct
  misc_feature 1..105: note = gRNA E2F2_a
  source 1..105: mol_type = other RNA
cggaaagaacgcgtcctcggtttaagagctatgctggaaacagcatagcaagtttaaataaggctagtccgttatcaacttgaaaaagtggcaccgagtcggtgc

SEQ ID NO: 312 - RNA, 105 nt, synthetic construct
  misc_feature 1..105: note = Synthetic construct
  misc_feature 1..105: note = gRNA ESRRB_a
  source 1..105: mol_type = other RNA
gaggggcaacccccgactcgtttaagagctatgctggaaacagcatagcaagtttaaataaggctagtccgttatcaacttgaaaaagtggcaccgagtcggtgc

SEQ ID NO: 313 - RNA, 105 nt, synthetic construct
  misc_feature 1..105: note = Synthetic construct
  misc_feature 1..105: note = gRNA FERD3L_a
  source 1..105: mol_type = other RNA
tgagtaggaggtaaaggtagtttaagagctatgctggaaacagcatagcaagtttaaataaggctagtccgttatcaacttgaaaaagtggcaccgagtcggtgc

SEQ ID NO: 314 - RNA, 105 nt, synthetic construct
  misc_feature 1..105: note = Synthetic construct
  misc_feature 1..105: note = gRNA FEV_a
  source 1..105: mol_type = other RNA
cgggagccgactggaagaagtttaagagctatgctggaaacagcatagcaagtttaaataaggctagtccgttatcaacttgaaaaagtggcaccgagtcggtgc

SEQ ID NO: 315 - RNA, 105 nt, synthetic construct
  misc_feature 1..105: note = Synthetic construct
  misc_feature 1..105: note = gRNA FLYWCH1_a
  source 1..105: mol_type = other RNA
gggacgtaactcacggaaagtttaagagctatgctggaaacagcatagcaagtttaaataaggctagtccgttatcaacttgaaaaagtggcaccgagtcggtgc

SEQ ID NO: 316 - RNA, 105 nt, synthetic construct
  misc_feature 1..105: note = Synthetic construct
  misc_feature 1..105: note = gRNA FOXJ3_a
  source 1..105: mol_type = other RNA
cagagcctcgggagtctcagtttaagagctatgctggaaacagcatagcaagtttaaataaggctagtccgttatcaacttgaaaaagtggcaccgagtcggtgc

SEQ ID NO: 317 - RNA, 105 nt, synthetic construct
  misc_feature 1..105: note = Synthetic construct
  misc_feature 1..105: note = gRNA GLIS3_a
  source 1..105: mol_type = other RNA
ggctcgcgggtcactaggcgtttaagagctatgctggaaacagcatagcaagtttaaataaggctagtccgttatcaacttgaaaaagtggcaccgagtcggtgc

SEQ ID NO: 318 - RNA, 105 nt, synthetic construct
  misc_feature 1..105: note = Synthetic construct
  misc_feature 1..105: note = gRNA HDAC9_a
  source 1..105: mol_type = other RNA
gtaaagcaagcttcgagaggtttaagagctatgctggaaacagcatagcaagtttaaataaggctagtccgttatcaacttgaaaaagtggcaccgagtcggtgc

SEQ ID NO: 319 - RNA, 105 nt, synthetic construct
  misc_feature 1..105: note = Synthetic construct
  misc_feature 1..105: note = gRNA HIVEP1_2
  source 1..105: mol_type = other RNA
cagtcacgcggccccgggagtttaagagctatgctggaaacagcatagcaagtttaaataaggctagtccgttatcaacttgaaaaagtggcaccgagtcggtgc

SEQ ID NO: 320 - RNA, 105 nt, synthetic construct
  misc_feature 1..105: note = Synthetic construct
  misc_feature 1..105: note = gRNA HOXA2_4
  source 1..105: mol_type = other RNA
ggaaggaacgcctcattgggtttaagagctatgctggaaacagcatagcaagtttaaataaggctagtccgttatcaacttgaaaaagtggcaccgagtcggtgc

SEQ ID NO: 321 - RNA, 105 nt, synthetic construct
  misc_feature 1..105: note = Synthetic construct
  misc_feature 1..105: note = gRNA HOXC9_1
  source 1..105: mol_type = other RNA
gagctaggagccgtcctgagtttaagagctatgctggaaacagcatagcaagtttaaataaggctagtccgttatcaacttgaaaaagtggcaccgagtcggtgc

SEQ ID NO: 322 - RNA, 105 nt, synthetic construct
  misc_feature 1..105: note = Synthetic construct
  misc_feature 1..105: note = gRNA HSFY1_5
  source 1..105: mol_type = other RNA
ggtaggttacgggttgtcagtttaagagctatgctggaaacagcatagcaagtttaaataaggctagtccgttatcaacttgaaaaagtggcaccgagtcggtgc

SEQ ID NO: 323 - RNA, 105 nt, synthetic construct
  misc_feature 1..105: note = Synthetic construct
  misc_feature 1..105: note = gRNA ISL2_2
  source 1..105: mol_type = other RNA
aacggccgcggagccagctgtttaagagctatgctggaaacagcatagcaagtttaaataaggctagtccgttatcaacttgaaaaagtggcaccgagtcggtgc

SEQ ID NO: 324 - RNA, 105 nt, synthetic construct
  misc_feature 1..105: note = Synthetic construct
  misc_feature 1..105: note = gRNA KAT2A_5
  source 1..105: mol_type = other RNA
tccggagccgcgtggggcagtttaagagctatgctggaaacagcatagcaagtttaaataaggctagtccgttatcaacttgaaaaagtggcaccgagtcggtgc

SEQ ID NO: 325 - RNA, 105 nt, synthetic construct
  misc_feature 1..105: note = Synthetic construct
  misc_feature 1..105: note = gRNA KDM5D_5
  source 1..105: mol_type = other RNA
gcaagagcgattttaccaggtttaagagctatgctggaaacagcatagcaagtttaaataaggctagtccgttatcaacttgaaaaagtggcaccgagtcggtgc

SEQ ID NO: 326 - RNA, 105 nt, synthetic construct
  misc_feature 1..105: note = Synthetic construct
  misc_feature 1..105: note = gRNA KLF13_a
  source 1..105: mol_type = other RNA
accgggcacgcccctctaagtttaagagctatgctggaaacagcatagcaagtttaaataaggctagtccgttatcaacttgaaaaagtggcaccgagtcggtgc

SEQ ID NO: 327 - RNA, 105 nt, synthetic construct
  misc_feature 1..105: note = Synthetic construct
  misc_feature 1..105: note = gRNA KLF6_a
  source 1..105: mol_type = other RNA
tgaccgagactctccatcagtttaagagctatgctggaaacagcatagcaagtttaaataaggctagtccgttatcaacttgaaaaagtggcaccgagtcggtgc

SEQ ID NO: 328 - RNA, 105 nt, synthetic construct
  misc_feature 1..105: note = Synthetic construct
  misc_feature 1..105: note = gRNA MEOX2_a
  source 1..105: mol_type = other RNA
aatcagggtgctaggttgggtttaagagctatgctggaaacagcatagcaagtttaaataaggctagtccgttatcaacttgaaaaagtggcaccgagtcggtgc

SEQ ID NO: 329 - RNA, 105 nt, synthetic construct
  misc_feature 1..105: note = Synthetic construct
  misc_feature 1..105: note = gRNA MLXIPL_a
  source 1..105: mol_type = other RNA
cgccagagctccagagcacgtttaagagctatgctggaaacagcatagcaagtttaaataaggctagtccgttatcaacttgaaaaagtggcaccgagtcggtgc

SEQ ID NO: 330 - RNA, 105 nt, synthetic construct
  misc_feature 1..105: note = Synthetic construct
  misc_feature 1..105: note = gRNA MNT_a
  source 1..105: mol_type = other RNA
gggccgcggcgtctcgcgagtttaagagctatgctggaaacagcatagcaagtttaaataaggctagtccgttatcaacttgaaaaagtggcaccgagtcggtgc

SEQ ID NO: 331 - RNA, 105 nt, synthetic construct
  misc_feature 1..105: note = Synthetic construct
  misc_feature 1..105: note = gRNA MYCN_a
  source 1..105: mol_type = other RNA
caagcccgggggtccaaaagtttaagagctatgctggaaacagcatagcaagtttaaataaggctagtccgttatcaacttgaaaaagtggcaccgagtcggtgc

SEQ ID NO: 332 - RNA, 105 nt, synthetic construct
  misc_feature 1..105: note = Synthetic construct
  misc_feature 1..105: note = gRNA MYT1L_a
  source 1..105: mol_type = other RNA
gcctcatagataatgatgagtttaagagctatgctggaaacagcatagcaagtttaaataaggctagtccgttatcaacttgaaaaagtggcaccgagtcggtgc

SEQ ID NO: 333 - RNA, 105 nt, synthetic construct
  misc_feature 1..105: note = Synthetic construct
  misc_feature 1..105: note = gRNA NEUROG3_a
  source 1..105: mol_type = other RNA
ctcgcgtggcgcggccccagtttaagagctatgctggaaacagcatagcaagtttaaataaggctagtccgttatcaacttgaaaaagtggcaccgagtcggtgc

SEQ ID NO: 334 - RNA, 105 nt, synthetic construct
  misc_feature 1..105: note = Synthetic construct
  misc_feature 1..105: note = gRNA NFE2_a
  source 1..105: mol_type = other RNA
aggagagccacggggaaaggtttaagagctatgctggaaacagcatagcaagtttaaataaggctagtccgttatcaacttgaaaaagtggcaccgagtcggtgc

SEQ ID NO: 335 - RNA, 105 nt, synthetic construct
  misc_feature 1..105: note = Synthetic construct
  misc_feature 1..105: note = gRNA NR2F1_a
  source 1..105: mol_type = other RNA
tttctctccccccgattcagtttaagagctatgctggaaacagcatagcaagtttaaataaggctagtccgttatcaacttgaaaaagtggcaccgagtcggtgc

SEQ ID NO: 336 - RNA, 105 nt, synthetic construct
  misc_feature 1..105: note = Synthetic construct
  misc_feature 1..105: note = gRNA NR2F2_a
  source 1..105: mol_type = other RNA
ccccctcgccgtcattggcgtttaagagctatgctggaaacagcatagcaagtttaaataaggctagtccgttatcaacttgaaaaagtggcaccgagtcggtgc

SEQ ID NO: 337 - RNA, 105 nt, synthetic construct
  misc_feature 1..105: note = Synthetic construct
  misc_feature 1..105: note = gRNA NR3C1_a
  source 1..105: mol_type = other RNA
gggcccgcgcggagggcgtgtttaagagctatgctggaaacagcatagcaagtttaaataaggctagtccgttatcaacttgaaaaagtggcaccgagtcggtgc

SEQ ID NO: 338 - RNA, 105 nt, synthetic construct
  misc_feature 1..105: note = Synthetic construct
  misc_feature 1..105: note = gRNA PHF20_a
  source 1..105: mol_type = other RNA
aggccgttgccacagtgatgtttaagagctatgctggaaacagcatagcaagtttaaataaggctagtccgttatcaacttgaaaaagtggcaccgagtcggtgc

SEQ ID NO: 339 - RNA, 105 nt, synthetic construct
  misc_feature 1..105: note = Synthetic construct
  misc_feature 1..105: note = gRNA PITX2_a
  source 1..105: mol_type = other RNA
caggacagtccctccaccggtttaagagctatgctggaaacagcatagcaagtttaaataaggctagtccgttatcaacttgaaaaagtggcaccgagtcggtgc

SEQ ID NO: 340 - RNA, 105 nt, synthetic construct
  misc_feature 1..105: note = Synthetic construct
  misc_feature 1..105: note = gRNA POU2AF1_a
  source 1..105: mol_type = other RNA
gccgctggggcgcgcacaggtttaagagctatgctggaaacagcatagcaagtttaaataaggctagtccgttatcaacttgaaaaagtggcaccgagtcggtgc

SEQ ID NO: 341 - RNA, 105 nt, synthetic construct
  misc_feature 1..105: note = Synthetic construct
  misc_feature 1..105: note = gRNA POU3F2_a
  source 1..105: mol_type = other RNA
gcggggcccatccgagagagtttaagagctatgctggaaacagcatagcaagtttaaataaggctagtccgttatcaacttgaaaaagtggcaccgagtcggtgc

SEQ ID NO: 342 - RNA, 105 nt, synthetic construct
  misc_feature 1..105: note = Synthetic construct
  misc_feature 1..105: note = gRNA POU3F3_a
  source 1..105: mol_type = other RNA
ggccacgaccccccctgaagtttaagagctatgctggaaacagcatagcaagtttaaataaggctagtccgttatcaacttgaaaaagtggcaccgagtcggtgc

SEQ ID NO: 343 - RNA, 105 nt, synthetic construct
  misc_feature 1..105: note = Synthetic construct
  misc_feature 1..105: note = gRNA PRDM11_a
  source 1..105: mol_type = other RNA
tgggtgtggaggggttccagtttaagagctatgctggaaacagcatagcaagtttaaataaggctagtccgttatcaacttgaaaaagtggcaccgagtcggtgc

SEQ ID NO: 344 - RNA, 105 nt, synthetic construct
  misc_feature 1..105: note = Synthetic construct
  misc_feature 1..105: note = gRNA PRMT3_a
  source 1..105: mol_type = other RNA
tgaagaggcgcgctgatgagtttaagagctatgctggaaacagcatagcaagtttaaataaggctagtccgttatcaacttgaaaaagtggcaccgagtcggtgc

SEQ ID NO: 345 - RNA, 105 nt, synthetic construct
  misc_feature 1..105: note = Synthetic construct
  misc_feature 1..105: note = gRNA PROP1_a
  source 1..105: mol_type = other RNA
tcggagggtccagggactggtttaagagctatgctggaaacagcatagcaagtttaaataaggctagtccgttatcaacttgaaaaagtggcaccgagtcggtgc

SEQ ID NO: 346 - RNA, 105 nt, synthetic construct
  misc_feature 1..105: note = Synthetic construct
  misc_feature 1..105: note = gRNA PURA_a
  source 1..105: mol_type = other RNA
ctcacgcgcgagaaggggggtttaagagctatgctggaaacagcatagcaagtttaaataaggctagtccgttatcaacttgaaaaagtggcaccgagtcggtgc

SEQ ID NO: 347 - RNA, 105 nt, synthetic construct
  misc_feature 1..105: note = Synthetic construct
  misc_feature 1..105: note = gRNA RELB_a
  source 1..105: mol_type = other RNA
tgggcgctcaggaggggttgtttaagagctatgctggaaacagcatagcaagtttaaataaggctagtccgttatcaacttgaaaaagtggcaccgagtcggtgc

SEQ ID NO: 348 - RNA, 105 nt, synthetic construct
  misc_feature 1..105: note = Synthetic construct
  misc_feature 1..105: note = gRNA RFX3_a
  source 1..105: mol_type = other RNA
actgacggcccccaaacccgtttaagagctatgctggaaacagcatagcaagtttaaataaggctagtccgttatcaacttgaaaaagtggcaccgagtcggtgc

SEQ ID NO: 349 - RNA, 105 nt, synthetic construct
  misc_feature 1..105: note = Synthetic construct
  misc_feature 1..105: note = gRNA SAFB_a
  source 1..105: mol_type = other RNA
ctgctaagtacgctagtgggtttaagagctatgctggaaacagcatagcaagtttaaataaggctagtccgttatcaacttgaaaaagtggcaccgagtcggtgc

SEQ ID NO: 350 - RNA, 105 nt, synthetic construct
  misc_feature 1..105: note = Synthetic construct
  misc_feature 1..105: note = gRNA SETBP1_a
  source 1..105: mol_type = other RNA
tatggagagagatatcaaggtttaagagctatgctggaaacagcatagcaagtttaaataaggctagtccgttatcaacttgaaaaagtggcaccgagtcggtgc

SEQ ID NO: 351 - RNA, 105 nt, synthetic construct
  misc_feature 1..105: note = Synthetic construct
  misc_feature 1..105: note = gRNA SMAD2_a
  source 1..105: mol_type = other RNA
aagccgcgcggggcgggtagtttaagagctatgctggaaacagcatagcaagtttaaataaggctagtccgttatcaacttgaaaaagtggcaccgagtcggtgc

SEQ ID NO: 352 - RNA, 105 nt, synthetic construct
  misc_feature 1..105: note = Synthetic construct
  misc_feature 1..105: note = gRNA SMAD6_a
  source 1..105: mol_type = other RNA
cccggggcgggccgagctcgtttaagagctatgctggaaacagcatagcaagtttaaataaggctagtccgttatcaacttgaaaaagtggcaccgagtcggtgc

SEQ ID NO: 353 - RNA, 105 nt, synthetic construct
  misc_feature 1..105: note = Synthetic construct
  misc_feature 1..105: note = gRNA SNAPC2_a
  source 1..105: mol_type = other RNA
agggagggcatgggatccagtttaagagctatgctggaaacagcatagcaagtttaaataaggctagtccgttatcaacttgaaaaagtggcaccgagtcggtgc

SEQ ID NO: 354 - RNA, 105 nt, synthetic construct
  misc_feature 1..105: note = Synthetic construct
  misc_feature 1..105: note = gRNA SNAPC4_a
  source 1..105: mol_type = other RNA
tctcagagcccaccctcgagtttaagagctatgctggaaacagcatagcaagtttaaataaggctagtccgttatcaacttgaaaaagtggcaccgagtcggtgc

SEQ ID NO: 355 - RNA, 105 nt, synthetic construct
  misc_feature 1..105: note = Synthetic construct
  misc_feature 1..105: note = gRNA SON_a
  source 1..105: mol_type = other RNA
ctgtacttggcctgtactagtttaagagctatgctggaaacagcatagcaagtttaaataaggctagtccgttatcaacttgaaaaagtggcaccgagtcggtgc

SEQ ID NO: 356 - RNA, 105 nt, synthetic construct
  misc_feature 1..105: note = Synthetic construct
  misc_feature 1..105: note = gRNA SOX11_a
  source 1..105: mol_type = other RNA
gggctgctttgcaaagatggtttaagagctatgctggaaacagcatagcaagtttaaataaggctagtccgttatcaacttgaaaaagtggcaccgagtcggtgc

SEQ ID NO: 357 - RNA, 105 nt, synthetic construct
  misc_feature 1..105: note = Synthetic construct
  misc_feature 1..105: note = gRNA SOX12_a
  source 1..105: mol_type = other RNA
ggcccggccgcagcccaaggtttaagagctatgctggaaacagcatagcaagtttaaataaggctagtccgttatcaacttgaaaaagtggcaccgagtcggtgc

SEQ ID NO: 358 - RNA, 105 nt, synthetic construct
  misc_feature 1..105: note = Synthetic construct
  misc_feature 1..105: note = gRNA SOX14_a
  source 1..105: mol_type = other RNA
caaaagcggcgaagttcaagtttaagagctatgctggaaacagcatagcaagtttaaataaggctagtccgttatcaacttgaaaaagtggcaccgagtcggtgc

SEQ ID NO: 359 - RNA, 105 nt, synthetic construct
  misc_feature 1..105: note = Synthetic construct
  misc_feature 1..105: note = gRNA SPIC_a
  source 1..105: mol_type = other RNA
tgcaacctaagttgggcgggtttaagagctatgctggaaacagcatagcaagtttaaataaggctagtccgttatcaacttgaaaaagtggcaccgagtcggtgc

SEQ ID NO: 360 - RNA, 105 nt, synthetic construct
  misc_feature 1..105: note = Synthetic construct
  misc_feature 1..105: note = gRNA THAP7_a
  source 1..105: mol_type = other RNA
aatcggctgggacggggttgtttaagagctatgctggaaacagcatagcaagtttaaataaggctagtccgttatcaacttgaaaaagtggcaccgagtcggtgc

SEQ ID NO: 361 - RNA, 105 nt, synthetic construct
  misc_feature 1..105: note = Synthetic construct
  misc_feature 1..105: note = gRNA TOX2_a
  source 1..105: mol_type = other RNA
gctcccgcgggcagtgcctgtttaagagctatgctggaaacagcatagcaagtttaaataaggctagtccgttatcaacttgaaaaagtggcaccgagtcggtgc

SEQ ID NO: 362 - RNA, 105 nt, synthetic construct
  misc_feature 1..105: note = Synthetic construct
  misc_feature 1..105: note = gRNA TRERF1_a
  source 1..105: mol_type = other RNA
gggtaccggggagcggatagtttaagagctatgctggaaacagcatagcaagtttaaataaggctagtccgttatcaacttgaaaaagtggcaccgagtcggtgc

SEQ ID NO: 363 - RNA, 105 nt, synthetic construct
  misc_feature 1..105: note = Synthetic construct
  misc_feature 1..105: note = gRNA TRIM3_a
  source 1..105: mol_type = other RNA
gggcgtgcgggtgcggtaggtttaagagctatgctggaaacagcatagcaagtttaaataaggctagtccgttatcaacttgaaaaagtggcaccgagtcggtgc

SEQ ID NO: 364 - RNA, 105 nt, synthetic construct
  misc_feature 1..105: note = Synthetic construct
  misc_feature 1..105: note = gRNA VDR_a
  source 1..105: mol_type = other RNA
tacagcgtggcctataggggtttaagagctatgctggaaacagcatagcaagtttaaataaggctagtccgttatcaacttgaaaaagtggcaccgagtcggtgc

SEQ ID NO: 365 - RNA, 105 nt, synthetic construct
  misc_feature 1..105: note = Synthetic construct
  misc_feature 1..105: note = gRNA YBX2_a
  source 1..105: mol_type = other RNA
gggcccaggtgtacttactgtttaagagctatgctggaaacagcatagcaagtttaaataaggctagtccgttatcaacttgaaaaagtggcaccgagtcggtgc

SEQ ID NO: 366 - RNA, 105 nt, synthetic construct
  misc_feature 1..105: note = Synthetic construct
  misc_feature 1..105: note = gRNA ZBTB14_a
  source 1..105: mol_type = other RNA
cgcgcgcgtgcgcaggccagtttaagagctatgctggaaacagcatagcaagtttaaataaggctagtccgttatcaacttgaaaaagtggcaccgagtcggtgc

SEQ ID NO: 367 - RNA, 105 nt, synthetic construct
  misc_feature 1..105: note = Synthetic construct
  misc_feature 1..105: note = gRNA ZBTB4_a
  source 1..105: mol_type = other RNA
ccgggacggttggagaagagtttaagagctatgctggaaacagcatagcaagtttaaataaggctagtccgttatcaacttgaaaaagtggcaccgagtcggtgc

SEQ ID NO: 368 - RNA, 105 nt, synthetic construct
  misc_feature 1..105: note = Synthetic construct
  misc_feature 1..105: note = gRNA ZFHX2_a
  source 1..105: mol_type = other RNA
ggtacaggtctcatgcctggtttaagagctatgctggaaacagcatagcaagtttaaataaggctagtccgttatcaacttgaaaaagtggcaccgagtcggtgc

SEQ ID NO: 369 - RNA, 105 nt, synthetic construct
  misc_feature 1..105: note = Synthetic construct
  misc_feature 1..105: note = gRNA ZFP57_a
  source 1..105: mol_type = other RNA
gcagctatcagttggtcgggtttaagagctatgctggaaacagcatagcaagtttaaataaggctagtccgttatcaacttgaaaaagtggcaccgagtcggtgc

SEQ ID NO: 370 - RNA, 105 nt, synthetic construct
  misc_feature 1..105: note = Synthetic construct
  misc_feature 1..105: note = gRNA ZFP62_a
  source 1..105: mol_type = other RNA
gagggcagagagacctctagtttaagagctatgctggaaacagcatagcaagtttaaataaggctagtccgttatcaacttgaaaaagtggcaccgagtcggtgc

SEQ ID NO: 371 - RNA, 105 nt, synthetic construct
  misc_feature 1..105: note = Synthetic construct
  misc_feature 1..105: note = gRNA ZFP69B_a
  source 1..105: mol_type = other RNA
tgatgatgctcgcagggatgtttaagagctatgctggaaacagcatagcaagtttaaataaggctagtccgttatcaacttgaaaaagtggcaccgagtcggtgc

SEQ ID NO: 372 - RNA, 105 nt, synthetic construct
  misc_feature 1..105: note = Synthetic construct
  misc_feature 1..105: note = gRNA ZIC1_a
  source 1..105: mol_type = other RNA
gggaatgcgggcgcaccaagtttaagagctatgctggaaacagcatagcaagtttaaataaggctagtccgttatcaacttgaaaaagtggcaccgagtcggtgc

SEQ ID NO: 373 - RNA, 105 nt, synthetic construct
  misc_feature 1..105: note = Synthetic construct
  misc_feature 1..105: note = gRNA ZIC4_a
  source 1..105: mol_type = other RNA
caacggggacgttgacaaggtttaagagctatgctggaaacagcatagcaagtttaaataaggctagtccgttatcaacttgaaaaagtggcaccgagtcggtgc

SEQ ID NO: 374 - RNA, 105 nt, synthetic construct
  misc_feature 1..105: note = Synthetic construct
  misc_feature 1..105: note = gRNA ZNF136_a
  source 1..105: mol_type = other RNA
ccccgacggcgcgcctgatgtttaagagctatgctggaaacagcatagcaagtttaaataaggctagtccgttatcaacttgaaaaagtggcaccgagtcggtgc

SEQ ID NO: 375 - RNA, 105 nt, synthetic construct
  misc_feature 1..105: note = Synthetic construct
  misc_feature 1..105: note = gRNA ZNF16_a
  source 1..105: mol_type = other RNA
gctagtgggaccaggtctggtttaagagctatgctggaaacagcatagcaagtttaaataaggctagtccgttatcaacttgaaaaagtggcaccgagtcggtgc

SEQ ID NO: 376 - RNA, 105 nt, synthetic construct
  misc_feature 1..105: note = Synthetic construct
  misc_feature 1..105: note = gRNA ZNF174_a
  source 1..105: mol_type = other RNA
caggggcgtgcgtaatggggtttaagagctatgctggaaacagcatagcaagtttaaataaggctagtccgttatcaacttgaaaaagtggcaccgagtcggtgc

SEQ ID NO: 377 - RNA, 105 nt, synthetic construct
  misc_feature 1..105: note = Synthetic construct
  misc_feature 1..105: note = gRNA ZNF253_a
  source 1..105: mol_type = other RNA
tgtcactcagcggctgggggtttaagagctatgctggaaacagcatagcaagtttaaataaggctagtccgttatcaacttgaaaaagtggcaccgagtcggtgc

SEQ ID NO: 378 - RNA, 105 nt, synthetic construct
  misc_feature 1..105: note = Synthetic construct
  misc_feature 1..105: note = gRNA ZNF26_a
  source 1..105: mol_type = other RNA
acagcctgacgggaaaggagtttaagagctatgctggaaacagcatagcaagtttaaataaggctagtccgttatcaacttgaaaaagtggcaccgagtcggtgc

SEQ ID NO: 379 - RNA, 105 nt, synthetic construct
  misc_feature 1..105: note = Synthetic construct
  misc_feature 1..105: note = gRNA ZNF263_a
  source 1..105: mol_type = other RNA
gggcgcgccctgaaattctgtttaagagctatgctggaaacagcatagcaagtttaaataaggctagtccgttatcaacttgaaaaagtggcaccgagtcggtgc

SEQ ID NO: 380 - RNA, 105 nt, synthetic construct
  misc_feature 1..105: note = Synthetic construct
  misc_feature 1..105: note = gRNA ZNF285_a
  source 1..105: mol_type = other RNA
aaagccccgcgcgcagcaggtttaagagctatgctggaaacagcatagcaagtttaaataaggctagtccgttatcaacttgaaaaagtggcaccgagtcggtgc

SEQ ID NO: 381 - RNA, 105 nt, synthetic construct
  misc_feature 1..105: note = Synthetic construct
  misc_feature 1..105: note = gRNA ZNF317_a
  source 1..105: mol_type = other RNA
tcccgcgagaattggaagggtttaagagctatgctggaaacagcatagcaagtttaaataaggctagtccgttatcaacttgaaaaagtggcaccgagtcggtgc

SEQ ID NO: 382 - RNA, 105 nt, synthetic construct
  misc_feature 1..105: note = Synthetic construct
  misc_feature 1..105: note = gRNA ZNF319_b
  source 1..105: mol_type = other RNA
catcttccggaagggaggggtttaagagctatgctggaaacagcatagcaagtttaaataaggctagtccgttatcaacttgaaaaagtggcaccgagtcggtgc

SEQ ID NO: 383 - RNA, 105 nt, synthetic construct
  misc_feature 1..105: note = Synthetic construct
  misc_feature 1..105: note = gRNA ZNF345_a
  source 1..105: mol_type = other RNA
cgcgccaggggaatcgggagtttaagagctatgctggaaacagcatagcaagtttaaataaggctagtccgttatcaacttgaaaaagtggcaccgagtcggtgc

SEQ ID NO: 384 - RNA, 105 nt, synthetic construct
  misc_feature 1..105: note = Synthetic construct
  misc_feature 1..105: note = gRNA ZNF420_a
  source 1..105: mol_type = other RNA
gagcccacgagcccctcgagtttaagagctatgctggaaacagcatagcaagtttaaataaggctagtccgttatcaacttgaaaaagtggcaccgagtcggtgc

SEQ ID NO: 385 - RNA, 105 nt, synthetic construct
  misc_feature 1..105: note = Synthetic construct
  misc_feature 1..105: note = gRNA ZNF423_a
  source 1..105: mol_type = other RNA
caggggatcagccatccaggtttaagagctatgctggaaacagcatagcaagtttaaataaggctagtccgttatcaacttgaaaaagtggcaccgagtcggtgc

SEQ ID NO: 386 - RNA, 105 nt, synthetic construct
  misc_feature 1..105: note = Synthetic construct
  misc_feature 1..105: note = gRNA ZNF470_b
  source 1..105: mol_type = other RNA
gaccaggagaggcccaaaagtttaagagctatgctggaaacagcatagcaagtttaaataaggctagtccgttatcaacttgaaaaagtggcaccgagtcggtgc

SEQ ID NO: 387 - RNA, 105 nt, synthetic construct
  misc_feature 1..105: note = Synthetic construct
  misc_feature 1..105: note = gRNA ZNF48_a
  source 1..105: mol_type = other RNA
gaggccggcggcccgaactgtttaagagctatgctggaaacagcatagcaagtttaaataaggctagtccgttatcaacttgaaaaagtggcaccgagtcggtgc

SEQ ID NO: 388 - RNA, 105 nt, synthetic construct
  misc_feature 1..105: note = Synthetic construct
  misc_feature 1..105: note = gRNA ZNF496_a
  source 1..105: mol_type = other RNA
ggagcaggaagtgtcccaggtttaagagctatgctggaaacagcatagcaagtttaaataaggctagtccgttatcaacttgaaaaagtggcaccgagtcggtgc

SEQ ID NO: 389 - RNA, 105 nt, synthetic construct
  misc_feature 1..105: note = Synthetic construct
  misc_feature 1..105: note = gRNA ZNF501_a
  source 1..105: mol_type = other RNA
aaacgggatggaagtcagagtttaagagctatgctggaaacagcatagcaagtttaaataaggctagtccgttatcaacttgaaaaagtggcaccgagtcggtgc

SEQ ID NO: 390 - RNA, 105 nt, synthetic construct
  misc_feature 1..105: note = Synthetic construct
  misc_feature 1..105: note = gRNA ZNF524_a
  source 1..105: mol_type = other RNA
gggtcctaacgaccggtcggtttaagagctatgctggaaacagcatagcaagtttaaataaggctagtccgttatcaacttgaaaaagtggcaccgagtcggtgc

SEQ ID NO: 391 - RNA, 105 nt, synthetic construct
  misc_feature 1..105: note = Synthetic construct
  misc_feature 1..105: note = gRNA ZNF557_a
  source 1..105: mol_type = other RNA
ctggcgcgggtctgtggcagtttaagagctatgctggaaacagcatagcaagtttaaataaggctagtccgttatcaacttgaaaaagtggcaccgagtcggtgc

SEQ ID NO: 392 - RNA, 105 nt, synthetic construct
  misc_feature 1..105: note = Synthetic construct
  misc_feature 1..105: note = gRNA ZNF735_a
  source 1..105: mol_type = other RNA
aaaaggcccggtaaaattagtttaagagctatgctggaaacagcatagcaagtttaaataaggctagtccgttatcaacttgaaaaagtggcaccgagtcggtgc

SEQ ID NO: 393 - RNA, 105 nt, synthetic construct
  misc_feature 1..105: note = Synthetic construct
  misc_feature 1..105: note = gRNA ZNF747_a
  source 1..105: mol_type = other RNA
ggaacaaagcagcgccctggtttaagagctatgctggaaacagcatagcaagtttaaataaggctagtccgttatcaacttgaaaaagtggcaccgagtcggtgc

SEQ ID NO: 394 - RNA, 105 nt, synthetic construct
  misc_feature 1..105: note = Synthetic construct
  misc_feature 1..105: note = gRNA ZNF783_a
  source 1..105: mol_type = other RNA
aggcgctgcaacagagtctgtttaagagctatgctggaaacagcatagcaagtttaaataaggctagtccgttatcaacttgaaaaagtggcaccgagtcggtgc

SEQ ID NO: 395 - RNA, 105 nt, synthetic construct
  misc_feature 1..105: note = Synthetic construct
  misc_feature 1..105: note = gRNA ZNF805_a
  source 1..105: mol_type = other RNA
ctggggccaatgatggtccgtttaagagctatgctggaaacagcatagcaagtttaaataaggctagtccgttatcaacttgaaaaagtggcaccgagtcggtgc

SEQ ID NO: 396 - RNA, 105 nt, synthetic construct
  misc_feature 1..105: note = Synthetic construct
  misc_feature 1..105: note = gRNA ZSCAN10_a
  source 1..105: mol_type = other RNA
ttacactctcacacaggctgtttaagagctatgctggaaacagcatagcaagtttaaataaggctagtccgttatcaacttgaaaaagtggcaccgagtcggtgc

SEQ ID NO: 397 - DNA, 86 bp, synthetic construct
  misc_feature 1..86: note = Synthetic construct
  misc_feature 1..86: note = SpCas9 gRNA scaffold (DNA)
  source 1..86: mol_type = other DNA
gtttaagagctatgctggaaacagcatagcaagtttaaataaggctagtccgttatcaacttgaaaaagtggcaccgagtcggtgc

SEQ ID NO: 398 - RNA, 86 nt, synthetic construct
  misc_feature 1..86: note = Synthetic construct
  misc_feature 1..86: note = SpCas9 gRNA scaffold (RNA)
  source 1..86: mol_type = other RNA
gtttaagagctatgctggaaacagcatagcaagtttaaataaggctagtccgttatcaacttgaaaaagtggcaccgagtcggtgc

SEQ ID NO: 399 - DNA, 4509 bp, synthetic construct
  misc_feature 1..4509: note = Synthetic construct
  misc_feature 1..4509: note = VP64-dSpCas9-NLS-VP64 (nt)
  source 1..4509: mol_type = other DNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatggttaacccaaagaagaagcggaaggtcggtatccacggagtcccagcagccgacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacaaaaggccggcggccacgaaaaaggccggccaggcaaaaaagaaaaaggctagccgcgccgacgcgctggacgatttcgatctcgacatgctgggttctgatgccctcgatgactttgacctggatatgttgggaagcgacgcattggatgactttgatctggacatgctcggctccgatgctctggacgatttcgatctcgatatgtta

SEQ ID NO: 400 - protein, 1503 aa, synthetic construct
  REGION 1..1503: note = Synthetic construct
  REGION 1..1503: note = VP64-dSpCas9-NLS-VP64 (AA)
DALDDFDLDMLGSDALDDFDLDMLGSDALDDFDLDMLGSDALDDFDLDMVNPKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKASRADALDDFDLDMLGSDALDDFDLDMLGSDALDDFDLDMLGSDALDDFDLDML

SEQ ID NO: 401 - DNA, 4509 bp, synthetic construct
  misc_feature 1..4509: note = Synthetic construct
  misc_feature 1..4509: note = dSpCas9-KRAB (nt)
  source 1..4509: mol_type = other DNA
ccaaagaagaagcggaaggtcggtatccacggagtcccagcagccgacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacaaaaggccggcggccacgaaaaaggccggccaggcaaaaaagaaaaaggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagtt

SEQ ID NO: 402 - protein, 1503 aa, synthetic construct
  REGION 1..1503: note = Synthetic construct
  REGION 1..1503: note = dSpCas9-KRAB (AA)
PKKKRKVGIHGVPAADKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKASDAKSLTAWSRTLVTFKDVFVDFTREEWKLLDTAQQILYRNVMLENYKNLVSLGYQLTKPDVILRLEKGEEPWLVEREIHQETHPDSETAFEIKSSVPKKKRKV

SEQ ID NO: 403-404 - skipped (2 entries)

SEQ ID NO: 405 - protein, 1053 aa, synthetic construct
  REGION 1..1053: note = Synthetic construct
  REGION 1..1053: note = SaCas9 (AA)
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 406 - protein, 1052 aa, synthetic construct
  REGION 1..1052: note = Synthetic construct
  REGION 1..1052: note = dSaCas9 (AA)
KRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEEASKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 407 - protein, 1368 aa, synthetic construct [sequence omitted]

SEQ ID NO: 408 - protein, 1367 aa, synthetic construct
  REGION 1..1367: note = Synthetic construct
  REGION 1..1367: note = dSpCas9 (AA)
DKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 409 - protein, 49 aa, synthetic construct
  REGION 1..49: note = Synthetic construct
  REGION 1..49: note = VP64 (AA)
DALDDFDLDMLGSDALDDFDLDMLGSDALDDFDLDMLGSDALDDFDLDM

SEQ ID NO: 410 - protein, 16 aa, synthetic construct
  REGION 1..16: note = Synthetic construct
  REGION 1..16: note = NLS - nucleoplasmin (AA)
KRPAATKKAGQAKKKK

SEQ ID NO: 411 - protein, 124 aa, synthetic construct
  REGION 1..124: note = Synthetic construct
  REGION 1..124: note = p65AD (AA)
PTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISS

SEQ ID NO: 412 - protein, 190 aa, synthetic construct
  REGION 1..190: note = Synthetic construct
  REGION 1..190: note = Rta domain (AA)
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 413 - protein, 2414 aa, synthetic construct
  REGION 1..2414: note = Synthetic construct
  REGION 1..2414: note = p300 domain (AA)
MAENVVEPGPPSAKRPKLSSPALSASASDGTDFGSLFDLEHDLPDELINSTELGLTNGGDINQLQTSLGMVQDAASKHKQLSELLRSGSSPNLNMGVGGPGQVMASQAQQSSPGLGLINSMVKSPMTQAGLTSPNMGMGTSGPNQGPTQSTGMMNSPVNQPAMGMNTGMNAGMNPGMLAAGNGQGIMPNQVMNGSIGAGRGRQNMQYPNPGMGSAGNLLTEPLQQGSPQMGGQTGLRGPQPLKMGMMNNPNPYGSPYTQNPGQQIGASGLGLQIQTKTVLSNNLSPFAMDKKAVPGGGMPNMGQQPAPQVQQPGLVTPVAQGMGSGAHTADPEKRKLIQQQLVLLLHAHKCQRREQANGEVRQCNLPHCRTMKNVLNHMTHCQSGKSCQVAHCASSRQIISHWKNCTRHDCPVCLPLKNAGDKRNQQPILTGAPVGLGNPSSLGVGQQSAPNLSTVSQIDPSSIERAYAALGLPYQVNQMPTQPQVQAKNQQNQQPGQSPQGMRPMSNMSASPMGVNGGVGVQTPSLLSDSMLHSAINSQNPMMSENASVPSMGPMPTAAQPSTTGIRKQWHEDITQDLRNHLVHKLVQAIFPTPDPAALKDRRMENLVAYARKVEGDMYESANNRAEYYHLLAEKIYKIQKELEEKRRTRLQKQNMLPNAAGMVPVSMNPGPNMGQPQPGMTSNGPLPDPSMIRGSVPNQMMPRITPQSGLNQFGQMSMAQPPIVPRQTPPLQHHGQLAQPGALNPPMGYGPRMQQPSNQGQFLPQTQFPSQGMNVTNIPLAPSSGQAPVSQAQMSSSSCPVNSPIMPPGSQGSHIHCPQLPQPALHQNSPSPVPSRTPTPHHTPPSIGAQQPPATTIPAPVPTPPAMPPGPQSQALHPPPRQTPTPPTTQLPQQVQPSLPAAPSADQPQQQPRSQQSTAASVPTPTAPLLPPQPATPLSQPAVSIEGQVSNPPSTSSTEVNSQAIAEKQPSQEVKMEAKMEVDQPEPADTQPEDISESKVEDCKMESTETEERSTELKTEIKEEEDQPSTSATQSSPAPGQSKKKIFKPEELRQALMPTLEALYRQDPESLPFRQPVDPQLLGIPDYFDIVKSPMDLSTIKRKLDTGQYQEPWQYVDDIWLMFNNAWLYNRKTSRVYKYCSKLSEVFEQEIDPVMQSLGYCCGRKLEFSPQTLCCYGKQLCTIPRDATYYSYQNRYHFCEKCFNEIQGESVSLGDDPSQPQTTINKEQFSKRKNDTLDPELFVECTECGRKMHQICVLHHEIIWPAGFVCDGCLKKSARTRKENKFSAKRLPSTRLGTFLENRVNDFLRRQNHPESGEVTVRVVHASDKTVEVKPGMKARFVDSGEMAESFPYRTKALFAFEEIDGVDLCFFGMHVQEYGSDCPPPNQRRVYISYLDSVHFFRPKCLRTAVYHEILIGYLEYVKKLGYTTGHIWACPPSEGDDYIFHCHPPDQKIPKPKRLQEWYKKMLDKAVSERIVHDYKDIFKQATEDRLTSAKELPYFEGDFWPNVLEESIKELEQEEEERKREENTSNESTDVTKGDSKNAKKKNNKKTSKNKSSLSRGNKKKPGMPNVSNDLSQKLYATMEKHKEVFFVIRLIAGPAANSLPPIVDPDPLIPCDLMDGRDAFLTLARDKHLEFSSLRRAQWSTMCMLVELHTQSQDRFVYTCNECKHHVETRWHCTVCEDYDLCITCYNTKNHDHKMEKLGLGLDDESNNQQAAATQSPGDSRRLSIQRCIQSLVHACQCRNANCSLPSCQKMKRVVQHTKGCKRKTNGGCPICKQLIALCCYHAKHCQENKCPVPFCLNIKQKLRQQQLQHRLQQAQMLRRRMASMQRTGVVGQQQGLPSPTPATPTTPTGQQPTTPQTPQPTSQPQPTPPNSMPPYLPRTQAAGPVSQGKAAGQVTPPTPPQTAQPPLPGPPPAAVEMAMQIQRAAETQRQMAHVQIFQRPIQHQMPPMTPMAPMGMNPPPMTRGPSGHLEPGMGPTGMQQQPPWSQGGLPQPQQLQSGMPRPAMMSVAQHGQPLNMAPQPGLGQVGISPLKPGTVSQQALQNLLRTLRSPSSPLQQQQVLSILHANPQLLAAFIKQRAAKYANSNPQPIPGQPGMPQGQPGLQPPTMPGQQGVHSNPAMQNMNPMQAGVQRAGLPQQQPQQQLQPPMGGMSPQAQQMNMNHNTMPSQFRDILRRQQMMQQQQQQGAGPGIGPGMANHNQFQQPQGVGYPPQQQQRMQHHMQQMQQGNMGQIGQLPQALGAEAGASLQAYQQRLLQQQMGSPVQPNPMSPQQHMLPNQAQSPHLQGQQIPNSLSNQVRSPQPVPSPRPQSQPPHSSPSPRMQPQPSPHHVSPQTSSPHPGLVAAQANPMEQGHFASPDQNSMLSQLASNPGMANLHGASATDLGLSTDNSDLNSNLSQSTLDIH

SEQ ID NO: 414 - protein, 124 aa, synthetic construct
  REGION 1..124: note = Synthetic construct
  REGION 1..124: note = HSF1 domain (AA)
GFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 415 - protein, 522 aa, synthetic construct
  REGION 1..522: note = Synthetic construct
  REGION 1..522: note = VPR (AA)
DALDDFDLDMLGSDALDDFDLDMLGSDALDDFDLDMLGSDALDDFDLDMLGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSQISSGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 416 - protein, 378 aa, synthetic construct
  REGION 1..378: note = Synthetic construct
  REGION 1..378: note = VPH (AA)
DALDDFDLDMLGSDALDDFDLDMLGSDALDDFDLDMLGSDALDDFDLDMLGSLPSASVEFEGSGGPSGQISNQALALAPSSAPVLAQTMVPSSAMVPLAQPPAPAPVLTPGPPQSLSAPVPKSTQAGEGTLSEALLHLQFDADEDLGALLGNSTDPGVFTDLASVDNSEFQQLLNQGVSMSHSTAEPMLMEYPEAITRLVTGSQRPPDPAPTPLGTSGLPNGLSGDEDFSSIADMDFSALLSQISSSGQGGGGSGFSVDTSALLDLFSPSVTVPDMSLPDLDSSLASIQELLSPQEPPRPPEAENSSPDSGKQLVHYTAQPLFLLDPGSVDTGSNDLPVLFELGEGSYFSEGDGFAEDPTISLLTGSEPPKAKDPTVS

SEQ ID NO: 417 - protein, 718 aa, synthetic construct
  REGION 1..718: note = Synthetic construct
  REGION 1..718: note = TET1 catalytic domain (AA)
LPTCSCLDRVIQKDKGPYYTHLGAGPSVAAVREIMENRYGQKGNAIRIEIVVYTGKEGKSSHGCPIAKWVLRRSSDEEKVLCLVRQRTGHHCPTAVMVVLIMVWDGIPLPMADRLYTELTENLKSYNGHPTDRRCTLNENRTCTCQGIDPETCGASFSFGCSWSMYFNGCKFGRSPSPRRFRIDPSSPLHEKNLEDNLQSLATRLAPIYKQYAPVAYQNQVEYENVARECRLGSKEGRPFSGVTACLDFCAHPHRDIHNMNNGSTVVCTLTREDNRSLGVIPQDEQLHVLPLYKLSDTDEFGSKEGMEAKIKSGAIEVLAPRRKKRTCFTQPVPRSGKKRAAMMTEVLAHKIRAVEKKPIPRIKRKNNSTTTNNSKPSSLPTLGSNTETVQPEVKSETEPHFILKSSDNTKTYSLMPSAPHPVKEASPGFSWSPKTASATPAPLKNDATASCGFSERSSTPHCTMPSGRLSGANAAAADGPGISQLGEVAPLPTLSAPVMEPLINSEPSTGVTEPLTPHQPNHQPSFLTSPQDLASSPMEEDEQHSEADEPPSDEPLSDDPLSPAEEKLPHIDEYWSDSEHIFLDANIGGVAIAPAHGSVLIECARRELHATTPVEHPNRNHPTRLSLVFYQHKNLNKPQHGFELNKIKFEAKEAKNKKMKASEQKDQAANEGPEQSSEVNELNQIPSHKALTLTHDNVVTVSPYALTHVAGPYNHWV

SEQ ID NO: 418 - protein, 5 aa, synthetic construct
  REGION 1..5: note = Synthetic construct
  REGION 1..5: note = GGGGS linker (AA)
GGGGS

SEQ ID NO: 419 - protein, 5 aa, synthetic construct
  REGION 1..5: note = Synthetic construct
  REGION 1..5: note = GGGGG linker (AA)
GGGGG

SEQ ID NO: 420 - protein, 5 aa, synthetic construct
  REGION 1..5: note = Synthetic construct
  REGION 1..5: note = GGAGG linker (AA)
GGAGG

SEQ ID NO: 421 - protein, 7 aa, synthetic construct
  REGION 1..7: note = Synthetic construct
  REGION 1..7: note = GGGGSSS linker (AA)
GGGGSSS

SEQ ID NO: 422 - protein, 7 aa, synthetic construct
  REGION 1..7: note = Synthetic construct
  REGION 1..7: note = GGGGAAA linker (AA)
GGGGAAA

SEQ ID NO: 423 - protein, 7 aa, synthetic construct
  REGION 1..7: note = Synthetic construct
  REGION 1..7: note = NLS - SV40 (AA)
PKKKRKV

SEQ ID NO: 424 - protein, 9 aa, synthetic construct
  REGION 1..9: note = Synthetic construct
  REGION 1..9: note = NLS - cMyc (AA)
PAAKRVKLD

SEQ ID NO: 425 - protein, 11 aa, synthetic construct
  REGION 1..11: note = Synthetic construct
  REGION 1..11: note = NLS - cMyc (AA)
RQRRNELKRSP

SEQ ID NO: 426 - protein, 38 aa, synthetic construct
  REGION 1..38: note = Synthetic construct
  REGION 1..38: note = NLS - hRNPA1 M9 (AA)
NQSSNFGPMKGGNFGGRSSGPYGGGGQYFAKPRNQGGY

SEQ ID NO: 427 - protein, 42 aa, synthetic construct
  REGION 1..42: note = Synthetic construct
  REGION 1..42: note = NLS - IBB domain importin-alpha (AA)
RMRIZFKNKGKDTAELRRRRVEVSVELRKAKKDEQILKRRNV

SEQ ID NO: 428 - protein, 8 aa, synthetic construct
  REGION 1..8: note = Synthetic construct
  REGION 1..8: note = NLS - myoma T protein (AA)
VSRKRPRP

SEQ ID NO: 429 - protein, 8 aa, synthetic construct
  REGION 1..8: note = Synthetic construct
  REGION 1..8: note = NLS - myoma T protein (AA)
PPKKARED

SEQ ID NO: 430 - protein, 8 aa, synthetic construct
  REGION 1..8: note = Synthetic construct
  REGION 1..8: note = NLS - human p53 (AA)
PQPKKKPL

SEQ ID NO: 431 - protein, 12 aa, synthetic construct
  REGION 1..12: note = Synthetic construct
  REGION 1..12: note = NLS - mouse c-abl IV (AA)
SALIKKKKKMAP

SEQ ID NO: 432 - protein, 5 aa, synthetic construct
  REGION 1..5: note = Synthetic construct
  REGION 1..5: note = NLS - influenza NS1 (AA)
DRLRR

SEQ ID NO: 433 - protein, 7 aa, synthetic construct
  REGION 1..7: note = Synthetic construct
  REGION 1..7: note = NLS - influenza NS1 (AA)
PKQKKRK

SEQ ID NO: 434 - protein, 10 aa, synthetic construct
  REGION 1..10: note = Synthetic construct
  REGION 1..10: note = NLS - hepatitis delta antigen (AA)
RKLKKKIKKL

SEQ ID NO: 435 - protein, 10 aa, synthetic construct
  REGION 1..10: note = Synthetic construct
  REGION 1..10: note = NLS - mouse Mx1 (AA)
REKKKFLKRR

SEQ ID NO: 436 - protein, 20 aa, synthetic construct
  REGION 1..20: note = Synthetic construct
  REGION 1..20: note = NLS - human poly(ADP-ribose) polymerase (AA)
KRKGDEVDGVDEVAKKKSKK

SEQ ID NO: 437 - protein, 17 aa, synthetic construct
  REGION 1..17: note = Synthetic construct
  REGION 1..17: note = NLS - glucocorticoid (AA)
RKCLQAGMNLEARKTKK

SEQ ID NO: 438 - protein, 22 aa, synthetic construct
  REGION 1..22: note = Synthetic construct
  REGION 1..22: note = SunTag GCN4 peptide (AA)
LLPKNYHLENEVARLKKLVGER

SEQ ID NO: 439 - protein, 5 aa, synthetic construct
  REGION 1..5: note = Synthetic construct
  REGION 1..5: note = GGSGG linker (AA)
GGSGG

SEQ ID NO: 440 - protein, 852 aa, synthetic construct
  REGION 1..852: note = Synthetic construct
  REGION 1..852: note = LSD1 (AA)
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 441-449 - skipped (9 entries)

(19) United States
(12) Patent Application Publication
(10) Pub. No.: US 2025/0134999 A1
(43) Pub. Date: May 1, 2025

(54) COMPOSITIONS, SYSTEMS, AND METHODS FOR PROGRAMMING T CELL PHENOTYPES THROUGH TARGETED GENE ACTIVATION

(71) Applicant: Tune Therapeutics, Inc., Seattle, WA (US)

(72) Inventors: Charles A. GERSBACH, Durham, NC (US); Tyler S. KLANN, Durham, NC (US); Akiko SEKI, Durham, NC (US); Nathaniel LAMBERT, Seattle, WA (US); Nathaniel CHARTRAND, Seattle, WA (US); Sheridan L. SWAN, Durham, NC (US)

(73) Assignee: Tune Therapeutics, Inc., Seattle, WA (US)

(21) Appl. No.: 18/728,436

(22) PCT Filed: Jan. 13, 2023
(86) PCT No.: PCT/US2023/060692

§ 371 Date: Jul. 11, 2024

Related U.S. Application Data
(60) Provisional application No. 63/299,903, filed on Jan. 14, 2022.
(60) Provisional application No. 63/299,904, filed on Jan. 14, 2022.

(51) Int. Cl. A61K 40/11; A61K 40/30; C12N 9/22; C12N 15/11

(52) U.S. Cl. CPC A61K 40/11; A61K 40/30; C12N 9/22; C12N 15/11; C12N 2310/20

(57) ABSTRACT

Provided in some aspects are epigenetic-modifying DNA-targeting systems, such as CRISPR-Cas/guide RNA systems, that bind to or target a target site in a gene or regulatory element thereof in a T cell. In some aspects, the provided epigenetic modifying DNA-targeting systems provided herein modulate a T cell phenotype or activity. In particular, the provided embodiments relate to the transcriptional activation of genes that promote a stem cell-like memory T (T_{SCM}) cell phenotype. In some aspects, also provided are compositions, polynucleotides, vectors, cells, and pluralities and combinations thereof, and methods and uses related to the provided epigenetic-modifying DNA-targeting systems, for example in modulating the phenotype in T cells including in connection with adoptive T cell therapy.

### CROSS-REFERENCE TO RELATED APPLICATIONS

This application claims priority from U.S. provisional application No. 63/299,903, filed Jan. 14, 2022, entitled “COMPOSITIONS, SYSTEMS, AND METHODS FOR PROGRAMMING T CELL PHENOTYPES THROUGH TARGETED GENE ACTIVATION,” and U.S. provisional application No. 63/299,904, filed Jan. 14, 2022, entitled “COMPOSITIONS, SYSTEMS, AND METHODS FOR PROGRAMMING T CELL PHENOTYPES THROUGH TARGETED GENE ACTIVATION,” the contents of which are incorporated by reference in their entireties.

### INCORPORATION BY REFERENCE OF SEQUENCE LISTING

The present application is being filed along with a Sequence Listing in electronic format. The Sequence Listing is provided as a file entitled 224742000340, created Jan. 13, 2023, which is 761,941 bytes in size. The information in the electronic format of the Sequence Listing is incorporated by reference in its entirety.

### FIELD

The present disclosure relates in some aspects to epigenetic-modifying DNA-targeting systems, such as CRISPR-Cas/guide RNA (gRNA) systems, that bind to or target a target site in a gene or regulatory element thereof in a T cell. In some aspects, the provided epigenetic modifying DNA-targeting systems of the present disclosure modulate a T cell phenotype or activity. In particular, the present disclosure relates to the transcriptional activation of genes that promote a stem cell-like memory T (T_{SCM}) cell-like phenotype. In some aspects, the present disclosure is directed to methods and uses related to the provided compositions, for example in modulating the phenotype of T cells including in connection with methods of adoptive T cell therapy.

### BACKGROUND

The administration of T cells targeting a specific antigen, also known as Adoptive Cell Therapy (ACT), is a promising approach for treating diseases such as cancer. However, current ACT treatments face challenges including suboptimal T cell function, expansion, and persistence. Therefore, there is a need for new and improved methods to overcome these challenges. The present disclosure addresses these and other needs.

### SUMMARY

In some embodiments, provided herein is an epigenetic-modifying DNA-targeting system comprising (a) a DNA-targeting domain capable of being targeted to a target site in a gene or regulatory DNA element thereof in a T cell; and (b) at least one effector domain capable of increasing transcription of the gene; wherein increased transcription of the gene promotes a stem cell-like memory T-cell phenotype. In some of any of the provided embodiments, the DNA-targeting system is not able to introduce a genetic disruption or a DNA break at or near the target site. In some of any of the provided embodiments, the DNA-targeting domain comprises a Clustered Regularly Interspaced Short Palindromic Repeats associated (Cas)-guide RNA (gRNA) combination comprising (a) a Cas protein or a variant thereof and (b) at least one gRNA; a zinc finger protein (ZFP); a transcription activator-like effector (TALE); a meganuclease; a homing endonuclease; or an I-SceI enzyme or a variant thereof, optionally wherein the DNA-targeting domain comprises a catalytically inactive variant of any of the foregoing. The DNA-targeting domain may comprise a Cas-gRNA combination comprising (a) a Cas protein or a variant thereof and (b) at least one gRNA.

In some embodiments, provided herein is an epigenetic-modifying DNA-targeting system comprising (a) a fusion protein comprising a Clustered Regularly Interspaced Short Palindromic Repeats associated (Cas) protein or variant thereof and at least one effector domain capable of increasing transcription of a gene is a T cell; and (b) at least one gRNA that targets the Cas protein or variant thereof of the fusion protein to a target site in the gene or regulatory DNA element thereof, wherein increased transcription of the gene promotes a stem cell-like memory T-cell phenotype.

In some of any of the provided embodiments of an epigenetic-modifying DNA-targeting system, the stem cell-like memory T cell phenotype comprises one or more cell-surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−, or combinations thereof. In some of any of the provided embodiments, the stem cell-like memory T cell phenotype comprises expression of CCR7 and/or CD27. In some of any of the provided embodiments, the stem cell-like memory T cell phenotype comprises expression of CCR7 and CD27. In some of any of the provided embodiments, the stem cell-like memory T cell phenotype is characterized by polyfunctional activity of the T cell to produce two or more cytokines following stimulation of the T cell with a stimulatory agent, optionally wherein the two or more cytokines are selected from among interferon-gamma (IFN-gamma), interleukin 2 (IL-2), and TNF-alpha.

In some of any of the provided embodiments of an epigenetic-modifying DNA-targeting system, the at least one gRNA is capable of complexing with the Cas protein or variant thereof, and targeting the Cas protein or the variant thereof to the target site. In some of any of the provided embodiments, the at least one gRNA comprises a gRNA spacer sequence that is capable of hybridizing to the target site or is complementary to the target site.

In some of any of the provided embodiments of an epigenetic-modifying DNA-targeting system, the Cas protein or a variant thereof is a Cas9 protein or a variant thereof. In some of any of the provided embodiments, the Cas protein or a variant thereof is a Cas12 protein or a variant thereof. In some of any of the provided embodiments, the Cas protein or a variant thereof is a variant Cas protein, wherein the variant Cas protein lacks nuclease activity or is a deactivated Cas (dCas) protein. In some of any of the provided embodiments, the variant Cas protein is a variant Cas9 protein that lacks nuclease activity or that is a deactivated Cas9 (dCas9) protein. In some of any of the provided embodiments, the Cas9 protein or a variant thereof is a Staphylococcus aureus Cas9 (SaCas9) protein or a variant thereof. In some of any of the provided embodiments, the variant Cas9 is a Staphylococcus aureus dCas9 protein (dSaCas9) that comprises at least one amino acid mutation selected from D10A and N580A, with reference to numbering of positions of SEQ ID NO: 405. In some of any of the provided embodiments, the variant Cas9 protein comprises the sequence set forth in SEQ ID NO: 406, or an amino acid sequence that has at least 90%, 91%, 92%, 93%, 94%, 95%, 96%, 97%, 98%, or 99% sequence identity thereto. In some of any of the provided embodiments, the Cas9 protein or variant thereof is a Streptococcus pyogenes Cas9 (SpCas9) protein or a variant thereof. In some of any of the provided embodiments, the variant Cas9 is a Streptococcus pyogenes dCas9 (dSpCas9) protein that comprises at least one amino acid mutation selected from D10A and H840A, with reference to numbering of positions of SEQ ID NO: 407. In some of any of the provided embodiments, the variant Cas9 protein comprises the sequence set forth in SEQ ID NO: 408, or an amino acid sequence that has at least 90%, 91%, 92%, 93%, 94%, 95%, 96%, 97%, 98%, or 99% sequence identity thereto.

In some of any of the provided embodiments of an epigenetic-modifying DNA-targeting system, the regulatory DNA element is an enhancer or a promoter. In some of any of the provided embodiments, the gene is a DNA-binding gene. In some of any of the provided embodiments, the gene is selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10.

In some of any of the provided embodiments of an epigenetic-modifying DNA-targeting system, the target site comprises the sequence set forth in any one of SEQ ID NOS: 1-132, a contiguous portion thereof of at least 14 nucleotides (nt), or a complementary sequence of any of the foregoing. In some of any of the provided embodiments, the at least one gRNA comprises a gRNA spacer sequence comprising the sequence set forth in SEQ ID NO:133-264, or a contiguous portion thereof of at least 14 nt. In some of any of the provided embodiments, the at least one gRNA further comprises the sequence set forth in SEQ ID NO:398. In some of any of the provided embodiments, the at least one gRNA comprises a gRNA that comprises the sequence set forth in any one of SEQ ID NOS: 265-396, optionally wherein the at least one gRNA is the gRNA set forth in any one of SEQ ID NOS: 265-396.

In some of any of the provided embodiments of an epigenetic-modifying DNA-targeting system, the gene is selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23. In some of any of the provided embodiments, the target site comprises the sequence set forth in any one of SEQ ID NOS: 1-28, a contiguous portion thereof of at least 14 nucleotides (nt), or a complementary sequence of any of the foregoing. In some of any of the provided embodiments, the at least one gRNA comprises a gRNA spacer sequence comprising the sequence set forth in SEQ ID NO:133-160, or a contiguous portion thereof of at least 14 nt. In some of any of the provided embodiments, the at least one gRNA further comprises the sequence set forth in SEQ ID NO:398. In some of any of the provided embodiments, the at least one gRNA comprises a gRNA that comprises the sequence set forth in any one of SEQ ID NOS: 265-292, optionally wherein the at least one gRNA is the gRNA set forth in any one of SEQ ID NOS: 265-292.

In some of any of the provided embodiments of an epigenetic-modifying DNA-targeting system, the gene is selected from the list consisting of: DDIT3, HES2, HKR1, PLAG1, PRDM4, TEF, TSHZ1, ZNF141, ZNF562, ZNF691, ZNF692, and ZSCAN5A. In some of any of the provided embodiments, the target site comprises the sequence set forth in any one of SEQ ID NOS: 1-12, a contiguous portion thereof of at least 14 nucleotides (nt), or a complementary sequence of any of the foregoing. In some of any of the provided embodiments, the at least one gRNA comprises a gRNA spacer sequence comprising the sequence set forth in SEQ ID NO: 133-144, or a contiguous portion thereof of at least 14 nt. In some of any of the provided embodiments, the at least one gRNA further comprises the sequence set forth in SEQ ID NO: 398. In some of any of the provided embodiments, the at least one gRNA comprises a gRNA that comprises the sequence set forth in any one of SEQ ID NOS: 265-276, optionally wherein the at least one gRNA is the gRNA set forth in any one of SEQ ID NOS: 265-276.

In some of any of the provided embodiments of an epigenetic-modifying DNA-targeting system, the gRNA spacer sequence is between 14 nt and 24 nt, or between 16 nt and 22 nt in length. In some of any of the provided embodiments, the gRNA spacer sequence is 18 nt, 19 nt, 20 nt, 21 nt or 22 nt in length. In some of any of the provided embodiments, the gRNA comprises modified nucleotides for increased stability.

In some of any of the provided embodiments of an epigenetic-modifying DNA-targeting system, the at least one effector domain induces, catalyzes, or leads to transcription activation, transcription co-activation, transcription elongation, or increased transcription of the gene. In some of any of the provided embodiments, the at least one effector domain induces transcription activation. In some of any of the provided embodiments, the at least one effector domain comprises at least one VP16 domain, or a VP16 tetramer (“VP64”) or a variant thereof. In some of any of the provided embodiments, the at least one effector domain comprises the sequence set forth in SEQ ID NO: 409, a portion thereof, or an amino acid sequence that has at least 90%, 91%, 92%, 93%, 94%, 95%, 96%, 97%, 98%, or 99% sequence identity to any of the foregoing. In some of any of the provided embodiments, at least one effector domain is selected from a p65 activation domain, a p300 domain, Rta, CBP, VPR, VPH, HSF1, a TET protein, optionally wherein the TET protein is TET1, an ERF protein, optionally wherein the ERF protein is ERF1 or ERF3, LSD1, SunTag, or a domain, portion, or variant of any of the foregoing. In some of any of the provided embodiments, at least one effector domain comprises a sequence selected from any one of SEQ ID NOS: 411-417, 438, or 440, or a domain thereof, a portion thereof, or an amino acid sequence that has at least 90%, 91%, 92%, 93%, 94%, 95%, 96%, 97%, 98%, or 99% sequence identity to any of the foregoing. In some of any of the provided embodiments, at least one effector domain is fused to the N-terminus, the C-terminus, or both the N-terminus and the C-terminus, of the DNA-targeting domain or a component thereof. In some of any of the provided embodiments, the DNA-targeting system further comprises one or more nuclear localization signals (NLS). In some of any of the provided embodiments, the DNA-targeting system further comprises one or more linkers connecting two or more of: the DNA-targeting domain, the at least one effector domain, and the one or more nuclear localization signals. In some of any of the provided embodiments, the fusion protein comprises the sequence set forth in SEQ ID NO:400, or an amino acid sequence that has at least 90%, 91%, 92%, 93%, 94%, 95%, 96%, 97%, 98%, or 99% sequence identity thereto.

In some of any of the provided embodiments of an epigenetic-modifying DNA-targeting system, increased transcription of the gene further promotes increased production of IL-2 by the T cell. In some of any of the provided embodiments, the epigenetic-modifying DNA-targeting system increases expression of the gene in a T cell by a log 2 fold-change of at or greater than 1.0. In some of any of the provided embodiments, the epigenetic-modifying DNA-targeting system reduces surface expression of a T cell exhaustion marker selected from the group consisting of PD-1, CTLA-4, TIM-3, TOX, LAG-3, BTLA, 2B4, CD160, CD39, VISTA, and TIGIT.

In some embodiments, provided herein is a guide RNA (gRNA) that binds a target site in a gene or regulatory DNA element thereof in a T cell, wherein increased transcription of the gene, when targeted by an epigenetic-modifying DNA-targeting system comprising the gRNA, promotes a stem cell-like memory T cell phenotype.

In some of any of the provided embodiments of a gRNA, the stem cell-like memory T cell phenotype comprises one or more cell-surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−. In some of any of the provided embodiments of a gRNA, the stem cell-like memory T cell phenotype comprises expression of CCR7 and/or CD27. In some of any of the provided embodiments of a gRNA, the stem cell-like memory T cell phenotype comprises expression of CCR7 and CD27. In some of any of the provided embodiments of a gRNA, the stem cell-like memory T cell phenotype is characterized by polyfunctional activity of the T cell to produce two or more cytokines following stimulation of the T cell with a stimulatory agent, optionally wherein the two or more cytokines are selected from among interferon-gamma (IFN-gamma), interleukin 2 (IL-2), and TNF-alpha.

In some of any of the provided embodiments of a gRNA, the gene is selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10.

In some embodiments, provided herein is a guide RNA (gRNA) that binds a target site in a gene or regulatory DNA element thereof in a T cell, wherein increased transcription of the gene, when targeted by an epigenetic-modifying DNA-targeting system comprising the gRNA, promotes a stem cell-like memory T cell phenotype, and wherein the gene is selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10.

In some of any of the provided embodiments of a gRNA, the target site is in a regulatory DNA element and the regulatory DNA element is an enhancer or a promoter. In some of any of the provided embodiments of a gRNA, the target site comprises the sequence set forth in any one of SEQ ID NOS: 1-132, a contiguous portion thereof of at least 14 nucleotides (nt), or a complementary sequence of any of the foregoing. In some of any of the provided embodiments, the gRNA comprises a gRNA spacer sequence comprising the sequence set forth in SEQ ID NO:133-264, or a contiguous portion thereof of at least 14 nt. In some of any of the provided embodiments, the gRNA further comprises the sequence set forth in SEQ ID NO:398. In some of any of the provided embodiments, the gRNA comprises a gRNA that comprises the sequence set forth in any one of SEQ ID NOS: 265-396, optionally wherein the gRNA is the gRNA set forth in any one of SEQ ID NOS: 265-396.

In some of any of the provided embodiments of a gRNA, the gene is selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23. In some of any of the provided embodiments of a gRNA, the target site comprises the sequence set forth in any one of SEQ ID NOS: 1-28, a contiguous portion thereof of at least 14 nucleotides (nt), or a complementary sequence of any of the foregoing. In some of any of the provided embodiments, the gRNA comprises a gRNA spacer sequence comprising the sequence set forth in SEQ ID NO:133-160, or a contiguous portion thereof of at least 14 nt. In some of any of the provided embodiments, the gRNA further comprises the sequence set forth in SEQ ID NO:398. In some of any of the provided embodiments, the gRNA comprises a gRNA that comprises the sequence set forth in any one of SEQ ID NOS: 265-292, optionally wherein the gRNA is the gRNA set forth in any one of SEQ ID NOS: 265-292.

In some of any of the provided embodiments of a gRNA, the gene is selected from the list consisting of: DDIT3, HES2, HKR1, PLAG1, PRDM4, TEF, TSHZ1, ZNF141, ZNF562, ZNF691, ZNF692, and ZSCAN5A. In some of any of the provided embodiments of a gRNA, the target site comprises the sequence set forth in any one of SEQ ID NOS: 1-12, a contiguous portion thereof of at least 14 nucleotides (nt), or a complementary sequence of any of the foregoing. In some of any of the provided embodiments, the gRNA comprises a gRNA spacer sequence comprising the sequence set forth in SEQ ID NO:133-144, or a contiguous portion thereof of at least 14 nt. In some of any of the provided embodiments, the gRNA further comprises the sequence set forth in SEQ ID NO:398. In some of any of the provided embodiments, the gRNA comprises a gRNA that comprises the sequence set forth in any one of SEQ ID NOS: 265-276, optionally wherein the gRNA is the gRNA set forth in any one of SEQ ID NOS: 265-276.

In some of any of the provided embodiments, the gRNA spacer sequence is between 14 nt and 24 nt, or between 16 nt and 22 nt in length. In some of any of the provided embodiments, the gRNA spacer sequence is 18 nt, 19 nt, 20 nt, 21 nt or 22 nt in length. In some of any of the provided embodiments, the gRNA comprises modified nucleotides for increased stability. In some of any of the provided embodiments, the gRNA is capable of complexing with a Cas protein or variant thereof. In some of any of the provided embodiments, the gRNA is capable of hybridizing to the target site or is complementary to the target site.

In some embodiments, provided herein is a CRISPR Cas-guide RNA (gRNA) combination comprising: (a) a Clustered Regularly Interspaced Short Palindromic Repeats associated (Cas) protein or variant thereof; and (b) at least one of any of the gRNAs provided herein that targets the Cas protein or variant thereof to a target site in a gene or regulatory DNA element thereof in a T cell.

In some of any of the provided embodiments of a CRISPR Cas-gRNA combination, the Cas protein or a variant thereof is a Cas9 protein or a variant thereof. In some of any of the provided embodiments of a CRISPR Cas-gRNA combination, the Cas protein or a variant thereof is a variant Cas protein, wherein the variant Cas protein lacks nuclease activity or is a deactivated Cas (dCas) protein. In some of any of the provided embodiments of a CRISPR Cas-gRNA combination, the variant Cas protein is a variant Cas9 protein that lacks nuclease activity or that is a deactivated Cas9 (dCas9) protein. In some of any of the provided embodiments of a CRISPR Cas-gRNA combination, the Cas9 protein or a variant thereof is a Staphylococcus aureus Cas9 (SaCas9) protein or a variant thereof. In some of any of the provided embodiments of a CRISPR Cas-gRNA combination, the variant Cas9 is a Staphylococcus aureus dCas9 protein (dSaCas9) that comprises at least one amino acid mutation selected from D10A and N580A, with reference to numbering of positions of SEQ ID NO: 405. In some of any of the provided embodiments of a CRISPR Cas-gRNA combination, the variant Cas9 protein comprises the sequence set forth in SEQ ID NO: 406, or an amino acid sequence that has at least 90%, 91%, 92%, 93%, 94%, 95%, 96%, 97%, 98%, or 99% sequence identity thereto. In some of any of the provided embodiments of a CRISPR Cas-gRNA combination, the Cas9 protein or variant thereof is a Streptococcus pyogenes Cas9 (SpCas9) protein or a variant thereof. In some of any of the provided embodiments of a CRISPR Cas-gRNA combination, the variant Cas9 is a Streptococcus pyogenes dCas9 (dSpCas9) protein that comprises at least one amino acid mutation selected from D10A and H840A, with reference to numbering of positions of SEQ ID NO: 407. In some of any of the provided embodiments of a CRISPR Cas-gRNA combination, the variant Cas9 protein comprises the sequence set forth in SEQ ID NO: 408, or an amino acid sequence that has at least 90%, 91%, 92%, 93%, 94%, 95%, 96%, 97%, 98%, or 99% sequence identity thereto.

In some embodiments, provided herein is a polynucleotide encoding any of the DNA-targeting systems, any of the fusion proteins of the DNA-targeting systems, any of the gRNAs, or any of the CRISPR Cas-gRNA combinations provided herein, or a portion or a component of any of the foregoing.

In some embodiments, provided herein is a plurality of polynucleotides encoding any of the DNA-targeting systems, any of the fusion proteins of the DNA-targeting systems, any of the gRNAs, or any of the CRISPR Cas-gRNA combinations provided herein, or a portion or a component of any of the foregoing.

In some embodiments, provided herein is a vector comprising any of the polynucleotides provided herein. In some embodiments, provided herein is a vector comprising any of the pluralities of polynucleotides provided herein. In some of any of the provided embodiments, the vector is a viral vector. In some of any of the provided embodiments, the vector is an adeno-associated virus (AAV) vector. In some of any of the provided embodiments, the vector is selected from among AAV1, AAV2, AAV3, AAV4, AAV5, AAV6, AAV7. AAV8, and AAV9. In some of any of the provided embodiments, the vector is a lentiviral vector. In some of any of the provided embodiments, the vector is a non-viral vector. In some of any of the provided embodiments, the non-viral vector is selected from: a lipid nanoparticle, a liposome, an exosome, or a cell penetrating peptide. In some of any of the provided embodiments, the vector exhibits immune cell or T-cell tropism. In some of any of the provided embodiments, the vector comprises one vector, or two or more vectors.

In some embodiments, provided herein is a modified T cell comprising any of the DNA-targeting systems, any of the gRNAs, any of the CRISPR Cas-gRNA combinations, any of the polynucleotide, any of the pluralities of polynucleotides, or any of the vectors provided herein, or a portion or a component of any of the foregoing. In some of any of the provided embodiments, the modified T cell comprises an epigenetic or phenotypic modification resulting from being contacted any of the DNA-targeting systems, any of the gRNAs, any of the CRISPR Cas-gRNA combinations, any of the polynucleotide, any of the pluralities of polynucleotides, or any of the vectors provided herein, or a portion or a component of any of the foregoing. In some of any of the provided embodiments, the modified T cell exhibits increased transcription of one or more genes that promote a stem cell-like memory T-cell phenotype, in comparison to a comparable unmodified T cell. In some of any of the provided embodiments of a modified T cell, the one or more genes are selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10. In some of any of the provided embodiments of a modified T cell, the one or more genes are selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT. HMGN3, PATZ1, PRDM8, SOX21, TBX15, ZBED5, ZBTB7A, ZNF219. ZNF319, ZNF470, ZNF816, and ZSCAN23. In some of any of the provided embodiments of a modified T cell, the one or more genes are selected from the list consisting of: DDIT3, HES2, HKR1, PLAG1, PRDM4, TEF, TSHZ1, ZNF141, ZNF562, ZNF691, ZNF692, and ZSCAN5A.

In some of any of the provided embodiments of a modified T cell, the transcription is increased by at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold.

In some of any of the provided embodiments, the modified T cell exhibits a stem cell-like memory T-cell phenotype. In some of any of the provided embodiments of a modified T cell, the stem cell-like memory T cell phenotype comprises expression of CCR7 and/or CD27.

In some of any of the provided embodiments of a modified T cell, the stem cell-like memory T cell phenotype comprises expression of CCR7 and CD27. In some of any of the provided embodiments of a modified T cell, the stem cell-like memory T cell phenotype comprises one or more cell-surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−. In some of any of the provided embodiments, the modified T cell is capable of a stronger and/or more persistent immune response, in comparison to a comparable unmodified T cell. In some of any of the provided embodiments, the modified T cell is characterized by polyfunctional activity of the T cell to produce two or more cytokines following stimulation of the T cell with a stimulatory agent, optionally wherein the two or more cytokines are selected from among interferon-gamma (IFN-gamma), interleukin 2 (IL-2), and TNF-alpha.

In some of any of the provided embodiments, the modified T cell is derived from a cell from a subject. In some of any of the provided embodiments, the modified T cell is derived from a primary T cell. In some of any of the provided embodiments, the modified T cell is derived from a T cell progenitor, a pluripotent stem cell, or an induced pluripotent stem cell. In some of any of the provided embodiments, the modified T cell further comprises an engineered T cell receptor (eTCR) or chimeric antigen receptor (CAR).

In some aspects, provided herein is a method of increasing the transcription of one or more genes in a T cell, the method comprising introducing into a T cell any of the DNA-targeting systems, any of the gRNAs, any of the CRISPR Cas-gRNA combinations, any of the polynucleotides, any of the pluralities of polynucleotides, or any of the vectors provided herein, or a portion or a component of any of the foregoing. In some of any of the provided methods, the one or more genes is a gene epigenetically modified by the DNA-targeting system. In some of any of the provided methods, the transcription of the one or more genes is increased in comparison to a comparable T cell not subjected to the method. In some of any of the provided methods, the transcription of the one or more genes is increased by at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold. In some of any of the provided methods, the increased transcription of the one or more genes promotes a stem cell-like memory T cell phenotype in the T cell.

In some aspects, provided herein is a method of promoting a stem cell-like memory T cell phenotype in a T cell, the method comprising introducing into the T cell any of the DNA-targeting systems, any of the gRNAs, any of the CRISPR Cas-gRNA combinations, any of the polynucleotides, any of the pluralities of polynucleotides, or any of the vectors provided herein, or a portion or a component of any of the foregoing. In some of any of the provided methods, the stem cell-like memory T cell phenotype comprises one or more cell-surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−. In some of any of the provided methods, the stem cell-like memory T cell phenotype comprises expression of CCR7 and/or CD27. In some of any of the provided methods, the stem cell-like memory T cell phenotype is characterized by polyfunctional activity of the T cell to produce two or more cytokines following stimulation of the T cell with a stimulatory agent, optionally wherein the two or more cytokines are selected from among interferon-gamma (IFN-gamma), interleukin 2 (IL-2), and TNF-alpha.

In some of any of the provided methods, the T cell is a T cell in a subject and the method is carried out in vivo. In some of any of the provided methods, the T cell is a T cell from a subject, or derived from a cell from the subject, and the method is carried out ex vivo. In some of any of the provided methods, the T cell is a primary T cell. In some of any of the provided methods, the T cell is derived from a T cell progenitor, a pluripotent stem cell, or an induced pluripotent stem cell.

In some embodiments, provided herein is a modified T cell produced by any of the methods provided herein.

In some aspects, provided herein is a method of cell therapy for treating a disease in a subject in need thereof, comprising administering to the subject a cellular composition that comprises any of the modified T cells provided herein. In some of any of the provided methods, the modified T cell is obtained from or derived from a cell from said subject in need thereof. In some of any of the provided methods, the subject is a first subject, and the modified T cell is obtained from or derived from a cell from a second subject. In some of any of the provided methods, the subject in need thereof is a human. In some of any of the provided methods, the administered modified T cell exhibits a stronger and/or more persistent immune response in the subject, in comparison to a comparable unmodified T cell. In some of any of the provided methods, the subject has or is suspected of having a disease, condition, or disorder, optionally wherein the disease, condition, or disorder is cancer, viral infection, autoimmune disease, or graft-versus-host disease, or the subject has undergone or is expected to undergo organ transplantation. In some of any of the provided methods, the subject has or is suspected of having cancer.

In some embodiments, provided herein is a pharmaceutical composition comprising any of the modified T cells provided herein. In some embodiments, provided herein is a pharmaceutical composition comprising any of the DNA-targeting systems, any of the gRNAs, any of the CRISPR Cas-gRNA combinations, any of the polynucleotides, any of the pluralities of polynucleotides, or any of the vectors provided herein, or a portion or a component of any of the foregoing. In some of any of the provided embodiments, the pharmaceutical composition is for use in treating a disease, condition, or disorder in a subject. In some of any of the provided embodiments, the pharmaceutical composition is for use in the manufacture of a medicament for treating a disease, condition, or disorder in a subject. In some of any of the provided embodiments, the subject has or is suspected of having a disease, condition, or disorder, optionally wherein the disease, condition, or disorder is cancer, viral infection, autoimmune disease, or graft-versus-host disease, or the subject has undergone or is expected to undergo organ transplantation. In some of any of the provided embodiments, the subject has or is suspected of having cancer. In some of any of the provided embodiments, the pharmaceutical composition is to be administered to the subject in vivo. In some of any of the provided embodiments, the subject is a first subject, and the pharmaceutical composition is to be administered ex vivo to T cells from the first subject, or to T cells from a second subject. In some of any of the provided embodiments, following administration to T cells from the first subject or second subject, the T cells are administered to the first subject. In some of any of the provided embodiments, following administration of the pharmaceutical composition, the expression of one or more genes is increased in T cells of the subject. In some of any of the provided embodiments, following administration of the pharmaceutical composition to the T cells from the first or second subject, the expression of one or more genes is increased in the T cells. In some of any of the provided embodiments of a pharmaceutical composition, the one or more genes are selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10. In some of any of the provided embodiments of a pharmaceutical composition, the one or more genes are selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23. In some of any of the provided embodiments of a pharmaceutical composition, the one or more genes are selected from the list consisting of: DDIT3, HES2, HKR1, PLAG1, PRDM4, TEF, TSHZ1, ZNF141, ZNF562, ZNF691, ZNF692, and ZSCAN5A.

In some aspects, provided herein is a method for treating a disease in a subject in need thereof, comprising administering to the subject any of the DNA-targeting systems, any of the gRNAs, any of the CRISPR Cas-gRNA combinations, any of the polynucleotides, any of the pluralities of polynucleotides, any of the vectors, any of the modified T cells, or any of the pharmaceutical compositions provided herein, or a portion or a component of any of the foregoing.

### BRIEF DESCRIPTION OF THE DRAWINGS

FIGS. 1A-1C show details of the gRNA screen as described in Example 1. FIG. 1A shows a timeline of the procedures carried out for the screen. FIG. 1B shows expression of cell CD90 in unenriched and CD90-enriched T cells, as assessed by flow cytometry. FIG. 1C shows expression of CCR7 and CD27 in pre-sorted cells and the CCR7+/CD27+ sorted population, as assessed by flow cytometry.

FIG. 2 shows a volcano plot of results from sequencing analysis in the gRNA screen as described in Example 1. Each point represents a single gRNA; circles represent gene-targeted gRNAs and triangles represent control gRNAs. x-axis represents log 2 fold change of gRNA abundance in the CCR7+/CD27+ population in comparison to the unsorted population. y-axis represents statistical significance of gRNA enrichment or depletion in-log 10 adjusted p-value. gRNAs were significantly depleted (left) or enriched (right) in the CCR7+/CD27+ population, based on a false discovery rate (FDR) of adjusted p-value <0.1 (significance threshold indicated by dashed horizontal line).

### DETAILED DESCRIPTION

Provided herein is an epigenetic-modifying DNA-targeting system, said DNA-targeting system comprising a fusion protein comprising: (a) a DNA-targeting domain capable of being targeted to a target site in a gene or regulatory DNA element thereof in a T cell; and (b) at least one effector domain capable of increasing transcription of the gene; wherein increased transcription of the gene promotes a stem cell-like memory T-cell (Tscm) phenotype. Hence, in some embodiments, the DNA-targeting systems are synthetic transcription factors that are able to modulate, such as increase or upregulate, transcription of a gene in a targeted manner. In some embodiments, the DNA-targeting domain of the DNA-targeting system is a nuclease-inactive Clustered Regularly Interspaced Short Palindromic Repeats associated (Cas) protein or variant thereof complexed with a guide RNA (gRNA). Also provided are gRNA for targeting to a target site in a gene or a regulatory DNA element thereof in a T cell, wherein the gene is one in which increased transcription of the gene promotes a Tscm phenotype, as well as CRISRP-Cas/gRNA combinations thereof. Also provided herein are polynucleotides encoding the DNA-targeting system or the fusion protein of the DNA-targeting system, and vectors and cells containing the same. Also provided herein are methods of using the epigenetic-modifying DNA-targeting system for modulating transcription or phenotype of T cells and the resulting modified cells. The provided embodiments relate to compositions and methods for promoting a Tscm phenotype in a T cell or in one or more T cells in a population by epigenetically modifying target sites in one or more target genes. In some embodiments, the methods can be used in connection with T cell therapies, such as in connection with adoptive T cell therapies.

The administration of T cells targeting a specific antigen, also known as Adoptive Cell Therapy (ACT), is a promising approach for treating diseases such as cancer. However, current ACT treatments face challenges including suboptimal T cell function, expansion, and persistence. Furthermore, the persistence and functionality of the transferred T cells can significantly differ between different T cell subsets and among T cells from different patients. Recent clinical trials for ACT suggest that the ability to persist long term in the circulation is dependent on the differentiation stage of the T cell, including the ability to retain a network of transcription factors and metabolic regulators (Pilipow K., et. al., Journal of Clinical Investigation Insight 2018; 3 (18): e122299). The T cells transferred into the patient are often terminally differentiated and therefore fail to persist in the long term, ultimately limiting effective anti-tumor response.

Strategies to mitigate these challenges and enhance the persistence, expansion, and anti-tumor activity of chimeric antigen receptor (CAR) engineered T cells have been tested in preclinical and clinical settings. For instance, strategies for optimizing ex vivo T cell culture conditions, including the addition of cytokines during manufacturing (Besser M. J., Cytotherapy 2009; 11 (2): 206-17), expression of cytokines and/receptors by the CAR T cells (Krenciute G., Cancer Immunol Res. 2017 07; 5 (7): 571-581), use of pharmacological inhibitors during expansion to inhibit signaling pathways such as AKT (Urak R. et. al., Journal of Immunotherapy Cancer 2017 Mar. 21; 5:26) or PI3K (Peterson C. T et. al., Blood Advances 2018 Feb. 13; 2 (3): 210-223), immune-depletion and checkpoint blockade (Cherkassky L. et. al., Journal of clinical investigation 2016 Aug. 1; 126 (8): 3130-44) have been so far explored. However, existing strategies have not been entirely satisfactory. In some cases, concerns regarding cytokine-induced toxicity or the emergence of lymphoproliferative diseases as a result of the above-mentioned strategies have raised questions for alternative approaches.

Clinical and preclinical results also have established that certain T cell subsets with a less differentiated phenotype akin to naïve-like T cells also may lead to greater persistence and functionality. The memory T cell compartment has been conventionally divided into two subsets based on the expression of CD62L and CCR7 (Sallusto F., et. al., Nature 1999 Oct. 14,401 (66754): Jul. 8, 2012). Central memory T cells (Tcm) express high levels of CD62L and CCR7 and are naive-like T cells, while effector memory T cells (Tem) do not express CD62L nor CCR7 and are committed progenitor cells that undergo terminal differentiation. A specialized subset within the naïve-like T cell (Tn) compartment exists that harbors superior multipotent capacities to regenerate central memory (Tcm), effector memory (Tem), and effector T cells (Gattinoni L., et. al., Nature Medicine 2009 July; 15 (7): 808-13, Gattinoni L., et. al., Nature Medicine 2012; 17 (10): 1290-1297). This early differentiated stem cell memory T (Tscm) cell subset expresses CD45RO−, CCR7+, CD45RA+, CD62L+, CD27+, CD28+ and IL-7Rα+ common to the naïve-like T cell compartment and in addition expresses increased levels of CD95. IL-2RB, CXCR3, and LFA-1 with distinctive attributes of conventional memory T cells. Tscm cells represent the least differentiated T-cell memory subset that retains a network of transcription factors and metabolic regulators, responsible for their multipotency and a heightened capacity to self-renew (Pilipow K., et. al., Journal of Clinical Investigation Insight 22018; 3 (18): e122299). Furthermore, the expression of the lymphoid-homing receptor CCR7 facilitates superior migration to secondary lymphoid organs, such as the spleen, which translates into longer persistence and constant replenishment of the circulating T cell pool.

Preclinical studies using adoptive transfer in tumor-bearing mice suggest that Tscm cells have enhanced proliferative, survival, and long-lasting anti-tumor capacities compared with the conventional Tem and Tem cells (Gattinoni L., et. al., Nature Medicine 2012; 17 (10): 1290-1297). Upon antigenic stimulation and TCR activation the Tscm cells clonally expand and display effector functions. Recent clinical trials using adoptive transfer of autologous T cells expressing CD19-specific chimeric antigen receptors have shown signs of complete regression in patients with lymphoid malignancies (#NCT00586391 and #NCT00709033), and analysis of the patients indicated that the T memory stem cell (or Tscm) subset within the infusion product expressing CD8+CD45RA+CCR7+ was responsible for the in vivo expansion resulting in complete tumor regression (Xu Y., et al., Blood 2014 Jun. 12; 123 (24): 3750-9).

Tscm cells are rare in the total pool of circulating T cells and therefore there is a need for increasing their numbers for use in ACT. Studies on preclinical mouse models have highlighted the significance of increasing the frequency of these Tscm cells in producing greater anti-tumor activity. In one such study, following repetitive encounters with the antigen and cytokine-mediated expansion, the anti-tumor activity of the Tscm cells correlated with enhanced persistence and increased resistance to cell death (Xu Y., et al., Blood 2014 Jun. 12; 123 (24): 3750-9). However, while the frequency of the CD8+CD45RA+CCR7+ subset doubled, the numbers of CAR+CD4+CD45RA+CCR7+ subset remained low, raising the need for better approaches for improving the Tsem cell numbers in the CAR-T product. Other studies also have demonstrated that CAR-T cells with Tsem properties mediate robust, long-lasting anti-tumor responses (Sabatino M., Blood 2016; 128 (4): 519-528, Capuis A. G., et. al., Proc Natl Acad Scie 2012 Mar. 20; 109 (12): 4592-97, Fraietta J. A., Nature medicine 2018; 24:563-571). However, the rareness of the Tscm cells within the circulating T cell population is a significant hurdle to their use in ACT, such as CAR-T therapy.

Studies indicate that changes in the epigenetic environment may contribute to favorable outcomes associated with CAR T cell therapy. For instance, an extensive analysis of a patient with advanced refractory CLL undergoing CAR-T therapy showed that a biallelic dysfunction in the epigenetic modifier TET2 gene lead to complete remission (Fraietta J. A., et. al.). The progeny of a single CAR T-cell with the epigenetic modification was sufficient to mediate potent anti-tumor effects. However, it is not known in what ways a CAR T cell may be epigenetically modified to improve CAR T cell persistence and activity. There is thus a need for targeted approaches for epigenetic modulation of T cells.

The provided embodiments relate to identification of genomic locations that are epigenetically modified in a T cell that has a Tscm phenotype, as demonstrated by assessment for cells surface positive for the exemplary Tscm markers CD27 and CCR7. Targeting such genomic locations would promote or increase the differentiation fate of T cells as Tscm. Thus, the provided embodiments herein relate to identification of target genes that can be epigenetically modified to promote (i.e. increase) a Tscm phenotype of T cells. In aspects, the provided embodiments include introducing into a T cell epigenetic modifications using effector domains that are activators of transcription (i.e. transcription activation domains), which can be directed to regions of a target gene (e.g. regulatory elements such as promoters or enhancers) for transcriptional activation and increased expression of the target gene. For instance, provided herein are epigenetic-modifying DNA binding systems combining a DNA-targeting domain (e.g. a dCas and gRNA combination) and an effector domain, in which the effector domain is able to target a target site of the gene or a regulatory element thereof to specifically increase transcription of the gene by epigenetic regulation. In provided embodiments, transcriptional activation, leading to increased or upregulated gene expression, reprograms the cell to a Tscm phenotype. Moreover, the epigenetic modification of the cell does not modify DNA at the sequence level, thereby avoiding safety concerns with gene editing approaches. The ability to epigenetically control the differentiation fate of T cells provides an advantageous approach for increasing the percentage or number of T cells in a population of T cells that have a Tscm phenotype, but without having to specifically select for (i.e. isolate) the population of Tscm cells, genetically engineer or edit the cell, or otherwise alter ex vivo T cell manufacturing to limit their differentiation to more mature T cell subsets. As a result, what was once a rare population of T cells can be efficiently increased to provide for a highly enriched population of Tscm T cells to sufficient numbers for cell therapy methods, including ACT.

Thus, provided herein is an epigenetic-modifying DNA-targeting system that binds to a target site in a gene or regulatory DNA element thereof in a T cell, such as any described herein, in which the DNA-targeting system includes a DNA binding domain and at least one effector domain capable of increasing transcription of the gene. In some embodiments, the DNA binding domain is a nuclease-inactive Clustered Regularly Interspaced Short Palindromic Repeats associated (Cas) protein or variant thereof, such as a dead Cas (dCas, e.g. dCas9), and the DNA-targeting system further includes at least one gRNA that can complex with the Cas and has a gRNA spacer sequence that is capable of hybridizing to the target site of the gene. In provided embodiments, the provided epigenetic-modifying DNA-targeting system increases transcription of the gene and thereby promotes a Tesm cell phenotype. Also provided herein are related gRNA, including Cas/gRNA combinations, polynucleotides, compositions and methods involving or related to the epigenetic-modifying DNA targeting system. The provided embodiments can be used to target genes that when transcriptionally activated can vastly facilitate the enrichment of a Tscm CCR7+/CD27+ TSCM cell-like phenotypes. This approach offers substantial clinical solutions to circumvent the problems with T cell persistence, suboptimal functionality, and/or exhaustion.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. DNA-TARGETING SYSTEMS

In some embodiments, provided are DNA-targeting systems capable of specifically targeting a target site in a gene (also called a target gene herein) or DNA regulatory element thereof, and increasing transcription of the gene. In provided embodiments, the DNA-targeting systems include a DNA-targeting domain that bind to a target site in a gene or regulatory DNA element thereof. In provided embodiments, the DNA-targeting systems additionally include at least one effector domain that is able to epigenetically modify one or more DNA bases of the gene or regulatory element thereof, in which the epigenetic modification results in an increase in transcription of the gene (e.g. activates transcription or increases transcription of the gene compared to the absence of the DNA-targeting system). Hence, the terms DNA-targeting system and epigenetic-modifying DNA targeting system may be used herein interchangeably. In some embodiments, the DNA-targeting systems includes a fusion protein comprising (a) a DNA-targeting domain capable of being targeted to the target site; and (b) at least one effector domain capable of increasing transcription of the gene. For instance, the at least one effector domain is a transcription activation domain.

In aspects of the provided embodiments, a DNA-targeting system provided herein targets a gene or a regulatory element thereof to increase transcription of the gene in an immune cell, such as a T cell, in which the increased transcription modulates one or more activities or functions of the immune cells, such as a phenotype of the immune cell. In some embodiments, increased transcription of the gene results in an increase in expression of the gene, i.e. increased gene expression, in the immune cell. In some embodiments, increased transcription of the gene, such as increased gene expression, promotes a stem cell-like memory T (T_{SCM}) cell phenotype, or a T_{SCM} cell-like phenotype.

In some aspects, the cell is an immune cell, such as a lymphocyte (e.g. a T cell, B cell, or Natural Killer (NK) cell). In some aspects, the cell is a T cell. For instance, provided herein is a DNA-targeting system that targets a gene or a regulatory element thereof to increase transcription of the gene in a T cell, in which the increased transcription modulates one or more activities or functions of the T cell, such as a phenotype of the T cell. In some embodiments, increased transcription of the gene results in an increase in expression of the gene, i.e. increased gene expression, in the T cell. In some aspects the cell is a primary T cell. In some aspects, the cell is a cell that can be differentiated into a T cell, such as a T cell progenitor, pluripotent stem cell, or induced pluripotent stem cell. In some aspects, the cell is an engineered T cell, such as a T cell comprising a recombinant T cell receptor or chimeric antigen receptor (CAR).

In some aspects, the cell is from a human subject. In some aspects the cell is a cell in a subject (i.e. a cell in vivo) or from a subject (i.e. a cell ex vivo).

In some embodiments, the DNA-targeting domain comprises or is derived from a CRISPR associated (Cas) protein, zinc finger protein (ZFP), transcription activator-like effectors (TALE), meganuclease, homing endonuclease, I-SceI enzyme, or variants thereof. In some embodiments, the DNA-targeting domain comprises a catalytically inactive (e.g. nuclease-inactive or nuclease-inactivated) variant of any of the foregoing. In some embodiments, the DNA-targeting domain comprises a deactivated Cas9 (dCas9) protein or variant thereof that is a catalytically inactivated so that it is inactive for nuclease activity and is not able to cleave the DNA.

In some embodiments, the DNA-targeting domain comprises or is derived from a Cas protein or variant thereof, such as a nuclease-inactive Cas or dCas (e.g. dCas9, and the DNA-targeting system comprises one or more guide RNAs (gRNAs). In some embodiments, the gRNA comprises a spacer sequence that is capable of targeting and/or hybridizing to the target site. In some embodiments, the gRNA is capable of complexing with the Cas protein or variant thereof. In some aspects, the gRNA directs or recruits the Cas protein or variant thereof to the target site.

In some embodiments, the effector domain comprises a transcription activation domain, and/or is capable of increasing transcription of the gene. In some embodiments, the effector domain induces, catalyzes or leads to transcription activation, transcription co-activation, or transcription elongation. In some aspects, the effector domain is selected from VP64, p65, Rta, p300, CBP, VPR, VPH, Rta, p300, HSF1, a TET protein (e.g. TET1), an ERF protein (e.g. ERF1, ERF3), LSD1, SunTag, a partially or fully functional fragment or domain thereof, or a combination of any of the foregoing. In some embodiments, the effector domain is VP64.

In some embodiments, the fusion protein of the DNA-targeting system comprises dCas9-VP64.

Exemplary components and features of the DNA-targeting systems are provided below in the following subsections.

### A. Target Genes and Target Sites

In some embodiments, the target gene is a gene in which increased expression of the gene regulates a cellular phenotype. In some embodiments, the target gene is capable of regulating a phenotype in a T cell. In some embodiments, the target gene is capable of regulating T cell differentiation. In some embodiments, increased transcription of the gene, such as increased gene expression, promotes a stem cell-like memory T (T_{SCM}) cell phenotype, or a T_{SCM} cell-like phenotype.

In some aspects, the T_{SCM} cell phenotype is one that is characterized by a cell surface phenotype of the cells. In some embodiments, the T_{SCM} cell phenotype comprises expression of one or more cell-surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−, or any combination thereof. In some aspects, the T_{SCM} cell phenotype comprises expression of CCR7+ and/or CD27+. In some aspects, the T_{SCM} cell phenotype comprises expression of CCR7+ and CD27+.

It is understood that embodiments of provided epigenetic-modifying DNA-targeting systems are not limited to modulating expression of target genes in T cells, but may also be used to modulate any one or more of the target gene as described herein in any lymphoid cell. In addition to T cells, lymphoid cells can include NK cells, NKT cells, any cells that have been differentiated from stem cells into such lymphoid cells and/or have been differentiated from progenitor cells, such as common lymphoid progenitors (CLPs). In some embodiments, the lymphoid cells are differentiated from stem cells, such as hematopoietic stem or progenitor cells, or progenitor cells. In some embodiments, the lymphoid cells are trans-differentiated from a non-pluripotent cell of non-hematopoietic lineage.

In some embodiments, the lymphoid cell for modulation is an isolated or enriched population of lymphoid immune cells, such as a population isolated or enriched in T, NK and/or NKT cells. In some embodiments, the cells for modulation are isolated or enriched T cells. In some embodiments, the cells for modulation are isolated or enriched NK cells. In some embodiments, the cells for modulation are isolated or enriched NK T cells. In some embodiments, isolated or enriched populations or subpopulations of immune cells comprising T, NK, and/or NKT cells for modulation can be obtained from a unit of blood using any number of techniques known to the skilled artisan, such as Ficoll™ separation. In one embodiment, T, NK or NKT cells from the circulating blood of an individual are obtained by apheresis and separated from other nucleated white blood cells, red blood cells and platelets, such as by Ficoll™ separation or affinity-based selection. In some embodiments, the cells are primary cells. In some embodiments, the primary cells are isolated or enriched from a peripheral blood sample from a subject, such as a human subject.

In some embodiments, the lymphoid cells for modulation is differentiated in vitro from a stem cell or progenitor cell. In some embodiments, the lymphoid cells, such as T, NK or NKT cells or lineages thereof, can be differentiated from a stem cell, a hematopoietic stem or progenitor cell (HSC), or a progenitor cell. The progenitor cell can be a CD34+ hemogenic endothelium cell, a multipotent progenitor cell, a T cell progenitor, an NK cell progenitor, or an NKT cell progenitor. In some embodiments, the progenitor cells is a lymphoid progenitor cells such as a common lymphoid progenitor cell, early thymic progeniotor cells, pre-T cell progenitor cells, pre-NK progenitor cell, T progenitor cell, NK progenitor cell or NKT progenitor cell. The stem cell can be a pluripotent stem cell, such as induced pluripotent stem cells (iPSCs) and embryonic stem cells (ESCs). The iPSC is a non-naturally occurring reprogrammed pluripotent cell. Once the cells of a subject have been reprogrammed to a pluripotent state, the cells can then be programmed or differentiated to a desired cell type or subtypes, such as T, NK, or NKT cells.

In some embodiments, the iPSC is differentiated to a T, NK or NKT cells by a multi-stage differentiation platform wherein cells from various stages of development can be induced to assume a hematopoietic phenotype, ranging from mesodermal stem cells, to fully differentiated T, NK or NKT cells (See e.g. U.S. Applications 62/107,517 and 62/251,016, the disclosures of which are incorporated herein in their entireties).

In some embodiments, the population or subpopulation of lymphoid cells is trans-differentiated in vitro from a non-pluripotent cell of non-hematopoietic fate to a hematopoietic lineage cell or from a non-pluripotent cell of a first hematopoietic cell type to a different hematopoictic cell type, which can be a T, NK, or NKT progenitor cell or a fully differentiated specific type of immune cell, such as T, NK, or NKT cell (See e.g. U.S. Pat. No. 9,376,664 and U.S. application Ser. No. 15/072,769, the disclosure of which is incorporated herein in their entirety). In some embodiments, the non-pluripotent cell of non-hematopoietic fate is a somatic cell, such as a skin fibroblast, an adipose tissue-derived cell and a human umbilical vein endothelial cell (HUVEC). Somatic cells useful for trans-differentiation may be immortalized somatic cells.

Various strategies are being pursued to induce pluripotency, or increase potency, in cells (Takahashi, K., and Yamanaka, S., Cell 126, 663-676 (2006); Takahashi et al., Cell 131, 861-872 (2007); Yu et al., Science 318, 1917-1920 (2007); Zhou et al., Cell Stem Cell 4, 381-384 (2009); Kim et al., Cell Stem Cell 4, 472-476 (2009); Yamanaka et al., 2009; Saha, K., Jaenisch, R., Cell Stem Cell 5, 584-595 (2009)), and improve the efficiency of reprogramming (Shi et al., Cell Stem Cell 2, 525-528 (2008a); Shi et al., Cell Stem Cell 3, 568-574 (2008b); Huangfu et al., Nat Biotechnol 26, 795-797 (2008a); Huangfu et al., Nat Biotechnol 26, 1269-1275 (2008b); Silva et al., Plos Bio 6, e253. Doi: 10.1371/journal. Pbio. 0060253 (2008); Lyssiotis et al., PNAS 106, 8912-8917 (2009); Ichida et al., Cell Stem Cell 5, 491-503 (2009); Maherali, N., Hochedlinger, K., Curr Biol 19, 1718-1723 (2009b); Esteban et al., Cell Stem Cell 6, 71-79 (2010); and Feng et al., Cell Stem Cell 4, 301-312 (2009)), the disclosures of which are hereby incorporated by reference in their entireties.

It is understood that a cell that is positive (+) for a particular cell surface marker is a cell that expresses the marker on its surface at a level that is detectable. Likewise, it is understood that a cell that is negative (−) for a particular cell surface marker is a cell that expresses the marker on its surface at a level that is not detectable. Antibodies and other binding entities can be used to detect expression levels of marker proteins to identify or detect a given cell surface marker. Suitable antibodies may include polyclonal, monoclonal, fragments (such as Fab fragments), single chain antibodies and other forms of specific binding molecules. Antibody reagents for cell surface markers above are readily known to a skilled artisan. A number of well-known methods for assessing expression level of surface markers or proteins may be used, such as detection by affinity-based methods, e.g., immunoaffinity-based methods, e.g., in the context of surface markers, such as by flow cytometry. In some embodiments, the label is a fluorophore and the method for detection or identification of cell surface markers on cells (e.g. T cells) is by flow cytometry. In some embodiments, different labels are used for each of the different markers by multicolor flow cytometry. In some embodiments, surface expression can be determined by flow cytometry, for example, by staining with an antibody that specifically binds to the marker and detecting the binding of the antibody to the marker.

In some embodiments, a cell (e.g. T cell) is positive (pos or +) for a particular marker if there is detectable presence on or in the cell of a particular marker, which can be an intracellular marker or a surface marker. In some embodiments, surface expression is positive if staining by flow cytometry is detectable at a level substantially above the staining detected carrying out the same procedures with an isotype-matched control under otherwise identical conditions and/or at a level substantially similar to, or in some cases higher than, a cell known to be positive for the marker and/or at a level higher than that for a cell known to be negative for the marker.

In some embodiments, a cell (e.g. T cell) is negative (neg or −) for a particular marker if there is an absence of detectable presence on or in the cell of a particular marker, which can be an intracellular marker or a surface marker. In some embodiments, surface expression is negative if staining is not detectable by flow cytometry at a level substantially above the staining detected carrying out the same procedures with an isotype-matched control under otherwise identical conditions and/or at a level substantially lower than a cell known to be positive for the marker and/or at a level substantially similar to a cell known to be negative for the marker.

In some aspects, the T_{SCM} cell phenotype can be characterized by one or more functions of the cells. In some aspects, the Tsem cell phenotype is characterized by polyfunctional activity of the T cells to produce more than one T cell stimulatory cytokine, such as determined in a polyfunctional cytokine secretion assay following stimulation of the T cells with a stimulatory agent. In some embodiments, the T cell is polyfunctional for producing two or more cytokines. In some embodiments, a T cell is polyfunctional for producing two or more cytokines selected from among interferon-gamma (IFN-gamma), interleukin 2 (IL-2) and TNF-alpha. In some embodiments, a polyfunctional T cell produces IFN-gamma, IL-2, and TNF-alpha. In some embodiments, the stimulatory agent is a non-specific or non-antigen-dependent T cell stimulatory agent. In some embodiments, the non-specific or non-antigen dependent T cell stimulatory agent is a polyclonal stimulatory agent. In some embodiments, the non-specific or non-antigen dependent stimulatory agent comprises PMA/ionomycin, anti-CD3/anti-CD28, phytohemagglutinin (PHA) or concanavalin A (ConA). In some embodiments, the non-specific or non-antigen dependent T cell stimulatory agent contains PMA/ionomycin.

In particular embodiments, the production of one or more cytokines is measured, detected, and/or quantified by intracellular cytokine staining. Intracellular cytokine staining (ICS) by flow cytometry is a technique well-suited for studying cytokine production at the single-cell level. It detects the production and accumulation of cytokines within the endoplasmic reticulum after cell stimulation, allowing for the identification of cell populations that are positive or negative for production of a particular cytokine or for the separation of high producing and low producing cells based on a threshold. In some embodiments, as described above, the stimulation can be performed using nonspecific stimulation, e.g., is not an antigen-specific stimulation. For example, PMA/ionomycin can be used for nonspecific cell stimulation. ICS can also be used in combination with other flow cytometry protocols for immunephenotyping using cell surface markers or with MHC multimers to access cytokine production in a particular subgroup of cells, making it an extremely flexible and versatile method. Other single-cell techniques for measuring or detecting cytokine production include, but are not limited to ELISPOT, limiting dilution, and T cell cloning. In some embodiments, the assays to assay polyfunctional cytokine secretion of multiple cytokines, can include multiplexed assays or other assays to assess polyfunctionality (see, e.g., Xue et al., (2017) Journal for ImmunoTherapy of Cancer 5:85).

The target genes for modulation by the provided epigenetic-modifying DNA-targeting systems herein include any whose transcription and expression are increased in cells with a particular or desired function or activity, such as cell phenotype (e.g. a T_{SCM} cell-like phenotype). Various methods may be utilized to characterize the transcription or expression levels of a gene in a cell (e.g. T cell) such as after the cell has been contacted or introduced with a provided epigenetic-modifying DNA-targeting system and selected for a desired activity or function, such as cell phenotype (e.g. a T_{SCM} cell-like phenotype). In some embodiments, the T_{SCM} cell-like phenotype can be a phenotype comprising one or more cell surface markers as described above. In some embodiments, the phenotype is CCR7+ and/or CD27+, such as a double positive CCR7+ and CD27+ phenotype. In some embodiments, analyzing the transcription activity or expression of a gene may be by RNA analysis. In some embodiments, the RNA analysis includes RNA quantification. In some embodiments, the RNA quantification occurs by reverse transcription quantitative PCR (RT-qPCR), multiplexed qRT-PCR, fluorescence in situ hybridization (FISH), or combinations thereof.

In some embodiments, the gene is one in which expression of the gene in the cell (e.g. T cell), is increased after having been contacted or introduced with a provided epigenetic-modifying DNA-targeting system. In some embodiments, the increase in gene expression in a cell (e.g. T cell) is about a log 2 fold change of greater than 1.0. For instance, the log 2 fold change is greater than at or about 1.5, at or about 2.0, at or about 2.5, at or about 3.0, at or about 4.0, at or about 5.0, at or about 6.0, at or about 7.0, at or about 8.0, at or about 9.0, at or about 10.0 or any value between any of the foregoing compared to the level of the gene in a control cell.

In some embodiments, the gene is selected from the list consisting of: DDIT3, EBF4, FOXD3, HELT, HES2, HKR1, HMGN3, PATZ1, PLAG1, PRDM4, PRDM8, SOX21, TSHZ1, ZBED5, ZBTB7A, ZNF141, ZNF219, ZNF319, ZNF470, ZNF562, ZNF691, ZNF692, ZNF816, ZSCAN23, ZSCAN5A, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF319, ZNF331, ZNF345, ZNF420, ZNF423, ZNF470, ZNF48, ZNF496, ZNF501, ZNF524, ZNF541, ZNF557, ZNF619, ZNF735, ZNF747, ZNF783, ZNF805, and ZSCAN10.

In some embodiments, the epigenetic-modifying DNA-targeting system targets to or binds to a target site in the gene, such as any described above. In some embodiments, the target site is located in a regulatory DNA element of the gene in the cell (e.g. T cell). In some embodiments, a regulatory DNA element is a sequence to which a gene regulatory protein may bind and affect transcription of the gene. In some embodiments, the regulatory DNA element is a cis, trans, distal, proximal, upstream, or downstream regulatory DNA element of a gene. In some embodiments, the regulatory DNA element is a promoter or enhancer of the gene. In some embodiments, the target site is located within a promoter, enhancer, exon, intron, untranslated region (UTR), 5′ UTR, or 3′ UTR of the gene. In some embodiments, a promoter is a nucleotide sequence to which RNA polymerase binds to begin transcription of the gene. In some embodiments, a promoter is a nucleotide sequence located within about 100 bp, about 500 bp, about 1000 bp, or more, of a transcriptional start site of the gene. In some embodiments the target site is located within a sequence of unknown or known function that is suspected of being able to control expression of a gene.

In some embodiments, the target site comprises a sequence selected from any one of SEQ ID NOS: 1-132, a contiguous portion thereof of at least 14 nucleotides of any one of SEQ ID NOS: 1-132, or a complementary sequence of any of the foregoing. In some embodiments, the target site is a contiguous portion of any one of SEQ ID NOS: 1-132 that is 15, 16, 17, 18 or 19 nucleotides, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a contiguous portion of a target site sequence described herein above. In some embodiments, the target site is the sequence set forth in any one of SEQ ID NOS: 1-132

In some embodiments, the gene is selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23.

In some embodiments, the target site comprises a sequence selected from any one of SEQ ID NOS: 1-28, a contiguous portion thereof of at least 14 nucleotides of any one of SEQ ID NOS: 1-28, or a complementary sequence of any of the foregoing. In some embodiments, the target site is a contiguous portion of any one of SEQ ID NOS: 1-28 that is 15, 16, 17, 18 or 19 nucleotides, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a portion of a target site sequence described herein above. In some embodiments, the target site is the sequence set forth in any one of SEQ ID NOS: 1-28.

In some embodiments, the gene is selected from the list consisting of: DDIT3, HES2, HKR1, PLAG1, PRDM4, TEF, TSHZ1, ZNF141, ZNF562, ZNF691, ZNF692, and ZSCAN5A.

In some embodiments, the target site comprises a sequence selected from any one of SEQ ID NOS: 1-12, a contiguous portion thereof of at least 14 nucleotides of any one of SEQ ID NOS: 1-12, or a complementary sequence of any of the foregoing. In some embodiments, the target site is a contiguous portion of any one of SEQ ID NOS: 1-12 that is 15, 16, 17, 18 or 19 nucleotides, or a complementary sequence of any of the foregoing. In some embodiments, the target site is a sequence having at or at least 80%, 85%, 90%, 91%, 92%, 93%, 94%, 95%, 96%, 97%, 98%, 99%, 99.5%, 99.9%, or 100% sequence identity to all or a portion of a target site sequence described herein above. In some embodiments, the target site is the sequence set forth in any one of SEQ ID NOS: 1-12.

### B. CRISPR-Based DNA-Targeting Systems

Provided herein are DNA-targeting systems based on CRISPR/Cas systems, i.e. CRISPR/Cas-based DNA-targeting systems, that are able to bind to a target site in a target gene. In some embodiments, the CRISPR/Cas DNA-targeting domain is nuclease inactive, such as includes a dCas (e.g. dCas9) so that the system binds to the target site in a target gene without mediating nucleic acid cleavage at the target site. The CRISPR/Cas-based DNA-targeting systems may be used to modulate expression of a target gene in a cell, such as a T cell. In some embodiments, the target gene may include any as described herein, including any described above in Section I.A. In some embodiments, the target site of the target gene may include any as described herein, including any described above in Section I.A. In some embodiments, the CRISPR/Cas-based DNA-targeting system can include any known Cas enzyme, and generally a nuclease-inactive or dCas. In some embodiments, the CRISPR/Cas-based DNA-targeting system includes a fusion protein of a nuclease-inactive Cas protein or a variant thereof and an effector domain that increases transcription of a gene (e.g. a transcription activation domain), and at least one gRNA.

The CRISPR system (also known as CRISPR/Cas system, or CRISPR-Cas system) refers to a conserved microbial nuclease system, found in the genomes of bacteria and archaea, that provides a form of acquired immunity against invading phages and plasmids. Clustered Regularly Interspaced Short Palindromic Repeats (CRISPR), refers to loci containing multiple repeating DNA elements that are separated by non-repeating DNA sequences called spacers. Spacers are short sequences of foreign DNA that are incorporated into the genome between CRISPR repeats, serving as a ‘memory’ of past exposures. Spacers encode the DNA-targeting portion of RNA molecules that confer specificity for nucleic acid cleavage by the CRISPR system. CRISPR loci contain or are adjacent to one or more CRISPR-associated (Cas) genes, which can act as RNA-guided nucleases for mediating the cleavage, as well as non-protein coding DNA elements that encode RNA molecules capable of programming the specificity of the CRISPR-mediated nucleic acid cleavage.

In Type II CRISPR/Cas systems with the Cas protein Cas9, two RNA molecules and the Cas9 protein form a ribonucleoprotein (RNP) complex to direct Cas9 nuclease activity. The CRISPR RNA (crRNA) contains a spacer sequence that is complementary to a target nucleic acid sequence (target site), and that encodes the sequence specificity of the complex. The trans-activating crRNA (tracrRNA) base-pairs to a portion of the crRNA and forms a structure that complexes with the Cas9 protein, forming a Cas/RNA RNP complex.

Naturally occurring CRISPR/Cas systems, such as those with Cas9, have been engineered to allow efficient programming of Cas/RNA RNPs to target desired sequences in cells of interest, both for gene-editing and modulation of gene expression. The tracrRNA and crRNA have been engineered to form a single chimeric guide RNA molecule, commonly referred to as a guide RNA (gRNA), for example as described in WO 2013/176772, WO 2014/093661, WO 2014/093655, Jinek, M. et al. Science 337 (6096): 816-21 (2012), or Cong. L. et al. Science 339 (6121): 819-23 (2013). The spacer sequence of the gRNA can be chosen by a user to target the Cas/gRNA RNP complex to a desired locus, e.g. a desired target site in the target gene.

Cas proteins have also been engineered to be catalytically inactivated or nuclease inactive to allow targeting of Cas/gRNA RNPs without inducing cleavage at the target site. Mutations in Cas proteins can reduce or abolish nuclease activity of the Cas protein, rendering the Cas protein catalytically inactive. Cas proteins with reduced or abolished nuclease activity are referred to as deactivated Cas (dCas), or nuclease-inactive Cas (iCas) proteins, as referred to interchangeably herein. Exemplary deactivated Cas9 (dCas9) derived from S. pyogenes contains silencing mutations of the RuvC and HNH nuclease domains (D10A and H840A), for example as described in WO 2013/176772, WO 2014/093661, Jinek, M. et al. Science 337 (6096): 816-21 (2012), and Qi. L. et al. Cell 152 (5): 1173-83 (2013). Exemplary dCas variants derived from the Cas12 system (i.e. Cpf1) are described, for example in WO 2017/189308 and Zetsche, B. et al. Cell 163 (3): 759-71 (2015). Conserved domains that mediate nucleic acid cleavage, such as RuvC and HNH endonuclease domains, are readily identifiable in Cas orthologues, and can be mutated to produce inactive variants, for example as described in Zetsche, B. et al. Cell 163 (3): 759-71 (2015).

dCas-fusion proteins with transcriptional regulators have been used as a versatile platform for ectopically regulating gene expression in target cells. These include fusion of the Cas with an effector domain, such as a transcription activation domain. Any of a variety of effector domains that increase transcription can be used as described further below. For example, fusing dCas9 with transcriptional activators such as VP64 (a polypeptide composed of four tandem copies of VP16, a 16 amino acid transactivation domain of the Herpes simplex virus) can result in robust induction of gene expression. A variety of dCas-fusion proteins with VP64 and other transcriptional regulators can be engineered for regulation of gene expression, for example as described in WO 2014/197748, WO 2016/130600, WO 2017/180915, WO 2021/226555, WO 2013/176772, WO 2014/152432, WO 2014/093661, Adli, M. Nat. Commun. 9, 1911 (2018), Percz-Pinera, P. et al. Nat. Methods 10, 973-976 (2013), Mali, P. et al. Nat. Biotechnol. 31, 833-838 (2013), and Maeder, M. L. et al. Nat. Methods 10, 977-979 (2013). In some aspects, provided is a DNA-targeting system comprising a fusion protein comprising a DNA-targeting domain comprising a nuclease-inactive Cas protein or variant thereof, and an effector domain for increasing or inducing transcriptional activation (i.e. a transcriptional activator) when targeted to the target gene in the cell (e.g. T cell). In such embodiments, the DNA-targeting system also includes one or more gRNA, provided in combination or as a complex with the dCas protein or variant thereof, for targeting of the DNA-targeting system to the target site of the target gene. In some embodiments, the fusion protein is guided to a specific target site sequence of the target gene by the guide RNA, wherein the effector domain mediates targeted epigenetic modification to increase or promote transcription of the target gene.

i. CRISPR-Based DNA-Targeting Domains

In some aspects, the DNA-targeting domain comprises a CRISPR-associated (Cas) protein or variant thereof, or is derived from a Cas protein or variant thereof. In particular embodiments here, the Cas protein nuclease-inactive (i.e. is a dCas protein).

In some embodiments, the Cas protein is derived from a Class 1 CRISPR system (i.e. multiple Cas protein system), such as a Type I, Type III, or Type IV CRISPR system. In some embodiments, the Cas protein is derived from a Class 2 CRISPR system (i.e. single Cas protein system), such as a Type II, Type V, or Type VI CRISPR system. In some embodiments, the Cas protein is from a Type V CRISPR system. In some embodiments, the Cas protein is derived from a Cas12 protein (i.e. Cpf1) or variant thereof, for example as described in WO 2017/189308 and Zetsche, B. et al. Cell. 163 (3): 759-71 (2015). In some embodiments, the Cas protein is derived from a Type II CRISPR system. In some embodiments, the Cas protein is derived from a Cas9 protein or variant thereof, for example as described in WO 2013/176772. WO 2014/152432, WO 2014/093661, WO 2014/093655, Jinek, M. et al. Science 337 (6096): 816-21 (2012), Mali, P. et al. Science 339 (6121): 823-6 (2013), Cong. L. et al. Science 339 (6121): 819-23 (2013), Perez-Pinera, P. et al. Nat. Methods 10, 973-976 (2013), or Mali, P. et al. Nat. Biotechnol. 31, 833-838 (2013). Various CRISPR/Cas systems and associated Cas proteins for use in gene editing and regulation have been described, for example in Moon, S. B. et al. Exp. Mol. Med. 51, 1-11 (2019), Zhang, F. Q. Rev. Biophys. 52, E6 (2019), and Makarova K. S. et al. Methods Mol. Biol. 1311:47-75 (2015).

In some embodiments, the dCas9 protein can comprise a sequence derived from a naturally occurring Cas9 molecule, or variant thereof. In some embodiments, the dCas9 protein can comprise a sequence derived from a naturally occurring Cas9 molecule of S. pyogenes, S. thermophilus, S. aureus, C. jejuni, N. meningitidis, F. novicida, S. canis, S. auricularis, or variant thereof. In some embodiments, the dCas9 protein comprises a sequence derived from a naturally occurring Cas9 molecule of S. aureus. In some embodiments, the dCas9 protein comprises a sequence derived from a naturally occurring Cas9 molecule of S. pyogenes.

Non-limiting examples of Cas9 orthologs from other bacterial strains include but are not limited to: Cas proteins identified in Acaryochloris marina MBIC11017; Acetohalobium arabaticum DSM 5501; Acidithiobacillus caldus; Acidithiobacillus ferrooxidans ATCC 23270; Alicyclobacillus acidocaldarius LAA1; Alicyclobacillus acidocaldarius subsp. acidocaldarius DSM 446; Allochromatium vinosum DSM 180; Ammonifex degensii KC4; Anabaena variabilis ATCC 29413; Arthrospira maxima CS-328; Arthrospira platensis str. Paraca; Arthrospira sp. PCC 8005; Bacillus pseudomycoides DSM 12442; Bacillus selenitireducens MLS10; Burkholderiales bacterium 1_1_47; Caldicelulosiruptor becscii DSM 6725; Candidatus Desulforudis audaxviator MP104C; Caldicellulosiruptor hydrothermalis 108; Clostridium phage c-st; Clostridium botulinum A3 str. Loch Marce; Clostridium botulinum Ba4 str. 657; Clostridium difficile QCD-63q42; Crocosphaera watsonii WH 8501; Cyanothece sp. ATCC 51142; Cyanothece sp. CCY0110; Cyanothece sp. PCC 7424; Cyanothece sp. PCC 7822; Exiguobacterium sibiricum 255-15; Finegoldia magna ATCC 29328; Ktedonobacter racemifer DSM 44963; Lactobacillus delbrueckii subsp. bulgaricus PB2003/044-T3-4; Lactobacillus salivarius ATCC 11741; Listeria innocua; Lyngbya sp. PCC 8106; Marinobacter sp. ELB17; Methanohalobium evestigatum Z-7303; Microcystis phage Ma-LMM01; Microcystis aeruginosa NIES-843; Microscilla marina ATCC 23134; Microcoleus chthonoplastes PCC 7420; Neisseria meningitidis; Nitrosococcus halophilus Nc4; Nocardiopsis dassonvillei subsp. dassonvillei DSM 43111; Nodularia spumigena CCY9414; Nostoc sp. PCC 7120; Oscillatoria sp. PCC 6506; Pelotomaculum_thermopropionicum SI; Petrotoga mobilis SJ95; Polaromonas naphthalenivorans CJ2; Polaromonas sp. JS666; Pseudoalteromonas haloplanktis TAC125; Streptomyces pristinaespiralis ATCC 25486; Streptomyces pristinaespiralis ATCC 25486; Streptococcus thermophilus; Streptomyces viridochromogenes DSM 40736; Streptosporangium roscum DSM 43021; Synechococcus sp. PCC 7335; and Thermosipho africanus TCF52B (Chylinski et al., RNA Biol., 2013; 10 (5): 726-737).

In some aspects, the Cas protein is a variant that lacks nuclease activity (i.e. is a dCas protein). In some embodiments, the Cas protein is mutated so that nuclease activity is reduced or eliminated. Such Cas proteins are referred to as deactivated Cas or dead Cas (dCas) or nuclease-inactive Cas (iCas) proteins, as referred to interchangeably herein. In some embodiments, the variant Cas protein is a variant Cas9 protein that lacks nuclease activity or that is a deactivated Cas9 (dCas9, or iCas9) protein.

In some embodiments, the Cas9 protein or a variant thereof is derived from a Staphylococcus aureus Cas9 (SaCas9) protein or a variant thereof. In some embodiments, the variant Cas9 is a Staphylococcus aureus dCas9 protein (dSaCas9) that comprises at least one amino acid mutation selected from D10A and N580A, with reference to numbering of positions of SEQ ID NO:405. In some embodiments, the variant Cas9 protein comprises the sequence set forth in SEQ ID NO:406, or an amino acid sequence that has at least 90%, 91%, 92%, 93%, 94%, 95%, 96%, 97%, 98%, or 99% sequence identity thereto.

In some embodiments, the Cas9 protein or variant thereof is derived from a Streptococcus pyogenes Cas9 (SpCas9) protein or a variant thereof. In some embodiments, the variant Cas9 is a Streptococcus pyogenes dCas9 (dSpCas9) protein that comprises at least one amino acid mutation selected from D10A and H840A, with reference to numbering of positions of SEQ ID NO:407. In some embodiments, the variant Cas9 protein comprises the sequence set forth in SEQ ID NO:408, or an amino acid sequence that has at least 90%, 91%, 92%, 93%, 94%, 95%, 96%, 97%, 98%, or 99% sequence identity thereto.

ii. Guide RNAs

In some embodiments, the Cas protein (e.g. dCas9) is provided in combination or as a complex with one or more guide RNA (gRNA). In some aspects, the gRNA is a nucleic acid that promotes the specific targeting or homing of the gRNA/Cas RNP complex to the target site of the target gene, such as any described above. In some embodiments, a target site of a gRNA may be referred to as a protospacer.

Provided herein are gRNAs, such as gRNAs that target or bind to a target gene or DNA regulatory element thereof, such as any described above in Section I.A. In some embodiments, the gRNA is capable of complexing with the Cas protein or variant thereof. In some embodiments, the gRNA comprises a gRNA spacer sequence (i.e. a spacer sequence or a guide sequence) that is capable of hybridizing to the target site, or that is complementary to the target site, such as any target site described in Section I.A or further below. In some embodiments, the gRNA comprises a scaffold sequence that complexes with or binds to the Cas protein.

In some embodiments, the gRNAs provided herein are chimeric gRNAs. In general, gRNAs can be unimolecular (i.e. composed of a single RNA molecule), or modular (comprising more than one, and typically two, separate RNA molecules). Modular gRNAs can be engineered to be unimolecular, wherein sequences from the separate modular RNA molecules are comprised in a single gRNA molecule, sometimes referred to as a chimeric gRNA, synthetic gRNA, or single gRNA. In some embodiments, the chimeric gRNA is a fusion of two non-coding RNA sequences: a crRNA sequence and a tracrRNA sequence, for example as described in WO 2013/176772, or Jinek, M. et al. Science 337 (6096): 816-21 (2012). In some embodiments, the chimeric gRNA mimics the naturally occurring crRNA: tracrRNA duplex involved in the Type II Effector system, wherein the naturally occurring crRNA: tracrRNA duplex acts as a guide for the Cas9 protein.

In some aspects, the spacer sequence of a gRNA is a polynucleotide sequence comprising at least a portion that has sufficient complementarity with the target gene or DNA regulatory element thereof (e.g. any described in Section I.A) to hybridize with a target site in the target gene and direct sequence-specific binding of a CRISPR complex to the sequence of the target site. Full complementarity is not necessarily required, provided there is sufficient complementarity to cause hybridization and promote formation of a CRISPR complex. In some embodiments, the gRNA comprises a spacer sequence that is complementary, e.g., at least 80%, 85%, 90%, 95%, 98%, 99%, or 100% (e.g., fully complementary), to the target site. The strand of the target nucleic acid comprising the target site sequence may be referred to as the “complementary strand” of the target nucleic acid.

In some embodiments, the gRNA spacer sequence is between about 14 nucleotides (nt) and about 26 nt, or between 16 nt and 22 nt in length. In some embodiments, the gRNA spacer sequence is 14 nt, 15 nt, 16 nt, 17 nt, 18 nt, 19 nt, 20 nt, 21 nt or 22 nt, 23 nt, 24 nt, 25 nt, or 26 nt in length. In some embodiments, the gRNA spacer sequence is 18 nt, 19 nt, 20 nt, 21 nt or 22 nt in length. In some embodiments, the gRNA spacer sequence is 19 nt in length.

A target site of a gRNA may be referred to as a protospacer. In some aspects, the spacer is designed to target a protospacer with a specific protospacer-adjacent motif (PAM), i.e. a sequence immediately adjacent to the protospacer that contributes to and/or is required for Cas binding specificity. Different CRISPR/Cas systems have different PAM requirements for targeting. For example, in some embodiments, S. pyogenes Cas9 uses the PAM 5′-NGG-3′ (SEQ ID NO: 403), where N is any nucleotide. In some embodiments, S. aureus Cas9 uses the PAM 5′-NNGRRT-3′ (SEQ ID NO: 404), where N is any nucleotide, and R is G or A. In some embodiments, N. meningitidis Cas9 uses the PAM 5′-NNNNGATT-3′ (SEQ ID NO: 441), where N is any nucleotide. In some embodiments, C. jejuni Cas9 uses the PAM 5′-NNNNRYAC-3′ (SEQ ID NO: 442), where N is any nucleotide, R is G or A, and Y is C or T. In some embodiments, S. thermophilus uses the PAM 5′-NNAGAAW-3′ (SEQ ID NO: 443), where N is any nucleotide and W is A or T. In some embodiments, F. Novicida Cas9 uses the PAM 5′-NGG-3′ (SEQ ID NO: 403), where N is any nucleotide. In some embodiments, T. denticola Cas9 uses the PAM 5′-NAAAAC-3′ (SEQ ID NO: 444), where N is any nucleotide. In some embodiments, Cas12a (also known as Cpf1) from various species, uses the PAM 5′-TTTV-3′ (SEQ ID NO: 445). In some embodiments, Cas proteins may use or be engineered to use different PAMs from those listed above. For example, mutated SpCas9 proteins may use the PAMs 5′-NGG-3′ (SEQ ID NO: 403), 5′-NGAN-3′ (SEQ ID NO: 446), 5′-NGNG-3′ (SEQ ID NO: 447), 5′-NGAG-3′ (SEQ ID NO: 448), or 5′-NGCG-3′ (SEQ ID NO: 449). In some embodiments, the protospacer-adjacent motif (PAM) of a gRNA for complexing with S. pyogenes Cas9 or variant thereof is set forth in SEQ ID NO: 403. In some embodiments, the PAM of a gRNA for complexing with S. aureus Cas9 or variant thereof is set forth in SEQ ID NO: 404.

A spacer sequence may be selected to reduce the degree of secondary structure within the spacer sequence. Secondary structure may be determined by any suitable polynucleotide folding algorithm.

In some embodiments, the gRNA (including the guide sequence) will comprise the base uracil (U), whereas DNA encoding the gRNA molecule will comprise the base thymine (T). While not wishing to be bound by theory, in some embodiments, it is believed that the complementarity of the guide sequence with the target sequence contributes to specificity of the interaction of the gRNA molecule/Cas molecule complex with a target nucleic acid. It is understood that in a guide sequence and target sequence pair, the uracil bases in the guide sequence will pair with the adenine bases in the target sequence.

In some embodiments, one, more than one, or all of the nucleotides of a gRNA can have a modification, e.g., to render the gRNA less susceptible to degradation and/or improve bio-compatibility. By way of non-limiting example, the backbone of the gRNA can be modified with a phosphorothioate, or other modification(s). In some cases, a nucleotide of the gRNA can comprise a 2′ modification, e.g., a 2-acetylation, e.g., a 2′ methylation, or other modification(s)

Methods for designing gRNAs and exemplary targeting domains can include those described in, e.g., International PCT Pub. Nos. WO 2014/197748, WO 2016/130600, WO 2017/180915, WO 2021/226555, WO 2013/176772, WO 2014/152432, WO 2014/093661, WO 2014/093655, WO 2015/089427, WO 2016/049258, WO 2016/123578, WO 2021/076744, WO 2014/191128, WO 2015/161276, WO 2017/193107, and WO 2017/093969.

In some embodiments, a gRNA provided herein targets a target site in a gene in a T cell or DNA regulatory element thereof, wherein the gene is selected from the list shown in Table 1, consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, ZSCAN23, EMX1, FOXO3, HIC2, SOX30, STAT1, ZBTB46, ZNF331, ZNF541, ZNF619, AEBP1, ARID3A, ARNTL2, BACH1, BATF2, BRD4, CDX4, CENPB, DMRT1, E2F2, ESRRB, FERD3L, FEV, FLYWCH1, FOXJ3, GLIS3, HDAC9, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SPIC, THAP7, TOX2, TRERF1, TRIM3, VDR, YBX2, ZBTB14, ZBTB4, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF345, ZNF420, ZNF423, ZNF48, ZNF496, ZNF501, ZNF524, ZNF557, ZNF735, ZNF747, ZNF783, ZNF805, and ZSCAN10.

In some embodiments, the gRNA targets a target site that comprises a sequence selected from any one of SEQ ID NOS: 1-132, as shown in Table 1, a contiguous portion thereof of at least 14 nucleotides, a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the target site is a contiguous portion of any one of SEQ ID NOS: 1-132 that is 14, 15, 16, 17, 18 or 19 nucleotides in length. In some embodiments, the target site is set forth in any on eof SEQ ID NOS: 1-132.

In some embodiments, the gRNA comprises a spacer sequence selected from any one of SEQ ID NOS: 133-264, as shown in Table 1, or a contiguous portion thereof of at least 14 nt, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the spacer sequence of the gRNA is a contiguous portion of any one of SEQ ID NOS: 133-264 that is 14, 15, 16, 17, 18 or 19 nucleotides in length. In some embodiments, the spacer sequence of the gRNA is set forth in any on eof SEQ ID NOS: 133-264.

In some embodiments, the gRNA further comprises a scaffold sequence. In some embodiments, the scaffold sequence comprises the sequence set forth in SEQ ID NO: 398 (GUUUAAGAGCUAUGCUGGAAACAGCAUAGCAAGUUUAAAUAAGGCUAGUCCGU UAUCAACUUGAAAAAGUGGCACCGAGUCGGUGC), or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the scaffold sequence is set forth in SEQ ID NO: 398.

In some embodiments, a gRNA provided herein comprises a spacer sequence selected from any one of SEQ ID NOS: 133-264, as shown in Table 1. In some embodiments, the gRNA further comprises a scaffold sequence set forth in SEQ ID NO: 398. In some embodiments, the gRNA comprises the sequence selected from any one of SEQ ID NOS: 265-396, as shown in Table 2, or a sequence having at or at least 80%, 85%, 90%, 91%, 92%, 93%, 94%, 95%, 96%, 97%, 98%, 99%, 99.5%, 99.9%, or 100% sequence identity to any one of SEQ ID NO: 265-396. In some embodiments, the gRNA is set forth in any one of SEQ ID NOS: 265-396.

In some embodiments, any of the provided gRNA sequences is complexed with or is provided in combination with a Cas9. In some embodiments, the Cas9 is a dCas9. In some embodiments, the dCas9 is a dSpCas9, such as a dSpCas9 set forth in SEQ ID NO: 408.

TABLE 1
Genes, target site sequences, and gRNA spacers

| | | | | RNA |
|---|---|---|---|---|
| | target site (protospacer) | target | | spacer |
| Gene | sequence | SEQ ID | RNA spacer sequence | SEQ ID |
| DDIT3 | CCCTAGCGAGAGGGAGCGA | 1 | CCCUAGCGAGAGGGAGCGA | 133 |
| HES2 | CCGTCGGGGATCTGCGGGC | 2 | CCGUCGGGGAUCUGCGGGC | 134 |
| HKR1 | CAGTCCCGCGATGCCTTCG | 3 | CAGUCCCGCGAUGCCUUCG | 135 |
| PLAG1 | AACACTTCGGCTTGACGGG | 4 | AACACUUCGGCUUGACGGG | 136 |
| PRDM4 | TGGCCGCGAGAGGCTCGGC | 5 | UGGCCGCGAGAGGCUCGGC | 137 |
| TEF | AGGCTGGGAGCCATAACTG | 6 | AGGCUGGGAGCCAUAACUG | 138 |
| TSHZ1 | GACCTATTTGTAATTGTCA | 7 | GACCUAUUUGUAAUUGUCA | 139 |
| ZNF141 | GAGAGCTGTGCACCCAACT | 8 | GAGAGCUGUGCACCCAACU | 140 |
| ZNF562 | TGGACGGAACCGTGGGCGA | 9 | UGGACGGAACCGUGGGCGA | 141 |
| ZNF691 | CGCCCTACTACTCCAGGAT | 10 | CGCCCUACUACUCCAGGAU | 142 |
| ZNF692 | CCCCGCGGCAGTTGTGGTT | 11 | CCCCGCGGCAGUUGUGGUU | 143 |
| ZSCAN5A | ACGTAATCGGCGTTGATTA | 12 | ACGUAAUCGGCGUUGAUUA | 144 |
| EBF4 | AAGACGCCGGGGCTTCGTC | 13 | AAGACGCCGGGGCUUCGUC | 145 |
| ETS1 | AGAAACGCCCTAAAGAGGA | 14 | AGAAACGCCCUAAAGAGGA | 146 |
| FOXD3 | GCTGCAAGGGCGGTGTTGG | 15 | GCUGCAAGGGCGGUGUUGG | 147 |
| HELT | CAAGCGCCTGCAGCAGTCG | 16 | CAAGCGCCUGCAGCAGUCG | 148 |
| HMGN3 | GCGTACCGCGCGCGGAGGT | 17 | GCGUACCGCGCGCGGAGGU | 149 |
| PATZ1 | GAAACGGCGAGCTCAGAGT | 18 | GAAACGGCGAGCUCAGAGU | 150 |
| PRDM8 | CCCAGCTCTCCATCCCCGG | 19 | CCCAGCUCUCCAUCCCCGG | 151 |
| SOX21 | CGGGAGAGGAAGACTCGAG | 20 | CGGGAGAGGAAGACUCGAG | 152 |
| TBX15 | GATCACAGGGAGGACGGGA | 21 | GAUCACAGGGAGGACGGGA | 153 |
| ZBED5 | AAAGGACCAAGCTCGGTTG | 22 | AAAGGACCAAGCUCGGUUG | 154 |
| ZBTB7A | TCCCTCCGCCGTTTGCAGC | 23 | UCCCUCCGCCGUUUGCAGC | 155 |
| ZNF219 | CCTAAACCCGGACATACCA | 24 | CCUAAACCCGGACAUACCA | 156 |
| ZNF319 | TACCACTCTAAGCCTTCTC | 25 | UACCACUCUAAGCCUUCUC | 157 |
| ZNF470 | TTCCCAGGGGCTATTGCAG | 26 | UUCCCAGGGGCUAUUGCAG | 158 |
| ZNF816 | AGCAGGAATGCGGGCGCGG | 27 | AGCAGGAAUGCGGGCGCGG | 159 |
| ZSCAN23 | CTCCGCGAAACAGCGGTTC | 28 | CUCCGCGAAACAGCGGUUC | 160 |
| EMX1 | AGCGCCGGGAGCCGTGGTT | 29 | AGCGCCGGGAGCCGUGGUU | 161 |
| FOXO3 | GCCGCGGGAGGCACTAGAG | 30 | GCCGCGGGAGGCACUAGAG | 162 |
| HIC2 | TGGGCTCCTGGGGAAGCCC | 31 | UGGGCUCCUGGGGAAGCCC | 163 |
| SOX30 | GCCGCGGAAAGCAGATCTG | 32 | GCCGCGGAAAGCAGAUCUG | 164 |
| STAT1 | CAGCGTGGCAACACGGGAC | 33 | CAGCGUGGCAACACGGGAC | 165 |
| ZBTB46 | CCGCGCCGCGGGGTCGCAG | 34 | CCGCGCCGCGGGGUCGCAG | 166 |
| ZNF331 | CAGGTGAGATTGGCGCCCA | 35 | CAGGUGAGAUUGGCGCCCA | 167 |
| ZNF541 | CAAGTCATGTGTCAGATAA | 36 | CAAGUCAUGUGUCAGAUAA | 168 |
| ZNF619 | CGCAGAGCGGTTGTGGAGA | 37 | CGCAGAGCGGUUGUGGAGA | 169 |
| AEBP1 | CGGGACCCCGAGTCCACCG | 38 | CGGGACCCCGAGUCCACCG | 170 |
| ARID3A | CCCGCCCCCGGCCTAGGGA | 39 | CCCGCCCCCGGCCUAGGGA | 171 |
| ARNTL2 | ACCCTCGGGGCAAAGGTAA | 40 | ACCCUCGGGGCAAAGGUAA | 172 |
| BACH1 | TCGCTGACAGAGGCTTCGA | 41 | UCGCUGACAGAGGCUUCGA | 173 |
| BATF2 | CAAAGGCCAGGTATGAGGA | 42 | CAAAGGCCAGGUAUGAGGA | 174 |
| BRD4 | TGCTGAAGTCATGAGGTGT | 43 | UGCUGAAGUCAUGAGGUGU | 175 |
| CDX4 | GGGAAGATCGGTGAAAGGG | 44 | GGGAAGAUCGGUGAAAGGG | 176 |
| CENPB | GCCCCCCCGCGGTATGTCC | 45 | GCCCCCCCGCGGUAUGUCC | 177 |
| DMRT1 | AGAGCGGTGGCTGGAGTCG | 46 | AGAGCGGUGGCUGGAGUCG | 178 |
| E2F2 | CGGAAAGAACGCGTCCTCG | 47 | CGGAAAGAACGCGUCCUCG | 179 |
| ESRRB | GAGGGGCAACCCCCGACTC | 48 | GAGGGGCAACCCCCGACUC | 180 |
| FERD3L | TGAGTAGGAGGTAAAGGTA | 49 | UGAGUAGGAGGUAAAGGUA | 181 |
| FEV | CGGGAGCCGACTGGAAGAA | 50 | CGGGAGCCGACUGGAAGAA | 182 |
| FLYWCH | GGGACGTAACTCACGGAAA | 51 | GGGACGUAACUCACGGAAA | 183 |
| 1 | | | | |
| FOXJ3 | CAGAGCCTCGGGAGTCTCA | 52 | CAGAGCCUCGGGAGUCUCA | 184 |
| GLIS3 | GGCTCGCGGGTCACTAGGC | 53 | GGCUCGCGGGUCACUAGGC | 185 |
| HDAC9 | GTAAAGCAAGCTTCGAGAG | 54 | GUAAAGCAAGCUUCGAGAG | 186 |
| HIVEP1 | CAGTCACGCGGCCCCGGGA | 55 | CAGUCACGCGGCCCCGGGA | 187 |
| HOXA2 | GGAAGGAACGCCTCATTGG | 56 | GGAAGGAACGCCUCAUUGG | 188 |
| HOXC9 | GAGCTAGGAGCCGTCCTGA | 57 | GAGCUAGGAGCCGUCCUGA | 189 |
| HSFY1 | GGTAGGTTACGGGTTGTCA | 58 | GGUAGGUUACGGGUUGUCA | 190 |
| ISL2 | AACGGCCGCGGAGCCAGCT | 59 | AACGGCCGCGGAGCCAGCU | 191 |
| KAT2A | TCCGGAGCCGCGTGGGGCA | 60 | UCCGGAGCCGCGUGGGGCA | 192 |
| KDM5D | GCAAGAGCGATTTTACCAG | 61 | GCAAGAGCGAUUUUACCAG | 193 |
| KLF13 | ACCGGGCACGCCCCTCTAA | 62 | ACCGGGCACGCCCCUCUAA | 194 |
| KLF6 | TGACCGAGACTCTCCATCA | 63 | UGACCGAGACUCUCCAUCA | 195 |
| MEOX2 | AATCAGGGTGCTAGGTTGG | 64 | AAUCAGGGUGCUAGGUUGG | 196 |
| MLXIPL | CGCCAGAGCTCCAGAGCAC | 65 | CGCCAGAGCUCCAGAGCAC | 197 |
| MNT | GGGCCGCGGCGTCTCGCGA | 66 | GGGCCGCGGCGUCUCGCGA | 198 |
| MYCN | CAAGCCCGGGGGTCCAAAA | 67 | CAAGCCCGGGGGUCCAAAA | 199 |
| MYTIL | GCCTCATAGATAATGATGA | 68 | GCCUCAUAGAUAAUGAUGA | 200 |
| NEUROG3 | CTCGCGTGGCGCGGCCCCA | 69 | CUCGCGUGGCGCGGCCCCA | 201 |
| NFE2 | AGGAGAGCCACGGGGAAAG | 70 | AGGAGAGCCACGGGGAAAG | 202 |
| NR2F1 | TTTCTCTCCCCCCGATTCA | 71 | UUUCUCUCCCCCCGAUUCA | 203 |
| NR2F2 | CCCCCTCGCCGTCATTGGC | 72 | CCCCCUCGCCGUCAUUGGC | 204 |
| NR3C1 | GGGCCCGCGCGGAGGGCGT | 73 | GGGCCCGCGCGGAGGGCGU | 205 |
| PHF20 | AGGCCGTTGCCACAGTGAT | 74 | AGGCCGUUGCCACAGUGAU | 206 |
| PITX2 | CAGGACAGTCCCTCCACCG | 75 | CAGGACAGUCCCUCCACCG | 207 |
| POU2AF1 | GCCGCTGGGGCGCGCACAG | 76 | GCCGCUGGGGCGCGCACAG | 208 |
| POU3F2 | GCGGGGCCCATCCGAGAGA | 77 | GCGGGGCCCAUCCGAGAGA | 209 |
| POU3F3 | GGCCACGACCCCCCCTGAA | 78 | GGCCACGACCCCCCCUGAA | 210 |
| PRDM11 | TGGGTGTGGAGGGGTTCCA | 79 | UGGGUGUGGAGGGGUUCCA | 211 |
| PRMT3 | TGAAGAGGCGCGCTGATGA | 80 | UGAAGAGGCGCGCUGAUGA | 212 |
| PROP1 | TCGGAGGGTCCAGGGACTG | 81 | UCGGAGGGUCCAGGGACUG | 213 |
| PURA | CTCACGCGCGAGAAGGGGG | 82 | CUCACGCGCGAGAAGGGGG | 214 |
| RELB | TGGGCGCTCAGGAGGGGTT | 83 | UGGGCGCUCAGGAGGGGUU | 215 |
| RFX3 | ACTGACGGCCCCCAAACCC | 84 | ACUGACGGCCCCCAAACCC | 216 |
| SAFB | CTGCTAAGTACGCTAGTGG | 85 | CUGCUAAGUACGCUAGUGG | 217 |
| SETBP1 | TATGGAGAGAGATATCAAG | 86 | UAUGGAGAGAGAUAUCAAG | 218 |
| SMAD2 | AAGCCGCGCGGGGGGGGTA | 87 | AAGCCGCGCGGGGCGGGUA | 219 |
| SMAD6 | CCCGGGGCGGGCCGAGCTC | 88 | CCCGGGGCGGGCCGAGCUC | 220 |
| SNAPC2 | AGGGAGGGCATGGGATCCA | 89 | AGGGAGGGCAUGGGAUCCA | 221 |
| SNAPC4 | TCTCAGAGCCCACCCTCGA | 90 | UCUCAGAGCCCACCCUCGA | 222 |
| SON | CTGTACTTGGCCTGTACTA | 91 | CUGUACUUGGCCUGUACUA | 223 |
| SOX11 | GGGCTGCTTTGCAAAGATG | 92 | GGGCUGCUUUGCAAAGAUG | 224 |
| SOX12 | GGCCCGGCCGCAGCCCAAG | 93 | GGCCCGGCCGCAGCCCAAG | 225 |
| SOX14 | CAAAAGCGGCGAAGTTCAA | 94 | CAAAAGCGGCGAAGUUCAA | 226 |
| SPIC | TGCAACCTAAGTTGGGCGG | 95 | UGCAACCUAAGUUGGGCGG | 227 |
| THAP7 | AATCGGCTGGGACGGGGTT | 96 | AAUCGGCUGGGACGGGGUU | 228 |
| TOX2 | GCTCCCGCGGGCAGTGCCT | 97 | GCUCCCGCGGGCAGUGCCU | 229 |
| TRERF1 | GGGTACCGGGGAGCGGATA | 98 | GGGUACCGGGGAGCGGAUA | 230 |
| TRIM3 | GGGCGTGCGGGTGCGGTAG | 99 | GGGCGUGCGGGUGCGGUAG | 231 |
| VDR | TACAGCGTGGCCTATAGGG | 100 | UACAGCGUGGCCUAUAGGG | 232 |
| YBX2 | GGGCCCAGGTGTACTTACT | 101 | GGGCCCAGGUGUACUUACU | 233 |
| ZBTB14 | CGCGCGCGTGCGCAGGCCA | 102 | CGCGCGCGUGCGCAGGCCA | 234 |
| ZBTB4 | CCGGGACGGTTGGAGAAGA | 103 | CCGGGACGGUUGGAGAAGA | 235 |
| ZFHX2 | GGTACAGGTCTCATGCCTG | 104 | GGUACAGGUCUCAUGCCUG | 236 |
| ZFP57 | GCAGCTATCAGTTGGTCGG | 105 | GCAGCUAUCAGUUGGUCGG | 237 |
| ZFP62 | GAGGGCAGAGAGACCTCTA | 106 | GAGGGCAGAGAGACCUCUA | 238 |
| ZFP69B | TGATGATGCTCGCAGGGAT | 107 | UGAUGAUGCUCGCAGGGAU | 239 |
| ZIC1 | GGGAATGCGGGCGCACCAA | 108 | GGGAAUGCGGGCGCACCAA | 240 |
| ZIC4 | CAACGGGGACGTTGACAAG | 109 | CAACGGGGACGUUGACAAG | 241 |
| ZNF136 | CCCCGACGGCGCGCCTGAT | 110 | CCCCGACGGCGCGCCUGAU | 242 |
| ZNF16 | GCTAGTGGGACCAGGTCTG | 111 | GCUAGUGGGACCAGGUCUG | 243 |
| ZNF174 | CAGGGGCGTGCGTAATGGG | 112 | CAGGGGCGUGCGUAAUGGG | 244 |
| ZNF253 | TGTCACTCAGCGGCTGGGG | 113 | UGUCACUCAGCGGCUGGGG | 245 |
| ZNF26 | ACAGCCTGACGGGAAAGGA | 114 | ACAGCCUGACGGGAAAGGA | 246 |
| ZNF263 | GGGCGCGCCCTGAAATTCT | 115 | GGGCGCGCCCUGAAAUUCU | 247 |
| ZNF285 | AAAGCCCCGCGCGCAGCAG | 116 | AAAGCCCCGCGCGCAGCAG | 248 |
| ZNF317 | TCCCGCGAGAATTGGAAGG | 117 | UCCCGCGAGAAUUGGAAGG | 249 |
| ZNF319 | CATCTTCCGGAAGGGAGGG | 118 | CAUCUUCCGGAAGGGAGGG | 250 |
| ZNF345 | CGCGCCAGGGGAATCGGGA | 119 | CGCGCCAGGGGAAUCGGGA | 251 |
| ZNF420 | GAGCCCACGAGCCCCTCGA | 120 | GAGCCCACGAGCCCCUCGA | 252 |
| ZNF423 | CAGGGGATCAGCCATCCAG | 121 | CAGGGGAUCAGCCAUCCAG | 253 |
| ZNF470 | GACCAGGAGAGGCCCAAAA | 122 | GACCAGGAGAGGCCCAAAA | 254 |
| ZNF48 | GAGGCCGGCGGCCCGAACT | 123 | GAGGCCGGCGGCCCGAACU | 255 |
| ZNF496 | GGAGCAGGAAGTGTCCCAG | 124 | GGAGCAGGAAGUGUCCCAG | 256 |
| ZNF501 | AAACGGGATGGAAGTCAGA | 125 | AAACGGGAUGGAAGUCAGA | 257 |
| ZNF524 | GGGTCCTAACGACCGGTCG | 126 | GGGUCCUAACGACCGGUCG | 258 |
| ZNF557 | CTGGCGCGGGTCTGTGGCA | 127 | CUGGCGCGGGUCUGUGGCA | 259 |
| ZNF735 | AAAAGGCCCGGTAAAATTA | 128 | AAAAGGCCCGGUAAAAUUA | 260 |
| ZNF747 | GGAACAAAGCAGCGCCCTG | 129 | GGAACAAAGCAGCGCCCUG | 261 |
| ZNF783 | AGGCGCTGCAACAGAGTCT | 130 | AGGCGCUGCAACAGAGUCU | 262 |
| ZNF805 | CTGGGGCCAATGATGGTCC | 131 | CUGGGGCCAAUGAUGGUCC | 263 |
| ZSCAN10 | TTACACTCTCACACAGGCT | 132 | UUACACUCUCACACAGGCU | 264 |

TABLE 2
Genes and gene-targeting gRNAs

| | | SEQ |
|---|---|---|
| Gene | Gene-targeting gRNA sequence | ID |
| DDIT3 | CCCUAGCGAGAGGGAGCGAGUUUAAGAGCUAUGCUGGAAACAGCAUAGCAAGU | 265 |
| | UUAAAUAAGGCUAGUCCGUUAUCAACUUGAAAAAGUGGCACCGAGUCGGUGC | |
| HES2 | CCGUCGGGGAUCUGCGGGCGUUUAAGAGCUAUGCUGGAAACAGCAUAGCAAGU | 266 |
| | UUAAAUAAGGCUAGUCCGUUAUCAACUUGAAAAAGUGGCACCGAGUCGGUGC | |
| HKR1 | CAGUCCCGCGAUGCCUUCGGUUUAAGAGCUAUGCUGGAAACAGCAUAGCAAGU | 267 |
| | UUAAAUAAGGCUAGUCCGUUAUCAACUUGAAAAAGUGGCACCGAGUCGGUGC | |
| PLAG1 | AACACUUCGGCUUGACGGGGUUUAAGAGCUAUGCUGGAAACAGCAUAGCAAGU | 268 |
| | UUAAAUAAGGCUAGUCCGUUAUCAACUUGAAAAAGUGGCACCGAGUCGGUGC | |
| PRDM4 | UGGCCGCGAGAGGCUCGGCGUUUAAGAGCUAUGCUGGAAACAGCAUAGCAAGU | 269 |
| | UUAAAUAAGGCUAGUCCGUUAUCAACUUGAAAAAGUGGCACCGAGUCGGUGC | |
| TEF | AGGCUGGGAGCCAUAACUGGUUUAAGAGCUAUGCUGGAAACAGCAUAGCAAGU | 270 |
| | UUAAAUAAGGCUAGUCCGUUAUCAACUUGAAAAAGUGGCACCGAGUCGGUGC | |
| TSHZ1 | GACCUAUUUGUAAUUGUCAGUUUAAGAGCUAUGCUGGAAACAGCAUAGCAAGU | 271 |
| | UUAAAUAAGGCUAGUCCGUUAUCAACUUGAAAAAGUGGCACCGAGUCGGUGC | |
| ZNF141 | GAGAGCUGUGCACCCAACUGUUUAAGAGCUAUGCUGGAAACAGCAUAGCAAGU | 272 |
| | UUAAAUAAGGCUAGUCCGUUAUCAACUUGAAAAAGUGGCACCGAGUCGGUGC | |
| ZNF562 | UGGACGGAACCGUGGGCGAGUUUAAGAGCUAUGCUGGAAACAGCAUAGCAAGU | 273 |
| | UUAAAUAAGGCUAGUCCGUUAUCAACUUGAAAAAGUGGCACCGAGUCGGUGC | |
| ZNF691 | CGCCCUACUACUCCAGGAUGUUUAAGAGCUAUGCUGGAAACAGCAUAGCAAGU | 274 |
| | UUAAAUAAGGCUAGUCCGUUAUCAACUUGAAAAAGUGGCACCGAGUCGGUGC | |
| ZNF692 | CCCCGCGGCAGUUGUGGUUGUUUAAGAGCUAUGCUGGAAACAGCAUAGCAAGU | 275 |
| | UUAAAUAAGGCUAGUCCGUUAUCAACUUGAAAAAGUGGCACCGAGUCGGUGC | |
| ZSCAN5A | ACGUAAUCGGCGUUGAUUAGUUUAAGAGCUAUGCUGGAAACAGCAUAGCAAGU | 276 |
| | UUAAAUAAGGCUAGUCCGUUAUCAACUUGAAAAAGUGGCACCGAGUCGGUGC | |
| EBF4 | AAGACGCCGGGGCUUCGUCGUUUAAGAGCUAUGCUGGAAACAGCAUAGCAAGU | 277 |
| | UUAAAUAAGGCUAGUCCGUUAUCAACUUGAAAAAGUGGCACCGAGUCGGUGC | |
| ETS1 | AGAAACGCCCUAAAGAGGAGUUUAAGAGCUAUGCUGGAAACAGCAUAGCAAGU | 278 |
| | UUAAAUAAGGCUAGUCCGUUAUCAACUUGAAAAAGUGGCACCGAGUCGGUGC | |
| FOXD3 | GCUGCAAGGGCGGUGUUGGGUUUAAGAGCUAUGCUGGAAACAGCAUAGCAAGU | 279 |
| | UUAAAUAAGGCUAGUCCGUUAUCAACUUGAAAAAGUGGCACCGAGUCGGUGC | |
| HELT | CAAGCGCCUGCAGCAGUCGGUUUAAGAGCUAUGCUGGAAACAGCAUAGCAAGU | 280 |
| | UUAAAUAAGGCUAGUCCGUUAUCAACUUGAAAAAGUGGCACCGAGUCGGUGC | |
| HMGN3 | GCGUACCGCGCGCGGAGGUGUUUAAGAGCUAUGCUGGAAACAGCAUAGCAAGU | 281 |
| | UUAAAUAAGGCUAGUCCGUUAUCAACUUGAAAAAGUGGCACCGAGUCGGUGC | |
| PATZ1 | GAAACGGCGAGCUCAGAGUGUUUAAGAGCUAUGCUGGAAACAGCAUAGCAAGU | 282 |
| | UUAAAUAAGGCUAGUCCGUUAUCAACUUGAAAAAGUGGCACCGAGUCGGUGC | |
| PRDM8 | CCCAGCUCUCCAUCCCCGGGUUUAAGAGCUAUGCUGGAAACAGCAUAGCAAGU | 283 |
| | UUAAAUAAGGCUAGUCCGUUAUCAACUUGAAAAAGUGGCACCGAGUCGGUGC | |
| SOX21 | CGGGAGAGGAAGACUCGAGGUUUAAGAGCUAUGCUGGAAACAGCAUAGCAAGU | 284 |
| | UUAAAUAAGGCUAGUCCGUUAUCAACUUGAAAAAGUGGCACCGAGUCGGUGC | |
| TBX15 | GAUCACAGGGAGGACGGGAGUUUAAGAGCUAUGCUGGAAACAGCAUAGCAAGU | 285 |
| | UUAAAUAAGGCUAGUCCGUUAUCAACUUGAAAAAGUGGCACCGAGUCGGUGC | |
| ZBED5 | AAAGGACCAAGCUCGGUUGGUUUAAGAGCUAUGCUGGAAACAGCAUAGCAAGU | 286 |
| | UUAAAUAAGGCUAGUCCGUUAUCAACUUGAAAAAGUGGCACCGAGUCGGUGC | |
| ZBTB7A | UCCCUCCGCCGUUUGCAGCGUUUAAGAGCUAUGCUGGAAACAGCAUAGCAAGU | 287 |
| | UUAAAUAAGGCUAGUCCGUUAUCAACUUGAAAAAGUGGCACCGAGUCGGUGC | |
| ZNF219 | CCUAAACCCGGACAUACCAGUUUAAGAGCUAUGCUGGAAACAGCAUAGCAAGU | 288 |
| | UUAAAUAAGGCUAGUCCGUUAUCAACUUGAAAAAGUGGCACCGAGUCGGUGC | |
| ZNF319 | UACCACUCUAAGCCUUCUCGUUUAAGAGCUAUGCUGGAAACAGCAUAGCAAGU | 289 |
| | UUAAAUAAGGCUAGUCCGUUAUCAACUUGAAAAAGUGGCACCGAGUCGGUGC | |
| ZNF470 | UUCCCAGGGGCUAUUGCAGGUUUAAGAGCUAUGCUGGAAACAGCAUAGCAAGU | 290 |
| | UUAAAUAAGGCUAGUCCGUUAUCAACUUGAAAAAGUGGCACCGAGUCGGUGC | |
| ZNF816 | AGCAGGAAUGCGGGCGCGGGUUUAAGAGCUAUGCUGGAAACAGCAUAGCAAGU | 291 |
| | UUAAAUAAGGCUAGUCCGUUAUCAACUUGAAAAAGUGGCACCGAGUCGGUGC | |
| ZSCAN23 | CUCCGCGAAACAGCGGUUCGUUUAAGAGCUAUGCUGGAAACAGCAUAGCAAGU | 292 |
| | UUAAAUAAGGCUAGUCCGUUAUCAACUUGAAAAAGUGGCACCGAGUCGGUGC | |
| EMX1 | AGCGCCGGGAGCCGUGGUUGUUUAAGAGCUAUGCUGGAAACAGCAUAGCAAGU | 293 |
| | UUAAAUAAGGCUAGUCCGUUAUCAACUUGAAAAAGUGGCACCGAGUCGGUGC | |
| FOXO3 | GCCGCGGGAGGCACUAGAGGUUUAAGAGCUAUGCUGGAAACAGCAUAGCAAGU | 294 |
| | UUAAAUAAGGCUAGUCCGUUAUCAACUUGAAAAAGUGGCACCGAGUCGGUGC | |
| HIC2 | UGGGCUCCUGGGGAAGCCCGUUUAAGAGCUAUGCUGGAAACAGCAUAGCAAGU | 295 |
| | UUAAAUAAGGCUAGUCCGUUAUCAACUUGAAAAAGUGGCACCGAGUCGGUGC | |
| SOX30 | GCCGCGGAAAGCAGAUCUGGUUUAAGAGCUAUGCUGGAAACAGCAUAGCAAGU | 296 |
| | UUAAAUAAGGCUAGUCCGUUAUCAACUUGAAAAAGUGGCACCGAGUCGGUGC | |
| STAT1 | CAGCGUGGCAACACGGGACGUUUAAGAGCUAUGCUGGAAACAGCAUAGCAAGU | 297 |
| | UUAAAUAAGGCUAGUCCGUUAUCAACUUGAAAAAGUGGCACCGAGUCGGUGC | |
| ZBTB46 | CCGCGCCGCGGGGUCGCAGGUUUAAGAGCUAUGCUGGAAACAGCAUAGCAAGU | 298 |
| | UUAAAUAAGGCUAGUCCGUUAUCAACUUGAAAAAGUGGCACCGAGUCGGUGC | |
| ZNF331 | CAGGUGAGAUUGGCGCCCAGUUUAAGAGCUAUGCUGGAAACAGCAUAGCAAGU | 299 |
| | UUAAAUAAGGCUAGUCCGUUAUCAACUUGAAAAAGUGGCACCGAGUCGGUGC | |
| ZNF541 | CAAGUCAUGUGUCAGAUAAGUUUAAGAGCUAUGCUGGAAACAGCAUAGCAAGU | 300 |
| | UUAAAUAAGGCUAGUCCGUUAUCAACUUGAAAAAGUGGCACCGAGUCGGUGC | |
| ZNF619 | CGCAGAGCGGUUGUGGAGAGUUUAAGAGCUAUGCUGGAAACAGCAUAGCAAGU | 301 |
| | UUAAAUAAGGCUAGUCCGUUAUCAACUUGAAAAAGUGGCACCGAGUCGGUGC | |
| AEBP1 | CGGGACCCCGAGUCCACCGGUUUAAGAGCUAUGCUGGAAACAGCAUAGCAAGU | 302 |
| | UUAAAUAAGGCUAGUCCGUUAUCAACUUGAAAAAGUGGCACCGAGUCGGUGC | |
| ARID3A | CCCGCCCCCGGCCUAGGGAGUUUAAGAGCUAUGCUGGAAACAGCAUAGCAAGU | 303 |
| | UUAAAUAAGGCUAGUCCGUUAUCAACUUGAAAAAGUGGCACCGAGUCGGUGC | |
| ARNTL2 | ACCCUCGGGGCAAAGGUAAGUUUAAGAGCUAUGCUGGAAACAGCAUAGCAAGU | 304 |
| | UUAAAUAAGGCUAGUCCGUUAUCAACUUGAAAAAGUGGCACCGAGUCGGUGC | |
| BACH1 | UCGCUGACAGAGGCUUCGAGUUUAAGAGCUAUGCUGGAAACAGCAUAGCAAGU | 305 |
| | UUAAAUAAGGCUAGUCCGUUAUCAACUUGAAAAAGUGGCACCGAGUCGGUGC | |
| BATF2 | CAAAGGCCAGGUAUGAGGAGUUUAAGAGCUAUGCUGGAAACAGCAUAGCAAGU | 306 |
| | UUAAAUAAGGCUAGUCCGUUAUCAACUUGAAAAAGUGGCACCGAGUCGGUGC | |
| BRD4 | UGCUGAAGUCAUGAGGUGUGUUUAAGAGCUAUGCUGGAAACAGCAUAGCAAGU | 307 |
| | UUAAAUAAGGCUAGUCCGUUAUCAACUUGAAAAAGUGGCACCGAGUCGGUGC | |
| CDX4 | GGGAAGAUCGGUGAAAGGGGUUUAAGAGCUAUGCUGGAAACAGCAUAGCAAGU | 308 |
| | UUAAAUAAGGCUAGUCCGUUAUCAACUUGAAAAAGUGGCACCGAGUCGGUGC | |
| CENPB | GCCCCCCCGCGGUAUGUCCGUUUAAGAGCUAUGCUGGAAACAGCAUAGCAAGU | 309 |
| | UUAAAUAAGGCUAGUCCGUUAUCAACUUGAAAAAGUGGCACCGAGUCGGUGC | |
| DMRT1 | AGAGCGGUGGCUGGAGUCGGUUUAAGAGCUAUGCUGGAAACAGCAUAGCAAGU | 310 |
| | UUAAAUAAGGCUAGUCCGUUAUCAACUUGAAAAAGUGGCACCGAGUCGGUGC | |
| E2F2 | CGGAAAGAACGCGUCCUCGGUUUAAGAGCUAUGCUGGAAACAGCAUAGCAAGU | 311 |
| | UUAAAUAAGGCUAGUCCGUUAUCAACUUGAAAAAGUGGCACCGAGUCGGUGC | |
| ESRRB | GAGGGGCAACCCCCGACUCGUUUAAGAGCUAUGCUGGAAACAGCAUAGCAAGU | 312 |
| | UUAAAUAAGGCUAGUCCGUUAUCAACUUGAAAAAGUGGCACCGAGUCGGUGC | |
| FERD3L | UGAGUAGGAGGUAAAGGUAGUUUAAGAGCUAUGCUGGAAACAGCAUAGCAAGU | 313 |
| | UUAAAUAAGGCUAGUCCGUUAUCAACUUGAAAAAGUGGCACCGAGUCGGUGC | |
| FEV | CGGGAGCCGACUGGAAGAAGUUUAAGAGCUAUGCUGGAAACAGCAUAGCAAGU | 314 |
| | UUAAAUAAGGCUAGUCCGUUAUCAACUUGAAAAAGUGGCACCGAGUCGGUGC | |
| FLYWCH1 | GGGACGUAACUCACGGAAAGUUUAAGAGCUAUGCUGGAAACAGCAUAGCAAGU | 315 |
| | UUAAAUAAGGCUAGUCCGUUAUCAACUUGAAAAAGUGGCACCGAGUCGGUGC | |
| FOXJ3 | CAGAGCCUCGGGAGUCUCAGUUUAAGAGCUAUGCUGGAAACAGCAUAGCAAGU | 316 |
| | UUAAAUAAGGCUAGUCCGUUAUCAACUUGAAAAAGUGGCACCGAGUCGGUGC | |
| GLIS3 | GGCUCGCGGGUCACUAGGCGUUUAAGAGCUAUGCUGGAAACAGCAUAGCAAGU | 317 |
| | UUAAAUAAGGCUAGUCCGUUAUCAACUUGAAAAAGUGGCACCGAGUCGGUGC | |
| HDAC9 | GUAAAGCAAGCUUCGAGAGGUUUAAGAGCUAUGCUGGAAACAGCAUAGCAAGU | 318 |
| | UUAAAUAAGGCUAGUCCGUUAUCAACUUGAAAAAGUGGCACCGAGUCGGUGC | |
| HIVEP1 | CAGUCACGCGGCCCCGGGAGUUUAAGAGCUAUGCUGGAAACAGCAUAGCAAGU | 319 |
| | UUAAAUAAGGCUAGUCCGUUAUCAACUUGAAAAAGUGGCACCGAGUCGGUGC | |
| HOXA2 | GGAAGGAACGCCUCAUUGGGUUUAAGAGCUAUGCUGGAAACAGCAUAGCAAGU | 320 |
| | UUAAAUAAGGCUAGUCCGUUAUCAACUUGAAAAAGUGGCACCGAGUCGGUGC | |
| HOXC9 | GAGCUAGGAGCCGUCCUGAGUUUAAGAGCUAUGCUGGAAACAGCAUAGCAAGU | 321 |
| | UUAAAUAAGGCUAGUCCGUUAUCAACUUGAAAAAGUGGCACCGAGUCGGUGC | |
| HSFY1 | GGUAGGUUACGGGUUGUCAGUUUAAGAGCUAUGCUGGAAACAGCAUAGCAAGU | 322 |
| | UUAAAUAAGGCUAGUCCGUUAUCAACUUGAAAAAGUGGCACCGAGUCGGUGC | |
| ISL2 | AACGGCCGCGGAGCCAGCUGUUUAAGAGCUAUGCUGGAAACAGCAUAGCAAGU | 323 |
| | UUAAAUAAGGCUAGUCCGUUAUCAACUUGAAAAAGUGGCACCGAGUCGGUGC | |
| KAT2A | UCCGGAGCCGCGUGGGGCAGUUUAAGAGCUAUGCUGGAAACAGCAUAGCAAGU | 324 |
| | UUAAAUAAGGCUAGUCCGUUAUCAACUUGAAAAAGUGGCACCGAGUCGGUGC | |
| KDM5D | GCAAGAGCGAUUUUACCAGGUUUAAGAGCUAUGCUGGAAACAGCAUAGCAAGU | 325 |
| | UUAAAUAAGGCUAGUCCGUUAUCAACUUGAAAAAGUGGCACCGAGUCGGUGC | |
| KLF13 | ACCGGGCACGCCCCUCUAAGUUUAAGAGCUAUGCUGGAAACAGCAUAGCAAGU | 326 |
| | UUAAAUAAGGCUAGUCCGUUAUCAACUUGAAAAAGUGGCACCGAGUCGGUGC | |
| KLF6 | UGACCGAGACUCUCCAUCAGUUUAAGAGCUAUGCUGGAAACAGCAUAGCAAGU | 327 |
| | UUAAAUAAGGCUAGUCCGUUAUCAACUUGAAAAAGUGGCACCGAGUCGGUGC | |
| MEOX2 | AAUCAGGGUGCUAGGUUGGGUUUAAGAGCUAUGCUGGAAACAGCAUAGCAAGU | 328 |
| | UUAAAUAAGGCUAGUCCGUUAUCAACUUGAAAAAGUGGCACCGAGUCGGUGC | |
| MLXIPL | CGCCAGAGCUCCAGAGCACGUUUAAGAGCUAUGCUGGAAACAGCAUAGCAAGU | 329 |
| | UUAAAUAAGGCUAGUCCGUUAUCAACUUGAAAAAGUGGCACCGAGUCGGUGC | |
| MNT | GGGCCGCGGCGUCUCGCGAGUUUAAGAGCUAUGCUGGAAACAGCAUAGCAAGU | 330 |
| | UUAAAUAAGGCUAGUCCGUUAUCAACUUGAAAAAGUGGCACCGAGUCGGUGC | |
| MYCN | CAAGCCCGGGGGUCCAAAAGUUUAAGAGCUAUGCUGGAAACAGCAUAGCAAGU | 331 |
| | UUAAAUAAGGCUAGUCCGUUAUCAACUUGAAAAAGUGGCACCGAGUCGGUGC | |
| MYT1L | GCCUCAUAGAUAAUGAUGAGUUUAAGAGCUAUGCUGGAAACAGCAUAGCAAGU | 332 |
| | UUAAAUAAGGCUAGUCCGUUAUCAACUUGAAAAAGUGGCACCGAGUCGGUGC | |
| NEUROG3 | CUCGCGUGGCGCGGCCCCAGUUUAAGAGCUAUGCUGGAAACAGCAUAGCAAGU | 333 |
| | UUAAAUAAGGCUAGUCCGUUAUCAACUUGAAAAAGUGGCACCGAGUCGGUGC | |
| NFE2 | AGGAGAGCCACGGGGAAAGGUUUAAGAGCUAUGCUGGAAACAGCAUAGCAAGU | 334 |
| | UUAAAUAAGGCUAGUCCGUUAUCAACUUGAAAAAGUGGCACCGAGUCGGUGC | |
| NR2F1 | UUUCUCUCCCCCCGAUUCAGUUUAAGAGCUAUGCUGGAAACAGCAUAGCAAGU | 335 |
| | UUAAAUAAGGCUAGUCCGUUAUCAACUUGAAAAAGUGGCACCGAGUCGGUGC | |
| NR2F2 | CCCCCUCGCCGUCAUUGGCGUUUAAGAGCUAUGCUGGAAACAGCAUAGCAAGU | 336 |
| | UUAAAUAAGGCUAGUCCGUUAUCAACUUGAAAAAGUGGCACCGAGUCGGUGC | |
| NR3C1 | GGGCCCGCGCGGAGGGCGUGUUUAAGAGCUAUGCUGGAAACAGCAUAGCAAGU | 337 |
| | UUAAAUAAGGCUAGUCCGUUAUCAACUUGAAAAAGUGGCACCGAGUCGGUGC | |
| PHF20 | AGGCCGUUGCCACAGUGAUGUUUAAGAGCUAUGCUGGAAACAGCAUAGCAAGU | 338 |
| | UUAAAUAAGGCUAGUCCGUUAUCAACUUGAAAAAGUGGCACCGAGUCGGUGC | |
| PITX2 | CAGGACAGUCCCUCCACCGGUUUAAGAGCUAUGCUGGAAACAGCAUAGCAAGU | 339 |
| | UUAAAUAAGGCUAGUCCGUUAUCAACUUGAAAAAGUGGCACCGAGUCGGUGC | |
| POU2AF1 | GCCGCUGGGGCGCGCACAGGUUUAAGAGCUAUGCUGGAAACAGCAUAGCAAGU | 340 |
| | UUAAAUAAGGCUAGUCCGUUAUCAACUUGAAAAAGUGGCACCGAGUCGGUGC | |
| POU3F2 | GCGGGGCCCAUCCGAGAGAGUUUAAGAGCUAUGCUGGAAACAGCAUAGCAAGU | 341 |
| | UUAAAUAAGGCUAGUCCGUUAUCAACUUGAAAAAGUGGCACCGAGUCGGUGC | |
| POU3F3 | GGCCACGACCCCCCCUGAAGUUUAAGAGCUAUGCUGGAAACAGCAUAGCAAGU | 342 |
| | UUAAAUAAGGCUAGUCCGUUAUCAACUUGAAAAAGUGGCACCGAGUCGGUGC | |
| PRDM11 | UGGGUGUGGAGGGGUUCCAGUUUAAGAGCUAUGCUGGAAACAGCAUAGCAAGU | 343 |
| | UUAAAUAAGGCUAGUCCGUUAUCAACUUGAAAAAGUGGCACCGAGUCGGUGC | |
| PRMT3 | UGAAGAGGCGCGCUGAUGAGUUUAAGAGCUAUGCUGGAAACAGCAUAGCAAGU | 344 |
| | UUAAAUAAGGCUAGUCCGUUAUCAACUUGAAAAAGUGGCACCGAGUCGGUGC | |
| PROP1 | UCGGAGGGUCCAGGGACUGGUUUAAGAGCUAUGCUGGAAACAGCAUAGCAAGU | 345 |
| | UUAAAUAAGGCUAGUCCGUUAUCAACUUGAAAAAGUGGCACCGAGUCGGUGC | |
| PURA | CUCACGCGCGAGAAGGGGGGUUUAAGAGCUAUGCUGGAAACAGCAUAGCAAGU | 346 |
| | UUAAAUAAGGCUAGUCCGUUAUCAACUUGAAAAAGUGGCACCGAGUCGGUGC | |
| RELB | UGGGCGCUCAGGAGGGGUUGUUUAAGAGCUAUGCUGGAAACAGCAUAGCAAGU | 347 |
| | UUAAAUAAGGCUAGUCCGUUAUCAACUUGAAAAAGUGGCACCGAGUCGGUGC | |
| RFX3 | ACUGACGGCCCCCAAACCCGUUUAAGAGCUAUGCUGGAAACAGCAUAGCAAGU | 348 |
| | UUAAAUAAGGCUAGUCCGUUAUCAACUUGAAAAAGUGGCACCGAGUCGGUGC | |
| SAFB | CUGCUAAGUACGCUAGUGGGUUUAAGAGCUAUGCUGGAAACAGCAUAGCAAGU | 349 |
| | UUAAAUAAGGCUAGUCCGUUAUCAACUUGAAAAAGUGGCACCGAGUCGGUGC | |
| SETBP1 | UAUGGAGAGAGAUAUCAAGGUUUAAGAGCUAUGCUGGAAACAGCAUAGCAAGU | 350 |
| | UUAAAUAAGGCUAGUCCGUUAUCAACUUGAAAAAGUGGCACCGAGUCGGUGC | |
| SMAD2 | AAGCCGCGCGGGGCGGGUAGUUUAAGAGCUAUGCUGGAAACAGCAUAGCAAGU | 351 |
| | UUAAAUAAGGCUAGUCCGUUAUCAACUUGAAAAAGUGGCACCGAGUCGGUGC | |
| SMAD6 | CCCGGGGGGGGCCGAGCUCGUUUAAGAGCUAUGCUGGAAACAGCAUAGCAAGU | 352 |
| | UUAAAUAAGGCUAGUCCGUUAUCAACUUGAAAAAGUGGCACCGAGUCGGUGC | |
| SNAPC2 | AGGGAGGGCAUGGGAUCCAGUUUAAGAGCUAUGCUGGAAACAGCAUAGCAAGU | 353 |
| | UUAAAUAAGGCUAGUCCGUUAUCAACUUGAAAAAGUGGCACCGAGUCGGUGC | |
| SNAPC4 | UCUCAGAGCCCACCCUCGAGUUUAAGAGCUAUGCUGGAAACAGCAUAGCAAGU | 354 |
| | UUAAAUAAGGCUAGUCCGUUAUCAACUUGAAAAAGUGGCACCGAGUCGGUGC | |
| SON | CUGUACUUGGCCUGUACUAGUUUAAGAGCUAUGCUGGAAACAGCAUAGCAAGU | 355 |
| | UUAAAUAAGGCUAGUCCGUUAUCAACUUGAAAAAGUGGCACCGAGUCGGUGC | |
| SOX11 | GGGCUGCUUUGCAAAGAUGGUUUAAGAGCUAUGCUGGAAACAGCAUAGCAAGU | 356 |
| | UUAAAUAAGGCUAGUCCGUUAUCAACUUGAAAAAGUGGCACCGAGUCGGUGC | |
| SOX12 | GGCCCGGCCGCAGCCCAAGGUUUAAGAGCUAUGCUGGAAACAGCAUAGCAAGU | 357 |
| | UUAAAUAAGGCUAGUCCGUUAUCAACUUGAAAAAGUGGCACCGAGUCGGUGC | |
| SOX14 | CAAAAGCGGCGAAGUUCAAGUUUAAGAGCUAUGCUGGAAACAGCAUAGCAAGU | 358 |
| | UUAAAUAAGGCUAGUCCGUUAUCAACUUGAAAAAGUGGCACCGAGUCGGUGC | |
| SPIC | UGCAACCUAAGUUGGGCGGGUUUAAGAGCUAUGCUGGAAACAGCAUAGCAAGU | 359 |
| | UUAAAUAAGGCUAGUCCGUUAUCAACUUGAAAAAGUGGCACCGAGUCGGUGC | |
| THAP7 | AAUCGGCUGGGACGGGGUUGUUUAAGAGCUAUGCUGGAAACAGCAUAGCAAGU | 360 |
| | UUAAAUAAGGCUAGUCCGUUAUCAACUUGAAAAAGUGGCACCGAGUCGGUGC | |
| TOX2 | GCUCCCGCGGGCAGUGCCUGUUUAAGAGCUAUGCUGGAAACAGCAUAGCAAGU | 361 |
| | UUAAAUAAGGCUAGUCCGUUAUCAACUUGAAAAAGUGGCACCGAGUCGGUGC | |
| TRERF1 | GGGUACCGGGGAGCGGAUAGUUUAAGAGCUAUGCUGGAAACAGCAUAGCAAGU | 362 |
| | UUAAAUAAGGCUAGUCCGUUAUCAACUUGAAAAAGUGGCACCGAGUCGGUGC | |
| TRIM3 | GGGCGUGCGGGUGCGGUAGGUUUAAGAGCUAUGCUGGAAACAGCAUAGCAAGU | 363 |
| | UUAAAUAAGGCUAGUCCGUUAUCAACUUGAAAAAGUGGCACCGAGUCGGUGC | |
| VDR | UACAGCGUGGCCUAUAGGGGUUUAAGAGCUAUGCUGGAAACAGCAUAGCAAGU | 364 |
| | UUAAAUAAGGCUAGUCCGUUAUCAACUUGAAAAAGUGGCACCGAGUCGGUGC | |
| YBX2 | GGGCCCAGGUGUACUUACUGUUUAAGAGCUAUGCUGGAAACAGCAUAGCAAGU | 365 |
| | UUAAAUAAGGCUAGUCCGUUAUCAACUUGAAAAAGUGGCACCGAGUCGGUGC | |
| ZBTB14 | CGCGCGCGUGCGCAGGCCAGUUUAAGAGCUAUGCUGGAAACAGCAUAGCAAGU | 366 |
| | UUAAAUAAGGCUAGUCCGUUAUCAACUUGAAAAAGUGGCACCGAGUCGGUGC | |
| ZBTB4 | CCGGGACGGUUGGAGAAGAGUUUAAGAGCUAUGCUGGAAACAGCAUAGCAAGU | 367 |
| | UUAAAUAAGGCUAGUCCGUUAUCAACUUGAAAAAGUGGCACCGAGUCGGUGC | |
| ZFHX2 | GGUACAGGUCUCAUGCCUGGUUUAAGAGCUAUGCUGGAAACAGCAUAGCAAGU | 368 |
| | UUAAAUAAGGCUAGUCCGUUAUCAACUUGAAAAAGUGGCACCGAGUCGGUGC | |
| ZFP57 | GCAGCUAUCAGUUGGUCGGGUUUAAGAGCUAUGCUGGAAACAGCAUAGCAAGU | 369 |
| | UUAAAUAAGGCUAGUCCGUUAUCAACUUGAAAAAGUGGCACCGAGUCGGUGC | |
| ZFP62 | GAGGGCAGAGAGACCUCUAGUUUAAGAGCUAUGCUGGAAACAGCAUAGCAAGU | 370 |
| | UUAAAUAAGGCUAGUCCGUUAUCAACUUGAAAAAGUGGCACCGAGUCGGUGC | |
| ZFP69B | UGAUGAUGCUCGCAGGGAUGUUUAAGAGCUAUGCUGGAAACAGCAUAGCAAGU | 371 |
| | UUAAAUAAGGCUAGUCCGUUAUCAACUUGAAAAAGUGGCACCGAGUCGGUGC | |
| ZIC1 | GGGAAUGCGGGCGCACCAAGUUUAAGAGCUAUGCUGGAAACAGCAUAGCAAGU | 372 |
| | UUAAAUAAGGCUAGUCCGUUAUCAACUUGAAAAAGUGGCACCGAGUCGGUGC | |
| ZIC4 | CAACGGGGACGUUGACAAGGUUUAAGAGCUAUGCUGGAAACAGCAUAGCAAGU | 373 |
| | UUAAAUAAGGCUAGUCCGUUAUCAACUUGAAAAAGUGGCACCGAGUCGGUGC | |
| ZNF136 | CCCCGACGGCGCGCCUGAUGUUUAAGAGCUAUGCUGGAAACAGCAUAGCAAGU | 374 |
| | UUAAAUAAGGCUAGUCCGUUAUCAACUUGAAAAAGUGGCACCGAGUCGGUGC | |
| ZNF16 | GCUAGUGGGACCAGGUCUGGUUUAAGAGCUAUGCUGGAAACAGCAUAGCAAGU | 375 |
| | UUAAAUAAGGCUAGUCCGUUAUCAACUUGAAAAAGUGGCACCGAGUCGGUGC | |
| ZNF174 | CAGGGGCGUGCGUAAUGGGGUUUAAGAGCUAUGCUGGAAACAGCAUAGCAAGU | 376 |
| | UUAAAUAAGGCUAGUCCGUUAUCAACUUGAAAAAGUGGCACCGAGUCGGUGC | |
| ZNF253 | UGUCACUCAGCGGCUGGGGGUUUAAGAGCUAUGCUGGAAACAGCAUAGCAAGU | 377 |
| | UUAAAUAAGGCUAGUCCGUUAUCAACUUGAAAAAGUGGCACCGAGUCGGUGC | |
| ZNF26 | ACAGCCUGACGGGAAAGGAGUUUAAGAGCUAUGCUGGAAACAGCAUAGCAAGU | 378 |
| | UUAAAUAAGGCUAGUCCGUUAUCAACUUGAAAAAGUGGCACCGAGUCGGUGC | |
| ZNF263 | GGGCGCGCCCUGAAAUUCUGUUUAAGAGCUAUGCUGGAAACAGCAUAGCAAGU | 379 |
| | UUAAAUAAGGCUAGUCCGUUAUCAACUUGAAAAAGUGGCACCGAGUCGGUGC | |
| ZNF285 | AAAGCCCCGCGCGCAGCAGGUUUAAGAGCUAUGCUGGAAACAGCAUAGCAAGU | 380 |
| | UUAAAUAAGGCUAGUCCGUUAUCAACUUGAAAAAGUGGCACCGAGUCGGUGC | |
| ZNF317 | UCCCGCGAGAAUUGGAAGGGUUUAAGAGCUAUGCUGGAAACAGCAUAGCAAGU | 381 |
| | UUAAAUAAGGCUAGUCCGUUAUCAACUUGAAAAAGUGGCACCGAGUCGGUGC | |
| ZNF319 | CAUCUUCCGGAAGGGAGGGGUUUAAGAGCUAUGCUGGAAACAGCAUAGCAAGU | 382 |
| | UUAAAUAAGGCUAGUCCGUUAUCAACUUGAAAAAGUGGCACCGAGUCGGUGC | |
| ZNF345 | CGCGCCAGGGGAAUCGGGAGUUUAAGAGCUAUGCUGGAAACAGCAUAGCAAGU | 383 |
| | UUAAAUAAGGCUAGUCCGUUAUCAACUUGAAAAAGUGGCACCGAGUCGGUGC | |
| ZNF420 | GAGCCCACGAGCCCCUCGAGUUUAAGAGCUAUGCUGGAAACAGCAUAGCAAGU | 384 |
| | UUAAAUAAGGCUAGUCCGUUAUCAACUUGAAAAAGUGGCACCGAGUCGGUGC | |
| ZNF423 | CAGGGGAUCAGCCAUCCAGGUUUAAGAGCUAUGCUGGAAACAGCAUAGCAAGU | 385 |
| | UUAAAUAAGGCUAGUCCGUUAUCAACUUGAAAAAGUGGCACCGAGUCGGUGC | |
| ZNF470 | GACCAGGAGAGGCCCAAAAGUUUAAGAGCUAUGCUGGAAACAGCAUAGCAAGU | 386 |
| | UUAAAUAAGGCUAGUCCGUUAUCAACUUGAAAAAGUGGCACCGAGUCGGUGC | |
| ZNF48 | GAGGCCGGCGGCCCGAACUGUUUAAGAGCUAUGCUGGAAACAGCAUAGCAAGU | 387 |
| | UUAAAUAAGGCUAGUCCGUUAUCAACUUGAAAAAGUGGCACCGAGUCGGUGC | |
| ZNF496 | GGAGCAGGAAGUGUCCCAGGUUUAAGAGCUAUGCUGGAAACAGCAUAGCAAGU | 388 |
| | UUAAAUAAGGCUAGUCCGUUAUCAACUUGAAAAAGUGGCACCGAGUCGGUGC | |
| ZNF501 | AAACGGGAUGGAAGUCAGAGUUUAAGAGCUAUGCUGGAAACAGCAUAGCAAGU | 389 |
| | UUAAAUAAGGCUAGUCCGUUAUCAACUUGAAAAAGUGGCACCGAGUCGGUGC | |
| ZNF524 | GGGUCCUAACGACCGGUCGGUUUAAGAGCUAUGCUGGAAACAGCAUAGCAAGU | 390 |
| | UUAAAUAAGGCUAGUCCGUUAUCAACUUGAAAAAGUGGCACCGAGUCGGUGC | |
| ZNF557 | CUGGCGCGGGUCUGUGGCAGUUUAAGAGCUAUGCUGGAAACAGCAUAGCAAGU | 391 |
| | UUAAAUAAGGCUAGUCCGUUAUCAACUUGAAAAAGUGGCACCGAGUCGGUGC | |
| ZNF735 | AAAAGGCCCGGUAAAAUUAGUUUAAGAGCUAUGCUGGAAACAGCAUAGCAAGU | 392 |
| | UUAAAUAAGGCUAGUCCGUUAUCAACUUGAAAAAGUGGCACCGAGUCGGUGC | |
| ZNF747 | GGAACAAAGCAGCGCCCUGGUUUAAGAGCUAUGCUGGAAACAGCAUAGCAAGU | 393 |
| | UUAAAUAAGGCUAGUCCGUUAUCAACUUGAAAAAGUGGCACCGAGUCGGUGC | |
| ZNF783 | AGGCGCUGCAACAGAGUCUGUUUAAGAGCUAUGCUGGAAACAGCAUAGCAAGU | 394 |
| | UUAAAUAAGGCUAGUCCGUUAUCAACUUGAAAAAGUGGCACCGAGUCGGUGC | |
| ZNF805 | CUGGGGCCAAUGAUGGUCCGUUUAAGAGCUAUGCUGGAAACAGCAUAGCAAGU | 395 |
| | UUAAAUAAGGCUAGUCCGUUAUCAACUUGAAAAAGUGGCACCGAGUCGGUGC | |
| ZSCAN10 | UUACACUCUCACACAGGCUGUUUAAGAGCUAUGCUGGAAACAGCAUAGCAAGU | 396 |
| | UUAAAUAAGGCUAGUCCGUUAUCAACUUGAAAAAGUGGCACCGAGUCGGUGC | |

In some embodiments, a gRNA provided herein targets a target site in a gene in a T cell or DNA regulatory element thereof, wherein the gene is selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23. In some embodiments, a gRNA provided herein comprises a spacer sequence selected from any one of SEQ ID NOS: 133-160, as shown in Table 1. In some embodiments, the gRNA further comprises a scaffold sequence set forth in SEQ ID NO: 398. In some embodiments, the gRNA comprises the sequence selected from any one of SEQ ID NOS: 265-292, as shown in Table 2, or a sequence having at or at least 80%, 85%, 90%, 91%, 92%, 93%, 94%, 95%, 96%, 97%, 98%, 99%, 99.5%, 99.9%, or 100% sequence identity to any one of SEQ ID NO: 265-292. In some embodiments, the gRNA is set forth in any one of SEQ ID NOS: 265-292. In some embodiments, any of the provided gRNA sequences is complexed with or is provided in combination with a Cas9. In some embodiments, the Cas9 is a dCas9. In some embodiments, the dCas9 is a dSpCas9, such as a dSpCas9 set forth in SEQ ID NO: 408.

In some embodiments, a gRNA provided herein targets a target site in a gene in a T cell or DNA regulatory element thereof, wherein the gene is selected from the list consisting of DDIT3, HES2, HKR1, PLAG1, PRDM4, TEF, TSHZ1, ZNF141, ZNF562, ZNF691, ZNF692, and ZSCAN5A. In some embodiments, a gRNA provided herein comprises a spacer sequence selected from any one of SEQ ID NOS: 133-144, as shown in Table 1. In some embodiments, the gRNA further comprises a scaffold sequence set forth in SEQ ID NO: 398. In some embodiments, the gRNA comprises the sequence selected from any one of SEQ ID NOS: 265-276, as shown in Table 2, or a sequence having at or at least 80%, 85%, 90%, 91%, 92%, 93%, 94%, 95%, 96%, 97%, 98%, 99%, 99.5%, 99.9%, or 100% sequence identity to any one of SEQ ID NO: 265-276. In some embodiments, the gRNA is set forth in any one of SEQ ID NOS: 265-276. In some embodiments, any of the provided gRNA sequences is complexed with or is provided in combination with a Cas9. In some embodiments, the Cas9 is a dCas9. In some embodiments, the dCas9 is a dSpCas9, such as a dSpCas9 set forth in SEQ ID NO: 408.

In some embodiments, a gRNA provided herein targets DDIT3 or a DNA regulatory element thereof. DDIT3 is a gene that encodes DNA damage-inducible transcript 3 (also known as C/EBP homologous protein or CHOP) and is a pro-apoptotic transcription factor. DDIT3 has been reported to have role in the cell stress response and has been reported to be a negative regulator of effector function of tumor-reactive CD8+ T cells (Cao et al. Nature Communications 10:1280 (2019)). In some embodiments, the gRNA targets a target site in DDIT3 or a DNA regulatory element thereof that comprises SEQ ID NO: 1, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 133, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 265, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting DDIT3 or a DNA regulatory element thereof, is set forth in SEQ ID NO:265. In some embodiments, a provided DNA-targeting system for epigenetic modification of DDIT3 includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets HES2 or a DNA regulatory element thereof. HES2 is a gene that encodes Hes family bHLH transcription factor 2 (also known as bHLHb40 and hes family bHLH transcription factor 2), and is a transcriptional repressor of genes that require a bHLH protein for their transcription. In some embodiments, the gRNA targets a target site in HES2 or a DNA regulatory element thereof that comprises SEQ ID NO: 2, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 134, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 266, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting HES2 or a DNA regulatory element thereof is set forth in SEQ ID NO: 266. In some embodiments, a provided DNA-targeting system for epigenetic modification of HES2 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets HKR1 or a DNA regulatory element thereof. HKR1 is a gene that encodes Krueppel-related zinc finger protein 1 (also known as ZNF875, GLI-Kruppel zinc finger family member, and zinc finger protein 875), and is involved in transcriptional regulation. In some embodiments, the gRNA targets a target site in HKR1 or a DNA regulatory element thereof that comprises SEQ ID NO: 3, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 135, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 267, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting HKR1 or a DNA regulatory element thereof is set forth in SEQ ID NO: 267. In some embodiments, a provided DNA-targeting system for epigenetic modification of HKR1 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets PLAG1 or a DNA regulatory element thereof. PLAG1 is a gene that encodes Zinc finger protein PLAG1 (also known as PSA, SGPA, ZNF912, PLAG1 zinc finger, and SRS4). PLAG1 is a transcription factor whose activation results in up-regulation of target genes. In some embodiments, the gRNA targets a target site in PLAG1 or a DNA regulatory element thereof that comprises SEQ ID NO: 4, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 136, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%. 99.5%, 99.9%, or 100% sequence identity to SEQ ID NO: 398. In some embodiments, the gRNA, including a spacer sequence and a scaffold sequence, comprises SEQ ID NO: 268, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting PLAG1 or a DNA regulatory element thereof is set forth in SEQ ID NO: 268. In some embodiments, a provided DNA-targeting system for epigenetic modification of PLAG1 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets PRDM4 or a DNA regulatory element thereof. PRDM4 is a gene that encodes PR domain zinc finger protein 4. PRDM4 may function as a transcription factor involved in cell differentiation. In some embodiments, the gRNA targets a target site in PRDM4 or a DNA regulatory element thereof that comprises SEQ ID NO: 5, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 137, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 269, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting PRDM4 or a DNA regulatory element thereof is set forth in SEQ ID NO: 269. In some embodiments, a provided DNA-targeting system for epigenetic modification of PRDM4 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets TEF or a DNA regulatory element thereof. TEF is a gene that encodes Thyrotroph embryonic factor (also known as PAR bZIP transcription factor, TEF transcription factor, and PAR bZIP family member). TEF is a member of the PAR (proline and acidic amino acid-rich) subfamily of basic region/leucine zipper (bZIP) transcription factors. It is expressed in a broad range of cells and tissues in adult animals, and has restricted expression in development. In some embodiments, the gRNA targets a target site in TEF or a DNA regulatory element thereof that comprises SEQ ID NO: 6, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 138, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 270, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting TEF or a DNA regulatory element thereof is set forth in SEQ ID NO: 270. In some embodiments, a provided DNA-targeting system for epigenetic modification of TEF or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets TSHZ1 or a DNA regulatory element thereof. TSHZ1 is a gene that encodes Teashirt homolog 1 (also known as CAA, NY-CO-33, SDCCAG33, TSH1, and teashirt zinc finger homeobox 1). TSHZ1 encodes a colon cancer antigen that was defined by serological analysis of recombinant cDNA expression libraries. The encoded protein is a member of the teashirt C2H2-type zinc-finger protein family and may be involved in transcriptional regulation of developmental processes. In some embodiments, the gRNA targets a target site in TSHZ1 or a DNA regulatory element thereof that comprises SEQ ID NO: 7, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 139, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 271, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting TSHZ1 or a DNA regulatory element thereof is set forth in SEQ ID NO: 271. In some embodiments, a provided DNA-targeting system for epigenetic modification of TSHZ1 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets ZNF141 or a DNA regulatory element thereof. ZNF141 is a gene that encodes Zinc finger protein 141, ZNF141 may be involved in transcriptional regulation as a repressor, and plays a role in limb development. In some embodiments, the gRNA targets a target site in ZNF141 or a DNA regulatory element thereof that comprises SEQ ID NO: 8, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 140, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 272, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ZNF141 or a DNA regulatory element thereof is set forth in SEQ ID NO: 272. In some embodiments, a provided DNA-targeting system for epigenetic modification of ZNF141 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets ZNF562 or a DNA regulatory element thereof. ZNF562 is a gene that encodes Zinc finger protein 562, and may function as a transcription factor. In some embodiments, the gRNA targets a target site in ZNF562 or a DNA regulatory element thereof that comprises SEQ ID NO: 9, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 141, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 273, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ZNF562 or a DNA regulatory element thereof is set forth in SEQ ID NO: 273. In some embodiments, a provided DNA-targeting system for epigenetic modification of ZNF562 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets ZNF691 or a DNA regulatory element thereof. ZNF691 is a gene that encodes Zinc finger protein 691, and may be involved in transcriptional regulation. In some embodiments, the gRNA targets a target site in ZNF691 or a DNA regulatory element thereof that comprises SEQ ID NO: 10, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 142, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 274, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ZNF691 or a DNA regulatory element thereof is set forth in SEQ ID NO: 274. In some embodiments, a provided DNA-targeting system for epigenetic modification of ZNF691 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets ZNF692 or a DNA regulatory element thereof. ZNF692 is a gene that encodes Zinc finger protein 692 (also known as AREBP and Zfp692). ZNF692 may act as an transcriptional repressor, including for PCK1 gene expression, and in turn may participate in the hepatic gluconeogenesis regulation through the activated AMPK signaling pathway. In some embodiments, the gRNA targets a target site in ZNF692 or a DNA regulatory element thereof that comprises SEQ ID NO: 11, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 143, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 275, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ZNF692 or a DNA regulatory element thereof is set forth in SEQ ID NO: 275. In some embodiments, a provided DNA-targeting system for epigenetic modification of ZNF692 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets ZSCAN5A or a DNA regulatory element thereof. ZSCAN5A is a gene that encodes Zinc finger and SCAN domain-containing protein 5A, and may function as a transcription factor. In some embodiments, the gRNA targets a target site in ZSCAN5A or a DNA regulatory element thereof that comprises SEQ ID NO: 12, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 144, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 276, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ZSCAN5A or a DNA regulatory element thereof is set forth in SEQ ID NO: 276. In some embodiments, a provided DNA-targeting system for epigenetic modification of ZSCAN5A or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets EBF4 or a DNA regulatory element thereof. EBF4 is a gene that encodes Transcription factor COE4, and which functions as a transcription factor. In some embodiments, the gRNA targets a target site in EBF4 or a DNA regulatory element thereof that comprises SEQ ID NO: 13, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 145, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 277, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting EBF4 or a DNA regulatory element thereof is set forth in SEQ ID NO: 277. In some embodiments, a provided DNA-targeting system for epigenetic modification of EBF4 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets ETS1 or a DNA regulatory element thereof. ETS1 is a gene that encodes Protein C-ets-1 (also known as ETS-1, EWSR2, p54, c-cts-1, ETS proto-oncogene 1). ETS1 belongs to the ETS family of transcription factors. ETS1 controls the expression of cytokine and chemokine genes in a variety of cellular contexts, and may control the differentiation, survival and proliferation of lymphoid cells. In some embodiments, the gRNA targets a target site in ETS1 or a DNA regulatory element thereof that comprises SEQ ID NO: 14, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 146, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 278, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ETS1 or a DNA regulatory element thereof is set forth in SEQ ID NO: 278. In some embodiments, a provided DNA-targeting system for epigenetic modification of ETS1 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets FOXD3 or a DNA regulatory element thereof. FOXD3 is a gene that encodes Forkhead box protein D3 (also known as AIS1, Genesis, HFH2, VAMAS2, forkhead box D3). FOXD3 belongs to the forkhead protein family of transcription factors which is characterized by a Dna-targeting forkhead domain. In some embodiments, the gRNA targets a target site in FOXD3 or a DNA regulatory element thereof that comprises SEQ ID NO: 15, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 147, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 279, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting FOXD3 or a DNA regulatory element thereof is set forth in SEQ ID NO: 279. In some embodiments, a provided DNA-targeting system for epigenetic modification of FOXD3 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets HELT or a DNA regulatory element thereof. HELT is a gene that encodes Hairy and enhancer of split-related protein HELT, and may act as a transcription factor or transcriptional repressor. In some embodiments, the gRNA targets a target site in HELT or a DNA regulatory element thereof that comprises SEQ ID NO: 16, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 148, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 280, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting HELT or a DNA regulatory element thereof is set forth in SEQ ID NO: 280. In some embodiments, a provided DNA-targeting system for epigenetic modification of HELT or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets HMGN3 or a DNA regulatory element thereof. HMGN3 is a gene that encodes High mobility group nucleosome-binding domain-containing protein 3 (also known as PNAS-25, TRIP7, PNAS-24). HMGN3 binds to nucleosomes, regulating chromatin structure and consequently, chromatin-dependent processes such as transcription, DNA replication and DNA repair. In some embodiments, the gRNA targets a target site in HMGN3 or a DNA regulatory element thereof that comprises SEQ ID NO: 17, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 149, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 281, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting HMGN3 or a DNA regulatory element thereof is set forth in SEQ ID NO: 281. In some embodiments, a provided DNA-targeting system for epigenetic modification of HMGN3 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets PATZ1 or a DNA regulatory element thereof. PATZ1 is a gene that encodes POZ-, AT hook-, and zinc finger-containing protein 1 (also known as MAZR, PATZ, RIAZ, ZBTB19, ZNF278, ZSG, and dJ400N23). PATZ1 is a transcriptional regulator that plays a role in many biological processes including embryogenesis, senescence, T-cell development, and neurogenesis. In some embodiments, the gRNA targets a target site in PATZ1 or a DNA regulatory element thereof that comprises SEQ ID NO: 18, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 150, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 282, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting PATZ1 or a DNA regulatory element thereof is set forth in SEQ ID NO: 282. In some embodiments, a provided DNA-targeting system for epigenetic modification of PATZ1 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets PRDM8 or a DNA regulatory element thereof. PRDM8 is a gene that encodes PR domain zinc finger protein 8, and may regulate chromatin accessibility in T cells. In some embodiments, the gRNA targets a target site in PRDM8 or a DNA regulatory element thereof that comprises SEQ ID NO: 19, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 151, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 283, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting PRDM8 or a DNA regulatory element thereof is set forth in SEQ ID NO: 283. In some embodiments, a provided DNA-targeting system for epigenetic modification of PRDM8 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets SOX21 or a DNA regulatory element thereof. SOX21 is a gene that encodes Transcription factor SOX-21 (also known as SOX25, SRY-box 21, SRY-box transcription factor 21). SOX21 is a member of the Sox gene family of transcription factors, and regulates hair, neural, and trophoblast stem cell differentiation. In some embodiments, the gRNA targets a target site in SOX21 or a DNA regulatory element thereof that comprises SEQ ID NO: 20, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 152, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 284, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting SOX21 or a DNA regulatory element thereof is set forth in SEQ ID NO: 284. In some embodiments, a provided DNA-targeting system for epigenetic modification of SOX21 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets TBX15 or a DNA regulatory element thereof. TBX15 is a gene that encodes T-box transcription factor TBX15. TBX15 is a transcription factor involved in a variety of developmental processes, including in the skeletal system. In some embodiments, the gRNA targets a target site in TBX15 or a DNA regulatory element thereof that comprises SEQ ID NO: 21, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 153, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 285, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting TBX15 or a DNA regulatory element thereof is set forth in SEQ ID NO: 285. In some embodiments, a provided DNA-targeting system for epigenetic modification of TBX15 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets ZBED5 or a DNA regulatory element thereof. ZBED5 is a gene that encodes Zinc finger BED domain-containing protein 5 (also known as Buster1). In some embodiments, the gRNA targets a target site in ZBED5 or a DNA regulatory element thereof that comprises SEQ ID NO: 22, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 154, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%. 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 286, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ZBED5 or a DNA regulatory element thereof is set forth in SEQ ID NO: 286. In some embodiments, a provided DNA-targeting system for epigenetic modification of ZBED5 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets ZBTB7A or a DNA regulatory element thereof. ZBTB7A is a gene that encodes Zinc finger and BTB domain-containing protein 7A (also known as FBI-1, FBI1, LRF, ZBTB7, ZNF857A, pokemon, TIP21). ZBTB7A is a transcription factor that represses the transcription of a wide range of genes involved in cell proliferation and differentiation. In some embodiments, the gRNA targets a target site in ZBTB7A or a DNA regulatory element thereof that comprises SEQ ID NO: 23, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 155, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 287, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ZBTB7A or a DNA regulatory element thereof is set forth in SEQ ID NO: 287. In some embodiments, a provided DNA-targeting system for epigenetic modification of ZBTB7A or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets ZNF219 or a DNA regulatory element thereof. ZNF219 is a gene that encodes Zinc finger protein 219 (also known as ZFP219), and may act as a transcriptional regulator. In some embodiments, the gRNA targets a target site in ZNF219 or a DNA regulatory element thereof that comprises SEQ ID NO: 24, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 156, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 288, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ZNF219 or a DNA regulatory element thereof is set forth in SEQ ID NO: 288. In some embodiments, a provided DNA-targeting system for epigenetic modification of ZNF219 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets ZNF319 or a DNA regulatory element thereof. ZNF319 is a gene that encodes Zinc finger protein 319, and may act as a transcriptional regulator. In some embodiments, the gRNA targets a target site in ZNF319 or a DNA regulatory element thereof that comprises SEQ ID NO: 25, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 157, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 289, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ZNF319 or a DNA regulatory element thereof is set forth in SEQ ID NO: 289. In some embodiments, a provided DNA-targeting system for epigenetic modification of ZNF319 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets ZNF470 or a DNA regulatory element thereof. ZNF470 is a gene that encodes Zinc finger protein 470, and may act as a transcriptional regulator. In some embodiments, the gRNA targets a target site in ZNF470 or a DNA regulatory element thereof that comprises SEQ ID NO: 26, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 158, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 290, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ZNF470 or a DNA regulatory element thereof is set forth in SEQ ID NO: 290. In some embodiments, a provided DNA-targeting system for epigenetic modification of ZNF470 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets ZNF816 or a DNA regulatory element thereof. ZNF816 is a gene that encodes Zinc finger protein 816, and may act as a transcriptional regulator. In some embodiments, the gRNA targets a target site in ZNF816 or a DNA regulatory element thereof that comprises SEQ ID NO: 27, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 159, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 291, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ZNF816 or a DNA regulatory element thereof is set forth in SEQ ID NO: 291. In some embodiments, a provided DNA-targeting system for epigenetic modification of ZNF816 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

In some embodiments, a gRNA provided herein targets ZSCAN23 or a DNA regulatory element thereof. ZSCAN is a gene that encodes Zinc finger and SCAN domain-containing protein 23, and may act as a transcriptional regulator. In some embodiments, the gRNA targets a target site in ZSCAN23 or a DNA regulatory element thereof that comprises SEQ ID NO: 28, a contiguous portion thereof of at least 14 nucleotides (e.g. 14, 15, 16, 17, 18 or 19 nucleotides), a complementary sequence of any of the foregoing, or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA comprises a spacer sequence comprising SEQ ID NO: 160, a contiguous portion thereof of at least 14 nt (e.g. 14, 15, 16, 17, 18 or 19 nucleotides), or a sequence having at or at least 80%, 85%, 90%, 91%, 92%, 93%, 94%, 95%, 96%, 97%, 98%, 99%, 99.5%, 99.9%, or 100% sequence identity to any of the foregoing. In some embodiments, the gRNA further comprises a scaffold sequence. In some embodiments, the scaffold sequence comprises the sequence set forth in SEQ ID NO: 398, or a sequence having at or at least 80%, 85%, 90%, 91%, 92%, 93%, 94%, 95%, 96%, 97%, 98%, 99%, 99.5%, 99.9%, or 100% sequence identity to SEQ ID NO: 398. In some embodiments, the gRNA, including a spacer sequence and a scaffold sequence, comprises SEQ ID NO: 292, or a sequence having at or at least 80%, 85%, 90%, 91%, 92%, 93%, 94%, 95%, 96%, 97%, 98%, 99%, 99.5%, 99.9%, or 100% sequence identity to all or a portion thereof. In some embodiments, the gRNA targeting ZSCAN23 or a DNA regulatory element thereof is set forth in SEQ ID NO: 292. In some embodiments, a provided DNA-targeting system for epigenetic modification of ZSCAN23 or a DNA regulatory element thereof includes any of the above gRNAs complexed with a Cas protein, such as a Cas9 protein. In some embodiments, the Cas9 is a dCas9. In some embodiments, the dCas9 is a dSpCas9, such as a dSpCas9 set forth in SEQ ID NO: 408.

### C. Other DNA-Targeting Domains

In some of any of the provided embodiments, the DNA-targeting domain comprises a zinc finger protein (ZFP); a transcription activator-like effector (TALE); a meganuclease; a homing endonuclease; or an I-SceI enzyme or a variant thereof. In some embodiments, the DNA-targeting domain comprises a catalytically inactive variant of any of the foregoing.

In some embodiments, a ZFP, a zinc finger DNA binding protein, or zinc finger DNA binding domain, is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP. Among the ZFPs are artificial, or engineered, ZFPs, comprising ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3, and 6) on a zinc finger recognition helix. Thus, for example, the ZFP or ZFP-containing molecule is non-naturally occurring, e.g., is engineered to bind to a target site of choice.

In some cases, the DNA-targeting system is or comprises a zinc-finger DNA binding domain fused to an effector domain. Some gene-specific engineered zinc fingers are available commercially. For example, a platform called CompoZr, for zinc-finger construction is available that provides specifically targeted zinc fingers for thousands of targets. Sec, e.g., Gaj et al., Trends in Biotechnology, 2013, 31 (7), 397-405. In some cases, commercially available zinc fingers are used, or are custom designed.

Transcription activator-like effectors (TALEs), are proteins naturally found in Xanthomonas bacteria. TALEs comprise a plurality of repeated amino acid sequences, each repeat having binding specificity for one base in a target sequence. Each repeat comprises a pair of variable residues in position 12 and 13 (repeat variable diresidue; RVD) that determine the nucleotide specificity of the repeat. In some embodiments, RVDs associated with recognition of the different nucleotides are HD for recognizing C. NG for recognizing T, NI for recognizing A, NN for recognizing G or A. NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T. TL for recognizing A, VT for recognizing A or G and SW for recognizing A. In some embodiments, RVDs can be mutated towards other amino acid residues in order to modulate their specificity towards nucleotides A. T. C and G and in particular to enhance this specificity. Binding domains with similar modular base-per-base nucleic acid binding properties can also be derived from different bacterial species. These alternative modular proteins may exhibit more sequence variability than TALE repeats.

In some embodiments, a “TALE DNA binding domain” or “TALE” is a polypeptide comprising one or more TALE repeat domains/units. The repeat domains, each comprising a repeat variable diresidue (RVD), are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. TALE proteins may be designed to bind to a target site using canonical or non-canonical RVDs within the repeat units. See, e.g., U.S. Pat. Nos. 8,586,526 and 9,458,205.

In some embodiments, a TALE is a fusion protein comprising a nucleic acid binding domain derived from a TALE and an effector domain. In some embodiments, one or more sites in the FXN locus can be targeted by engineered TALEs.

Zinc finger and TALE Dna-targeting domains can be engineered to bind to a predetermined nucleotide sequence, for example via engineering (altering one or more amino acids) of the recognition helix region of a naturally occurring zinc finger protein, by engineering of the amino acids in a TALE repeat involved in DNA binding (the repeat variable diresidue or RVD region), or by systematic ordering of modular Dna-targeting domains, such as TALE repeats or ZFP domains. Therefore, engineered zinc finger proteins or TALE proteins are proteins that are non-naturally occurring. Non-limiting examples of methods for engineering zinc finger proteins and TALEs are design and selection. A designed protein is a protein not occurring in nature whose design/composition results principally from rational criteria. Rational criteria for design include application of substitution rules and computerized algorithms for processing information in a database storing information of existing ZFP or TALE designs (canonical and non-canonical RVDs) and binding data. See, for example, U.S. Pat. Nos. 9,458,205; 8,586,526; 6,140,081; 6,453,242; and 6,534,261; see also WO 98/53058; WO 98/53059; WO 98/53060; WO 02/016536 and WO 03/016496.

### D. Effector Domains

In some aspects, the DNA-targeting systems provided herein further include one or more effector domains. In some embodiments, provided herein is a DNA-targeting system comprising a fusion protein comprising: (a) a DNA-targeting domain capable of being targeted to a target site in a gene or regulatory DNA element thereof, such as any described above, and (b) at least one effector domain. In some aspects, the effector domain is capable of increasing transcription of the gene. In some aspects, the effector domain comprises a transcription activation domain.

In some aspects, the effector domain activates, induces, catalyzes, or leads to increased transcription of a gene when ectopically recruited to the gene or DNA regulatory element thereof. In some embodiments, the effector domain activates, induces, catalyzes, or leads to: transcription activation, transcription co-activation, transcription elongation, transcription de-repression, transcription factor release, polymerization, histone modification, histone acetylation, histone deacetylation, nucleosome remodeling, chromatin remodeling, reversal of heterochromatin formation, proteolysis, ubiquitination, deubiquitination, phosphorylation, dephosphorylation, DNA methylation, DNA demethylation, histone methylation, histone demethylation, or DNA base oxidation. In some embodiments, the effector domain activates, induces, catalyzes or leads to transcription activation, transcription co-activation, or transcription elongation. In some embodiments, the effector domain induces transcription activation. In some embodiments, the effector domain has one of the aforementioned activities itself (i.e. acts directly). In some embodiments, the effector domain recruits and/or interacts with a polypeptide domain that has one of the aforementioned activities (i.e. acts indirectly).

Gene expression of endogenous mammalian genes, such as human genes, can be achieved by targeting a fusion protein comprising a DNA-targeting domain, such as a dCas9, and an effector domain, such as a transcription activation domain, to mammalian genes or regulatory DNA elements thereof (e.g. a promoter or enhancer) via one or more gRNAs. Any of a variety of effector domains for transcriptional activation (e.g. transcription activation domains) are known and can be used in accord with the provided embodiments. Transcription activation domains, as well as activation of target genes by Cas fusion proteins (with a variety of Cas molecules) and the transcription activation domains, are described, for example, in WO 2014/197748, WO 2016/130600, WO 2017/180915, WO 2021/226555, WO 2021/226077, WO 2013/176772, WO 2014/152432, WO 2014/093661, Adli, M. Nat. Commun. 9, 1911 (2018), Perez-Pinera, P. et al. Nat. Methods 10, 973-976 (2013), Mali, P. et al. Nat. Biotechnol. 31, 833-838 (2013), and Maeder, M. L. et al. Nat. Methods 10, 977-979 (2013).

In some embodiments, the effector domain may comprise VP64, p65, Rta, p300, CBP. VPR, VPH, HSF1, a TET protein (e.g. TET1), an ERF protein (e.g. ERF1, ERF3), LSD1, SunTag, a partially or fully functional fragment or domain thereof, or a combination of any of the foregoing.

In some embodiments, the effector domain comprises a transcriptional activator domain described in WO 2021/226077.

In some embodiments, the effector domain may comprise a VP64 domain. For example, dCas9-VP64 can be targeted to a target site by one or more gRNAs to activate a gene. VP64 is a polypeptide composed of four tandem copies of VP16, a 16 amino acid transactivation domain of the Herpes simplex virus. VP64 domains, including in dCas fusion proteins, have been described, for example, in WO 2014/197748, WO 2013/176772, WO 2014/152432, and WO 2014/093661. In some embodiments, the effector domain comprises at least one VP16 domain, or a VP16 tetramer (“VP64”) or a variant thereof. An exemplary VP64 domain is set forth in SEQ ID NO: 409. In some embodiments, the effector domain comprises the sequence set forth in SEQ ID NO: 409, or a portion thereof, or an amino acid sequence that has at least 90%, 91%, 92%, 93%, 94%, 95%, 96%, 97%, 98%, or 99% sequence identity to any of the foregoing.

In some embodiments, the effector domain may comprise a p65 activation domain (p65AD). p65AD is the principal transactivation domain of the 65 kDa polypeptide of the nuclear form of the NF-KB transcription factor. An exemplary sequence of human transcription factor p65 is available at the Uniprot database under accession number Q04206. p65 domains, including in dCas fusion proteins, have been described, for example in WO 2017/180915 and Chavez, A. et al. Nat. Methods 12, 326-328 (2015). An exemplary p65 activation domain is set forth in SEQ ID NO: 411. In some embodiments, the effector domain comprises the sequence set forth in SEQ ID NO: 411, or a portion thereof, or an amino acid sequence that has at least 90%, 91%, 92%, 93%, 94%, 95%, 96%, 97%, 98%, or 99% sequence identity to any of the foregoing.

In some embodiments, the effector domain may comprise a R transactivator (Rta) domain. Rta is an immediate-early protein of Epstein-Barr virus (EBV), and is a transcriptional activator that induces lytic gene expression and triggers virus reactivation. The Rta domain, including in dCas fusion proteins, has been described, for example in WO 2017/180915 and Chavez, A. et al. Nat. Methods 12, 326-328 (2015). An exemplary Rta domain is set forth in SEQ ID NO: 412. In some embodiments, the effector domain comprises the sequence set forth in SEQ ID NO: 412, or a portion thereof, or an amino acid sequence that has at least 90%, 91%, 92%, 93%, 94%, 95%, 96%, 97%, 98%, or 99% sequence identity to any of the foregoing.

In some embodiments, the effector domain may have histone acetyltransferase activity. For example, the effector domain may comprise a domain from p300 or CREB-binding protein (CBP) protein. The effector domain may comprise a p300 domain. p300 functions as a histone acetyltransferase that regulates transcription via chromatin remodeling and is involved with the processes of cell proliferation and differentiation. The p300 domain, including in dCas fusion proteins for gene activation, has been described, for example, in WO 2016/130600 and WO 2017/180915. An exemplary p300 domain is set forth in SEQ ID NO: 413. In some embodiments, the effector domain comprises the sequence set forth in SEQ ID NO: 413, or a portion thereof, or an amino acid sequence that has at least 90%, 91%, 92%, 93%, 94%, 95%, 96%, 97%, 98%, or 99% sequence identity to any of the foregoing.

In some embodiments, the effector domain may comprise a HSF1 domain. HSF1 is a gene that encodes Heat shock factor protein 1. HSF1, including in dCas fusion proteins for gene activation, has been described, for example, in WO 2021/226555, WO 2015/089427, and Konermann et al. Nature 517 (7536): 583-8 (2015). An exemplary HSF1 domain is set forth in SEQ ID NO: 414. In some embodiments, the effector domain comprises the sequence set forth in SEQ ID NO: 414, or a portion thereof, or an amino acid sequence that has at least 90%, 91%, 92%, 93%, 94%, 95%, 96%, 97%, 98%, or 99% sequence identity to any of the foregoing.

In some embodiments, the effector domain may comprise a eukaryotic release factor domain, for example from eukaryotic release factor 1 (ERF1) or eukaryotic release factor 3 (ERF3).

In some embodiments, the effector domain may comprise the tripartite activator VP64-p65-Rta (also known as VPR). VPR comprises three transcription activation domains (VP64, p65, and Rta) fused by short amino acid linkers, and can effectively upregulate target gene expression. VPR, including in dCas fusion proteins for gene activation, has been described, for example, in WO 2021/226555 and Chavez, A. et al. Nat. Methods 12, 326-328 (2015). An exemplary VPR polypeptide is set forth in SEQ ID NO: 415. In some embodiments, the effector domain comprises the sequence set forth in SEQ ID NO: 415, or a portion thereof, or an amino acid sequence that has at least 90%, 91%, 92%, 93%, 94%, 95%, 96%, 97%, 98%, or 99% sequence identity to any of the foregoing.

In some embodiments, the effector domain may comprise VPH. VPH is a polypeptide comprising VP64, mouse p65, and HSF1. VPH, including in dCas fusion proteins for gene activation, has been described, for example, in WO 2021/226555. An exemplary VPH polypeptide is set forth in SEQ ID NO: 416. In some embodiments, the effector domain comprises the sequence set forth in SEQ ID NO: 416, or a portion thereof, or an amino acid sequence that has at least 90%, 91%, 92%, 93%, 94%, 95%, 96%, 97%, 98%, or 99% sequence identity to any of the foregoing.

In some embodiments, the effector domain may have demethylase activity. The effector domain can include an enzyme that removes methyl (CH3-) groups from nucleic acids, proteins (in particular histones), and other molecules. Alternatively, the effector can convert the methyl group to hydroxymethylcytosine in a mechanism for demethylating DNA. The effector domain can catalyze this reaction. For example, the effector domain that catalyzes this reaction may comprise a domain from a TET protein, for example TET1 (Ten-eleven translocation methylcytosine dioxygenase 1). TET1, including in dCas fusion proteins for gene activation, has been described, for example, in WO 2021/226555. An exemplary TET1 catalytic domain is set forth in SEQ ID NO: 417. In some embodiments, the effector domain comprises the sequence set forth in SEQ ID NO: 417, or a portion thereof, or an amino acid sequence that has at least 90%, 91%, 92%, 93%, 94%, 95%, 96%, 97%, 98%, or 99% sequence identity to any of the foregoing.

In some embodiments, the effector domain may comprise a LSD1 domain. LSD1 (also known as Lysine-specific histone demethylase 1A) is a histone demethylase that can demethylate lysine residues of histone H3, thereby acting as a coactivator or a corepressor, depending on the context. LSD1, including in dCas fusion proteins, has been described, for example, in WO 2013/176772, WO 2014/152432, and Kearns, N. A. et al. Nat. Methods. 12 (5): 401-403 (2015). An exemplary LSD1 polypeptide is set forth in SEQ ID NO: 440. In some embodiments, the effector domain comprises the sequence set forth in SEQ ID NO: 440, or a portion thereof, or an amino acid sequence that has at least 90%, 91%, 92%, 93%, 94%, 95%, 96%, 97%, 98%, or 99% sequence identity to any of the foregoing.

In some embodiments, the effector domain may comprise a SunTag domain. SunTag is a repeating peptide array, which can recruit multiple copies of an antibody-fusion protein that binds the repeating peptide. The antibody-fusion protein may comprise an additional effector domain, such as a transcription activation domain (e.g. VP64), to induce increased transcription of the target gene. SunTag, including in dCas fusion proteins for gene activation, has been described, for example, in WO 2016/011070 and Tanenbaum, M. et al. Cell. 159 (3): 635-646 (2014). An exemplary SunTag effector domain includes a repeating GCN4 peptide having the amino acid sequence LLPKNYHLENEVARLKKLVGER (SEQ ID NO: 438) separated by linkers having the amino acid sequence GGSGG (SEQ ID NO: 439). In some embodiments, the effector domain comprises the sequence set forth in SEQ ID NO: 438, or a portion thereof, or an amino acid sequence that has at least 90%, 91%, 92%, 93%, 94%, 95%, 96%, 97%, 98%, or 99% sequence identity to any of the foregoing. In some embodiments, the SunTag effector domain recruits an antibody-fusion protein that comprises VP64 and binds the GCN4 peptide.

### E. Fusion Proteins

In some aspects, the DNA-targeting systems provided herein are fusion proteins. In some embodiments, provided herein is a DNA-targeting system that is a fusion protein comprising: (a) a DNA-targeting domain capable of being targeted to a target site in a gene or regulatory DNA element thereof, and (b) at least one effector domain. In some aspects, the fusion protein comprises at least one of any of the DNA-targeting domains described herein, and at least one of any of the effector domains described herein. For instance, in some embodiments, the fusion protein contains a CRISPR-Cas DNA-targeting domain, such as described in Section II.B, and at least one effector domain described herein. In some aspects, the fusion protein is targeted to a target site in a gene or regulatory element thereof, and leads to increased transcription of the gene.

In some embodiments, the DNA-targeting domain and effector domain of the fusion protein are heterologous, i.e. the domains are from different species, or at least one of the domains is not found in nature. In some aspects, the fusion protein is an engineered fusion protein, i.e. the fusion protein is not found in nature.

In some embodiments, the at least one effector domain is fused to the N-terminus, the C-terminus, or both the N-terminus and the C-terminus, of the DNA-targeting domain or a component thereof. The at least one effector domain may be fused to the DNA-targeting domain directly, or via any intervening amino acid sequence, such as a linker sequence or a nuclear localization sequence (NLS).

In some embodiments, the fusion protein comprises one or more linkers. In some embodiments, the one or more linkers connect the DNA-targeting domain or a component thereof to the at least one effector domain. A linker may be included anywhere in the polypeptide sequence of the fusion protein, for example, between the effector domain and the DNA-targeting domain or a component thereof. A linker may be of any length and designed to promote or restrict the mobility of components in the fusion protein. A linker may comprise any amino acid sequence of about 2 to about 100, about 5 to about 80, about 10 to about 60, or about 20 to about 50 amino acids. A linker may comprise an amino acid sequence of at least about 2, 3, 4, 5, 10, 15, 20, 25, or 30 amino acids. A linker may comprise an amino acid sequence of less than about 100, 90, 80, 70, 60, 50, or 40 amino acids. A linker may include sequential or tandem repeats of an amino acid sequence that is 2 to 20 amino acids in length. Linkers may be rich in amino acids glycine (G), serine(S), and/or alanine (A). Linkers may include, for example, a GS linker. An exemplary GS linker is represented by the sequence GGGGS (SEQ ID NO: 418), or the formula (GGGGS)n, wherein n is an integer that represents the number of times the GGGGS sequence is repeated (e.g. between 1 and 10 times). The number of times a linker sequence is repeated can be adjusted to optimize the linker length and achieve appropriate separation of the functional domains. Other examples of linkers may include, for example,

(SEQ ID NO: 419)
GGGGG,
(SEQ ID NO: 420)
GGAGG,
(SEQ ID NO: 421)
GGGGSSS,
or
(SEQ ID NO: 422)
GGGGAAA.

In some embodiments, the DNA-targeting system comprises one or more nuclear localization signals (NLS). In some embodiments, a fusion protein described herein comprises one or more nuclear localization sequences (NLSs), such as about or more than about 1, 2, 3, 4, 5, 6, 7, 8, 9, 10, or more NLSs. When more than one NLS is present, each may be selected independently of the others, such that a single NLS may be present in more than one copy and/or in combination with one or more other NLSs present in one or more copies. Non-limiting examples of NLSs include an NLS sequence derived from: the NLS of the SV40 virus large T-antigen, having the amino acid sequence PKKKRKV (SEQ ID NO: 423); the NLS from nucleoplasmin (e.g. the nucleoplasmin bipartite NLS with the sequence KRPAATKKAGQAKKKK (SEQ ID NO: 410)); the c-myc NLS having the amino acid sequence PAAKRVKLD (SEQ ID NO: 424) or RQRRNELKRSP (SEQ ID NO: 425); the hRNPA1 M9 NLS having the sequence NQSSNFGPMKGGNFGGRSSGPYGGGGQYFAKPRNQGGY (SEQ ID NO: 426); the sequence RMRIZFKNKGKDTAELRRRRVEVSVELRKAKKDEQILKRRNV (SEQ ID NO: 427) of the IBB domain from importin-alpha; the sequences VSRKRPRP (SEQ ID NO: 428) and PPKKARED (SEQ ID NO: 429) of the myoma T protein; the sequence PQPKKKPL (SEQ ID NO: 430) of human p53; the sequence SALIKKKKKMAP (SEQ ID NO: 431) of mouse c-abl IV; the sequences DRLRR (SEQ ID NO: 432) and PKQKKRK (SEQ ID NO: 433) of the influenza virus NS1; the sequence RKLKKKIKKL (SEQ ID NO: 434) of the Hepatitis virus delta antigen; the sequence REKKKFLKRR (SEQ ID NO: 435) of the mouse Mx1 protein; the sequence KRKGDEVDGVDEVAKKKSKK (SEQ ID NO: 436) of the human poly (ADP-ribose) polymerase; and the sequence RKCLQAGMNLEARKTKK (SEQ ID NO: 437) of the steroid hormone receptors (human) glucocorticoid. In general, the one or more NLSs are of sufficient strength to drive accumulation of the fusion protein in a detectable amount in the nucleus of a eukaryotic cell. In general, strength of nuclear localization activity may derive from the number of NLSs in the fusion protein, the particular NLS(s) used, or a combination of these factors. Detection of accumulation in the nucleus may be performed by any suitable technique. For example, a detectable marker may be fused to the fusion protein, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of the fusion protein (e.g. an assay for altered gene expression activity in a cell transformed with the DNA-targeting system comprising the fusion protein), as compared to a control condition (e.g. an untransformed cell). In some embodiments, the NLS comprises the sequence set forth in SEQ ID NO: 410 (KRPAATKKAGQAKKKK), or a portion thereof.

In some embodiments, a fusion protein provided herein comprises dCas9 and VP64. In some embodiments, a fusion protein provided herein comprises VP64-dSpCas9-NLS-VP64. In some embodiments, a fusion protein provided herein comprises the sequence set forth in SEQ ID NO: 400, or an amino acid sequence that has at least 90%, 91%, 92%, 93%, 94%, 95%, 96%, 97%, 98%, or 99% sequence identity thereto.

### II. POLYNUCLEOTIDES, VECTORS, AND RELATED METHODS FOR DELIVERY

In some aspects, provided are polynucleotides encoding any of the DNA-targeting systems described herein or a portion or a component of any of the foregoing. In some aspects, the polynucleotides can encode any of the components of the DNA-targeting systems, and/or any nucleic acid or proteinaceous molecule necessary to carry out aspects of the methods of the disclosure. In particular embodiments, provided are polynucleotides encoding any of the fusion proteins described herein. Also provided herein are polynucleotides encoding any of the gRNAs described herein.

In some embodiments, provided are polynucleotides comprising the gRNAs described herein. In some embodiments, the gRNA is transcribed from a genetic construct (i.e. vector or plasmid) in the target cell. In some embodiments, the gRNA is produced by in vitro transcription and delivered to the target cell. In some embodiments, the gRNA comprises one or more modified nucleotides for increased stability. In some embodiments, the gRNA is delivered to the target cell pre-complexed as a RNP with the fusion protein.

In some embodiments, a provided polynucleotide encodes a fusion protein as described herein that includes (a) a DNA-targeting domain capable of being targeted to a target site of a target gene as described; and (b) at least one effector domain capable of increasing transcription of the gene. In some embodiments, the fusion protein includes a fusion protein of a Cas protein or variant thereof and at least one effector domain capable of increasing transcription of a gene. In particular example, the Cas is a dCas, such as dCas9. In some embodiments, the dCas9 is a dSpCas9, such as polynucleotide encoding a dSpCas9 set forth in SEQ ID NO: 408. Examples of such domains and fusion proteins include any as described in Section I.

In some embodiments, the polynucleotide comprises the sequence set forth in SEQ ID NO: 399, or a sequence having at least about 80%, 85%, 90%, 91%, 92%, 93%, 94%, 95%, 96%, 97%, 98%, 99%, or 100% sequence identity thereto. In some embodiments, the polynucleotide is set forth in SEQ ID NO:399. In some embodiments, the polynucleotide encodes an amino acid sequence comprising SEQ ID NO: 400, or a sequence having at least about 80%, 85%, 90%, 91%, 92%, 93%, 94%, 95%, 96%, 97%, 98%, 99%, or 100% sequence identity thereto. In some embodiments, the polynucleotide encodes the amino acid sequence set forth in SEQ ID NO:400.

In some embodiments, the polynucleotide is RNA or DNA. In some embodiments, the polynucleotide, such as a polynucleotide encoding a provided fusion protein, is mRNA. The mRNA can be 5′ capped and/or 3′ polyadenylated. In another embodiment, a polynucleotide provided herein, such as a polynucleotide encoding a provided fusion protein, is DNA. The DNA can be present in a vector.

Also provided herein is a vector that contains any of the provided polynucleotides. In some embodiments, the vector comprises a genetic construct, such as a plasmid or an expression vector.

In some embodiments, the expression vector comprising the sequence encoding the fusion protein of a DNA-targeting system provided herein can further comprise a polynucleotide sequence encoding at least one gRNA. The sequence encoding the gRNA can be operably linked to at least one transcriptional control sequence for expression of the gRNA in the cell. For example, DNA encoding the gRNA can be operably linked to a promoter sequence that is recognized by RNA polymerase III (Pol III). Examples of suitable Pol III promoters include, but are not limited to, mammalian U6, U3, H1, and 7SL RNA promoters.

In some embodiments, provided is a vector containing a polynucleotide that encodes a fusion protein comprising a DNA-targeting domain comprising a dCas and at least one effector domain capable of increasing transcription of a gene, and a polynucleotide(s) encoding at least one gRNA. In some embodiments, the dCas is a dCas9, such as dSpCas9. In some embodiments, the polynucleotide encodes a fusion protein that includes a dSpCas9 set forth in SEQ ID NO: 408. In some embodiments, the polynucleotide encoding at least one gRNA encodes a gRNA as described in Section II.B.ii. For example, the polynucleotide can encode a gRNA comprising a spacer sequence selected from any one of SEQ ID NOS: 133-264, or a contiguous portion thereof of at least 14 nt. In some embodiments the polynucleotide encoding the at least one gRNA encodes a gRNA that comprises the sequence set forth in any one of SEQ ID NOS: 265-396.

In some embodiments, the effector domain is VP64. In some embodiments, the vector includes a polynucleotide that encodes the amino acid sequence comprising SEQ ID NO: 400, or a sequence having at least about 80%, 85%, 90%, 91%, 92%, 93%, 94%, 95%, 96%, 97%, 98%, 99%, or 100% sequence identity thereto, and a polynucleotide that encodes a gRNA such as any described in Section II.B.ii. In some embodiments, the polynucleotide encoding the at least one gRNA encodes a gRNA comprising a spacer sequence selected from any one of SEQ ID NOS: 133-264, or a contiguous portion thereof of at least 14 nt. In some embodiments, the gRNA further comprises the sequence set forth in SEQ ID NO: 398. In some embodiments the polynucleotide encoding the at least one gRNA encodes a gRNA that comprises the sequence set forth in any one of SEQ ID NOS: 265-396.

In some embodiments, the polynucleotide encodes the fusion protein and the at least one gRNA.

In some embodiments, the polynucleotide as provided herein can be codon optimized for efficient translation into protein in the eukaryotic cell or animal of interest. For example, codons can be optimized for expression in humans, mice, rats, hamsters, cows, pigs, cats, dogs, fish, amphibians, plants, yeast, insects, and so forth. Programs for codon optimization are available as freeware. Commercial codon optimization programs are also available.

In some embodiments, a polynucleotide described herein can comprise one or more transcription and/or translation control elements. Depending on the host/vector system utilized, any of a number of suitable transcription and translation control elements, including constitutive and inducible promoters, transcription enhancer elements, transcription terminators, etc. can be used in the expression vector.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct comprising the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs used in connection with the DNA-targeting systems, various promoters such as RNA polymerase III promoters, including for example U6 and H1, can be advantageous. Descriptions of and parameters for enhancing the use of such promoters are known in the art, and additional information and approaches are regularly being described; see, e.g., Ma, H. et al., Molecular Therapy—Nucleic Acids 3, e161 (2014) doi:10.1038/mtna.2014.12.

The expression vector can also contain a ribosome binding site for translation initiation and a transcription terminator. The expression vector can also comprise appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

A promoter can be an inducible promoter (e.g., a heat shock promoter, tetracycline-regulated promoter, steroid-regulated promoter, metal-regulated promoter, estrogen receptor-regulated promoter, etc.). The promoter can be a constitutive promoter (e.g., CMV promoter, UBC promoter). In some cases, the promoter can be a spatially restricted and/or temporally restricted promoter (e.g., a tissue specific promoter, a cell type specific promoter (e.g. a T cell specific promoter), etc.).

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Other vectors can be used so long as they are compatible with the host cell.

In some embodiments, the vector is a viral vector, such as an adeno-associated virus (AAV) vector, a retroviral vector, a lentiviral vector, or a gammaretroviral vector. In some embodiments In some embodiments, the viral vector is an adeno-associated virus (AAV) vector. In some embodiments, the AAV vector is selected from among an AAV1, AAV2, AAV3, AAV4, AAV5, AAV6, AAV7, AAV8, or AAV9 vector. In some embodiments, the vector is a lentiviral vector. In some embodiments, the vector is a non-viral vector, for example a lipid nanoparticle, a liposome, an exosome, or a cell penetrating peptide. In some embodiments, the vector comprises one vector, or two or more vectors.

In some embodiments, the vector exhibits immune cell or T cell tropism.

In some aspects, provided herein are pluralities of vectors that comprise any of the vectors described herein, and one or more additional vectors comprising one or more additional polynucleotides encoding an additional portion or an additional component of any of the DNA-targeting systems described herein, any of the gRNAs described herein, any of the fusion proteins described herein, or a portion or a component of any of the foregoing.

Provided are pluralities of vectors, that include: a first vector comprising any of the polynucleotides described herein; and a second vector comprising any of the polynucleotides described herein.

In some aspects, vectors provided herein may be referred to as delivery vehicles. In some aspects, any of the DNA-targeting systems, components thereof, or polynucleotides disclosed herein can be packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Methods of introducing a nucleic acid into a host cell are known in the art, and any known method can be used to introduce a nucleic acid (e.g., an expression construct) into a cell. Suitable methods include, include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery, and the like. In some embodiments, the composition may be delivered by mRNA delivery and ribonucleoprotein (RNP) complex delivery. Direct delivery of the RNP complex, including the DNA-targeting domain complexed with the sgRNA, can eliminate the need for intracellular transcription and translation and can offer a robust platform for host cells with low transcriptional and translational activity. The RNP complexes can be introduced into the host cell by any of the methods known in the art.

Nucleic acids or RNPs of the disclosure can be incorporated into a host using virus-like particles (VLP). VLPs contain normal viral vector components, such as envelope and capsids, but lack the viral genome. For instance, nucleic acids expressing the Cas and sgRNA can be fused to the viral vector components such as gag and introduced into producer cells. The resulting virus-like particles containing the sgRNA-expressing vectors can infect the host cell for efficient editing.

Introduction of the complexes, polypeptides, and nucleic acids of the disclosure can occur by protein transduction domains (PTDs). PTDs, including the human immunodeficiency virus-1 TAT, herpes simplex virus-1 VP22, Drsophila Antennapedia Antp, and the poluarginines, are peptide sequences that can cross the cell membrane, enter a host cell, and deliver the complexes, polypeptides, and nucleic acids into the cell.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like, for example as described in WO 2017/193107, WO 2016/123578, WO 2014/152432, WO 2014/093661, WO 2014/093655, or WO 2021/226555.

Various methods for the introduction of polynucleotides are well known and may be used with the provided methods and compositions. Exemplary methods include those for transfer of polynucleotides encoding the DNA targeting systems provided herein, including via viral, e.g., retroviral or lentiviral, transduction, transposons, and electroporation.

In some embodiments, polynucleotides can be cloned into a suitable vector, such as an expression vector or vectors. The expression vector can be any suitable recombinant expression vector, and can be used to transform or transfect any suitable cell. Suitable vectors include those designed for propagation and expansion or for expression or both, such as plasmids and viruses.

In some embodiments, the vector can a vector of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), or the pEX series (Clontech, Palo Alto, Calif.). In some embodiments, animal expression vectors include pEUK-Cl, pMAM and pMAMneo (Clontech). In some embodiments, a viral vector is used, such as a lentiviral or retroviral vector. In some embodiments, the recombinant expression vectors can be prepared using standard recombinant DNA techniques. In some embodiments, vectors can contain regulatory sequences, such as transcription and translation initiation and termination codons, which are specific to the type of host into which the vector is to be introduced, as appropriate and taking into consideration whether the vector is DNA- or RNA-based. In some embodiments, the vector can contain a nonnative promoter operably linked to the nucleotide sequence encoding the recombinant receptor. In some embodiments, the promoter can be a non-viral promoter or a viral promoter, such as a cytomegalovirus (CMV) promoter, an SV40 promoter, an RSV promoter, and a promoter found in the long-terminal repeat of the murine stem cell virus. Other promoters known to a skilled artisan also are contemplated.

In some embodiments, recombinant nucleic acids are transferred into cells using recombinant infectious virus particles, such as, e.g., vectors derived from simian virus 40 (SV40), adenoviruses, or adeno-associated virus (AAV). In some embodiments, recombinant nucleic acids are transferred into cells (e.g. T cells) using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28 (10): 1137-46; Alonso-Camino et al. (2013) Mol Ther Nucl Acids 2, e93; Park et al., Trends Biotechnol. 2011 Nov. 29 (11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

In some embodiments, the vector is a lentiviral vector. In some embodiments, the lentiviral vector is an integrase-deficient lentiviral vector. In some embodiments, the lentiviral vector is a recombinant lentiviral vector. In some embodiments, the lentivirus is selected or engineered for a desired tropism (e.g. for T cell or immune cell tropism). Methods of lentiviral production, transduction, and engineering are known, for example as described in Kasaraneni, N. et al. Sci. Rep. 8 (1): 10990 (2018), Ghalch, H. E. G. et al. Biomed. Pharmacother. 128:110276 (2020), and Milone, M. C. et al. Leukemia. 32 (7): 1529-1541 (2018). Additional methods for lentiviral transduction are described, for example in Wang et al. (2012) J. Immunother. 35 (9): 689-701; Cooper et al. (2003) Blood. 101:1637-1644; Verhoeyen et al. (2009) Methods Mol Biol. 506:97-114; and Cavalieri et al. (2003) Blood. 102 (2): 497-505.

In some embodiments, recombinant nucleic acids are transferred into cells (e.g. T cells) via electroporation {see, e.g., Chicaybam et al, (2013) PLOS ONE 8 (3): e60298 and Van Tedeloo et al. (2000) Gene Therapy 7 (16): 1431-1437). In some embodiments, recombinant nucleic acids are transferred into cells via transposition (see, e.g., Manuri et al. (2010) Hum Gene Ther 21 (4): 427-437; Sharma et al. (2013) Molec Ther Nucl Acids 2, e74; and Huang et al. (2009) Methods Mol Biol 506:115-126). Other methods of introducing and expressing genetic material into immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346:776-777 (1990)); and strontium phosphate DNA co-precipitation (Brash et al., Mol. Cell Biol., 7:2031-2034 (1987)).

### III. PHARMACEUTICAL COMPOSITIONS AND FORMULATIONS

In some aspects, provided herein are compositions, such as pharmaceutical compositions and formulations for administration, that include any of the DNA-targeting systems described herein, or any of the polynucleotides or vectors encoding the same. In some aspects, the pharmaceutical composition contains one or more DNA-targeting systems provided herein or a component thereof. In some aspects, the pharmaceutical composition comprises one or more vectors, e.g., viral vectors that contain polynucleotides that encode one or more components of the DNA-targeting systems provided herein. Such compositions can be used in accord with the provided methods, and/or with the provided articles of manufacture or compositions, such as in the prevention or treatment of diseases, conditions, and disorders, or in detection, diagnostic, and prognostic methods.

The term “pharmaceutical formulation” refers to a preparation which is in such form as to permit the biological activity of an active ingredient contained therein to be effective, and which contains no additional components which are unacceptably toxic to a subject or a cell to which the formulation would be administered.

In some embodiments, the pharmaceutical composition may further comprise a pharmaceutically acceptable excipient. The pharmaceutically acceptable excipient may be functional molecules as vehicles, adjuvants, carriers, or diluents.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular agent and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, or nanoparticles, or other known transfection facilitating agents.

In some embodiments, the transfection facilitating agent is a polyanion, polycation, including poly-L-glutamate (LGS), or lipid. In some embodiments, the transfection facilitating agent is poly-L-glutamate. In some embodiments, the transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the DNA-targeting system may also include a transfection facilitating agent such as lipids, liposomes, including lecithin liposomes or other liposomes known in the art, as a DNA-liposome mixture (see for example WO9324640), calcium ions, viral proteins, polyanions, polycations, or nanoparticles, or other known transfection facilitating agents. In some embodiments, the transfection facilitating agent is a polyanion, polycation, including poly-L-glutamate (LGS), or lipid.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the agent in a solvent, such as in admixture with a suitable carrier, diluent, or excipient such as sterile water, physiological saline, glucose, dextrose, or the like. The formulations to be used for in vivo or ex vivo administration or use are generally sterile. Sterility may be readily accomplished, e.g., by filtration through sterile filtration membranes.

The pharmaceutical composition in some embodiments contains components in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, the composition can be administered to a subject by any suitable means, for example, by bolus infusion or by injection, e.g., by intravenous or subcutaneous injection. In some embodiments, a given dose is administered by a single bolus administration of the composition. In some embodiments, the composition is administered by multiple bolus administrations of the composition, for example, over a period of no more than 3 days, or by continuous infusion administration of the composition. In some embodiments, the composition is administered parenterally, for example by intravenous, intramuscular, subcutaneous, or intraperitoneal administration. In some embodiments, the composition is administered to a subject using peripheral systemic delivery by intravenous, intraperitoneal, or subcutaneous injection.

In some embodiments, the composition is contacted with our introduced into cells (e.g. primary T cells) from a subject ex vivo, and the cells are subsequently administered to the same subject or to a different subject.

For the prevention or treatment of disease, the appropriate dosage may depend on the type of disease to be treated, the type of agent or agents, the type of cells or recombinant receptors, the severity and course of the disease, whether the agent or cells are administered for preventive or therapeutic purposes, previous therapy, the subject's clinical history and response to the agent or the cells, and the discretion of the attending physician. The compositions are in some embodiments suitably administered to the subject at one time or over a series of treatments.

### IV. METHODS OF EPIGENETICALLY MODIFYING LYMPHOID CELLS, SUCH AS T CELLS, AND MODIFIED CELLS AND COMPOSITIONS THEREOF

Provided herein are modified lymphoid cells (e.g. T cells) that have one or more modifications (also referred to as changes or alterations) in their epigenome. In some embodiments, the epigenetic change is a change relative to a comparable unmodified lymphoid cell. Reference to a comparable unmodified cell is understood to refer to the same or similar cell but that has not been introduced with a provided epigenome-modifying DNA-targeting system or that or that does not contain the same epigenetic changes (e.g. methylation or histone modification) of the target gene or regulatory region thereof.

In some embodiments, the lymphoid cells that are modified by the provided DNA-binding systems with an epigenetic change can include T cells, NK cells, or NKT cells. Such cells can include cells that have been enriched or isolated from a primary population of cells from a subject, or can include any cells that have been differentiated from stem cells into such lymphoid cells and/or have been differentiated from progenitor cells, such as common lymphoid progenitors (CLPs). In some embodiments, the lymphoid cells are differentiated from stem cells, such as hematopoietic stem or progenitor cells, or progenitor cells. In some embodiments, the lymphoid cells are trans-differentiated from a non-pluripotent cell of non-hematopoictic lineage. In particular embodiments, the cells are modified T cells that have been modified by the provided DNA-binding systems with an epigenetic change of one or more target genes.

Provided herein are modified T cells (e.g. CD4+ T cell or CD8+ T cell) that have one or more modifications (also referred to as changes or alterations) in their epigenome. In some embodiments, the modification increases or promotes a Tsem phenotype in the T cell. In some embodiments, the modified cell is a modified T cell that has a Tscm phenotype or a Tscm-like phenotype. In some embodiments, the epigenetic change is a change relative to a comparable unmodified T cell. Reference to a comparable unmodified T cells is understood to refer to the same or similar T cell but that has not been introduced with a provided epigenome-modifying DNA-targeting system or that or that does not contain the same epigenetic changes (e.g. methylation or histone modification) of the target gene or regulatory region thereof.

In some embodiments, the epigenetic change comprises a change in at least one of: DNA accessibility, histone methylation, acetylation, phosphorylation, ubiquitylation, sumoylation, ribosylation, citrullination, and DNA methylation. In some embodiments, the epigenetic change is an altered DNA methylation of a target site in a target gene or a regulatory element thereof as described herein. In some embodiments, the epigenetic change is a histone modification of a target site in a target gene or a regulatory element thereof as described herein.

Provided herein are methods of epigenetically modifying a lymphoid cell or a population of lymphoid cells. The methods provided herein include use of one or more epigenome-modifying DNA-targeting system provided herein, or polynucleotide or vector for delivery of same to the lymphoid cell or compositions of any of the foregoing. In some embodiments, the epigenome-modifying DNA-targeting system (or polynucleotides or vectors for delivery of same to the lymphoid cell or compositions of any of the foregoing) is contacted with a lymphoid cell or a population of lymphoid cells. In some embodiments, the contacting introduces the epigenome-modifying DNA-targeting system (or polynucleotides or vectors for delivery of same to the lymphoid cell or compositions of any of the foregoing) into the lymphoid cell, such as where it is able to translocate or localize to the nucleus of the lymphoid cell. In some embodiments, the methods increase the expression of one or more of the described target genes in lymphoid cells (e.g. T cells) in the population of cells. Also provided herein is a population of lymphoid cells containing a plurality of any of the provided modified lymphoid cells.

Provided herein are methods of epigenetically modifying a T cell or a population of T cells. In some embodiments, such methods promote a Tscm phenotype, such as by altering the differentiation fate of the T cell to a Tsem phenotype. In some embodiments, such methods increase or enrich a Tscm phenotype among a population of T cells. Also provided herein are methods of promoting a Tscm phenotype in a T cell or a population of T cells. The methods provided herein include use of one or more epigenome-modifying DNA-targeting system provided herein, or polynucleotide or vector for delivery of same to the T cell or compositions of any of the foregoing. In some embodiments, the epigenome-modifying DNA-targeting system (or polynucleotides or vectors for delivery of same to the T cell or compositions of any of the foregoing) is contacted with a T cell or a population of T cells. In some embodiments, the contacting introduces the epigenome-modifying DNA-targeting system (or polynucleotides or vectors for delivery of same to the T cell or compositions of any of the foregoing) into the T cell, such as where it is able to translocate or localize to the nucleus of the T cell. In some embodiments, the methods promote a Tscm phenotype by the T cell or one or more T cells in the population. In some embodiments, the methods increase the percentage of Tscm T cells in the population of T cells.

In some embodiments, the epigenome-modifying DNA-targeting system (or polynucleotides or vectors for delivery of same to the T cell or compositions of any of the foregoing) can be introduced into a T cell or a population of T cells. In some embodiments, the epigenome-modifying DNA-targeting system (or polynucleotides or vectors for delivery of same to the T cell or compositions of any of the foregoing) can be cultured with a T cell or a population of T cells under conditions in which the epigenome-modifying DNA-targeting system (or polynucleotides or vectors for delivery of same to the T cell or compositions of any of the foregoing) will be introduced into or delivered to the T cell or one or more T cells in the population.

In some embodiments, the methods can be carried out in vitro. In other embodiments, the methods can be carried out ex vivo on T cells or a population containing T cells isolated from a subject. In other embodiments the epigenome-modifying DNA-targeting system (or polynucleotides or vectors for delivery of same to the T cell or compositions of any of the foregoing) can be administered to a subject, and then T cells can be isolated from the subject, such as for subsequent engineering. In still other embodiments the epigenome-modifying DNA-targeting system (or polynucleotides or vectors for delivery of same to the T cell or compositions of any of the foregoing) can be administered to a subject, and the T cells modified in vivo in the subject.

In any of the provided methods, the T cells can be T cells for use as a T cell based immunotherapy, such as for ACT. In certain embodiments, the population of lymphocytes is derived from peripheral blood mononuclear cells (PBMCs) isolated from the circulation of a subject. In certain embodiments, the population of lymphocytes is derived from lymphocytes isolated from a tumor (tumor infiltrating lymphocytes) of an individual. In certain embodiments, the population of lymphocytes comprises T lymphocytes (T cells). These cell populations can be heterogeneous comprised of a variety of lymphocytes, or they can be further subject to isolation/purification using density centrifugation (e.g., Percoll), fluorescently activated cell sorting (FACS), leukapheresis, or antibody based selection methods (positive or negative). T cells can be generally marked by expression of CD3, and further subdivided into cytotoxic (CD8+) or helper (CD4+) populations. When isolated/purified the cell population can comprise CD3+ cells at least 80%, 90%, or 95% pure. In certain embodiments, the population comprises CD3+, CD4+ T cells at least 80%, 90%, or 95% pure. In certain embodiments, the population comprises CD3+, CD8+ T cells at least 80%, 90%, or 95% pure.

In some embodiments, an isolated or purified cell population containing T cells can be further stimulated and, in some cases, expanded using standard methods, such as, incubation with anti-CD3 or CD28 antibody and/or co-culture with cytokines such as IL-2, IL-7 and/or IL-15. For instance, a population of isolated cells containing T cells can be further expanded using standard methods such as incubation with anti-CD3 or CD28 antibody and/or co-culture with cytokines such as IL-2, IL-7 and/or IL-15.

After the cells have been expanded the cells can comprise greater than 60%, 70%, 80%, 90%, or 95% CD3+ cells, CD3+CD4+ cells, or CD3+CD8+ cells. In certain embodiments, an aliquot of the cells can be tested for efficacy after expansion.

There are numerous methods available for isolating or expanding T cells or T-cell populations taken from an individual. Certain non-limiting methods of expanding and/or isolating T-cell populations are disclosed in U.S. Pat. Nos. 5,827,642; 6,316,257; 6,399,054; 7,745,140; 8,383,099; US 2003/0134341; US 2004/0241162; all of which are incorporated by reference herein in their entireties.

In some embodiments, the cells, such as T cells, may be further engineered with a recombinant antigen receptor, such as a chimeric antigen receptor (CAR) or an engineered TCR. In some embodiments, the cells may be stimulated (e.g. with anti-CD3 or CD28 antibody and/or IL-2. IL-7 and/or IL-15 cytokines) prior to engineering the cells, such as T cells, with the recombinant receptor. In some embodiments, the cells may be further expanded after engineering the cells, such as T cells, with the recombinant receptor.

In some embodiments, the cells, such as T cells, are engineered with a CAR. In some embodiments, the CAR is a chimeric receptor that contains an extracellular antigen targeting domain (e.g., an antibody Fab or single chain variable fragment) fused to a transmembrane domain, and an intracellular signaling domain that induces activation of the cell, such as T cell, upon interaction of the CD3 zeta signaling domain and a costimulatory signaling domain. Non-limiting examples of a costimulatory signaling domain include a CD28 intracellular domain or a 4-1BB intracellular domain. In some embodiments, the extracellular targeting domain is specific for a tumor associated antigen (TAA). Non-limiting examples of TAAs include, for example, CD19, glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, MART-1, Lewis Y antigen (LeY), tyrosinase and GP 100, prostatic acid phosphatase (PAP) prostate-specific antigen (PSA), ROR1, MUC16, CD171 (LICAM), B-cell maturation antigen (BCMA), WT1, HER-2/Ncu/ErbB-2, CD19, CD20, or CD37. Current FDA approved CAR T cell therapies include CAR-T cell therapies for targeting CD19, such as for treating lymphoma, including axicabtagene ciloleucel (Yescarta™), tisagenlecleucel (Kymriah™) and lisocabtagene maralcucel (Breyanzi®). Current FDA approved CAR T cell therapies also include CAR-T cell therapies for targeting BCMA, such as for treating multiple myeloma, including idecabtagene vicleucel (Abecma®), CAR constructs and methods of their use are described in, by way of non-limiting example, US20130287748A1; US 2014/0234348A1; or US 2014/0050708, all of which are incorporated by reference herein in their entirety.

In some embodiments, the T cells are engineered with a TCR. In some embodiments, the TCR is specific for a TAA. In particular embodiments, the TCR is a recombinant TCR that is introduced into the T cell and is heterologous to the T cell. The TCR can be specific for a TAA, such as, glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, MART-1, Lewis Y antigen (LeY), tyrosinase and GP 100, prostatic acid phosphatase (PAP) prostate-specific antigen (PSA), ROR1, MUC16, CD171 (LICAM), B-cell maturation antigen (BCMA), WT1, HER-2/Neu/ErbB-2, CD19, CD20, or CD37. In some embodiments, the TAA is cancer-testis (CT) antigen. In some embodiments, the TAA is neoantigen or oncoviral antigen. Exemplary target antigens of a TCR include, but are not limited to, a MAGE (e.g. MAGE-A1, MAGE-A2, MAGE-A3, MAGE-A4, MAGE-A6, MAGE-A10, and MAGE-A12, such as MAGE-A4, MAGE-A10, MAGE-A3/MAGE-A6), glycoprotein (gp100), melanoma antigen recognized by T cells (MART-1), Wilms tumor 1 (WT1), PRAME, NY-ESO-1, mesothelin, a-fetoprotein (AFP) or Human papillomavirus (HPV) E6 protein and HPV E7 protein. TCRs and method of their use in ACT are known and described in, by way of non-limiting example, Tsimberidou, A M., et al. J Hematol Oncol 14, 102 (2021).

In some embodiments, the recombinant antigen receptor, such as a CAR or TCR, can be engineered into the cell, such as T cell, by viral transduction of a nucleic acid encoding the recombinant antigen receptor into a primary T-cell population, using for example a retroviral, adenoviral, or AAV-vector; or transfection via a lipid-based reagent or electroporation. In some embodiments, the methods described herein involve engineering a population of lymphoid cells, such as a T-cell population, with the recombinant antigen receptor (e.g. CAR or TCR) before contacting the population with the epigenome-modifying DNA-targeting system (or polynucleotides or vectors for delivery of same to the T cell or compositions of any of the foregoing). In certain embodiments, the methods involve engineering a population of lymphoid cells, such as a T-cell population, with the recombinant antigen receptor (e.g. CAR or TCR) after contacting the cells with the epigenome-modifying DNA-targeting system (or polynucleotides or vectors for delivery of same to the T cell or compositions of any of the foregoing). In some embodiments, when the engineered lymphocytes, such as T cells (e.g. CAR-T cells or eTCR-T cells), are generated from a primary lymphocyte population the cells are often autologous to the patient being treated. In some embodiments, a process for engineering T cells with a recombinant receptor (e.g. CAR or TCR) includes isolating the T cells from a subject, stimulating the T cells in culture using a conventional method such as CD3/CD28 antibodies prior to transduction with a viral vector encoding the recombinant antigen receptor (e.g. CAR or TCR) and, if necessary, expanding the cells to generate sufficient cells for subsequent administration to the subject. In some embodiments, contacting the T cells with the epigenome-modifying DNA-targeting system (or polynucleotides or vectors for delivery of same to the T cell or compositions of any of the foregoing) can be prior to or during any step of stimulating, transducing or expanding the T cells.

In certain embodiments, an isolated or purified cell population containing T cells is incubated with peptide antigen and, in some cases also irradiated feeder cells or other agents, to expand one or more T cells of a certain antigen specificity. In certain embodiments, the peptide antigen comprises a tumor associated antigen. In some embodiments, such an isolated or purified cell population includes tumor infiltrating lymphocytes (TILs) such as for TIL therapy. In some embodiments, the population can be stimulated or activated by a specific tumor-associated antigen either before or after contact with epigenome-modifying DNA-targeting system (or polynucleotides or vectors for delivery of same to the T cell or compositions of any of the foregoing). A tumor associated antigen (TAA) is one that is exclusively expressed or highly expressed by a neoplastic cell compared to a normal cell of the same origin. Known tumor-associated antigens include, for example, glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP. NY-ESO-1, LAGE-la, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin, MART-1, Lewis Y antigen (LeY), tyrosinase and GP 100, prostatic acid phosphatase (PAP) prostate-specific antigen (PSA), ROR1, MUC16, CD171 (LICAM), B-cell maturation antigen (BCMA), WT1, HER-2/Neu/ErbB-2, CD19, CD20, CD37, or patient specific idiotype. In certain embodiments, greater than 50%, 60%, 70%, 80%, 90%, or 95% of the T-cell population can be specific for a tumor associated antigen (as defined by tetramer staining for example). In certain embodiments, the T-cell population may not be stimulated with TAA, but may possess specificity for the TAA, as indicated for example, by tetramer staining.

In some embodiments, the population of cells, such as T cells, may be autologous to a subject to be treated. For instance, the population of lymphoid cells, such as T-cell populations, to be contacted with an epigenome-modifying DNA-targeting system (or polynucleotides or vectors for delivery of same to the T cell or compositions of any of the foregoing) can be derived from an individual that will ultimately be treated with the cell-based immunotherapeutic (e.g., an autologous population). In certain embodiments, when an autologous cell population is used the cell population has been contacted in vitro with the epigenome-modifying DNA-targeting system (or polynucleotides or vectors for delivery of same to the cells, such as T cell, or compositions of any of the foregoing). In certain embodiments, when an autologous cell population is used a subject to be treated has been administered the epigenome-modifying DNA-targeting system (or polynucleotides or vectors for delivery of same to the cell, such as T cell, or compositions of any of the foregoing) on one or more occasions prior to isolation of the cell population.

In other embodiments, the population of lymphoid cells, such as population of T cells, may be for allogeneic therapy. In such an example, the population of lymphoid cells, such as T-cell population, to be contacted with an epigenome-modifying DNA-targeting system (or polynucleotides or vectors for delivery of same to the cells, such as T cell, or compositions of any of the foregoing) can be derived from a different individual (e.g., a heterologous population) than is to be treated. In certain embodiments, when a heterologous cell population is used it is from an HLA matched individual (e.g., syngeneic) or an HLA mismatched individual (e.g., allogeneic). In certain embodiments, when a heterologous cell population is used it is from an HLA mismatched donor. In certain embodiments, when a heterologous cell population is used it is a T cell line that can be established from an autologous or heterologous source.

T cell populations can also be derived from hematopoietic stem cells (HSCs) or induced pluripotent stem cells (iPSCs) using methods known in the art. In certain embodiments, T-cell populations are derived/differentiated from iPSCs. The source of the iPSCs can be either autologous or heterologous. In certain embodiments, T-cell populations are derived/differentiated from (HSCs) cells. The source of the HSCs can be either autologous or heterologous.

In some embodiments, the modified T cell comprises an epigenetic or phenotypic modification resulting from being contacted by any of the DNA-targeting systems described herein, including any including any gRNA described herein.

In some embodiments, the modified T cell is derived from a cell from a subject, such as a primary T cell, a T cell progenitor, a pluripotent stem cell, or an induced pluripotent stem cell. In some embodiments, the modified T cell is derived from a primary T cell.

In some embodiments, the modified T cell is derived from a subject. In some embodiments, the subject has or is suspected of having cancer.

In some aspects, provided herein are methods for modulating (e.g. increasing transcription of) the expression of a gene in a cell (e.g. a T cell), such as a target gene as described in Section I.A. In some embodiments, the method includes: introducing into the cell any of the DNA-targeting systems described herein, or a polynucleotide or vector containing or encoding the same. In some embodiments, the expression of the one or more genes, such as one or more target genes described in Section I.A, is increased in comparison to a comparable unmodified cell (e.g. T cell) not subjected to the method, i.e. not contacted or introduced with the DNA-targeting system described herein. In some embodiments, the expression of the one or more genes is increased by at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, 5-fold, 10-fold, 20-fold, 30-fold, 40-fold, 50-fold, 100-fold, 200-fold, 300-fold, 400-fold, 500-fold, 1000-fold or greater. In some embodiments, the expression is stably increased or transiently increased. In some embodiments, the increased expression of the one or more genes promotes a T_{SCM} cell-like phenotype in a T cell.

In some embodiments, the one or more modifications in the epigenome of the modified lymphoid cells, such as a T cell, NK cell or NK T cell, or any cells that have been differentiated from stem cells into such lymphoid cells and/or have been differentiated from progenitor cells, such as common lymphoid progenitors (CLPs), is by targeting one or more genes as described herein with a provided epigenome-modifying DNA-targeting system to change the epigenome of the cell. In some embodiments, the one or more modifications in the epigenome of the modified T cell is by targeting one or more genes as described herein with a provided epigenome-modifying DNA-targeting system to change the epigenome of the T cell. In some embodiments, the modified cell, such as modified T cell, includes an epigenetic change in a gene selected from the list consisting of: DDIT3, EBF4, FOXD3, HELT, HES2, HKR1, HMGN3, PATZ1, PLAG1, PRDM4, PRDM8, SOX21, TSHZ1, ZBED5, ZBTB7A, ZNF141, ZNF219, ZNF319, ZNF470, ZNF562, ZNF691, ZNF692, ZNF816, ZSCAN23, ZSCAN5A, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF319, ZNF331, ZNF345, ZNF420, ZNF423, ZNF470, ZNF48, ZNF496, ZNF501, ZNF524, ZNF541, ZNF557, ZNF619, ZNF735, ZNF747, ZNF783, ZNF805, and ZSCAN10. In some embodiments, the gene is selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23. In some embodiments, the gene is selected from the list consisting of: DDIT3, HES2, HKR1, PLAG1, PRDM4, TEF, TSHZ1, ZNF141, ZNF562, ZNF691, ZNF692, and ZSCAN5A. In some embodiments, the epigenetic change of any of the above target genes is a change in at least one of: DNA accessibility, histone methylation, acetylation, phosphorylation, ubiquitylation, sumoylation, ribosylation, citrullination, and DNA methylation, compared to a comparable unmodified cell (e.g. T cell) not subjected to the method, i.e. not contacted or introduced with the DNA-targeting system described herein.

In some embodiments, the modified cell, such as modified T cell has increased expression of one or more genes selected from the list consisting of: DDIT3, EBF4, FOXD3, HELT, HES2, HKR1, HMGN3, PATZ1, PLAG1, PRDM4, PRDM8, SOX21, TSHZ1, ZBED5, ZBTB7A, ZNF141, ZNF219, ZNF319, ZNF470, ZNF562, ZNF691, ZNF692, ZNF816, ZSCAN23, ZSCAN5A, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF319, ZNF331, ZNF345, ZNF420, ZNF423, ZNF470, ZNF48, ZNF496, ZNF501, ZNF524, ZNF541, ZNF557, ZNF619, ZNF735, ZNF747, ZNF783, ZNF805, and ZSCAN10. In some embodiments, the gene is selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23. In some embodiments, the gene is selected from the list consisting of: DDIT3, HES2, HKR1, PLAG1, PRDM4, TEF, TSHZ1, ZNF141, ZNF562, ZNF691, ZNF692, and ZSCAN5A. In some embodiments, the expression of the gene in the modified cell is increased 1.5-fold or more compared to the expression of the same gene in a comparable unmodified T cell, such as increased by at or about or greater than 2-fold, 3-fold, 4-fold, 5-fold, 6-fold, 7-fold, 8-fold, 9-fold, 10-fold or more.

In some embodiments, the modified T cell exhibits increased expression of one or more genes that promote a stem cell-like memory T (T_{SCM}) cell phenotype, in comparison to a comparable unmodified T cell, such as a T cell not subjected to the method, i.e. not contacted or introduced with the DNA-targeting system described herein. In some embodiments, the modified T cell has increased expression of one more genes selected from the list consisting of: DDIT3, EBF4, FOXD3, HELT, HES2, HKR1, HMGN3, PATZ1, PLAG1, PRDM4, PRDM8, SOX21, TSHZ1, ZBED5, ZBTB7A, ZNF141, ZNF219, ZNF319, ZNF470, ZNF562, ZNF691, ZNF692, ZNF816, ZSCAN23, ZSCAN5A, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF319, ZNF331, ZNF345, ZNF420, ZNF423, ZNF470, ZNF48, ZNF496, ZNF501, ZNF524, ZNF541, ZNF557, ZNF619, ZNF735, ZNF747, ZNF783, ZNF805, and ZSCAN10. In some embodiments, the gene is selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23. In some embodiments, the gene is selected from the list consisting of: DDIT3, HES2, HKR1, PLAG1, PRDM4, TEF, TSHZ1, ZNF141, ZNF562, ZNF691, ZNF692, and ZSCAN5A. In some embodiments, the expression of the gene in the modified T cell is increased 1.5-fold or more compared to the expression of the same gene in a comparable unmodified T cell, such as increased by at or about or greater than 2-fold, 3-fold, 4-fold, 5-fold, 6-fold, 7-fold, 8-fold, 9-fold, 10-fold or more.

In some embodiments, the modified T cell exhibits a Tscm cell phenotype, or a Tscm cell-like phenotype. In some aspects, a Tsem phenotype comprises expression of one or more cell surface markers selected from CCR7+, CD27+, CD45RA+, CD45RO−, CCR7+, CD62L+, CD28+. CD27+, IL-7Rα+, CXCR3+, CD95+, CD11a+, IL-2Rβ+, CD58+, and CD57−. In some aspects, a Tscm phenotype comprises expression of CCR7 and/or CD27. In some aspects, a Tscm phenotype comprises expression of CCR7 and CD27.

Also provided herein is a population of T cells containing a plurality of any of the provided modified T cells. In some embodiments, the population of T cells is enriched for cells that have a Tscm phenotype. In some embodiments, the population of T cells contains greater than at or about 40%, greater than at or about 50%, greater than at or about 60%, greater than at or about 70%, greater than at or about 80% or greater than at or about 90% of cells that have a Tscm phenotype. In some embodiments, the population of T cells has an increased percentage of cells of a Tscm phenotype compared to a comparable population of unmodified cell (e.g. T cell) not subjected to the method, i.e. not contacted or introduced with the DNA-targeting system described herein. In some embodiments, the population of T cells has an increased percentage of cells of a Tscm phenotype compared to the percentage of cells of the Tscm phenotype prior to contacting the population of T cells with the DNA-targeting system described herein. In some embodiments, the increased percentage is by at or about or greater than 1.5-fold, 2-food, 3-fold, 4-fold, 5-fold or more. In some aspects, a Tscm phenotype comprises expression of one or more cell surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−. In some aspects, a Tscm phenotype comprises expression of CCR7 and/or CD27. In some aspects, a Tscm phenotype comprises expression of CCR7 and CD27.

In some embodiments, the population of cells, such as population of T cells, contains at least at or about 40%, at least at or about 50%, at least at or about 60%, at least at or about 70%, at least at or about 80% or at least at or about 90% of cells that have an epigenetic change (e.g. methylation or histone modification) at or near a target site in a target gene. In some embodiments, the population of cells, such as population of T cells, has an increased percentage of cells (e.g. T cells) that have an epigenetic change at or near a target site in a target gene compared to a comparable population of unmodified cell (e.g. T cell) not subjected to the method, i.e. not contacted or introduced with the DNA-targeting system described herein. In some embodiments, the epigenetic change is a change, such as on average in cells in the population, of at least one of: DNA accessibility, histone methylation, acetylation, phosphorylation, ubiquitylation, sumoylation, ribosylation, citrullination, and DNA methylation, compared to a comparable population of unmodified cell (e.g. T cell) not subjected to the method, i.e. not contacted or introduced with the DNA-targeting system described herein. In some embodiments the population of cells is a population of T cells. In some embodiments, the population of T cells contains at least at or about 40%, at least at or about 50%, at least at or about 60%, at least at or about 70%, at least at or about 80% or at least at or about 90% of cells that have an epigenetic change (e.g. methylation or histone modification) at or near a target site in a target gene and exhibits a Tscm phenotype. In some aspects, a Tscm phenotype comprises expression of one or more cell surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−. In some aspects, a Tscm phenotype comprises expression of CCR7 and/or CD27. In some aspects, a Tscm phenotype comprises expression of CCR7 and CD27.

In some embodiments, provided herein is a population of cells that contains at least at or about 40%, at least at or about 50%, at least at or about 60%, at least at or about 70%, at least at or about 80% or at least at or about 90% of cells that have an epigenetic change (e.g. methylation or histone modification) at a target gene and that are double positive for CCR7 and CD27. In some embodiments, the population of cells is a population of T cells.

In some embodiments, the modified T cell or a composition containing a plurality of modified T cells is capable of a stronger and/or more persistent immune response (e.g. an anti-tumor immune response in vivo), in comparison to a comparable unmodified T cell or composition of unmodified T cells. In some embodiments, a subject having received administration of a composition of T cells containing provided modified T cells as a T cell therapy, e.g. CAR-T cell, is monitored for the presence, absence or level of T cells of the therapy in the subject, such as in a biological sample of the subject, e.g. in the blood of the subject. In some embodiments, the provided methods result in T cells of the adoptive T cell therapy with increased persistence and/or better potency in a subject to which it is administered. In some embodiments, the persistence of the adoptively transferred T cells, such as CAR-expressing T cells, in the subject is greater as compared to that which would be achieved by alternative methods, such as those involving administration of a T cell therapy but without having been treated or contacted with a provided DNA-targeting system. In some embodiments, the persistence is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In some embodiments, the degree or extent of persistence of administered cells can be detected or quantified after administration to a subject. For example, in some aspects, quantitative PCR (qPCR) is used to assess the quantity of cells expressing the recombinant receptor (e.g., CAR or recombinant TCR) or other surrogate marker expressed by T cells of the therapy in the blood or serum or organ or tissue (e.g., disease site) of the subject. In some aspects, persistence is quantified as copies of DNA or plasmid encoding the recombinant receptor (e.g., CAR or recombinant TCR) or surrogate marker per microgram of DNA or per microliter of the sample, e.g., of blood or serum, or per total number of peripheral blood mononuclear cells (PBMCs) or white blood cells or T cells per microliter of the sample. In some embodiments, flow cytometric assays using antibodies specific for the recombinant receptor or surrogate marker also can be performed to detect the adoptively transferred cells. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of any marker (e.g. surrogate marker, CAR, recombinant TCR) known to be expressed by the adoptively transferred T cells but not endogenous T cells can be used to distinguish the administered cells from endogenous cells in a subject.

In some embodiments, the modified T cell or a composition containing a plurality of modified T cells, such a produced by any of the provided methods, exhibits a reduction in features associated with T cell exhaustion in comparison to a comparable unmodified T cell or composition of unmodified T cells. In some embodiments, the T cells, such as a composition containing a modified T cell or a composition of modified T cell provided herein, exhibits reduced exhaustion following long-term stimulation with antigen, either in vitro or in vivo. For example, an assay for assessing long-term stimulation with antigen may include a serial restimulation assay (see e.g. Jensen et al. Immunol. Rev. 2014; 257:127-144; Win et al. Journal of Immunotherapy, 2020; 43:107-120). In some embodiments, the percentage of T cells that exhibit an exhausted phenotype is reduced 2-fold, 3-fold, 4-fold, 5-fold, 6-fold, 7-fold, 8-fold, 9-fold, 10-fold or more.

Various assays are known and can be used to assess or determine if the T cells exhibit features of exhaustion or a reduction in features of exhaustion in comparison to a comparable unmodified T cell or composition of unmodified T cells. In some cases, exhaustion can be assessed by monitoring loss of T cell function, such as reduced or decreased antigen-specific or antigen receptor-driven activity, such as a reduced or decreased ability to produce cytokines or to drive cytolytic activity against target antigen. In some cases, exhaustion also can be assessed by monitoring expression of surface markers on T cells (e.g. CD4 and/or CD4 T cells) that are associated with an exhaustion phenotype. In some embodiments, the exhaustion marker is any one or more of PD-1, CTLA-4, TIM-3, LAG-3, BTLA, 2B4, CD160, CD39, VISTA, and TIGIT Among exhaustion markers are inhibitory receptors such as PD-1, CTLA-4, LAG-3 and TIM-3. In certain embodiments, the biological activity of the cells is measured by assaying expression and/or secretion of one or more cytokines, such as CD107a, IFNγ, IL-2, GM-CSF and TNFα, and/or by assessing cytolytic activity. In some embodiments, assays for the activity, phenotypes, proliferation and/or function of the T cells include, but are not limited to, ELISPOT, ELISA, cellular proliferation, cytotoxic lymphocyte (CTL) assay, binding to the T cell epitope, antigen or ligand, or intracellular cytokine staining, proliferation assays, lymphokine secretion assays, direct cytotoxicity assays, and limiting dilution assays. In some embodiments, proliferative responses of the T cells can be measured, e.g. by incorporation of ³H-thymidine, BrdU (5-Bromo-2′-Deoxyuridine) or 2′-deoxy-5-ethynyluridine (EdU) into their DNA or dye dilution assays, using dyes such as carboxyfluorescein diacetate succinimidyl ester (CFSE), CellTrace Violet, or membrane dye PKH26.

Also provided herein are compositions containing a modified lymphoid cell or a plurality of or population of modified lymphoid cells provided herein, such as modified T cells, NK cell, NKT cell, or such cells that are modified and have been differentiated from stem cells into such lymphoid cells and/or have been differentiated from progenitor cells, such as common lymphoid progenitors (CLPs). Also provided herein are compositions containing a modified T cell or a plurality of or population of modified T cells provided herein. In some embodiments, the composition is a pharmaceutical composition and further contains a pharmaceutically acceptable carrier. Such compositions can be used in accord with the provided methods, and/or with the provided articles of manufacture or compositions, such as in the prevention or treatment of diseases, conditions, and disorders, or in detection, diagnostic, and prognostic methods.

Among the compositions are pharmaceutical compositions and formulations for administration, such as for adoptive cell therapy. In some embodiments, the engineered cells are formulated with a pharmaceutically acceptable carrier.

A pharmaceutically acceptable carrier can include all solvents, dispersion media, coatings, antibacterial and antifungal agents, isotonic and absorption delaying agents, and the like, compatible with pharmaceutical administration (Gennaro, 2000, Remington: The science and practice of pharmacy, Lippincott, Williams & Wilkins, Philadelphia, PA). Examples of such carriers or diluents include, but are not limited to, water, saline, Ringer's solutions, dextrose solution, and 5% human serum albumin. Liposomes and non-aqueous vehicles such as fixed oils may also be used. Supplementary active compounds can also be incorporated into the compositions. The pharmaceutical carrier should be one that is suitable for T cells, such as a saline solution, a dextrose solution or a solution comprising human serum albumin.

In some embodiments, the pharmaceutically acceptable carrier or vehicle for such compositions is any non-toxic aqueous solution in which the cells, such as T cells, can be maintained, or remain viable, for a time sufficient to allow administration of live cells, such as live T cells. For example, the pharmaceutically acceptable carrier or vehicle can be a saline solution or buffered saline solution. The pharmaceutically acceptable carrier or vehicle can also include various bio materials that may increase the efficiency of the cells, such as T cells. Cell vehicles and carriers can, for example, include polysaccharides such as methylcellulose (M. C. Tate, D. A. Shear, S. W. Hoffman, D. G. Stein, M. C. LaPlaca, Biomaterials 22, 1113, 2001, which is incorporated herein by reference in its entirety), chitosan (Suh J K F, Matthew H W T. Biomaterials, 21, 2589, 2000; Lahiji A, Sohrabi A, Hungerford D S, et al., J Biomed Mater Res, 51, 586, 2000, each of which is incorporated herein by reference in its entirety), N-isopropylacrylamide copolymer P (NIPAM-co-AA) (Y. H. Bac, B. Vernon, C. K. Han, S. W. Kim, J. Control. Release 53, 249, 1998; H. Gappa, M. Baudys, J. J. Koh, S. W. Kim, Y. H. Bac, Tissue Eng. 7, 35, 2001, each of which is incorporated herein by reference in its entirety), as well as Poly (oxyethylenc)/poly (D,L-lactic acid-co-glycolic acid) (B. Jeong, K. M. Lec, A. Gutowska, Y. H. An, Biomacromolecules 3, 865, 2002, which is incorporated herein by reference in its entirety), P (PF-co-EG) (Suggs L J, Mikos A G. Cell Trans, 8, 345, 1999, which is incorporated herein by reference in its entirety), PEO/PEG (Mann B K, Gobin A S, Tsai A T, Schmedlen R H, West J L., Biomaterials, 22, 3045, 2001; Bryant S J, Anseth K S. Biomaterials, 22, 619, 2001, each of which is incorporated herein by reference in its entirety), PVA (Chih-Ta Lec, Po-Han Kung and Yu-Der Lee, Carbohydrate Polymers, 61, 348, 2005, which is incorporated herein by reference in its entirety), collagen (Lee C R. Grodzinsky A J, Spector M., Biomaterials 22, 3145, 2001, which is incorporated herein by reference in its entirety), alginate (Bouhadir K H, Lec K Y, Alsberg E, Damm K L, Anderson K W. Mooney D J. Biotech Prog 17, 945, 2001; Smidsrd O, Skjak-Brack G., Trends Biotech, 8, 71, 1990, each of which is incorporated herein by reference in its entirety).

In some embodiments, the cells, such as T cells, can be present in the composition in an effective amount. In some embodiments, the composition contains an effective amount of T cells, such as containing modified T cells produced by the provided methods. In some embodiments, the composition of T cells are enriched in T cells with a Tsem phenotype. An effective amount of cells can vary depending on the patient, as well as the type, severity and extent of disease. Thus, a physician can determine what an effective amount is after considering the health of the subject, the extent and severity of disease, and other variables.

In some embodiments, the composition, including pharmaceutical composition, is sterile. In some embodiments, isolation, enrichment, or culturing of the cells is carried out in a closed or sterile environment, for example and for instance in a sterile culture bag, to minimize error, user handling and/or contamination. In some embodiments, sterility may be readily accomplished, e.g., by filtration through sterile filtration membranes. In some embodiments, culturing is carried out using a gas permeable culture vessel. In some embodiments, culturing is carried out using a bioreactor.

Also provided herein are compositions that are suitable for cryopreserving the provided lymphoid cells, such as modified cells including such lymphoid cells produced by any of the provided methods. In some embodiments, the lymphoid cells are cryopreserved in a serum-free cryopreservation medium.

Also provided herein are compositions that are suitable for cryopreserving the provided T cells, such as modified T cells including T cells produced by any of the provided methods. In some embodiments, the T cells are cryopreserved in a serum-free cryopreservation medium.

In some embodiments, the composition comprises a cryoprotectant. In some embodiments, the cryoprotectant is or comprises DMSO and/or s glycerol. In some embodiments, the cryopreservation medium is between at or about 5% and at or about 10% DMSO (v/v). In some embodiments, the cryopreservation medium is at or about 5% DMSO (v/v). In some embodiments, the cryopreservation medium is at or about 6% DMSO (v/v). In some embodiments, the cryopreservation medium is at or about 7% DMSO (v/v). In some embodiments, the cryopreservation medium is at or about 8% DMSO (v/v). In some embodiments, the cryopreservation medium is at or about 9% DMSO (v/v). In some embodiments, the cryopreservation medium is at or about 10% DMSO (v/v). In some embodiments, the cryopreservation medium contains a commercially available cryopreservation solution (CryoStor™ CS10). CryoStor™ CS10 is a cryopreservation medium containing 10% dimethyl sulfoxide (DMSO). In some embodiments, compositions formulated for cryopreservation can be stored at low temperatures, such as ultra low temperatures, for example, storage with temperature ranges from −40° C. to −150° C., such as or about 80° C.±6.0° C.

In some embodiments, the cryopreserved cells, such as T cells, are prepared for administration by thawing. In some cases, the cells, such as T cells, can be administered to a subject immediately after thawing. In such an embodiment, the composition is ready-to-use without any further processing. In other cases, the cells, such as T cells, are further processed after thawing, such as by resuspension with a pharmaceutically acceptable carrier, incubation with an activating or stimulating agent, or are activated washed and resuspended in a pharmaceutically acceptable buffer prior to administration to a subject.

### V. METHODS OF TREATMENT

Provided herein are methods of treatment, e.g., including administering any of the compositions, such as pharmaceutical compositions described herein. In some aspects, also provided are methods of administering any of the compositions described herein to a subject, such as a subject that has a disease or disorder. The compositions, such as pharmaceutical compositions, described herein are useful in a variety of therapeutic, diagnostic and prophylactic indications. For example, the compositions are useful in treating a variety of diseases and disorders in a subject. Such methods and uses include therapeutic methods and uses, for example, involving administration of the compositions, to a subject having a disease, condition, or disorder, such as a tumor or cancer. In some embodiments, the compositions are administered in an effective amount to effect treatment of the disease or disorder. Uses include uses of the compositions in such methods and treatments, and in the preparation of a medicament in order to carry out such therapeutic methods. In some embodiments, the methods are carried out by administering the compositions to the subject having or suspected of having the disease or condition. In some embodiments, the methods thereby treat the disease or condition or disorder in the subject. Also provided are therapeutic methods for administering the cells and compositions to subjects, e.g., patients.

In some embodiments, the compositions include a DNA-targeting system provided herein, or a polynucleotide or vector encoding the same, in which delivery of the composition to a subject modulates one or more activities or function of lymphoid cells in a subject to thereby treat a disease or condition. For instance, in some embodiments, the subject has been previously treated with an adoptive cell therapy involving administration of a population of lymphoid cells (e.g. T cell, NK or NKT cell therapy, including primary cells or cells differentiated from stem cells or progenitor cells such as common lymphoid cells) for treating a disease or disorder, and administration of a provided DNA-targeting system, or a polynucleotide or vector encoding the same, modulates a phenotype or function of the adoptively transferred cells in the subject for treating the disease or condition. In some embodiments, the cells may include a T cell infiltrating lymphocyte (TIL) therapy. In some embodiments, the cells are engineered with an antigen receptor, such as a chimeric antigen receptor or T cell receptor, targeting an antigen associated with the disease or condition. In some embodiments, administration or use of a composition that includes a DNA-targeting system provided herein, or a polynucleotide or vector encoding the same, increases expression of one or more target genes as described herein in the lymphoid cell.

In some embodiments, the compositions include a DNA-targeting system provided herein, or a polynucleotide or vector encoding the same, in which delivery of the composition to a subject modulates one or more activities or function of T cells in a subject to thereby treat a disease or condition. For instance, in some embodiments, the subject has been previously treated with an adoptive T cell therapy for treating a disease or disorder, such as a TIL therapy or a CAR- or TCR-engineered T cell therapy, and administration of a provided DNA-targeting system, or a polynucleotide or vector encoding the same, modulates a phenotype or function of the adoptively transferred T cells in the subject for treating the disease or condition. In some embodiments, administration or use of a composition that includes a DNA-targeting system provided herein, or a polynucleotide or vector encoding the same, increases expression of one or more genes that promotes a T_{SCM} cell-like phenotype in a T cell. In some embodiments, the percentage of T cells of the adoptive cell therapy in the subject that has a T_{SCM} cell-like phenotype, e.g. CCR7+ and/or CD27+, is increased in the subject compared to prior to the administration of the composition that includes the DNA-targeting system or a polynucleotide or vector encoding the same.

In some aspects, also provided herein are methods of promoting a T_{SCM} cell phenotype in a T cell or in T cells in a subject, according to any description of a T_{SCM} cell phenotype provided herein. For instance, the T_{SCM} phenotype includes T cells that are CCR7+ and/or CD27+, such as CCR7+ and CD27+. In some embodiments, the percentage of T cells that have a T_{SCM} cell-like phenotype, e.g. CCR7+ and/or CD27+, is increased in the subject compared to prior to the administration of the composition containing the DNA-targeting system or a polynucleotide or vector encoding the same. In some embodiments, the T cells include T cells of a previously administered adoptive cell therapy, such as CAR-expressing or recombinant TCR-expressing T cells.

In some embodiments, the methods of administering a composition containing the DNA-targeting system or a polynucleotide or vector encoding the same to a subject as provided herein are carried out in vivo (i.e. in a subject).

In some embodiments, methods of contacting a cell (e.g. T cell) with a composition containing the DNA-targeting system or a polynucleotide or vector encoding the same provided herein are carried out ex vivo on a cell from a subject, for example a primary T cell, a T cell progenitor, a pluripotent stem cell, or an induced pluripotent stem cell, such as by methods described in Section IV. In some embodiments, the methods provided herein are carried out ex vivo on a primary T cell. In some embodiments, when the methods are carried out ex vivo, such as by methods described in Section IV, the provided methods of treatment include administering a dose of the modified cells (e.g. T cells) to the subject for treating a disease or disorder. In some embodiments, the modified cells are modified T cells that have been epigenetically modified by the provided methods and enriched in T cells that have a Tscm phenotype.

In some embodiments, also provided herein are methods include administering to a subject a composition containing an epigenetically modified cells (e.g. epigenetically modified T cells) provided herein. In some embodiments, administration of an effective dose of epigenetically modified cells treats a disease or condition in the subject. In some embodiments, the dose of epigenetically modified cells (e.g. T cells) is for use in adoptive cell therapy. In some embodiments, the epigenetically modified cell is a tumor infiltrating lymphocyte (TIL) therapy. In some embodiments, the epigenetically modified cell is a T cell that has been engineered with a recombinant antigen receptor, such as a chimeric antigen receptor or a T cell receptor (TCR) in which targeting of the antigen by the recombinant receptor (e.g. CAR or TCR)-engineered T cell treats the disease or condition.

In some aspects, provided is a method for treating a disease in a subject, comprising administering to the subject a cellular composition that comprises any of the modified T cells described herein. In some aspects, the modified cell (e.g. T cell) is one that has been obtained from or derived from a cell from a subject and modified by contacting the cells with a provided DNA-targeting system or a polynucleotide or vector encoding the same. In some aspects, the modified cell (e.g. T cell) is obtained from or derived from a cell from a subject, and administered to the same subject (i.e. autologous adoptive cell therapy). In some aspects, the modified cell (e.g. T cell) is obtained from or derived from a cell from a subject, and administered to a different subject (i.e. allogeneic adoptive cell therapy).

In some embodiments, the methods of treatment or uses involve administration to a subject of an effective amount of a composition containing modified cells (e.g. T cells) provided herein. In some embodiments, the effective amount may include a dose of cells (e.g. T cells) of the composition from at or about 10⁵ to at about 10¹², or from at or about 10⁵ and at or about 10⁸, or from at or about 10⁶ and at or about 10¹², or from at or about 10⁸ and at or about 10¹¹, or from at or about 10⁹ and at or about 10¹⁰ of such. In some embodiments, the provided compositions containing modified cells (e.g. T cells) provided herein can be administered to a subject by any convenient route including parenteral routes such as subcutaneous, intramuscular, intravenous, and/or epidural routes of administration. In particular embodiments, the modified T cells are administered by intravenous infusion to the subject.

In some embodiments, the methods of treatment or uses involve administration to a subject of an effective amount of a composition containing modified cells T cells provided herein, including any such composition that is enriched in T cells of a Tsem phenotype as produced by the provided methods. In some embodiments, the effective amount may include a dose of T cells of the composition from at or about 10⁵ to at about 10¹², or from at or about 10⁵ and at or about 10⁸, or from at or about 10⁶ and at or about 10¹², or from at or about 10⁸ and at or about 10¹¹, or from at or about 10⁹ and at or about 10¹⁰ of such. In some embodiments, the provided compositions containing modified T cells provided herein can be administered to a subject by any convenient route including parenteral routes such as subcutaneous, intramuscular, intravenous, and/or epidural routes of administration. In particular embodiments, the modified T cells are administered by intravenous infusion to the subject.

In some embodiments, the provided methods can be used to treat any disease or disorder in which treatment is contemplated by the adoptive cell therapy. For instance, in the case of a CAR or a TCR, the disease or condition to be treated is any disease or condition that is associated with expression of an antigen that is recognized or targeted by the CAR- or TCR-cell therapy. In other embodiments, for the case of a TIL therapy the disease or condition is a tumor, and typically is a tumor present in the subject from which the TIL therapy was derived. Methods for adoptive T cell therapy are known, see e.g. for CAR-T cell therapy: U.S. Pat. Nos. 7,446,190, 7,741,465, WO2016109410, WO2012079000, WO2017015427, WO2017040930, WO2017149515, WO201716568; WO2017181119; for TCR-T cell therapy: US20160137715, US20190321478; WO2015184228, WO2017158103; for TIL therapy: US2003194804, US20120244133, US20210220457, US20210189339, U.S. Pat. Nos. 5,126,132, and 11,083,752. Any of such methods or other similar methods can be used in connection with the present disclosure. In some embodiments, the provided methods are performed ex vivo during the process of manufacturing or preparing the T cells for adoptive transfer to a subject, such as using methods described in Section IV, and then the modified T cells are administered to the subject for treating a disease or disorder. In other embodiments, the provided methods are performed by administering to the subject a composition containing the DNA-targeting system or a polynucleotide or vector encoding the same in combination with adoptive transfer of a T cell therapy. In such methods, the composition containing the DNA-targeting system or a polynucleotide or vector encoding the same is administered prior to, simultaneously with or after administration of the adoptive T cell therapy.

In some embodiments, the disease, condition, or disorder to be treated is cancer, viral infection, autoimmune disease, or graft-versus-host disease. In some embodiments, the subject to be treated has undergone or is expected to undergo organ transplantation.

In some embodiments, the disease or condition to be treated is a cancer. In some embodiments, the cancer is a hematologic cancer. In some embodiments, the cancer is a B cell malignancy. In some embodiments, the cancer is a myeloma, a lymphoma or a leukemia. In some embodiments, the methods can be used to treat a non-Hodgkin lymphoma (NHL), an acute lymphoblastic leukemia (ALL), a chronic lymphocytic leukemia (CLL), a diffuse large B-cell lymphoma (DLBCL), acute myeloid leukemia (AML), or a myeloma, e.g., a multiple myeloma (MM).

In some embodiments, the cancer is a solid tumor cancer. In some embodiments, the cancer is a bladder, lung, brain, melanoma (e.g. small-cell lung, melanoma), breast, cervical, ovarian, colorectal, pancreatic, endometrial, esophageal, kidney, liver, prostate, skin, thyroid, or uterine cancers. In some embodiments, the cancer is a pancreatic cancer, bladder cancer, colorectal cancer, breast cancer, prostate cancer, renal cancer, hepatocellular cancer, lung cancer, ovarian cancer, cervical cancer, pancreatic cancer, rectal cancer, thyroid cancer, uterine cancer, gastric cancer, esophageal cancer, head and neck cancer, melanoma, neuroendocrine cancers, CNS cancers, brain tumors, bone cancer, or soft tissue sarcoma.

In some aspects, the provided methods can further include administering one or more lymphodepleting therapies, such as prior to or simultaneous with initiation of administration of the adoptive T cell therapy, such as a composition containing modified T cells provided herein. In some embodiments, the lymphodepleting therapy comprises administration of a phosphamide, such as cyclophosphamide. In some embodiments, the lymphodepleting therapy can include administration of fludarabine.

In some aspects, preconditioning subjects with immunodepleting (e.g., lymphodepleting) therapies can improve the effects of adoptive cell therapy (ACT). In some embodiments, the lymphodepleting therapy includes combinations of cyclosporine and fludarabine.

Thus in some embodiments, the provided method further involves administering a lymphodepleting therapy to the subject. In some embodiments, the method involves administering the lymphodepleting therapy to the subject prior to the administration of the dose of cells. In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof, to a subject prior to the administration of the dose of cells. For example, the subject may be administered a preconditioning agent, such as a lymphodepleting or chemotherapeutic agent, such as cyclophosphamide, fludarabine, or combinations thereof, at least 2 days prior, such as at least 3, 4, 5, 6, or 7 days prior, to the first or subsequent dose. In some embodiments, the subject is administered a preconditioning agent, such as a lymphodepleting or chemotherapeutic agent, such as cyclophosphamide, fludarabine, or combinations thereof, no more than 7 days prior, such as no more than 6, 5, 4, 3, or 2 days prior, to the administration of the dose of cells. In some embodiments, the subject is administered a preconditioning agent, such as a lymphodepleting or chemotherapeutic agent, such as cyclophosphamide, fludarabine, or combinations thereof, no more than 14 days prior, such as no more than 13, 12, 11, 10, 9 or 8 days prior, to the administration of the dose of cells.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the fludarabine can be administered in a single dose or can be administered in a plurality of doses, such as given daily, every other day or every three days. In some embodiments, the cyclophosphamide is administered once daily for one or two days.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 30 mg/m², or 24 mg/m² and 26 mg/m². In some instances, the subject is administered 25 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the dose of cells.

In some embodiments, prior to the administration of adoptive T cell therapy, such as a composition containing modified T cells described herein, the subject has received a lymphodepleting therapy. In some embodiments, the lymphodepleting therapy includes fludarabine and/or cyclophosphamide. In some embodiments, the lymphodepleting includes the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.

In some embodiments, the lymphodepleting therapy includes fludarabine and cyclophosphamide. In some embodiments, the lymphodepleting therapy includes the administration of fludarabine at or about 30 mg/m² body surface area of the subject, daily, and cyclophosphamide at or about 300 mg/m² body surface area of the subject, daily, each for 2-4 days, optionally 3 days.

In some embodiments, the administration of the preconditioning agent prior to infusion of the dose of cells improves an outcome of the treatment. For example, in some aspects, preconditioning, such as a lymphodepleting or chemotherapeutic agent, such as cyclophosphamide, fludarabine, or combinations thereof, improves the efficacy of treatment with the dose or increases the persistence of the T cells in the subject. In some embodiments, preconditioning treatment increases disease-free survival, such as the percent of subjects that are alive and exhibit no minimal residual or molecularly detectable disease after a given period of time following the dose of cells. In some embodiments, the time to median disease-free survival is increased.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the T cells to destroy target cells can be measured using any suitable method known in the art, such as cytotoxicity assays described in, for example, Kochenderfer et al., J. Immunotherapy, 32 (7): 689-702 (2009), and Herman et al. J. Immunological Methods, 285 (1): 25-40 (2004). In certain embodiments, the biological activity of the cells also can be measured by assaying expression and/or secretion of certain cytokines or other effector molecules, such as IFNγ and TNF.

In some aspects the biological activity is measured by assessing clinical outcome, such as reduction in tumor burden or load. In some aspects, toxic outcomes, persistence and/or expansion of the cells, and/or presence or absence of a host immune response, are assessed. In some embodiments, exemplary parameters for determination include particular clinical outcomes indicative of amelioration or improvement in the disease or condition, e.g., tumor. Such parameters include: duration of disease control, including complete response (CR), partial response (PR) or stable disease (SD) (see, e.g., Response Evaluation Criteria In Solid Tumors (RECIST) guidelines), objective response rate (ORR), progression-free survival (PFS) and overall survival (OS). Specific thresholds for the parameters can be set to determine the efficacy of the method of combination therapy provided herein.

### VI. KITS AND ARTICLES OF MANUFACTURE

Also provided are articles of manufacture, systems, apparatuses, and kits useful in performing the provided embodiments. In some embodiments, the provided articles of manufacture or kits contain any of the DNA-targeting systems described herein, any of the gRNAs described herein, any of the fusion proteins described herein, any of the polynucleotides described herein, any of the pluralities of polynucleotides described herein, any of the vectors described herein, any of the pluralities of vectors described herein, any of the cells (e.g. modified T cells) described herein, or a portion or a component of any of the foregoing, or any combination thereof. In some embodiments, the articles of manufacture or kits include polypeptides, polynucleotides, nucleic acids, vectors, and/or cells useful in performing the provided methods.

In some embodiments, the articles of manufacture or kits include one or more containers, typically a plurality of containers, packaging material, and a label or package insert on or associated with the container or containers and/or packaging, generally including instructions for use, e.g., instructions for introducing or administering.

Also provided are articles of manufacture, systems, apparatuses, and kits useful in administering the provided compositions, e.g., pharmaceutical compositions, e.g., for use in therapy or treatment. In some embodiments, the articles of manufacture or kits provided herein contain vectors and/or plurality of vectors, such as any vectors and/or plurality of vectors described herein. In some aspects, the articles of manufacture or kits provided herein can be used for administration of the vectors and/or plurality of vectors, and can include instructions for use.

The articles of manufacture and/or kits containing cells or cell compositions for therapy, may include a container and a label or package insert on or associated with the container. Suitable containers include, for example, bottles, vials, syringes, IV solution bags, etc. The containers may be formed from a variety of materials such as glass or plastic. The container in some embodiments holds a composition which is by itself or combined with another composition effective for treating, preventing and/or diagnosing the condition. In some embodiments, the container has a sterile access port. Exemplary containers include an intravenous solution bags, vials, including those with stoppers pierceable by a needle for injection, or bottles or vials for orally administered agents. The label or package insert may indicate that the composition is used for treating a disease or condition. The article of manufacture may further include a package insert indicating that the compositions can be used to treat a particular condition. Alternatively, or additionally, the article of manufacture may further include another or the same container comprising a pharmaceutically-acceptable buffer. It may further include other materials such as other buffers, diluents, filters, needles, and/or syringes.

### VII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known. Reference to “about” a value or parameter herein includes (and describes) embodiments that are directed to that value or parameter per se. For example, description referring to “about X” includes description of “X”. In some embodiments, “about” may refer to +25%, +20%, +15%, +10%, +5%, or +1%.

As used herein, recitation that nucleotides or amino acid positions “correspond to” nucleotides or amino acid positions in a disclosed sequence, such as set forth in the Sequence listing, refers to nucleotides or amino acid positions identified upon alignment with the disclosed sequence to maximize identity using a standard alignment algorithm, such as the GAP algorithm. By aligning the sequences, corresponding residues can be identified, for example, using conserved and identical amino acid residues as guides. In general, to identify corresponding positions, the sequences of amino acids are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carrillo et al. (1988) SIAM J Applied Math 48:1073).

A “gene,” includes a DNA region encoding a gene product. Thus, the gene typically refers to coding and/or transcribed sequences. The sequence of a gene is typically present at a fixed chromosomal position or locus on a chromosome in the cell.

A “regulatory element” or “DNA regulatory element,” which terms are used interchangeably herein, in reference to a gene refers to DNA regions which regulate the production of a gene product, whether or not such regulatory sequences are adjacent to coding and/or transcribed sequences. Accordingly, a regulatory element includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

As used herein, a “target site” or “target nucleic acid sequence” is a nucleic acid sequence that defines a portion of a nucleic acid to which a binding molecule (e.g. a DNA-targeting domain disclosed herein) will bind, provided sufficient conditions for binding exist.

The term “expression” with reference to a gene or “gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or can be a protein produced by translation of an mRNA. For instance, expression includes the transcription and/or translation of a particular nucleotide sequence drive by its promoter. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation. Hence, reference to expression or gene expression includes protein (or polypeptide) expression or expression of a transcribable product of or a gene such as mRNA. The protein expression may include intracellular expression or surface expression of a protein. Typically, expression of a gene product, such as mRNA or protein, is at a level that is detectable in the cell.

As used herein, a “detectable” expression level, means a level that is detectable by standard techniques known to a skilled artisan, and include for example, differential display. RT (reverse transcriptase)-coupled polymerase chain reaction (PCR), Northern Blot, and/or RNase protection analyses as well as immunoaffinity-based methods for protein detection, such as flow cytometry. ELISA, or western blot. The degree of expression levels need only be large enough to be visualized or measured via standard characterization techniques.

As used herein, the term “increased expression”, “enhanced expression” or “overexpression” means any form of expression that is additional to the expression in an original or source cell that does not contain the modification for modulating a particular gene expression by a DNA-targeting system, for instance a wild-type expression level (which can be absence of expression or immeasurable expression as well). Reference herein to “increased expression.” “enhanced expression” or “overexpression” is taken to mean an increase in gene expression relative to the level in a cell that does not contain the modification, such as the original source cell prior to contacting with, or engineering to introduce, the Dna-targeting system into the T cell, such as an unmodified cell or a wild-type T cell. The increase in expression can be at least 5%, 10%, 20%, 30%, 40% or 50%, 60%, 70%, 80%, 85%, 90%, or 100% or even more. In some cases, the increase in expression can be at least 2-fold, 5-fold, 10-fold, 20-fold, 30-fold, 40-fold, 50-fold, 60-fold, 70-fold, 80-fold, 90-fold, 100-fold, 200-fold, 300-fold, 400-food, 500)-fold, 1000-fold or more.

As used herein, the term “increased transcription” refers to the level of transcription of a gene that is additional to the transcription of the gene in an original or source cell that does not contain the modification for modulating transcription by a DNA-targeting system, for instance a wild-type transcription level of a gene. Reference to increased transcription can refer to an increase in the levels of a transcribable product of a gene such as mRNA. Any of a variety of methods can be used to monitor or quantitate a level of a transcribable product such as mRNA, including but not limited to, real-time quantitative RT (reverse transcriptase)-polymerase chain reaction (qRT-PCR), Northern Blot, microarray analysis, or RNA sequencing (RNA-Seq). The increase in transcription can be at least 5%, 10%, 20%, 30%, 40% or 50%, 60%, 70%, 80%, 85%, 90%, or 100% or even more. In some cases, the increase in transcription can be at least 2-fold, 5-fold, 10-fold, 20-fold, 30-fold, 40-fold, 50-fold, 60-fold, 70-fold, 80-fold, 90-fold, 100-fold, 200-fold or more.

As used herein, an “epigenetic modification” refers to changes in the gene expression that are non-genetic modifications, i.e. not caused by changes in the DNA sequences, but are due to epigenetic changes such as events like DNA methylations or histone modifications. An epigenetic modification may result in a heritable change in gene activity and expression that occur without alteration in DNA sequence. For instance, epigenetic modifications include non-genetic modifications such as chemical modifications to the cytosine residues of DNA (DNA methylation) and histone proteins associated with DNA (histone modifications).

As used herein, the term “modification” or “modified” with reference to a T cell refers to any change or alteration in a cell that impacts gene expression in the cell. In some embodiments, the modification is an epigenetic modification that directly changes the epigenetic state of a gene or regulatory elements thereof to alter (e.g. increase) expression of a gene product. In some embodiments, a modification described herein results in increased expression of a target gene or selected polynucleotide sequence.

As used herein, a “fusion” molecule is a molecule in which two or more subunit molecules are linked, such as covalently. Examples of a fusion molecule include, but are not limited to, fusion proteins (for example, a fusion between a DNA-binding domain such as a ZFP. TALE DNA-binding domain or CRISPR-Cas protein and one or more effector domains, such as a transactivation domain). The fusion molecule also may be part of a system in which a polynucleotide component associates with a polypeptide component to form a functional molecule (e.g., a CRISPR/Cas system in which a single guide RNA associates with a functional domain to modulate gene expression). Fusion molecules also include fusion nucleic acids, for example, a nucleic acid encoding the fusion protein. Expression of a fusion protein in a cell can result from delivery of the fusion protein to the cell or by delivery of a polynucleotide encoding the fusion protein to a cell, where the polynucleotide is transcribed, and the transcript is translated, to generate the fusion protein.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as adenoviral vectors or lentiviral vectors.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include, but are not limited to, cosmids, plasmids (e.g., naked or contained in liposomes) and viruses (e.g., lentiviruses, retroviruses, adenoviruses, and adeno-associated viruses) that incorporate the recombinant polynucleotide.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “polynucleotide” refers to a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomelic “nucleotides.” The monomelic nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “percent (%) amino acid sequence identity” and “percent identity” when used with respect to an amino acid sequence (reference polypeptide sequence) is defined as the percentage of amino acid residues in a candidate sequence (e.g., the subject antibody or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various known ways, in some embodiments, using publicly available computer software such as BLAST, BLAST-2, ALIGN or Megalign (DNASTAR) software. Appropriate parameters for aligning sequences can be determined, including any algorithms needed to achieve maximal alignment over the full length of the sequences being compared.

In some embodiments, “operably linked” may include the association of components, such as a DNA sequence, (e.g. a heterologous nucleic acid) and a regulatory sequence(s), in such a way as to permit gene expression when the appropriate molecules (e.g. transcriptional activator proteins) are bound to the regulatory sequence. Hence, it means that the components described are in a relationship permitting them to function in their intended manner.

An amino acid substitution may include replacement of one amino acid in a polypeptide with another amino acid. The substitution may be a conservative amino acid substitution or a non-conservative amino acid substitution. Amino acid substitutions may be introduced into a binding molecule, e.g., antibody, of interest and the products screened for a desired activity, e.g., retained/improved antigen binding, decreased immunogenicity, or improved ADCC or CDC.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

As used herein, a composition refers to any mixture of two or more products, substances, or compounds, including cells. It may be a solution, a suspension, liquid, powder, a paste, aqueous, non-aqueous or any combination thereof.

As used herein, a “subject” or an “individual,” which are terms that are used interchangeably, is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject or individual is human. In some embodiments, the subject is a patient that is known or suspected of having a disease, disorder or condition.

As used herein, the term “treating” and “treatment” includes administering to a subject an effective amount of a biological molecule, such as a therapeutic agent, so that the subject has a reduction in at least one symptom of the disease or an improvement in the disease, for example, beneficial or desired clinical results. For instance, a biological molecule may include cells (e.g. T cells), such as cells that have been modified by a DNA-targeting system or polynucleotide(s) encoding the DNA-targeting system described herein. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a disease or disorder are alleviated by at least 5%, at least 10%, at least 20%, at least 30%, at least 40%, or at least 50% upon treatment of the disease.

For purposes of this technology, beneficial or desired clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a biological molecule, such as a compound or cells, that, when administered, is sufficient to prevent development of, or alleviate to some extent, one or more of the signs or symptoms of the disorder or disease being treated. The therapeutically effective amount will vary depending on the biological molecule, the disease and its severity and the age, weight, etc., of the subject to be treated.

As used herein, “adoptive cell therapy” (ACT) refers to the administration of T cells targeting a specific antigen to a subject.

As used herein, the term “autologous” is meant to refer to any material derived from the same individual to which it is later to be re-introduced into the individual.

“Allogeneic” refers to a graft derived from a different animal of the same species.

### VIII. EXEMPLARY EMBODIMENTS

Among the provided embodiments are:

1. An epigenetic-modifying DNA-targeting system,
said DNA-targeting system comprising a fusion protein comprising:
(a) a DNA-targeting domain capable of being targeted to a target site in a gene or regulatory DNA element thereof in a T cell; and
(b) at least one effector domain capable of increasing transcription of the gene;
wherein increased transcription of the gene promotes a stem cell-like memory T-cell phenotype.

2. The epigenetic-modifying DNA-targeting system of embodiment 1, wherein the DNA-targeting system is not able to introduce a genetic disruption or a DNA break at or near the target site.

3. The epigenetic-modifying DNA-targeting system of embodiment 1 or embodiment 2, wherein the DNA-targeting domain comprises a Clustered Regularly Interspaced Short Palindromic Repeats associated (Cas)-guide RNA (gRNA) combination comprising (a) a Cas protein or a variant thereof and (b) at least one gRNA; a zinc finger protein (ZFP); a transcription activator-like effector (TALE); a meganuclease; a homing endonuclease; or an I-SceI enzyme or a variant thereof, optionally wherein the DNA-targeting domain comprises a catalytically inactive variant of any of the foregoing.

4. The epigenetic-modifying DNA-targeting system of any of embodiments 1-3, wherein the DNA-targeting domain comprises a Cas-gRNA combination comprising (a) a Cas protein or a variant thereof and (b) at least one gRNA.

5. An epigenetic-modifying DNA-targeting system,
said DNA-targeting system comprising:
(a) a fusion protein comprising a Clustered Regularly Interspaced Short Palindromic Repeats associated (Cas) protein or variant thereof and at least one effector domain capable of increasing transcription of a gene is a T cell; and
(b) at least one gRNA that targets the Cas protein or variant thereof of the fusion protein to a target site in the gene or regulatory DNA element thereof,
wherein increased transcription of the gene promotes a stem cell-like memory T-cell phenotype.

6. The epigenetic-modifying DNA-targeting system of any of embodiments 1-5, wherein the stem cell-like memory T cell phenotype comprises one or more cell-surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−, or combinations thereof.

7. The epigenetic-modifying DNA-targeting system of any of embodiments 1-6, wherein the stem cell-like memory T cell phenotype comprises expression of CCR7 and/or CD27.

8. The epigenetic-modifying DNA-targeting system of any of embodiments 1-7, wherein the stem cell-like memory T cell phenotype comprises expression of CCR7 and CD27.

9. The epigenetic-modifying DNA-targeting system of any of embodiments 1-8, wherein the stem cell-like memory T cell phenotype is characterized by polyfunctional activity of the T cell to produce two or more cytokines following stimulation of the T cell with a stimulatory agent, optionally wherein the two or more cytokines are selected from among interferon-gamma (IFN-gamma), interleukin 2 (IL-2), and TNF-alpha.

10. The epigenetic-modifying DNA-targeting system of any of embodiments 3-9, wherein at least one gRNA is capable of complexing with the Cas protein or variant thereof, and targeting the Cas protein or the variant thereof to the target site.

11. The epigenetic-modifying DNA-targeting system of any of embodiments 3-10, wherein the at least one gRNA comprises a gRNA spacer sequence that is capable of hybridizing to the target site or is complementary to the target site.

12. The epigenetic-modifying DNA-targeting system of any of embodiments 3-11, wherein the Cas protein or a variant thereof is a Cas9 protein or a variant thereof.

13. The epigenetic-modifying DNA-targeting system of any of embodiments 3-11, wherein the Cas protein or a variant thereof is a Cas12 protein or a variant thereof.

14. The epigenetic-modifying DNA-targeting system of any of embodiments 3-13, wherein the Cas protein or a variant thereof is a variant Cas protein, wherein the variant Cas protein lacks nuclease activity or is a deactivated Cas (dCas) protein.

15. The epigenetic-modifying DNA-targeting system of embodiment 14, wherein the variant Cas protein is a variant Cas9 protein that lacks nuclease activity or that is a deactivated Cas9 (dCas9) protein.

16. The epigenetic-modifying DNA-targeting system of embodiment 12, wherein the Cas9 protein or a variant thereof is a Staphylococcus aureus Cas9 (SaCas9) protein or a variant thereof.

17. The epigenetic-modifying DNA-targeting system of embodiment 15, wherein the variant Cas9 is a Staphylococcus aureus dCas9 protein (dSaCas9) that comprises at least one amino acid mutation selected from D10A and N580A, with reference to numbering of positions of SEQ ID NO: 405.

18. The epigenetic-modifying DNA-targeting system of embodiment 15 or embodiment 17, wherein the variant Cas9 protein comprises the sequence set forth in SEQ ID NO: 406, or an amino acid sequence that has at least 90%, 91%, 92%, 93%, 94%, 95%, 96%, 97%, 98%, or 99% sequence identity thereto.

19. The epigenetic-modifying DNA-targeting system of embodiment 12, wherein the Cas9 protein or variant thereof is a Streptococcus pyogenes Cas9 (SpCas9) protein or a variant thereof.

20. The epigenetic-modifying DNA-targeting system of embodiment 15, wherein the variant Cas9 is a Streptococcus pyogenes dCas9 (dSpCas9) protein that comprises at least one amino acid mutation selected from D10A and H840A, with reference to numbering of positions of SEQ ID NO: 407.

21. The epigenetic-modifying DNA-targeting system of embodiment 15 or embodiment 20, wherein the variant Cas9 protein comprises the sequence set forth in SEQ ID NO: 408, or an amino acid sequence that has at least 90%, 91%, 92%, 93%, 94%, 95%, 96%, 97%, 98%, or 99% sequence identity thereto.

22. The epigenetic-modifying DNA-targeting system of any of embodiments 1-21, wherein the regulatory DNA element is an enhancer or a promoter.

23. The epigenetic-modifying DNA-targeting system of any of embodiments 1-22, wherein the gene is a DNA-binding gene.

24. The DNA-targeting system of any of embodiments 1-23, wherein the gene is selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10.

25. The epigenetic-modifying DNA-targeting system of any of embodiments 1-24, wherein the target site comprises the sequence set forth in any one of SEQ ID NOS: 1-132, a contiguous portion thereof of at least 14 nucleotides (nt), or a complementary sequence of any of the foregoing.

26. The epigenetic-modifying DNA-targeting system of any of embodiments 3-25, wherein the at least one gRNA comprises a gRNA spacer sequence comprising the sequence set forth in SEQ ID NO:133-264, or a contiguous portion thereof of at least 14 nt.

27. The epigenetic-modifying DNA-targeting system of embodiment 26, wherein the at least one gRNA further comprises the sequence set forth in SEQ ID NO:398.

28. The epigenetic-modifying DNA-targeting system of any of embodiments 3-27, wherein the at least one gRNA comprises a gRNA that comprises the sequence set forth in any one of SEQ ID NOS: 265-396, optionally wherein the at least one gRNA is the gRNA set forth in any one of SEQ ID NOS: 265-396.

29. The epigenetic-modifying DNA-targeting system of any of embodiments 1-24, wherein the gene is selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23.

30. The epigenetic-modifying DNA-targeting system of any of embodiments 1-24 and 29, wherein the target site comprises the sequence set forth in any one of SEQ ID NOS: 1-28, a contiguous portion thereof of at least 14 nucleotides (nt), or a complementary sequence of any of the foregoing.

31. The epigenetic-modifying DNA-targeting system of any of embodiments 3-24, 29, and 30, wherein the at least one gRNA comprises a gRNA spacer sequence comprising the sequence set forth in SEQ ID NO:133-160, or a contiguous portion thereof of at least 14 nt.

32. The epigenetic-modifying DNA-targeting system of embodiment 31, wherein the at least one gRNA further comprises the sequence set forth in SEQ ID NO:398.

33. The epigenetic-modifying DNA-targeting system of any of embodiments 3-24 and 29-32, wherein the at least one gRNA comprises a gRNA that comprises the sequence set forth in any one of SEQ ID NOS: 265-292, optionally wherein the at least one gRNA is the gRNA set forth in any one of SEQ ID NOS: 265-292.

34. The epigenetic-modifying DNA-targeting system of any of embodiments 1-24, wherein the gene is selected from the list consisting of: DDIT3, HES2, HKR1, PLAG1, PRDM4, TEF, TSHZ1, ZNF141, ZNF562, ZNF691, ZNF692, and ZSCAN5A.

35. The DNA-targeting system of any of embodiments 1-24 and 34, wherein the target site comprises the sequence set forth in any one of SEQ ID NOS: 1-12, a contiguous portion thereof of at least 14 nucleotides (nt), or a complementary sequence of any of the foregoing.

36. The DNA-targeting system of any of embodiments 3-24, 34, and 35, wherein the at least one gRNA comprises a gRNA spacer sequence comprising the sequence set forth in SEQ ID NO: 133-144, or a contiguous portion thereof of at least 14 nt.

37. The DNA-targeting system of embodiment 36, wherein the at least one gRNA further comprises the sequence set forth in SEQ ID NO:398.

38. The DNA-targeting system of any of embodiments 3-24 and 34-37, wherein the at least one gRNA comprises a gRNA that comprises the sequence set forth in any one of SEQ ID NOS: 265-276, optionally wherein the at least one gRNA is the gRNA set forth in any one of SEQ ID NOS: 265-276.

39. The DNA-targeting system of any of embodiments 3-38, wherein the gRNA spacer sequence is between 14 nt and 24 nt, or between 16 nt and 22 nt in length.

40. The DNA-targeting system of any of embodiments 3-39, wherein the gRNA spacer sequence is 18 nt, 19 nt, 20 nt, 21 nt or 22 nt in length.

41. The DNA-targeting system of any of embodiments 3-40, wherein the gRNA comprises modified nucleotides for increased stability.

42. The DNA-targeting system of any of embodiments 1-41, wherein the at least one effector domain induces, catalyzes, or leads to transcription activation, transcription co-activation, transcription elongation, or increased transcription of the gene.

43. The DNA-targeting system of any of embodiments 1-42, wherein the at least one effector domain induces transcription activation.

44. The DNA-targeting system of any of embodiments 1-43, wherein the at least one effector domain comprises at least one VP16 domain, or a VP16 tetramer (“VP64”) or a variant thereof.

45. The DNA-targeting system of any of embodiments 1-44, wherein the at least one effector domain comprises the sequence set forth in SEQ ID NO: 409, a portion thereof, or an amino acid sequence that has at least 90%, 91%, 92%, 93%, 94%, 95%, 96%, 97%, 98%, or 99% sequence identity to any of the foregoing.

46. The DNA-targeting system of any of embodiments 1-43, wherein at least one effector domain is selected from a p65 activation domain, a p300 domain, Rta, CBP, VPR, VPH, HSF1, a TET protein, optionally wherein the TET protein is TET1, an ERF protein, optionally wherein the ERF protein is ERF1 or ERF3, LSD1, SunTag, or a domain, portion, or variant of any of the foregoing.

47. The DNA-targeting system of any of embodiments 1-43 and 46, wherein at least one effector domain comprises a sequence selected from any one of SEQ ID NOS: 411-417, 438, or 440, or a domain thereof, a portion thereof, or an amino acid sequence that has at least 90%, 91%, 92%, 93%, 94%, 95%, 96%, 97%, 98%, or 99% sequence identity to any of the foregoing.

48. The DNA-targeting system of any of embodiments 1-47, wherein the at least one effector domain is fused to the N-terminus, the C-terminus, or both the N-terminus and the C-terminus, of the DNA-targeting domain or a component thereof.

49. The DNA-targeting system of any of embodiments 1-48, further comprising one or more nuclear localization signals (NLS).

50. The DNA-targeting system of embodiment 49, further comprising one or more linkers connecting two or more of: the DNA-targeting domain, the at least one effector domain, and the one or more nuclear localization signals.

51. The DNA-targeting system of any of embodiments 1-50, wherein the fusion protein comprises the sequence set forth in SEQ ID NO:400, or an amino acid sequence that has at least 90%, 91%, 92%, 93%, 94%, 95%, 96%, 97%, 98%, or 99% sequence identity thereto.

52. The DNA-targeting system of any of embodiments 1-51, wherein increased transcription of the gene further promotes increased production of IL-2 by the T cell.

53. The DNA-targeting system of any of embodiments 1-52, wherein the epigenetic-modifying DNA-targeting system increases expression of the gene in a T cell by a log 2 fold-change of at or greater than 1.0.

54. The DNA-targeting system of any of embodiments 1-53, wherein the epigenetic-modifying DNA-targeting system reduces surface expression of a T cell exhaustion marker selected from the group consisting of PD-1, CTLA-4, TIM-3, TOX, LAG-3, BTLA, 2B4, CD160, CD39, VISTA, and TIGIT.

55. A guide RNA (gRNA) that binds a target site in a gene or regulatory DNA element thereof in a T cell, wherein increased transcription of the gene, when targeted by an epigenetic-modifying DNA-targeting system comprising the gRNA, promotes a stem cell-like memory T cell phenotype.

56. The gRNA of embodiment 55, wherein the stem cell-like memory T cell phenotype comprises one or more cell-surface markers selected from CCR7+, CD27+, CD45RA+, CD45RO−, CCR7+, CD62L+, CD28+, CD27+, IL-7Rα+, CXCR3+, CD95+, CD11a+. IL-2Rβ+, CD58+, and CD57−.

57. The gRNA of embodiment 55 or embodiment 56, wherein the stem cell-like memory T cell phenotype comprises expression of CCR7 and/or CD27.

58. The gRNA of embodiment 55 or embodiment 56, wherein the stem cell-like memory T cell phenotype comprises expression of CCR7 and CD27.

59. The gRNA of any of embodiments 55-58, wherein the stem cell-like memory T cell phenotype is characterized by polyfunctional activity of the T cell to produce two or more cytokines following stimulation of the T cell with a stimulatory agent, optionally wherein the two or more cytokines are selected from among interferon-gamma (IFN-gamma), interleukin 2 (IL-2), and TNF-alpha.

60. The gRNA of any of embodiments 55-59, wherein the gene is selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10.

61. A guide RNA (gRNA) that binds a target site in a gene or regulatory DNA element thereof in a T cell, wherein increased transcription of the gene, when targeted by an epigenetic-modifying DNA-targeting system comprising the gRNA, promotes a stem cell-like memory T cell phenotype, and wherein the gene is selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10.

62. The gRNA of any of embodiments 55-61, wherein the target site is in a regulatory DNA element and the regulatory DNA element is an enhancer or a promoter.

63. The gRNA of any of embodiments 55-62, wherein the target site comprises the sequence set forth in any one of SEQ ID NOS: 1-132, a contiguous portion thereof of at least 14 nucleotides (nt), or a complementary sequence of any of the foregoing.

64. The gRNA of any of embodiments 55-63, wherein the gRNA comprises a gRNA spacer sequence comprising the sequence set forth in SEQ ID NO:133-264, or a contiguous portion thereof of at least 14 nt.

65. The gRNA of embodiment 64, wherein the gRNA further comprises the sequence set forth in SEQ ID NO:398.

66. The gRNA of any of embodiments 55-65, wherein the gRNA comprises a gRNA that comprises the sequence set forth in any one of SEQ ID NOS: 265-396, optionally wherein the gRNA is the gRNA set forth in any one of SEQ ID NOS: 265-396.

67. The gRNA of embodiment 60 or embodiment 61, wherein the gene is selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23.

68. The gRNA of embodiment 60, embodiment 61, or embodiment 67, wherein the target site comprises the sequence set forth in any one of SEQ ID NOS: 1-28, a contiguous portion thereof of at least 14 nucleotides (nt), or a complementary sequence of any of the foregoing.

69. The gRNA of embodiment 60, 61, 67 or 68, wherein the gRNA comprises a gRNA spacer sequence comprising the sequence set forth in SEQ ID NO:133-160, or a contiguous portion thereof of at least 14 nt.

70. The gRNA of embodiment 69, wherein the gRNA further comprises the sequence set forth in SEQ ID NO:398.

71. The gRNA of embodiment 60, embodiment 61 or any of embodiments 67-70, wherein the gRNA comprises a gRNA that comprises the sequence set forth in any one of SEQ ID NOS: 265-292, optionally wherein the gRNA is the gRNA set forth in any one of SEQ ID NOS: 265-292.

72. The gRNA of embodiment 60 or embodiment 61, wherein the gene is selected from the list consisting of: DDIT3, HES2, HKR1, PLAG1, PRDM4, TEF, TSHZ1, ZNF141, ZNF562, ZNF691, ZNF692, and ZSCAN5A.

73. The gRNA of embodiment 60, embodiment 61 or embodiment 72, wherein the target site comprises the sequence set forth in any one of SEQ ID NOS: 1-12, a contiguous portion thereof of at least 14 nucleotides (nt), or a complementary sequence of any of the foregoing.

74. The gRNA of embodiment 60, 61, 72 or 73, wherein the gRNA comprises a gRNA spacer sequence comprising the sequence set forth in SEQ ID NO:133-144, or a contiguous portion thereof of at least 14 nt.

75. The gRNA of embodiment 74, wherein the gRNA further comprises the sequence set forth in SEQ ID NO:398.

76. The gRNA of any of embodiments 60, 61, and 72-75, wherein the gRNA comprises a gRNA that comprises the sequence set forth in any one of SEQ ID NOS: 265-276, optionally wherein the gRNA is the gRNA set forth in any one of SEQ ID NOS: 265-276.

77. The gRNA of any of embodiments 55-76, wherein the gRNA spacer sequence is between 14 nt and 24 nt, or between 16 nt and 22 nt in length.

78. The gRNA of any of embodiments 55-77, wherein the gRNA spacer sequence is 18 nt, 19 nt, 20 nt, 21 nt or 22 nt in length.

79. The gRNA of any of embodiments 55-78, wherein the gRNA comprises modified nucleotides for increased stability.

80. The gRNA of any of embodiments 55-79, wherein the gRNA is capable of complexing with a Cas protein or variant thereof.

81. The gRNA of any of embodiments 55-80, wherein the gRNA is capable of hybridizing to the target site or is complementary to the target site.

82. A CRISPR Cas-guide RNA (gRNA) combination comprising:
(a) a Clustered Regularly Interspaced Short Palindromic Repeats associated (Cas) protein or variant thereof; and
(b) at least one gRNA of any of embodiments 55-81 that targets the Cas protein or variant thereof to a target site in a gene or regulatory DNA element thereof in a T cell.

83. The CRISPR Cas-gRNA combination of embodiment 82, wherein the Cas protein or a variant thereof is a Cas9 protein or a variant thereof.

84. The CRISPR Cas-gRNA combination of embodiment 82 or embodiment 83, wherein the Cas protein or a variant thereof is a variant Cas protein, wherein the variant Cas protein lacks nuclease activity or is a deactivated Cas (dCas) protein.

85. The CRISPR Cas-gRNA combination of embodiment 83 or embodiment 84, wherein the variant Cas protein is a variant Cas9 protein that lacks nuclease activity or that is a deactivated Cas9 (dCas9) protein.

86. The CRISPR Cas-gRNA combination of embodiment 83, wherein the Cas9 protein or a variant thereof is a Staphylococcus aureus Cas9 (SaCas9) protein or a variant thereof.

87. The CRISPR Cas-gRNA combination of embodiment 83 or embodiment 84, wherein the variant Cas9 is a Staphylococcus aureus dCas9 protein (dSaCas9) that comprises at least one amino acid mutation selected from D10A and N580A, with reference to numbering of positions of SEQ ID NO: 405.

88. The CRISPR Cas-gRNA combination of embodiment 83, 84 or 87, wherein the variant Cas9 protein comprises the sequence set forth in SEQ ID NO: 406, or an amino acid sequence that has at least 90%, 91%, 92%, 93%, 94%, 95%, 96%, 97%, 98%, or 99% sequence identity thereto.

89. The CRISPR Cas-gRNA combination of embodiment 83, wherein the Cas9 protein or variant thereof is a Streptococcus pyogenes Cas9 (SpCas9) protein or a variant thereof.

90. The CRISPR Cas-gRNA combination of any of embodiment 83 or embodiment 84, wherein the variant Cas9 is a Streptococcus pyogenes dCas9 (dSpCas9) protein that comprises at least one amino acid mutation selected from D10A and H840A, with reference to numbering of positions of SEQ ID NO: 407.

91. The CRISPR Cas-gRNA combination of embodiment 83, embodiment 84 or embodiment 90, wherein the variant Cas9 protein comprises the sequence set forth in SEQ ID NO: 408, or an amino acid sequence that has at least 90%, 91%, 92%, 93%, 94%, 95%, 96%, 97%, 98%, or 99% sequence identity thereto.

92. A polynucleotide encoding the DNA-targeting system of any of embodiments 1-54, the fusion protein of the DNA-targeting system of any of embodiments 1-54, the gRNA of any of embodiments 55-81, the CRISPR Cas-gRNA combination of any of embodiments 82-91, or a portion or a component of any of the foregoing.

93. A plurality of polynucleotides encoding the DNA-targeting system of any of embodiments 1-54, the fusion protein of the DNA-targeting system of any of embodiments 1-54, the gRNA of any of embodiments 55-81, the CRISPR Cas-gRNA combination of any of embodiments 82-91, or a portion or a component of any of the foregoing.

94. A vector comprising the polynucleotide of embodiment 92.

95. A vector comprising the plurality of polynucleotides of embodiment 93.

96. The vector of embodiment 94 or embodiment 95, wherein the vector is a viral vector.

97. The vector of embodiment 96, wherein the vector is an adeno-associated virus (AAV) vector.

98. The vector of embodiment 97, wherein the vector is selected from among AAV1, AAV2, AAV3, AAV4, AAV5, AAV6, AAV7, AAV8, and AAV9.

99. The vector of embodiment 96, wherein the vector is a lentiviral vector.

100. The vector of embodiment 94 or embodiment 95, wherein the vector is a non-viral vector.

101. The vector of embodiment 100, wherein the non-viral vector is selected from: a lipid nanoparticle, a liposome, an exosome, or a cell penetrating peptide.

102. The vector of any of embodiments 94-101, wherein the vector exhibits immune cell or T-cell tropism.

103. The vector of any of embodiments 94-102, wherein the vector comprises one vector, or two or more vectors.

104. A modified T cell comprising the DNA-targeting system of any of embodiments 1-54, the gRNA of any of embodiments 55-81, the CRISPR Cas-gRNA combination of any of embodiments 82-91, the polynucleotide of embodiment 92, the plurality of polynucleotides of embodiment 93, the vector of any of embodiments 94-103, or a portion or a component of any of the foregoing.

105. A modified T cell comprising an epigenetic or phenotypic modification resulting from being contacted by the DNA-targeting system of any of embodiments 1-54, the gRNA of any of embodiments 55-81, the CRISPR Cas-gRNA combination of any of embodiments 82-91, the polynucleotide of embodiment 92, the plurality of polynucleotides of embodiment 93, the vector of any of embodiments 94-103, or a portion or a component of any of the foregoing.

106. The modified T cell of embodiment 104 or embodiment 105, wherein the modified T cell exhibits increased transcription of one or more genes that promote a stem cell-like memory T-cell phenotype, in comparison to a comparable unmodified T cell.

107. The modified T cell of embodiment 106, wherein the one or more genes are selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10.

108. The modified T cell of embodiment 106, wherein the one or more genes are selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23.

109. The modified T cell of embodiment 106, wherein the one or more genes are selected from the list consisting of: DDIT3, HES2, HKR1, PLAG1, PRDM4, TEF, TSHZ1, ZNF141, ZNF562, ZNF691, ZNF692, and ZSCAN5A.

110. The modified T cell of any of embodiments 106-109, wherein the transcription is increased by at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold.

111. The modified T cell of any of embodiments 104-110, wherein the modified T cell exhibits a stem cell-like memory T-cell phenotype.

112. The modified T cell of embodiment 111, wherein the stem cell-like memory T cell phenotype comprises expression of CCR7 and/or CD27.

113. The modified T cell of embodiment 111, wherein the stem cell-like memory T cell phenotype comprises expression of CCR7 and CD27.

114. The modified T cell of any of embodiments 111-113, wherein the stem cell-like memory T cell phenotype comprises one or more cell-surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−.

115. The modified T cell of any of embodiments 104-114, wherein the modified T cell is capable of a stronger and/or more persistent immune response, in comparison to a comparable unmodified T cell.

116. The modified T cell of any of embodiments 104-115, wherein the modified T cell is characterized by polyfunctional activity of the T cell to produce two or more cytokines following stimulation of the T cell with a stimulatory agent, optionally wherein the two or more cytokines are selected from among interferon-gamma (IFN-gamma), interleukin 2 (IL-2), and TNF-alpha.

117. The modified T cell of any of embodiments 104-116, wherein the modified T cell is derived from a cell from a subject.

118. The modified T cell of any of embodiments 104-117, wherein the modified T cell is derived from a primary T cell.

119. The modified T cell of any of embodiments 104-117, wherein the modified T cell is derived from a T cell progenitor, a pluripotent stem cell, or an induced pluripotent stem cell.

120. The modified T cell of any of embodiments 104-119, wherein the modified T cell further comprises an engineered T cell receptor (eTCR) or chimeric antigen receptor (CAR).

121. A method of increasing the transcription of one or more genes in a T cell, the method comprising introducing into a T cell the DNA-targeting system of any of embodiments 1-54, the gRNA of any of embodiments 55-81, the CRISPR Cas-gRNA combination of any of embodiments 82-91, the polynucleotide of embodiment 92, the plurality of polynucleotides of embodiment 93, the vector of any of embodiments 94-103, or a portion or a component of any of the foregoing.

122. The method of embodiment 121, wherein the one or more genes is a gene epigenetically modified by the DNA-targeting system.

123. The method of embodiment 121 or embodiment 122, wherein the transcription of the one or more genes is increased in comparison to a comparable T cell not subjected to the method.

124. The method of any of embodiments 121-123, wherein the transcription of the one or more genes is increased by at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold.

125. The method of any of embodiments 121-124, wherein the increased transcription of the one or more genes promotes a stem cell-like memory T cell phenotype in the T cell.

126. A method of promoting a stem cell-like memory T cell phenotype in a T cell, the method comprising introducing into the T cell the DNA-targeting system of any of embodiments 1-54, the gRNA of any of embodiments 55-81, the CRISPR Cas-gRNA combination of any of embodiments 82-91, the polynucleotide of embodiment 92, the plurality of polynucleotides of embodiment 93, the vector of any of embodiments 94-103, or a portion or a component of any of the foregoing.

127. The method of embodiment 125 or embodiment 126, wherein the stem cell-like memory T cell phenotype comprises one or more cell-surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−.

128. The method of any of embodiments 125-127, wherein the stem cell-like memory T cell phenotype comprises expression of CCR7 and/or CD27.

129. The method of any of embodiments 125-128, wherein the stem cell-like memory T cell phenotype is characterized by polyfunctional activity of the T cell to produce two or more cytokines following stimulation of the T cell with a stimulatory agent, optionally wherein the two or more cytokines are selected from among interferon-gamma (IFN-gamma), interleukin 2 (IL-2), and TNF-alpha.

130. The method of any of embodiments 121-129, wherein the T cell is a T cell in a subject and the method is carried out in vivo.

131. The method of any of embodiments 121-129, wherein the T cell is a T cell from a subject, or derived from a cell from the subject, and the method is carried out ex vivo.

132. The method of embodiment 131, wherein the T cell is a primary T cell.

133. The method of embodiment 131, wherein the T cell is derived from a T cell progenitor, a pluripotent stem cell, or an induced pluripotent stem cell.

134. A modified T cell produced by the method of any of embodiments 121-133.

135. A method of cell therapy for treating a disease in a subject in need thereof, comprising administering to the subject a cellular composition that comprises the modified T cell of any of embodiments 104-120 and 134.

136. The method of embodiment 135, wherein the modified T cell is obtained from or derived from a cell from said subject in need thereof.

137. The method of embodiment 135, wherein the subject is a first subject, and the modified T cell is obtained from or derived from a cell from a second subject.

138. The method of any of embodiments 135-137, wherein the subject in need thereof is a human.

139. The method of any of embodiments 135-138, wherein the administered modified T cell exhibits a stronger and/or more persistent immune response in the subject, in comparison to a comparable unmodified T cell.

140. The method of any of embodiments 135-139, wherein the subject has or is suspected of having a disease, condition, or disorder, optionally wherein the disease, condition, or disorder is cancer, viral infection, autoimmune disease, or graft-versus-host disease, or the subject has undergone or is expected to undergo organ transplantation.

141. The method of any of embodiments 135-140, wherein the subject has or is suspected of having cancer.

142. A pharmaceutical composition comprising the modified T cell of any of embodiments 104-120 and 134.

143. A pharmaceutical composition comprising the DNA-targeting system of any of embodiments 1-54, the gRNA of any of embodiments 55-81, the CRISPR Cas-gRNA combination of any of embodiments 82-91, the polynucleotide of embodiment 92, the plurality of polynucleotides of embodiment 93, the vector of any of embodiments 94-103, or a portion or a component of any of the foregoing.

144. The pharmaceutical composition of embodiment 142 or embodiment 143, for use in treating a disease, condition, or disorder in a subject.

145. The pharmaceutical composition of embodiment 142 or embodiment 143, for use in the manufacture of a medicament for treating a disease, condition, or disorder in a subject.

146. The pharmaceutical composition of embodiment 144 or embodiment 145, wherein the subject has or is suspected of having a disease, condition, or disorder, optionally wherein the disease, condition, or disorder is cancer, viral infection, autoimmune disease, or graft-versus-host disease, or the subject has undergone or is expected to undergo organ transplantation.

147. The pharmaceutical composition of any of embodiments 144-146, wherein the subject has or is suspected of having cancer.

148. The pharmaceutical composition of any of embodiments 144-147, wherein the pharmaceutical composition is to be administered to the subject in vivo.

149. The pharmaceutical composition of any of embodiments 144-147, wherein the subject is a first subject, and the pharmaceutical composition is to be administered ex vivo to T cells from the first subject, or to T cells from a second subject.

150. The pharmaceutical composition of embodiment 149, wherein following administration to T cells from the first subject or second subject, the T cells are administered to the first subject.

151. The pharmaceutical composition of any of embodiments 143-148, wherein following administration of the pharmaceutical composition, the expression of one or more genes is increased in T cells of the subject.

152. The pharmaceutical composition of embodiment 149 or embodiment 150, wherein following administration of the pharmaceutical composition to the T cells from the first or second subject, the expression of one or more genes is increased in the T cells.

153. The pharmaceutical composition of embodiment 151 or embodiment 152, wherein the one or more genes are selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10.

154. The pharmaceutical composition of embodiment 151 or embodiment 152, wherein the one or more genes are selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23.

155. The pharmaceutical composition of embodiment 151 or embodiment 152, wherein the one or more genes are selected from the list consisting of: DDIT3, HES2, HKR1, PLAG1, PRDM4, TEF, TSHZ1, ZNF141, ZNF562, ZNF691, ZNF692, and ZSCAN5A.

156. A method for treating a disease in a subject in need thereof, comprising administering to the subject the DNA-targeting system of any of embodiments 1-54, the gRNA of any of embodiments 55-81, the CRISPR Cas-gRNA combination of any of embodiments 82-91, the polynucleotide of embodiment 92, the plurality of polynucleotides of embodiment 93, the vector of any of embodiments 94-103, the modified T cell of any of embodiments 104-120 and 134, the pharmaceutical composition of any of embodiments 142-155, or a portion or a component of any of the foregoing.

### IX. EXAMPLES

The following examples are included for illustrative purposes only and are not intended to limit the scope of the invention.

### Example 1: A Screen for gRNAs Targeting Genes Affecting T Cell Phenotype

A library of gRNAs targeting DNA-targeting genes was screened in a pooled format in primary human T-cells expressing an exemplary dCas9-transcriptional activator fusion protein, to identify gRNAs that facilitate enrichment of stem cell-like memory T (T_{SCM}) cell-like phenotypes.

A. Screen of gRNA Library for gRNAs that Promote T_{SCM} Cell-Like Phenotype Via CRISPR-Based Transcriptional Activation (CRISPRa)

A library of 9,715 gRNAs was generated. The library consisted of 9,465 gRNAs targeted to 1,702 human genes, and 250 control gRNAs with spacers not aligned to the human genome. gRNAs were designed according to the protospacer adjacent motif (PAM) sequence for SpCas9 (5′-NGG-3′).

The library was screened in a CRISPR-activation (CRISPRa) screen to identify gRNAs that facilitate enrichment of a CCR7+/CD27+T_{SCM} cell-like phenotype in primary T cells expressing dSpCas9-VP64 (SEQ ID NO: 400), an exemplary DNA-targeting fusion protein for transcriptional activation of gRNA-targeted genes, as described below.

Primary human CD4+ and CD8+ T cells were isolated from a leukapheresis pack (Stemcell technologies) obtained from a human subject, using EasySep human CD4+ T cell isolation kit (Stemcell technologies Cat #17952) and EasySep CD8+ T cell isolation kit (Stemcell technologies Cat #17953), respectively. Isolated cells were aliquoted and cryopreserved.

On day 0, CD4+ and CD8+ T cells were thawed and pooled at a 1:1 ratio. Then, cells were activated using CTS Dynabeads CD3/CD28 (ThermoFisher Scientific Cat #40203D) at a beads-to-cell ratio of 1:1, and cultured in CTS OpTmizer T Cell Expansion SFM (ThermoFisher Scientific Cat #A1048501) with human IL-7, IL-15, and IL-2.

On day 1, T cells were transduced with lentiviral constructs encoding dSpCas9-VP64 and the pooled gRNA library with 10 μg/ml of protamine sulfate in CTS OpTmizer T Cell Expansion SFM (ThermoFisher Scientific Cat #A1048501) with human IL-7, IL-15, and IL-2, without antibiotic selection. Each individual construct encoded the dSpCas9-VP64 and a single gRNA from the library.

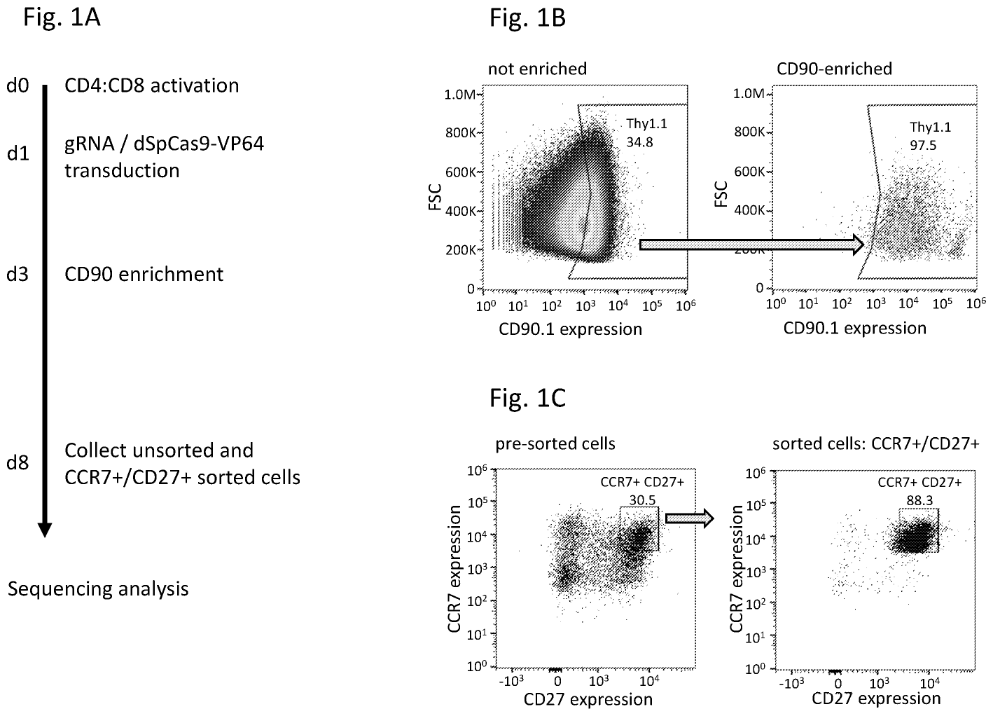

On day 3, CD90+ cells were enriched using CD90.1 MicroBeads (Miltenyi Biotec Cat #: 130-094-523), and enrichment was confirmed by flow cytometry, as shown in FIG. 1B.

On day 8, unfixed cells were immunostained with anti-CCR7 and anti-CD27 antibodies and sorted by FACS into (a) a “double-positive” CCR7+/CD27+ TSCM cell-like population, (b) a “double negative” CCR7−/CD27− population, and (c) an unsorted population with all cells regardless of CCR7 and CD27 expression (FIG. 1C). Unsorted, double-positive, and double-negative populations were collected.

gRNAs that facilitate activation of genes that promote a CCR7+/CD27+T_{SCM} cell-like phenotype were expected to be enriched in the CCR7+/CD27+ population in comparison to the unsorted population. To identify gRNAs enriched in the CCR7+/CD27+ population, sequencing was performed to compare the abundance of each gRNA between the CCR7+/CD27+ population and the unsorted population. Genomic DNA was isolated from the two populations. Targeted PCR was performed to amplify the gRNA spacers and append sequencing adapters. Each sample was barcoded separately. Samples were then sequenced using an Illumina NextSeq System. Three sequencing replicates of the CCR7+/CD27+ population were compared to three replicates of the unsorted population using DEseq2, a method for detecting differentially expressed transcripts.

Identification of gRNAs and Genes Promoting a CCR7+/CD27+T_{SCM} Cell-Like Phenotype

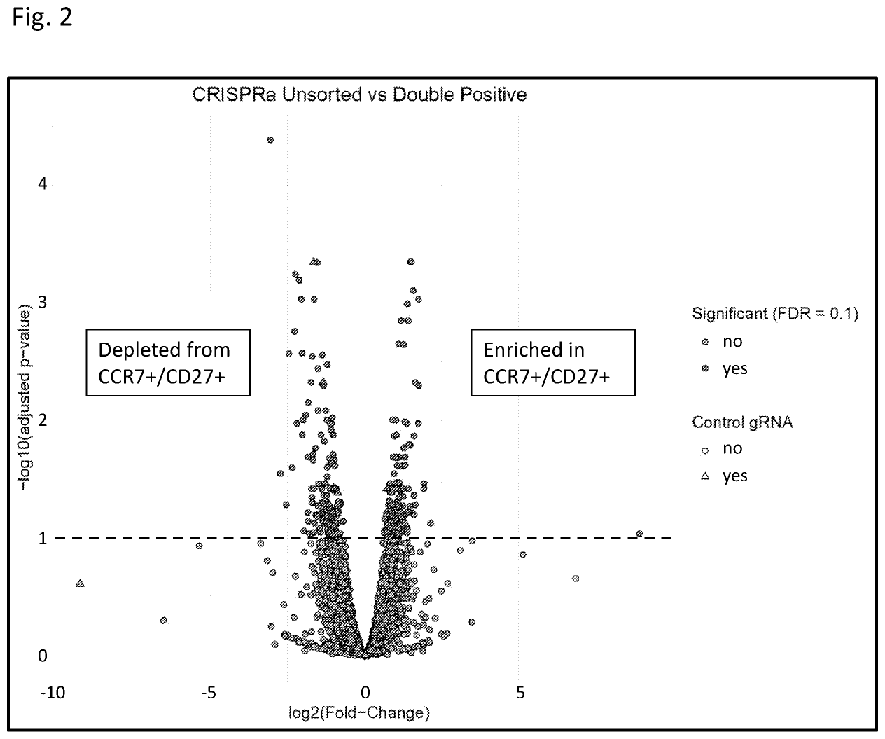

gRNAs enriched in the CCR7+/CD27+ population in comparison to the unsorted population were identified based on sequencing analysis (FIG. 2). A false discovery rate (FDR) cutoff of adjusted p-value <0.1 was used to define significantly enriched gRNAs. 132 gene-targeting gRNAs (Table E1; SEQ ID NOs: 1-132) were enriched in the CCR7+/CD27+ population.

These results support that genes targeted by the enriched gRNAs would be expected to promote the assessed T_{SCM} cell-like phenotype when activated. The enriched gene-targeting gRNAs targeted 130 different genes. 2 of the 130 genes, ZNF319 and ZNF470, were each targeted by two separate gRNAs identified in the screen.

TABLE E1
gRNAs enriched in CCR7+/CD27+ population in CRISPRa screen

| Target | | | |
|---|---|---|---|
| site SEQ | | | |
| ID NO: | gRNA name | gRNA target site | Target gene |
| 1 | DDIT3_a | CCCTAGCGAGAGGGAGCGA | DDIT3 |
| 2 | HES2_a | CCGTCGGGGATCTGCGGGC | HES2 |
| 3 | HKR1_a | CAGTCCCGCGATGCCTTCG | HKR1 |
| 4 | PLAG1_a | AACACTTCGGCTTGACGGG | PLAG1 |
| 5 | PRDM4_a | TGGCCGCGAGAGGCTCGGC | PRDM4 |
| 6 | TEF_a | AGGCTGGGAGCCATAACTG | TEF |
| 7 | TSHZ1_a | GACCTATTTGTAATTGTCA | TSHZ1 |
| 8 | ZNF141_a | GAGAGCTGTGCACCCAACT | ZNF141 |
| 9 | ZNF562_a | TGGACGGAACCGTGGGCGA | ZNF562 |
| 10 | ZNF691_a | CGCCCTACTACTCCAGGAT | ZNF691 |
| 11 | ZNF692_a | CCCCGCGGCAGTTGTGGTT | ZNF692 |
| 12 | ZSCAN5A_a | ACGTAATCGGCGTTGATTA | ZSCAN5A |
| 13 | EBF4_a | AAGACGCCGGGGCTTCGTC | EBF4 |
| 14 | ETS1_a | AGAAACGCCCTAAAGAGGA | ETS1 |
| 15 | FOXD3_a | GCTGCAAGGGCGGTGTTGG | FOXD3 |
| 16 | HELT_a | CAAGCGCCTGCAGCAGTCG | HELT |
| 17 | HMGN3_a | GCGTACCGCGCGCGGAGGT | HMGN3 |
| 18 | PATZ1_a | GAAACGGCGAGCTCAGAGT | PATZ1 |
| 19 | PRDM8_a | CCCAGCTCTCCATCCCCGG | PRDM8 |
| 20 | SOX21_a | CGGGAGAGGAAGACTCGAG | SOX21 |
| 21 | TBX15_a | GATCACAGGGAGGACGGGA | TBX15 |
| 22 | ZBED5_a | AAAGGACCAAGCTCGGTTG | ZBED5 |
| 23 | ZBTB7A_a | TCCCTCCGCCGTTTGCAGC | ZBTB7A |
| 24 | ZNF219_a | CCTAAACCCGGACATACCA | ZNF219 |
| 25 | ZNF319_a | TACCACTCTAAGCCTTCTC | ZNF319 |
| 26 | ZNF470_a | TTCCCAGGGGCTATTGCAG | ZNF470 |
| 27 | ZNF816_a | AGCAGGAATGCGGGCGCGG | ZNF816 |
| 28 | ZSCAN23_a | CTCCGCGAAACAGCGGTTC | ZSCAN23 |
| 29 | EMX1_a | AGCGCCGGGAGCCGTGGTT | EMX1 |
| 30 | FOXO3_a | GCCGCGGGAGGCACTAGAG | FOXO3 |
| 31 | HIC2_a | TGGGCTCCTGGGGAAGCCC | HIC2 |
| 32 | SOX30_a | GCCGCGGAAAGCAGATCTG | SOX30 |
| 33 | STAT1_a | CAGCGTGGCAACACGGGAC | STAT1 |
| 34 | ZBTB46_a | CCGCGCCGCGGGGTCGCAG | ZBTB46 |
| 35 | ZNF331_a | CAGGTGAGATTGGCGCCCA | ZNF331 |
| 36 | ZNF541_a | CAAGTCATGTGTCAGATAA | ZNF541 |
| 37 | ZNF619_a | CGCAGAGCGGTTGTGGAGA | ZNF619 |
| 38 | AEBP1_a | CGGGACCCCGAGTCCACCG | AEBP1 |
| 39 | ARID3A_a | CCCGCCCCCGGCCTAGGGA | ARID3A |
| 40 | ARNTL2_a | ACCCTCGGGGCAAAGGTAA | ARNTL2 |
| 41 | BACH1_a | TCGCTGACAGAGGCTTCGA | BACH1 |
| 42 | BATF2_a | CAAAGGCCAGGTATGAGGA | BATF2 |
| 43 | BRD4_a | TGCTGAAGTCATGAGGTGT | BRD4 |
| 44 | CDX4_a | GGGAAGATCGGTGAAAGGG | CDX4 |
| 45 | CENPB_a | GCCCCCCCGCGGTATGTCC | CENPB |
| 46 | DMRT1_a | AGAGCGGTGGCTGGAGTCG | DMRT1 |
| 47 | E2F2_a | CGGAAAGAACGCGTCCTCG | E2F2 |
| 48 | ESRRB_a | GAGGGGCAACCCCCGACTC | ESRRB |
| 49 | FERD3L_a | TGAGTAGGAGGTAAAGGTA | FERD3L |
| 50 | FEV_a | CGGGAGCCGACTGGAAGAA | FEV |
| 51 | FLYWCH1_a | GGGACGTAACTCACGGAAA | FLYWCH1 |
| 52 | FOXJ3_a | CAGAGCCTCGGGAGTCTCA | FOXJ3 |
| 53 | GLIS3_a | GGCTCGCGGGTCACTAGGC | GLIS3 |
| 54 | HDAC9_a | GTAAAGCAAGCTTCGAGAG | HDAC9 |
| 55 | HIVEP1_2 | CAGTCACGCGGCCCCGGGA | HIVEP1 |
| 56 | HOXA2_4 | GGAAGGAACGCCTCATTGG | HOXA2 |
| 57 | HOXC9_1 | GAGCTAGGAGCCGTCCTGA | HOXC9 |
| 58 | HSFY1_5 | GGTAGGTTACGGGTTGTCA | HSFY1 |
| 59 | ISL2_2 | AACGGCCGCGGAGCCAGCT | ISL2 |
| 60 | KAT2A_5 | TCCGGAGCCGCGTGGGGCA | KAT2A |
| 61 | KDM5D_5 | GCAAGAGCGATTTTACCAG | KDM5D |
| 62 | KLF13_a | ACCGGGCACGCCCCTCTAA | KLF13 |
| 63 | KLF6_a | TGACCGAGACTCTCCATCA | KLF6 |
| 64 | MEOX2_a | AATCAGGGTGCTAGGTTGG | MEOX2 |
| 65 | MLXIPL_a | CGCCAGAGCTCCAGAGCAC | MLXIPL |
| 66 | MNT_a | GGGCCGCGGCGTCTCGCGA | MNT |
| 67 | MYCN_a | CAAGCCCGGGGGTCCAAAA | MYCN |
| 68 | MYTIL_a | GCCTCATAGATAATGATGA | MYTIL |
| 69 | NEUROG3_a | CTCGCGTGGCGCGGCCCCA | NEUROG3 |
| 70 | NFE2_a | AGGAGAGCCACGGGGAAAG | NFE2 |
| 71 | NR2F1_a | TTTCTCTCCCCCCGATTCA | NR2F1 |
| 72 | NR2F2_a | CCCCCTCGCCGTCATTGGC | NR2F2 |
| 73 | NR3C1_a | GGGCCCGCGCGGAGGGCGT | NR3C1 |
| 74 | PHF20_a | AGGCCGTTGCCACAGTGAT | PHF20 |
| 75 | PITX2_a | CAGGACAGTCCCTCCACCG | PITX2 |
| 76 | POU2AF1_a | GCCGCTGGGGCGCGCACAG | POU2AF1 |
| 77 | POU3F2_a | GCGGGGCCCATCCGAGAGA | POU3F2 |
| 78 | POU3F3_a | GGCCACGACCCCCCCTGAA | POU3F3 |
| 79 | PRDM11_a | TGGGTGTGGAGGGGTTCCA | PRDM11 |
| 80 | PRMT3_a | TGAAGAGGCGCGCTGATGA | PRMT3 |
| 81 | PROP1_a | TCGGAGGGTCCAGGGACTG | PROP1 |
| 82 | PURA_a | CTCACGCGCGAGAAGGGGG | PURA |
| 83 | RELB_a | TGGGCGCTCAGGAGGGGTT | RELB |
| 84 | RFX3_a | ACTGACGGCCCCCAAACCC | RFX3 |
| 85 | SAFB_a | CTGCTAAGTACGCTAGTGG | SAFB |
| 86 | SETBP1_a | TATGGAGAGAGATATCAAG | SETBP1 |
| 87 | SMAD2_a | AAGCCGCGCGGGGGGGGTA | SMAD2 |
| 88 | SMAD6_a | CCCGGGGGGGGCCGAGCTC | SMAD6 |
| 89 | SNAPC2_a | AGGGAGGGCATGGGATCCA | SNAPC2 |
| 90 | SNAPC4_a | TCTCAGAGCCCACCCTCGA | SNAPC4 |
| 91 | SON_a | CTGTACTTGGCCTGTACTA | SON |
| 92 | SOX11_a | GGGCTGCTTTGCAAAGATG | SOX11 |
| 93 | SOX12_a | GGCCCGGCCGCAGCCCAAG | SOX12 |
| 94 | SOX14_a | CAAAAGCGGCGAAGTTCAA | SOX14 |
| 95 | SPIC_a | TGCAACCTAAGTTGGGCGG | SPIC |
| 96 | THAP7_a | AATCGGCTGGGACGGGGTT | THAP7 |
| 97 | TOX2_a | GCTCCCGCGGGCAGTGCCT | TOX2 |
| 98 | TRERF1_a | GGGTACCGGGGAGCGGATA | TRERF1 |
| 99 | TRIM3_a | GGGCGTGCGGGTGCGGTAG | TRIM3 |
| 100 | VDR_a | TACAGCGTGGCCTATAGGG | VDR |
| 101 | YBX2_a | GGGCCCAGGTGTACTTACT | YBX2 |
| 102 | ZBTB14_a | CGCGCGCGTGCGCAGGCCA | ZBTB14 |
| 103 | ZBTB4_a | CCGGGACGGTTGGAGAAGA | ZBTB4 |
| 104 | ZFHX2_a | GGTACAGGTCTCATGCCTG | ZFHX2 |
| 105 | ZFP57_a | GCAGCTATCAGTTGGTCGG | ZFP57 |
| 106 | ZFP62_a | GAGGGCAGAGAGACCTCTA | ZFP62 |
| 107 | ZFP69B_a | TGATGATGCTCGCAGGGAT | ZFP69B |
| 108 | ZIC1_a | GGGAATGCGGGCGCACCAA | ZIC1 |
| 109 | ZIC4_a | CAACGGGGACGTTGACAAG | ZIC4 |
| 110 | ZNF136_a | CCCCGACGGCGCGCCTGAT | ZNF136 |
| 111 | ZNF16_a | GCTAGTGGGACCAGGTCTG | ZNF16 |
| 112 | ZNF174_a | CAGGGGCGTGCGTAATGGG | ZNF174 |
| 113 | ZNF253_a | TGTCACTCAGCGGCTGGGG | ZNF253 |
| 114 | ZNF26_a | ACAGCCTGACGGGAAAGGA | ZNF26 |
| 115 | ZNF263_a | GGGCGCGCCCTGAAATTCT | ZNF263 |
| 116 | ZNF285_a | AAAGCCCCGCGCGCAGCAG | ZNF285 |
| 117 | ZNF317_a | TCCCGCGAGAATTGGAAGG | ZNF317 |
| 118 | ZNF319_b | CATCTTCCGGAAGGGAGGG | ZNF319 |
| 119 | ZNF345_a | CGCGCCAGGGGAATCGGGA | ZNF345 |
| 120 | ZNF420_a | GAGCCCACGAGCCCCTCGA | ZNF420 |
| 121 | ZNF423_a | CAGGGGATCAGCCATCCAG | ZNF423 |
| 122 | ZNF470_b | GACCAGGAGAGGCCCAAAA | ZNF470 |
| 123 | ZNF48_a | GAGGCCGGCGGCCCGAACT | ZNF48 |
| 124 | ZNF496_a | GGAGCAGGAAGTGTCCCAG | ZNF496 |
| 125 | ZNF501_a | AAACGGGATGGAAGTCAGA | ZNF501 |
| 126 | ZNF524_a | GGGTCCTAACGACCGGTCG | ZNF524 |
| 127 | ZNF557_a | CTGGCGCGGGTCTGTGGCA | ZNF557 |
| 128 | ZNF735_a | AAAAGGCCCGGTAAAATTA | ZNF735 |
| 129 | ZNF747_a | GGAACAAAGCAGCGCCCTG | ZNF747 |
| 130 | ZNF783_a | AGGCGCTGCAACAGAGTCT | ZNF783 |
| 131 | ZNF805_a | CTGGGGCCAATGATGGTCC | ZNF805 |
| 132 | ZSCAN10_a | TTACACTCTCACACAGGCT | ZSCAN10 |

28 of the 130 genes targeted by the enriched gRNAs were prioritized for further analysis based on several criteria, including whether the gene was targeted by more than one gRNA, and degree of enrichment of the gene-targeting gRNA based on the above-described sequencing analysis. Table E2 shows the 28 prioritized genes with exemplary gene-targeting gRNAs.

TABLE E2
Prioritized genes and exemplary targeting gRNAs

| | Exemplary gene- | | |
|---|---|---|---|
| Target | targeting gRNA | | Target site |
| gene | name | gRNA target site | SEQ ID NO: |
| DDIT3 | DDIT3_a | CCCTAGCGAGAGGGAGCGA | 1 |
| HES2 | HES2_a | CCGTCGGGGATCTGCGGGC | 2 |
| HKR1 | HKR1_a | CAGTCCCGCGATGCCTTCG | 3 |
| PLAG1 | PLAG1_a | AACACTTCGGCTTGACGGG | 4 |
| PRDM4 | PRDM4_a | TGGCCGCGAGAGGCTCGGC | 5 |
| TEF | TEF_a | AGGCTGGGAGCCATAACTG | 6 |
| TSHZ1 | TSHZ1_a | GACCTATTTGTAATTGTCA | 7 |
| ZNF141 | ZNF141_a | GAGAGCTGTGCACCCAACT | 8 |
| ZNF562 | ZNF562_a | TGGACGGAACCGTGGGCGA | 9 |
| ZNF691 | ZNF691_a | CGCCCTACTACTCCAGGAT | 10 |
| ZNF692 | ZNF692_a | CCCCGCGGCAGTTGTGGTT | 11 |
| ZSCAN5A | ZSCAN5A_a | ACGTAATCGGCGTTGATTA | 12 |
| EBF4 | EBF4_a | AAGACGCCGGGGCTTCGTC | 13 |
| ETS1 | ETS1_a | AGAAACGCCCTAAAGAGGA | 14 |
| FOXD3 | FOXD3_a | GCTGCAAGGGCGGTGTTGG | 15 |
| HELT | HELT_a | CAAGCGCCTGCAGCAGTCG | 16 |
| HMGN3 | HMGN3_a | GCGTACCGCGCGCGGAGGT | 17 |
| PATZ1 | PATZ1_a | GAAACGGCGAGCTCAGAGT | 18 |
| PRDM8 | PRDM8_a | CCCAGCTCTCCATCCCCGG | 19 |
| SOX21 | SOX21_a | CGGGAGAGGAAGACTCGAG | 20 |
| TBX15 | TBX15_a | GATCACAGGGAGGACGGGA | 21 |
| ZBED5 | ZBED5_a | AAAGGACCAAGCTCGGTTG | 22 |
| ZBTB7A | ZBTB7A_a | TCCCTCCGCCGTTTGCAGC | 23 |
| ZNF219 | ZNF219_a | CCTAAACCCGGACATACCA | 24 |
| ZNF319 | ZNF319_a | TACCACTCTAAGCCTTCTC | 25 |
| ZNF470 | ZNF470_a | TTCCCAGGGGCTATTGCAG | 26 |
| ZNF816 | ZNF816_a | AGCAGGAATGCGGGCGCGG | 27 |
| ZSCAN23 | ZSCAN23_a | CTCCGCGAAACAGCGGTTC | 28 |

B. Complementary CRISPR-Interference (CRISPRi) Screen to Identify of Genes that Positively Regulate a CCR7+/CD27+T_{SCM} Cell-Like Phenotype

A complementary CRISPR-interference (CRISPRi) screen was performed in which the gRNA library was screened in primary T cells expressing dSpCas9-KRAB (SEQ ID NO: 402), an exemplary DNA-targeting fusion protein for transcriptional repression of gRNA-targeted genes (as opposed to activation by dSpCas9-VP64). gRNAs were identified that were depleted from the CCR7+/CD27+ population in comparison to the unsorted population. Repression of the genes targeted by the depleted gRNAs would be expected to inhibit the assessed T_{SCM} cell-like phenotype. 21 of these genes identified in the CRISPRi screen overlapped with genes identified in the CRISPRa screen, as described above. The results from the complementary CRISPRa and CRISPRi screens suggest that the 21 overlapping genes both promote the CCR7+/CD27+T_{SCM} cell-like phenotype when activated and inhibit the CCR7+/CD27+T_{SCM} cell-like phenotype when repressed. These 21 genes are therefore highly likely to positively regulate the T_{SCM} cell-like phenotype. In addition, 12 of these 21 genes also overlapped with genes from the prioritized group of genes (as shown in Table E2) from the CRISPRa screen. Table E3 shows these 12 “CRISPRa/CRISPRi overlap” genes with exemplary activating gene-targeting gRNAs.

TABLE E3
CRISPRa/CRISPRi overlap genes and exemplary targeting gRNAs

| | Exemplary gene- | | |
|---|---|---|---|
| Target | targeting gRNA | | Target site |
| gene | name | gRNA target site | SEQ ID NO: |
| DDIT3 | DDIT3_a | CCCTAGCGAGAGGGAGCGA | 1 |
| HES2 | HES2_a | CCGTCGGGGATCTGCGGGC | 2 |
| HKR1 | HKR1_a | CAGTCCCGCGATGCCTTCG | 3 |
| PLAG1 | PLAG1_a | AACACTTCGGCTTGACGGG | 4 |
| PRDM4 | PRDM4_a | TGGCCGCGAGAGGCTCGGC | 5 |
| TEF | TEF_a | AGGCTGGGAGCCATAACTG | 6 |
| TSHZ1 | TSHZ1_a | GACCTATTTGTAATTGTCA | 7 |
| ZNF141 | ZNF141_a | GAGAGCTGTGCACCCAACT | 8 |
| ZNF562 | ZNF562_a | TGGACGGAACCGTGGGCGA | 9 |
| ZNF691 | ZNF691_a | CGCCCTACTACTCCAGGAT | 10 |
| ZNF692 | ZNF692_a | CCCCGCGGCAGTTGTGGTT | 11 |
| ZSCAN5A | ZSCAN5A_a | ACGTAATCGGCGTTGATTA | 12 |

In summary, the results show that gene-targeting gRNAs, along with an exemplary Cas9 fusion protein with transcriptional activation activity, can facilitate enrichment of CCR7+/CD27+T_{SCM} cell-like phenotypes in primary T cells. The results support the utility of the identified gRNAs and modulation of the targeted genes for modifying T cell phenotypes, which may be advantageous for adoptive cell therapy

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ | | |
|---|---|---|
| ID | | |
| NO. | Sequence | Annotation |
| 397 | GTTTAAGAGCTATGCTGGAAACAGCATAGCAAGTTTAAATAAGGCTAGTCCGTTA | SpCas9 |
| | TCAACTTGAAAAAGTGGCACCGAGTCGGTGC | gRNA |
| | | scaffold |
| | | (DNA) |
| 398 | GUUUAAGAGCUAUGCUGGAAACAGCAUAGCAAGUUUAAAUAAGGCUAGUCCGU | SpCas9 |
| | UAUCAACUUGAAAAAGUGGCACCGAGUCGGUGC | gRNA |
| | | scaffold |
| | | (RNA) |
| 399 | gacgcattggacgattttgatctggatatgctgggaagtgacgccctcgatgattttgaccttgacatgc | VP64- |
| | ttggttcggatgcccttgatgactttgacctcgacatgctcggcagtgacgcccttgatgatttcgacct | dSpCas9- |
| | ggacatgGTTAACCCAAAGAAGAAGCGGAAGGTCGGTATCCACGGAGTCCCAGCAGCCGACAAGAAGTAC | NLS-VP64 |
| | TCCATTGGGCTCGCCATCGGCACAAACAGCGTCGGCTGGGCCGTCATTACGGACGAGTACAAGGTGCCG | (nt) |
| | AGCAAAAAATTCAAAGTTCTGGGCAATACCGATCGCCACAGCATAAAGAAGAAC | |
| | CTCATTGGCGCCCTCCTGTTCGACTCCGGGGAAACCGCCGAAGCCACGCGGCTCA | |
| | AAAGAACAGCACGGCGCAGATATACCCGCAGAAAGAATCGGATCTGCTACCtgcaG | |
| | GAGATCTTTAGTAATGAGATGGCTAAGGTGGATGACTCTTTCTTCCATAGGCTGGA | |
| | GGAGTCCTTTTTGGTGGAGGAGGATAAAAAGCACGAGCGCCACCCAATCTTTGGC | |
| | AATATCGTGGACGAGGTGGCGTACCATGAAAAGTACCCAACCATATATCATCTGA | |
| | GGAAGAAGCTTGTAGACAGTACTGATAAGGCTGACTTGCGGTTGATCTATCTCGC | |
| | GCTGGCGCATATGATCAAATTTCGGGGACACTTCCTCATCGAGGGGGACCTGAAC | |
| | CCAGACAACAGCGATGTCGACAAACTCTTTATCCAACTGGTTCAGACTTACAATC | |
| | AGCTTTTCGAAGAGAACCCGATCAACGCATCCGGAGTTGACGCCAAAGCAATCCT | |
| | GAGCGCTAGGCTGTCCAAATCCCGGCGGCTCGAAAACCTCATCGCACAGCTCCCT | |
| | GGGGAGAAGAAGAACGGCCTGTTTGGTAATCTTATCGCCCTGTCACTCGGGCTGA | |
| | CCCCCAACTTTAAATCTAACTTCGACCTGGCCGAAGATGCCAAGCTTCAACTGAGC | |
| | AAAGACACCTACGATGATGATCTCGACAATCTGCTGGCCCAGATCGGCGACCAGT | |
| | ACGCAGACCTTTTTTTGGCGGCAAAGAACCTGTCAGACGCCATTCTGCTGAGTGAT | |
| | ATTCTGCGAGTGAACACGGAGATCACCAAAGCTCCGCTGAGCGCTAGTATGATCA | |
| | AGCGCTATGATGAGCACCACCAAGACTTGACTTTGCTGAAGGCCCTTGTCAGACA | |
| | GCAACTGCCTGAGAAGTACAAGGAAATTTTCTTCGATCAGTCTAAAAATGGCTAC | |
| | GCCGGATACATTGACGGCGGAGCAAGCCAGGAGGAATTTTACAAATTTATTAAGC | |
| | CCATCTTGGAAAAAATGGACGGCACCGAGGAGCTGCTGGTAAAGCTTAACAGAG | |
| | AAGATCTGTTGCGCAAACAGCGCACTTTCGACAATGGAAGCATCCCCCACCAGAT | |
| | TCACCTGGGCGAACTGCACGCTATCCTCAGGCGGCAAGAGGATTTCTACCCCTTTT | |
| | TGAAAGATAACAGGGAAAAGATTGAGAAAATCCTCACATTTCGGATACCCTACTA | |
| | TGTAGGCCCCCTCGCCCGGGGAAATTCCAGATTCGCGTGGATGACTCGCAAATCA | |
| | GAAGAGACCATCACTCCCTGGAACTTCGAGGAAGTCGTGGATAAGGGGGCCTCTG | |
| | CCCAGTCCTTCATCGAAAGGATGACTAACTTTGATAAAAATCTGCCTAACGAAAA | |
| | GGTGCTTCCTAAACACTCTCTGCTGTACGAGTACTTCACAGTTTATAACGAGCTCA | |
| | CCAAGGTCAAATACGTCACAGAAGGGATGAGAAAGCCAGCATTCCTGTCTGGAGA | |
| | GCAGAAGAAAGCTATCGTGGACCTCCTCTTCAAGACGAACCGGAAAGTTACCGTG | |
| | AAACAGCTCAAAGAAGACTATTTCAAAAAGATTGAATGTTTCGACTCTGTTGAAA | |
| | TCAGCGGAGTGGAGGATCGCTTCAACGCATCCCTGGGAACGTATCACGATCTCCT | |
| | GAAAATCATTAAAGACAAGGACTTCCTGGACAATGAGGAGAACGAGGACATTCTT | |
| | GAGGACATTGTCCTCACCCTTACGTTGTTTGAAGATAGGGAGATGATTGAAGAAC | |
| | GCTTGAAAACTTACGCTCATCTCTTCGACGACAAAGTCATGAAACAGCTCAAGAG | |
| | GCGCCGATATACAGGATGGGGGCGGCTGTCAAGAAAACTGATCAATGGgatcCGAG | |
| | ACAAGCAGAGTGGAAAGACAATCCTGGATTTTCTTAAGTCCGATGGATTTGCCAA | |
| | CCGGAACTTCATGCAGTTGATCCATGATGACTCTCTCACCTTTAAGGAGGACATCC | |
| | AGAAAGCACAAGTTTCTGGCCAGGGGGACAGTCTTCACGAGCACATCGCTAATCT | |
| | TGCAGGTAGCCCAGCTATCAAAAAGGGAATACTGCAGACCGTTAAGGTCGTGGAT | |
| | GAACTCGTCAAAGTAATGGGAAGGCATAAGCCCGAGAATATCGTTATCGAGATGG | |
| | CCCGAGAGAACCAAACTACCCAGAAGGGACAGAAGAACAGTAGGGAAAGGATGA | |
| | AGAGGATTGAAGAGGGTATAAAAGAACTGGGGTCCCAAATCCTTAAGGAACACC | |
| | CAGTTGAAAACACCCAGCTTCAGAATGAGAAGCTCTACCTGTACTACCTGCAGAA | |
| | CGGCAGGGACATGTACGTGGATCAGGAACTGGACATCAATCGGCTCTCCGACTAC | |
| | GACGTGGATGCCATCGTGCCCCAGTCTTTTCTCAAAGATGATTCTATTGATAATAA | |
| | AGTGTTGACAAGATCCGATAAAAATAGAGGGAAGAGTGATAACGTCCCCTCAGA | |
| | AGAAGTTGTCAAGAAAATGAAAAATTATTGGCGGCAGCTGCTGAACGCCAAACTG | |
| | ATCACACAACGGAAGTTCGATAATCTGACTAAGGCTGAACGAGGTGGCCTGTCTG | |
| | AGTTGGATAAAGCCGGCTTCATCAAAAGGCAGCTTGTTGAGACACGCCAGATCAC | |
| | CAAgcacGTGGCCCAAATTCTCGATTCACGCATGAACACCAAGTACGATGAAAATG | |
| | ACAAACTGATTCGAGAGGTGAAAGTTATTACTCTGAAGTCTAAGCTGGTCTCAGA | |
| | TTTCAGAAAGGACTTTCAGTTTTATAAGGTGAGAGAGATCAACAATTACCACCAT | |
| | GCGCATGATGCCTACCTGAATGCAGTGGTAGGCACTGCACTTATCAAAAAATATC | |
| | CCAAGCTTGAATCTGAATTTGTTTACGGAGACTATAAAGTGTACGATGTTAGGAA | |
| | AATGATCGCAAAGTCTGAGCAGGAAATAGGCAAGGCCACCGCTAAGTACTTCTTT | |
| | TACAGCAATATTATGAATTTTTTCAAGACCGAGATTACACTGGCCAATGGAGAGA | |
| | TTCGGAAGCGACCACTTATCGAAACAAACGGAGAAACAGGAGAAATCGTGTGGG | |
| | ACAAGGGTAGGGATTTCGCGACAGTCCGGAAGGTCCTGTCCATGCCGCAGGTGAA | |
| | CATCGTTAAAAAGACCGAAGTACAGACCGGAGGCTTCTCCAAGGAAAGTATCCTC | |
| | CCGAAAAGGAACAGCGACAAGCTGATCGCACGCAAAAAAGATTGGGACCCCAAG | |
| | AAATACGGCGGATTCGATTCTCCTACAGTCGCTTACAGTGTACTGGTTGTGGCCAA | |
| | AGTGGAGAAAGGGAAGTCTAAAAAACTCAAAAGCGTCAAGGAACTGCTGGGCAT | |
| | CACAATCATGGAGCGATCAAGCTTCGAAAAAAACCCCATCGACTTTCTCGAGGCG | |
| | AAAGGATATAAAGAGGTCAAAAAAGACCTCATCATTAAGCTTCCCAAGTACTCTC | |
| | TCTTTGAGCTTGAAAACGGCCGGAAACGAATGCTCGCTAGTGCGGGCGAGCTGCA | |
| | GAAAGGTAACGAGCTGGCACTGCCCTCTAAATACGTTAATTTCTTGTATCTGGCCA | |
| | GCCACTATGAAAAGCTCAAAGGGTCTCCCGAAGATAATGAGCAGAAGCAGCTGTT | |
| | CGTGGAACAACACAAACACTACCTTGATGAGATCATCGAGCAAATAAGCGAATTC | |
| | TCCAAAAGAGTGATCCTCGCCGACGCTAACCTCGATAAGGTGCTTTCTGCTTACAA | |
| | TAAGCACAGGGATAAGCCCATCAGGGAGCAGGCAGAAAACATTATCCACTTGTTT | |
| | ACTCTGACCAACTTGGGCGCGCCTGCAGCCTTCAAGTACTTCGACACCACCATAG | |
| | ACAGAAAGCGGTACACCTCTACAAAGGAGGTCCTGGACGCCACACTGATTCATCA | |
| | GTCAATTACGGGGCTCTATGAAACAAGAATCGACCTCTCTCAGCTCGGTGGAGAC | |
| | AAAAGGCCGGCGGCCACGAAAAAGGCCGGCCAGGCAAAAAAGAAAAAGGctagCcg | |
| | cgccgacgcgctggacgatttcgatctcgacatgctgggttctgatgccctcgatgactttgacctgga | |
| | tatgttgggaagcgacgcattggatgactttgatctggacatgctcggctccgatgctctggacgatttc | |
| | gatctcgatatgtta | |
| 400 | DALDDFDLDMLGSDALDDFDLDMLGSDALDDFDLDMLGSDALDDFDLDMVNPKKK | VP64- |
| | RKVGIHGVPAADKKYSIGLAIGTNSVGWAVITDEYKVPSKKFKVLGNTDRHSIKKNLI | dSpCas9- |
| | GALLFDSGETAEATRLKRTARRRYTRRKNRICYLQEIFSNEMAKVDDSFFHRLEESFL | NLS-VP64 |
| | VEEDKKHERHPIFGNIVDEVAYHEKYPTIYHLRKKLVDSTDKADLRLIYLALAHMIKF | (AA) |
| | RGHFLIEGDLNPDNSDVDKLFIQLVQTYNQLFEENPINASGVDAKAILSARLSKSRRLE | |
| | NLIAQLPGEKKNGLFGNLIALSLGLTPNFKSNFDLAEDAKLQLSKDTYDDDLDNLLAQ | |
| | IGDQYADLFLAAKNLSDAILLSDILRVNTEITKAPLSASMIKRYDEHHQDLTLLKALVR | |
| | QQLPEKYKEIFFDQSKNGYAGYIDGGASQEEFYKFIKPILEKMDGTEELLVKLNREDLL | |
| | RKQRTFDNGSIPHQIHLGELHAILRRQEDFYPFLKDNREKIEKILTFRIPYYVGPLARGN | |
| | SRFAWMTRKSEETITPWNFEEVVDKGASAQSFIERMTNFDKNLPNEKVLPKHSLLYEY | |
| | FTVYNELTKVKYVTEGMRKPAFLSGEQKKAIVDLLFKTNRKVTVKQLKEDYFKKIEC | |
| | FDSVEISGVEDRFNASLGTYHDLLKIIKDKDFLDNEENEDILEDIVLTLTLFEDREMIEE | |
| | RLKTYAHLFDDKVMKQLKRRRYTGWGRLSRKLINGIRDKQSGKTILDFLKSDGFANR | |
| | NFMQLIHDDSLTFKEDIQKAQVSGQGDSLHEHIANLAGSPAIKKGILQTVKVVDELVK | |
| | VMGRHKPENIVIEMARENQTTQKGQKNSRERMKRIEEGIKELGSQILKEHPVENTQLQ | |
| | NEKLYLYYLQNGRDMYVDQELDINRLSDYDVDAIVPQSFLKDDSIDNKVLTRSDKNR | |
| | GKSDNVPSEEVVKKMKNYWRQLLNAKLITQRKFDNLTKAERGGLSELDKAGFIKRQ | |
| | LVETRQITKHVAQILDSRMNTKYDENDKLIREVKVITLKSKLVSDFRKDFQFYKVREI | |
| | NNYHHAHDAYLNAVVGTALIKKYPKLESEFVYGDYKVYDVRKMIAKSEQEIGKATA | |
| | KYFFYSNIMNFFKTEITLANGEIRKRPLIETNGETGEIVWDKGRDFATVRKVLSMPQVN | |
| | IVKKTEVQTGGFSKESILPKRNSDKLIARKKDWDPKKYGGFDSPTVAYSVLVVAKVE | |
| | KGKSKKLKSVKELLGITIMERSSFEKNPIDFLEAKGYKEVKKDLIIKLPKYSLFELENGR | |
| | KRMLASAGELQKGNELALPSKYVNFLYLASHYEKLKGSPEDNEQKQLFVEQHKHYL | |
| | DEIIEQISEFSKRVILADANLDKVLSAYNKHRDKPIREQAENIIHLFTLTNLGAPAAFKY | |
| | FDTTIDRKRYTSTKEVLDATLIHQSITGLYETRIDLSQLGGDKRPAATKKAGQAKKKK | |
| | ASRADALDDFDLDMLGSDALDDFDLDMLGSDALDDFDLDMLGSDALDDFDLDML | |
| 401 | CCAAAGAAGAAGCGGAAGGTCGGTATCCACGGAGTCCCAGCAGCCGACAAGAAG | dSpCas9- |
| | TACTCCATTGGGCTCGCCATCGGCACAAACAGCGTCGGCTGGGCCGTCATTACGG | KRAB (nt) |
| | ACGAGTACAAGGTGCCGAGCAAAAAATTCAAAGTTCTGGGCAATACCGATCGCCA | |
| | CAGCATAAAGAAGAACCTCATTGGCGCCCTCCTGTTCGACTCCGGGGAAACCGCC | |
| | GAAGCCACGCGGCTCAAAAGAACAGCACGGCGCAGATATACCCGCAGAAAGAAT | |
| | CGGATCTGCTACCtgcaGGAGATCTTTAGTAATGAGATGGCTAAGGTGGATGACTCT | |
| | TTCTTCCATAGGCTGGAGGAGTCCTTTTTGGTGGAGGAGGATAAAAAGCACGAGC | |
| | GCCACCCAATCTTTGGCAATATCGTGGACGAGGTGGCGTACCATGAAAAGTACCC | |
| | AACCATATATCATCTGAGGAAGAAGCTTGTAGACAGTACTGATAAGGCTGACTTG | |
| | CGGTTGATCTATCTCGCGCTGGCGCATATGATCAAATTTCGGGGACACTTCCTCAT | |
| | CGAGGGGGACCTGAACCCAGACAACAGCGATGTCGACAAACTCTTTATCCAACTG | |
| | GTTCAGACTTACAATCAGCTTTTCGAAGAGAACCCGATCAACGCATCCGGAGTTG | |
| | ACGCCAAAGCAATCCTGAGCGCTAGGCTGTCCAAATCCCGGCGGCTCGAAAACCT | |
| | CATCGCACAGCTCCCTGGGGAGAAGAAGAACGGCCTGTTTGGTAATCTTATCGCC | |
| | CTGTCACTCGGGCTGACCCCCAACTTTAAATCTAACTTCGACCTGGCCGAAGATGC | |
| | CAAGCTTCAACTGAGCAAAGACACCTACGATGATGATCTCGACAATCTGCTGGCC | |
| | CAGATCGGCGACCAGTACGCAGACCTTTTTTTGGCGGCAAAGAACCTGTCAGACG | |
| | CCATTCTGCTGAGTGATATTCTGCGAGTGAACACGGAGATCACCAAAGCTCCGCT | |
| | GAGCGCTAGTATGATCAAGCGCTATGATGAGCACCACCAAGACTTGACTTTGCTG | |
| | AAGGCCCTTGTCAGACAGCAACTGCCTGAGAAGTACAAGGAAATTTTCTTCGATC | |
| | AGTCTAAAAATGGCTACGCCGGATACATTGACGGCGGAGCAAGCCAGGAGGAAT | |
| | TTTACAAATTTATTAAGCCCATCTTGGAAAAAATGGACGGCACCGAGGAGCTGCT | |
| | GGTAAAGCTTAACAGAGAAGATCTGTTGCGCAAACAGCGCACTTTCGACAATGGA | |
| | AGCATCCCCCACCAGATTCACCTGGGCGAACTGCACGCTATCCTCAGGCGGCAAG | |
| | AGGATTTCTACCCCTTTTTGAAAGATAACAGGGAAAAGATTGAGAAAATCCTCAC | |
| | ATTTCGGATACCCTACTATGTAGGCCCCCTCGCCCGGGGAAATTCCAGATTCGCGT | |
| | GGATGACTCGCAAATCAGAAGAGACCATCACTCCCTGGAACTTCGAGGAAGTCGT | |
| | GGATAAGGGGGCCTCTGCCCAGTCCTTCATCGAAAGGATGACTAACTTTGATAAA | |
| | AATCTGCCTAACGAAAAGGTGCTTCCTAAACACTCTCTGCTGTACGAGTACTTCAC | |
| | AGTTTATAACGAGCTCACCAAGGTCAAATACGTCACAGAAGGGATGAGAAAGCC | |
| | AGCATTCCTGTCTGGAGAGCAGAAGAAAGCTATCGTGGACCTCCTCTTCAAGACG | |
| | AACCGGAAAGTTACCGTGAAACAGCTCAAAGAAGACTATTTCAAAAAGATTGAAT | |
| | GTTTCGACTCTGTTGAAATCAGCGGAGTGGAGGATCGCTTCAACGCATCCCTGGG | |
| | AACGTATCACGATCTCCTGAAAATCATTAAAGACAAGGACTTCCTGGACAATGAG | |
| | GAGAACGAGGACATTCTTGAGGACATTGTCCTCACCCTTACGTTGTTTGAAGATAG | |
| | GGAGATGATTGAAGAACGCTTGAAAACTTACGCTCATCTCTTCGACGACAAAGTC | |
| | ATGAAACAGCTCAAGAGGCGCCGATATACAGGATGGGGGCGGCTGTCAAGAAAA | |
| | CTGATCAATGGgatcCGAGACAAGCAGAGTGGAAAGACAATCCTGGATTTTCTTAA | |
| | GTCCGATGGATTTGCCAACCGGAACTTCATGCAGTTGATCCATGATGACTCTCTCA | |
| | CCTTTAAGGAGGACATCCAGAAAGCACAAGTTTCTGGCCAGGGGGACAGTCTTCA | |
| | CGAGCACATCGCTAATCTTGCAGGTAGCCCAGCTATCAAAAAGGGAATACTGCAG | |
| | ACCGTTAAGGTCGTGGATGAACTCGTCAAAGTAATGGGAAGGCATAAGCCCGAGA | |
| | ATATCGTTATCGAGATGGCCCGAGAGAACCAAACTACCCAGAAGGGACAGAAGA | |
| | ACAGTAGGGAAAGGATGAAGAGGATTGAAGAGGGTATAAAAGAACTGGGGTCCC | |
| | AAATCCTTAAGGAACACCCAGTTGAAAACACCCAGCTTCAGAATGAGAAGCTCTA | |
| | CCTGTACTACCTGCAGAACGGCAGGGACATGTACGTGGATCAGGAACTGGACATC | |
| | AATCGGCTCTCCGACTACGACGTGGATGCCATCGTGCCCCAGTCTTTTCTCAAAGA | |
| | TGATTCTATTGATAATAAAGTGTTGACAAGATCCGATAAAAATAGAGGGAAGAGT | |
| | GATAACGTCCCCTCAGAAGAAGTTGTCAAGAAAATGAAAAATTATTGGCGGCAGC | |
| | TGCTGAACGCCAAACTGATCACACAACGGAAGTTCGATAATCTGACTAAGGCTGA | |
| | ACGAGGTGGCCTGTCTGAGTTGGATAAAGCCGGCTTCATCAAAAGGCAGCTTGTT | |
| | GAGACACGCCAGATCACCAAgcacGTGGCCCAAATTCTCGATTCACGCATGAACAC | |
| | CAAGTACGATGAAAATGACAAACTGATTCGAGAGGTGAAAGTTATTACTCTGAAG | |
| | TCTAAGCTGGTCTCAGATTTCAGAAAGGACTTTCAGTTTTATAAGGTGAGAGAGAT | |
| | CAACAATTACCACCATGCGCATGATGCCTACCTGAATGCAGTGGTAGGCACTGCA | |
| | CTTATCAAAAAATATCCCAAGCTTGAATCTGAATTTGTTTACGGAGACTATAAAGT | |
| | GTACGATGTTAGGAAAATGATCGCAAAGTCTGAGCAGGAAATAGGCAAGGCCAC | |
| | CGCTAAGTACTTCTTTTACAGCAATATTATGAATTTTTTCAAGACCGAGATTACAC | |
| | TGGCCAATGGAGAGATTCGGAAGCGACCACTTATCGAAACAAACGGAGAAACAG | |
| | GAGAAATCGTGTGGGACAAGGGTAGGGATTTCGCGACAGTCCGGAAGGTCCTGTC | |
| | CATGCCGCAGGTGAACATCGTTAAAAAGACCGAAGTACAGACCGGAGGCTTCTCC | |
| | AAGGAAAGTATCCTCCCGAAAAGGAACAGCGACAAGCTGATCGCACGCAAAAAA | |
| | GATTGGGACCCCAAGAAATACGGCGGATTCGATTCTCCTACAGTCGCTTACAGTG | |
| | TACTGGTTGTGGCCAAAGTGGAGAAAGGGAAGTCTAAAAAACTCAAAAGCGTCA | |
| | AGGAACTGCTGGGCATCACAATCATGGAGCGATCAAGCTTCGAAAAAAACCCCAT | |
| | CGACTTTCTCGAGGCGAAAGGATATAAAGAGGTCAAAAAAGACCTCATCATTAAG | |
| | CTTCCCAAGTACTCTCTCTTTGAGCTTGAAAACGGCCGGAAACGAATGCTCGCTAG | |
| | TGCGGGCGAGCTGCAGAAAGGTAACGAGCTGGCACTGCCCTCTAAATACGTTAAT | |
| | TTCTTGTATCTGGCCAGCCACTATGAAAAGCTCAAAGGGTCTCCCGAAGATAATG | |
| | AGCAGAAGCAGCTGTTCGTGGAACAACACAAACACTACCTTGATGAGATCATCGA | |
| | GCAAATAAGCGAATTCTCCAAAAGAGTGATCCTCGCCGACGCTAACCTCGATAAG | |
| | GTGCTTTCTGCTTACAATAAGCACAGGGATAAGCCCATCAGGGAGCAGGCAGAAA | |
| | ACATTATCCACTTGTTTACTCTGACCAACTTGGGCGCGCCTGCAGCCTTCAAGTAC | |
| | TTCGACACCACCATAGACAGAAAGCGGTACACCTCTACAAAGGAGGTCCTGGACG | |
| | CCACACTGATTCATCAGTCAATTACGGGGCTCTATGAAACAAGAATCGACCTCTCT | |
| | CAGCTCGGTGGAGACAAAAGGCCGGCGGCCACGAAAAAGGCCGGCCAGGCAAAA | |
| | AAGAAAAAGGctagCgatgctaagtcactgactgcctggtcccggacactggtgaccttcaaggatgtgt | |
| | ttgtggacttcaccagggaggagtggaagctgctggacactgctcagcagatcctgtacagaaatgtgat | |
| | gctggagaactataagaacctggtttccttgggttatcagcttactaagccagatgtgatcctccggttgg | |
| | agaagggagaagagccctggctggtggagagagaaattcaccaagagacccatcctgattcagagactgca | |
| | tttgaaatcaaatcatcagttCCGAAAAAGAAACGCAAAGTT | |
| 402 | PKKKRKVGIHGVPAADKKYSIGLAIGTNSVGWAVITDEYKVPSKKFKVLGNTDRHSIK | dSpCas9- |
| | KNLIGALLFDSGETAEATRLKRTARRRYTRRKNRICYLQEIFSNEMAKVDDSFFHRLE | KRAB |
| | ESFLVEEDKKHERHPIFGNIVDEVAYHEKYPTIYHLRKKLVDSTDKADLRLIYLALAH | (AA) |
| | MIKFRGHFLIEGDLNPDNSDVDKLFIQLVQTYNQLFEENPINASGVDAKAILSARLSKS | |
| | RRLENLIAQLPGEKKNGLFGNLIALSLGLTPNFKSNFDLAEDAKLQLSKDTYDDDLDN | |
| | LLAQIGDQYADLFLAAKNLSDAILLSDILRVNTEITKAPLSASMIKRYDEHHQDLTLLK | |
| | ALVRQQLPEKYKEIFFDQSKNGYAGYIDGGASQEEFYKFIKPILEKMDGTEELLVKLN | |
| | REDLLRKQRTFDNGSIPHQIHLGELHAILRRQEDFYPFLKDNREKIEKILTFRIPYYVGP | |
| | LARGNSRFAWMTRKSEETITPWNFEEVVDKGASAQSFIERMTNFDKNLPNEKVLPKH | |
| | SLLYEYFTVYNELTKVKYVTEGMRKPAFLSGEQKKAIVDLLFKTNRKVTVKQLKEDY | |
| | FKKIECFDSVEISGVEDRFNASLGTYHDLLKIIKDKDFLDNEENEDILEDIVLTLTLFED | |
| | REMIEERLKTYAHLFDDKVMKQLKRRRYTGWGRLSRKLINGIRDKQSGKTILDFLKS | |
| | DGFANRNFMQLIHDDSLTFKEDIQKAQVSGQGDSLHEHIANLAGSPAIKKGILQTVKV | |
| | VDELVKVMGRHKPENIVIEMARENQTTQKGQKNSRERMKRIEEGIKELGSQILKEHPV | |
| | ENTQLQNEKLYLYYLQNGRDMYVDQELDINRLSDYDVDAIVPQSFLKDDSIDNKVLT | |
| | RSDKNRGKSDNVPSEEVVKKMKNYWRQLLNAKLITQRKFDNLTKAERGGLSELDKA | |
| | GFIKRQLVETRQITKHVAQILDSRMNTKYDENDKLIREVKVITLKSKLVSDFRKDFQFY | |
| | KVREINNYHHAHDAYLNAVVGTALIKKYPKLESEFVYGDYKVYDVRKMIAKSEQEIG | |
| | KATAKYFFYSNIMNFFKTEITLANGEIRKRPLIETNGETGEIVWDKGRDFATVRKVLS | |
| | MPQVNIVKKTEVQTGGFSKESILPKRNSDKLIARKKDWDPKKYGGFDSPTVAYSVLV | |
| | VAKVEKGKSKKLKSVKELLGITIMERSSFEKNPIDFLEAKGYKEVKKDLIIKLPKYSLF | |
| | ELENGRKRMLASAGELQKGNELALPSKYVNFLYLASHYEKLKGSPEDNEQKQLFVEQ | |
| | HKHYLDEIIEQISEFSKRVILADANLDKVLSAYNKHRDKPIREQAENIIHLFTLTNLGAP | |
| | AAFKYFDTTIDRKRYTSTKEVLDATLIHQSITGLYETRIDLSQLGGDKRPAATKKAGQ | |
| | AKKKKASDAKSLTAWSRTLVTFKDVFVDFTREEWKLLDTAQQILYRNVMLENYKNL | |
| | VSLGYQLTKPDVILRLEKGEEPWLVEREIHQETHPDSETAFEIKSSVPKKKRKV | |
| 403 | NGG | SpCas9 |
| | | PAM |
| 404 | NNGRRT | SaCas9 |
| | | PAM |
| 405 | MKRNYILGLDIGITSVGYGIIDYETRDVIDAGVRLFKEANVENNEGRRSKRGARRLKR | SaCas9 |
| | RRRHRIQRVKKLLFDYNLLTDHSELSGINPYEARVKGLSQKLSEEEFSAALLHLAKRR | (AA) |
| | GVHNVNEVEEDTGNELSTKEQISRNSKALEEKYVAELQLERLKKDGEVRGSINRFKTS | |
| | DYVKEAKQLLKVQKAYHQLDQSFIDTYIDLLETRRTYYEGPGEGSPFGWKDIKEWYE | |
| | MLMGHCTYFPEELRSVKYAYNADLYNALNDLNNLVITRDENEKLEYYEKFQIIENVF | |
| | KQKKKPTLKQIAKEILVNEEDIKGYRVTSTGKPEFTNLKVYHDIKDITARKEIIENAELL | |
| | DQIAKILTIYQSSEDIQEELTNLNSELTQEEIEQISNLKGYTGTHNLSLKAINLILDELWH | |
| | TNDNQIAIFNRLKLVPKKVDLSQQKEIPTTLVDDFILSPVVKRSFIQSIKVINAIIKKYGL | |
| | PNDIIIELAREKNSKDAQKMINEMQKRNRQTNERIEEIIRTTGKENAKYLIEKIKLHDM | |
| | QEGKCLYSLEAIPLEDLLNNPFNYEVDHIIPRSVSFDNSFNNKVLVKQEENSKKGNRTP | |
| | FQYLSSSDSKISYETFKKHILNLAKGKGRISKTKKEYLLEERDINRFSVQKDFINRNLVD | |
| | TRYATRGLMNLLRSYFRVNNLDVKVKSINGGFTSFLRRKWKFKKERNKGYKHHAED | |
| | ALIIANADFIFKEWKKLDKAKKVMENQMFEEKQAESMPEIETEQEYKEIFITPHQIKHI | |
| | KDFKDYKYSHRVDKKPNRELINDTLYSTRKDDKGNTLIVNNLNGLYDKDNDKLKKLI | |
| | NKSPEKLLMYHHDPQTYQKLKLIMEQYGDEKNPLYKYYEETGNYLTKYSKKDNGPV | |
| | IKKIKYYGNKLNAHLDITDDYPNSRNKVVKLSLKPYRFDVYLDNGVYKFVTVKNLDV | |
| | IKKENYYEVNSKCYEEAKKLKKISNQAEFIASFYNNDLIKINGELYRVIGVNNDLLNRI | |
| | EVNMIDITYREYLENMNDKRPPRIIKTIASKTQSIKKYSTDILGNLYEVKSKKHPQIIKK | |
| | G | |
| 406 | KRNYILGLAIGITSVGYGIIDYETRDVIDAGVRLFKEANVENNEGRRSKRGARRLKRRR | dSaCas9 |
| | RHRIQRVKKLLFDYNLLTDHSELSGINPYEARVKGLSQKLSEEEFSAALLHLAKRRGV | (AA) |
| | HNVNEVEEDTGNELSTKEQISRNSKALEEKYVAELQLERLKKDGEVRGSINRFKTSDY | |
| | VKEAKQLLKVQKAYHQLDQSFIDTYIDLLETRRTYYEGPGEGSPFGWKDIKEWYEML | |
| | MGHCTYFPEELRSVKYAYNADLYNALNDLNNLVITRDENEKLEYYEKFQIIENVFKQ | |
| | KKKPTLKQIAKEILVNEEDIKGYRVTSTGKPEFTNLKVYHDIKDITARKEIIENAELLDQ | |
| | IAKILTIYQSSEDIQEELTNLNSELTQEEIEQISNLKGYTGTHNLSLKAINLILDELWHTN | |
| | DNQIAIFNRLKLVPKKVDLSQQKEIPTTLVDDFILSPVVKRSFIQSIKVINAIIKKYGLPN | |
| | DIIIELAREKNSKDAQKMINEMQKRNRQTNERIEEIIRTTGKENAKYLIEKIKLHDMQE | |
| | GKCLYSLEAIPLEDLLNNPFNYEVDHIIPRSVSFDNSFNNKVLVKQEEASKKGNRTPFQ | |
| | YLSSSDSKISYETFKKHILNLAKGKGRISKTKKEYLLEERDINRFSVQKDFINRNLVDTR | |
| | YATRGLMNLLRSYFRVNNLDVKVKSINGGFTSFLRRKWKFKKERNKGYKHHAEDAL | |
| | IIANADFIFKEWKKLDKAKKVMENQMFEEKQAESMPEIETEQEYKEIFITPHQIKHIKD | |
| | FKDYKYSHRVDKKPNRELINDTLYSTRKDDKGNTLIVNNLNGLYDKDNDKLKKLINK | |
| | SPEKLLMYHHDPQTYQKLKLIMEQYGDEKNPLYKYYEETGNYLTKYSKKDNGPVIK | |
| | KIKYYGNKLNAHLDITDDYPNSRNKVVKLSLKPYRFDVYLDNGVYKFVTVKNLDVIK | |
| | KENYYEVNSKCYEEAKKLKKISNQAEFIASFYNNDLIKINGELYRVIGVNNDLLNRIEV | |
| | NMIDITYREYLENMNDKRPPRIIKTIASKTQSIKKYSTDILGNLYEVKSKKHPQIIKKG | |
| 407 | MDKKYSIGLDIGTNSVGWAVITDEYKVPSKKFKVLGNTDRHSIKKNLIGALLFDSGET | SpCas9 |
| | AEATRLKRTARRRYTRRKNRICYLQEIFSNEMAKVDDSFFHRLEESFLVEEDKKHERH | (AA) |
| | PIFGNIVDEVAYHEKYPTIYHLRKKLVDSTDKADLRLIYLALAHMIKFRGHFLIEGDLN | |
| | PDNSDVDKLFIQLVQTYNQLFEENPINASGVDAKAILSARLSKSRRLENLIAQLPGEKK | |
| | NGLFGNLIALSLGLTPNFKSNFDLAEDAKLQLSKDTYDDDLDNLLAQIGDQYADLFLA | |
| | AKNLSDAILLSDILRVNTEITKAPLSASMIKRYDEHHQDLTLLKALVRQQLPEKYKEIF | |
| | FDQSKNGYAGYIDGGASQEEFYKFIKPILEKMDGTEELLVKLNREDLLRKQRTFDNGS | |
| | IPHQIHLGELHAILRRQEDFYPFLKDNREKIEKILTFRIPYYVGPLARGNSRFAWMTRKS | |
| | EETITPWNFEEVVDKGASAQSFIERMTNFDKNLPNEKVLPKHSLLYEYFTVYNELTKV | |
| | KYVTEGMRKPAFLSGEQKKAIVDLLFKTNRKVTVKQLKEDYFKKIECFDSVEISGVED | |
| | RFNASLGTYHDLLKIIKDKDFLDNEENEDILEDIVLTLTLFEDREMIEERLKTYAHLFD | |
| | DKVMKQLKRRRYTGWGRLSRKLINGIRDKQSGKTILDFLKSDGFANRNFMQLIHDDS | |
| | LTFKEDIQKAQVSGQGDSLHEHIANLAGSPAIKKGILQTVKVVDELVKVMGRHKPENI | |
| | VIEMARENQTTQKGQKNSRERMKRIEEGIKELGSQILKEHPVENTQLQNEKLYLYYLQ | |
| | NGRDMYVDQELDINRLSDYDVDHIVPQSFLKDDSIDNKVLTRSDKNRGKSDNVPSEE | |
| | VVKKMKNYWRQLLNAKLITQRKFDNLTKAERGGLSELDKAGFIKRQLVETRQITKHV | |
| | AQILDSRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYSNIMNF | |
| | FKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSV | |
| | KELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKR | |
| | VILADANLDKVLSAYNKHRDKPIREQAENIIHLFTLTNLGAPAAFKYFDTTIDRKRYTS | |
| | TKEVLDATLIHQSITGLYETRIDLSQLGGD | |
| 408 | DKKYSIGLAIGTNSVGWAVITDEYKVPSKKFKVLGNTDRHSIKKNLIGALLFDSGETA | dSpCas9 |
| | EATRLKRTARRRYTRRKNRICYLQEIFSNEMAKVDDSFFHRLEESFLVEEDKKHERHPI | (AA) |
| | FGNIVDEVAYHEKYPTIYHLRKKLVDSTDKADLRLIYLALAHMIKFRGHFLIEGDLNP | |
| | DNSDVDKLFIQLVQTYNQLFEENPINASGVDAKAILSARLSKSRRLENLIAQLPGEKKN | |
| | GLFGNLIALSLGLTPNFKSNFDLAEDAKLQLSKDTYDDDLDNLLAQIGDQYADLFLAA | |
| | KNLSDAILLSDILRVNTEITKAPLSASMIKRYDEHHQDLTLLKALVRQQLPEKYKEIFF | |
| | DQSKNGYAGYIDGGASQEEFYKFIKPILEKMDGTEELLVKLNREDLLRKQRTFDNGSI | |
| | PHQIHLGELHAILRRQEDFYPFLKDNREKIEKILTFRIPYYVGPLARGNSRFAWMTRKS | |
| | EETITPWNFEEVVDKGASAQSFIERMTNFDKNLPNEKVLPKHSLLYEYFTVYNELTKV | |
| | KYVTEGMRKPAFLSGEQKKAIVDLLFKTNRKVTVKQLKEDYFKKIECFDSVEISGVED | |
| | RFNASLGTYHDLLKIIKDKDFLDNEENEDILEDIVLTLTLFEDREMIEERLKTYAHLFD | |
| | DKVMKQLKRRRYTGWGRLSRKLINGIRDKQSGKTILDFLKSDGFANRNFMQLIHDDS | |
| | LTFKEDIQKAQVSGQGDSLHEHIANLAGSPAIKKGILQTVKVVDELVKVMGRHKPENI | |
| | VIEMARENQTTQKGQKNSRERMKRIEEGIKELGSQILKEHPVENTQLQNEKLYLYYLQ | |
| | NGRDMYVDQELDINRLSDYDVDAIVPQSFLKDDSIDNKVLTRSDKNRGKSDNVPSEE | |
| | VVKKMKNYWRQLLNAKLITQRKFDNLTKAERGGLSELDKAGFIKRQLVETRQITKHV | |
| | AQILDSRMNTKYDENDKLIREVKVITLKSKLVSDFRKDFQFYKVREINNYHHAHDAY | |
| | LNAVVGTALIKKYPKLESEFVYGDYKVYDVRKMIAKSEQEIGKATAKYFFYSNIMNF | |
| | FKTEITLANGEIRKRPLIETNGETGEIVWDKGRDFATVRKVLSMPQVNIVKKTEVQTG | |
| | GFSKESILPKRNSDKLIARKKDWDPKKYGGFDSPTVAYSVLVVAKVEKGKSKKLKSV | |
| | KELLGITIMERSSFEKNPIDFLEAKGYKEVKKDLIIKLPKYSLFELENGRKRMLASAGEL | |
| | QKGNELALPSKYVNFLYLASHYEKLKGSPEDNEQKQLFVEQHKHYLDEIIEQISEFSKR | |
| | VILADANLDKVLSAYNKHRDKPIREQAENIIHLFTLTNLGAPAAFKYFDTTIDRKRYTS | |
| | TKEVLDATLIHQSITGLYETRIDLSQLGGD | |
| 409 | DALDDFDLDMLGSDALDDFDLDMLGSDALDDFDLDMLGSDALDDFDLDM | VP64 (AA) |
| 410 | KRPAATKKAGQAKKKK | NLS- |
| | | nucleo- |
| | | plasmin (AA) |
| 411 | PTQAGEGTLSEALLQLQFDDEDLGALLGNSTDPAVFTDLASVDNSEFQQLLNQGIPVA | p65AD |
| | PHTTEPMLMEYPEAITRLVTGAQRPPDPAPAPLGAPGLPNGLLSGDEDFSSIADMDFSA | (AA) |
| | LLSQISS | |
| 412 | RDSREGMFLPKPEAGSAISDVFEGREVCQPKRIRPFHPPGSPWANRPLPASLAPTPTGP | Rta domain |
| | VHEPVGSLTPAPVPQPLDPAPAVTPEASHLLEDPDEETSQAVKALREMADTVIPQKEE | (AA) |
| | AAICGQMDLSHPPPRGHLDELTTTLESMTEDLNLDSPLTPELNEILDTFLNDECLLHAM | |
| | HISTGLSIFDTSLF | |
| 413 | MAENVVEPGPPSAKRPKLSSPALSASASDGTDFGSLFDLEHDLPDELINSTELGLTNGG | p300 |
| | DINQLQTSLGMVQDAASKHKQLSELLRSGSSPNLNMGVGGPGQVMASQAQQSSPGL | domain |
| | GLINSMVKSPMTQAGLTSPNMGMGTSGPNQGPTQSTGMMNSPVNQPAMGMNTGMN | (AA) |
| | AGMNPGMLAAGNGQGIMPNQVMNGSIGAGRGRQNMQYPNPGMGSAGNLLTEPLQQ | |
| | GSPQMGGQTGLRGPQPLKMGMMNNPNPYGSPYTQNPGQQIGASGLGLQIQTKTVLS | |
| | NNLSPFAMDKKAVPGGGMPNMGQQPAPQVQQPGLVTPVAQGMGSGAHTADPEKRK | |
| | LIQQQLVLLLHAHKCQRREQANGEVRQCNLPHCRTMKNVLNHMTHCQSGKSCQVA | |
| | HCASSRQIISHWKNCTRHDCPVCLPLKNAGDKRNQQPILTGAPVGLGNPSSLGVGQQS | |
| | APNLSTVSQIDPSSIERAYAALGLPYQVNQMPTQPQVQAKNQQNQQPGQSPQGMRPM | |
| | SNMSASPMGVNGGVGVQTPSLLSDSMLHSAINSQNPMMSENASVPSMGPMPTAAQP | |
| | STTGIRKQWHEDITQDLRNHLVHKLVQAIFPTPDPAALKDRRMENLVAYARKVEGD | |
| | MYESANNRAEYYHLLAEKIYKIQKELEEKRRTRLQKQNMLPNAAGMVPVSMNPGPN | |
| | MGQPQPGMTSNGPLPDPSMIRGSVPNQMMPRITPQSGLNQFGQMSMAQPPIVPRQTPP | |
| | LQHHGQLAQPGALNPPMGYGPRMQQPSNQGQFLPQTQFPSQGMNVTNIPLAPSSGQA | |
| | PVSQAQMSSSSCPVNSPIMPPGSQGSHIHCPQLPQPALHQNSPSPVPSRTPTPHHTPPSIG | |
| | AQQPPATTIPAPVPTPPAMPPGPQSQALHPPPRQTPTPPTTQLPQQVQPSLPAAPSADQP | |
| | QQQPRSQQSTAASVPTPTAPLLPPQPATPLSQPAVSIEGQVSNPPSTSSTEVNSQAIAEK | |
| | QPSQEVKMEAKMEVDQPEPADTQPEDISESKVEDCKMESTETEERSTELKTEIKEEED | |
| | QPSTSATQSSPAPGQSKKKIFKPEELRQALMPTLEALYRQDPESLPFRQPVDPQLLGIPD | |
| | YFDIVKSPMDLSTIKRKLDTGQYQEPWQYVDDIWLMFNNAWLYNRKTSRVYKYCSK | |
| | LSEVFEQEIDPVMQSLGYCCGRKLEFSPQTLCCYGKQLCTIPRDATYYSYQNRYHFCE | |
| | KCFNEIQGESVSLGDDPSQPQTTINKEQFSKRKNDTLDPELFVECTECGRKMHQICVLH | |
| | HEIIWPAGFVCDGCLKKSARTRKENKFSAKRLPSTRLGTFLENRVNDFLRRQNHPESG | |
| | EVTVRVVHASDKTVEVKPGMKARFVDSGEMAESFPYRTKALFAFEEIDGVDLCFFGM | |
| | HVQEYGSDCPPPNQRRVYISYLDSVHFFRPKCLRTAVYHEILIGYLEYVKKLGYTTGHI | |
| | WACPPSEGDDYIFHCHPPDQKIPKPKRLQEWYKKMLDKAVSERIVHDYKDIFKQATE | |
| | DRLTSAKELPYFEGDFWPNVLEESIKELEQEEEERKREENTSNESTDVTKGDSKNAKK | |
| | KNNKKTSKNKSSLSRGNKKKPGMPNVSNDLSQKLYATMEKHKEVFFVIRLIAGPAAN | |
| | SLPPIVDPDPLIPCDLMDGRDAFLTLARDKHLEFSSLRRAQWSTMCMLVELHTQSQDR | |
| | FVYTCNECKHHVETRWHCTVCEDYDLCITCYNTKNHDHKMEKLGLGLDDESNNQQ | |
| | AAATQSPGDSRRLSIQRCIQSLVHACQCRNANCSLPSCQKMKRVVQHTKGCKRKTNG | |
| | GCPICKQLIALCCYHAKHCQENKCPVPFCLNIKQKLRQQQLQHRLQQAQMLRRRMAS | |
| | MQRTGVVGQQQGLPSPTPATPTTPTGQQPTTPQTPQPTSQPQPTPPNSMPPYLPRTQA | |
| | AGPVSQGKAAGQVTPPTPPQTAQPPLPGPPPAAVEMAMQIQRAAETQRQMAHVQIFQ | |
| | RPIQHQMPPMTPMAPMGMNPPPMTRGPSGHLEPGMGPTGMQQQPPWSQGGLPQPQQ | |
| | LQSGMPRPAMMSVAQHGQPLNMAPQPGLGQVGISPLKPGTVSQQALQNLLRTLRSPS | |
| | SPLQQQQVLSILHANPQLLAAFIKQRAAKYANSNPQPIPGQPGMPQGQPGLQPPTMPG | |
| | QQGVHSNPAMQNMNPMQAGVQRAGLPQQQPQQQLQPPMGGMSPQAQQMNMNHN | |
| | TMPSQFRDILRRQQMMQQQQQQGAGPGIGPGMANHNQFQQPQGVGYPPQQQQRMQ | |
| | HHMQQMQQGNMGQIGQLPQALGAEAGASLQAYQQRLLQQQMGSPVQPNPMSPQQ | |
| | HMLPNQAQSPHLQGQQIPNSLSNQVRSPQPVPSPRPQSQPPHSSPSPRMQPQPSPHHVS | |
| | PQTSSPHPGLVAAQANPMEQGHFASPDQNSMLSQLASNPGMANLHGASATDLGLSTD | |
| | NSDLNSNLSQSTLDIH | |
| 414 | GFSVDTSALLDLFSPSVTVPDMSLPDLDSSLASIQELLSPQEPPRPPEAENSSPDSGKQL | HSF1 |
| | VHYTAQPLFLLDPGSVDTGSNDLPVLFELGEGSYFSEGDGFAEDPTISLLTGSEPPKAK | domain |
| | DPTVS | (AA) |
| 415 | DALDDFDLDMLGSDALDDFDLDMLGSDALDDFDLDMLGSDALDDFDLDMLGSPKK | VPR (AA) |
| | KRKVGSQYLPDTDDRHRIEEKRKRTYETFKSIMKKSPFSGPTDPRPPPRRIAVPSRSSAS | |
| | VPKPAPQPYPFTSSLSTINYDEFPTMVFPSGQISQASALAPAPPQVLPQAPAPAPAPAM | |
| | VSALAQAPAPVPVLAPGPPQAVAPPAPKPTQAGEGTLSEALLQLQFDDEDLGALLGNS | |
| | TDPAVFTDLASVDNSEFQQLLNQGIPVAPHTTEPMLMEYPEAITRLVTGAQRPPDPAP | |
| | APLGAPGLPNGLLSGDEDFSSIADMDFSALLSQISSGSGSGSRDSREGMFLPKPEAGSAI | |
| | SDVFEGREVCQPKRIRPFHPPGSPWANRPLPASLAPTPTGPVHEPVGSLTPAPVPQPLD | |
| | PAPAVTPEASHLLEDPDEETSQAVKALREMADTVIPQKEEAAICGQMDLSHPPPRGHL | |
| | DELTTTLESMTEDLNLDSPLTPELNEILDTFLNDECLLHAMHISTGLSIFDTSLF | |
| 416 | DALDDFDLDMLGSDALDDFDLDMLGSDALDDFDLDMLGSDALDDFDLDMLGSLPSA | VPH (AA) |
| | SVEFEGSGGPSGQISNQALALAPSSAPVLAQTMVPSSAMVPLAQPPAPAPVLTPGPPQS | |
| | LSAPVPKSTQAGEGTLSEALLHLQFDADEDLGALLGNSTDPGVFTDLASVDNSEFQQL | |
| | LNQGVSMSHSTAEPMLMEYPEAITRLVTGSQRPPDPAPTPLGTSGLPNGLSGDEDFSSI | |
| | ADMDFSALLSQISSSGQGGGGSGFSVDTSALLDLFSPSVTVPDMSLPDLDSSLASIQEL | |
| | LSPQEPPRPPEAENSSPDSGKQLVHYTAQPLFLLDPGSVDTGSNDLPVLFELGEGSYFS | |
| | EGDGFAEDPTISLLTGSEPPKAKDPTVS | |
| 417 | LPTCSCLDRVIQKDKGPYYTHLGAGPSVAAVREIMENRYGQKGNAIRIEIVVYTGKEG | TET1 |
| | KSSHGCPIAKWVLRRSSDEEKVLCLVRQRTGHHCPTAVMVVLIMVWDGIPLPMADRL | catalytic |
| | YTELTENLKSYNGHPTDRRCTLNENRTCTCQGIDPETCGASFSFGCSWSMYFNGCKFG | domain |
| | RSPSPRRFRIDPSSPLHEKNLEDNLQSLATRLAPIYKQYAPVAYQNQVEYENVARECRL | (AA) |
| | GSKEGRPFSGVTACLDFCAHPHRDIHNMNNGSTVVCTLTREDNRSLGVIPQDEQLHVL | |
| | PLYKLSDTDEFGSKEGMEAKIKSGAIEVLAPRRKKRTCFTQPVPRSGKKRAAMMTEV | |
| | LAHKIRAVEKKPIPRIKRKNNSTTTNNSKPSSLPTLGSNTETVQPEVKSETEPHFILKSSD | |
| | NTKTYSLMPSAPHPVKEASPGFSWSPKTASATPAPLKNDATASCGFSERSSTPHCTMP | |
| | SGRLSGANAAAADGPGISQLGEVAPLPTLSAPVMEPLINSEPSTGVTEPLTPHQPNHQP | |
| | SFLTSPQDLASSPMEEDEQHSEADEPPSDEPLSDDPLSPAEEKLPHIDEYWSDSEHIFLD | |
| | ANIGGVAIAPAHGSVLIECARRELHATTPVEHPNRNHPTRLSLVFYQHKNLNKPQHGF | |
| | ELNKIKFEAKEAKNKKMKASEQKDQAANEGPEQSSEVNELNQIPSHKALTLTHDNVV | |
| | TVSPYALTHVAGPYNHWV | |
| 418 | GGGGS | GGGGS |
| | | linker (AA) |
| 419 | GGGGG | GGGGG |
| | | linker (AA) |
| 420 | GGAGG | GGAGG |
| | | linker (AA) |
| 421 | GGGGSSS | GGGGSSS |
| | | linker (AA) |
| 422 | GGGGAAA | GGGGAAA |
| | | linker |
| | | (AA) |
| 423 | PKKKRKV | NLS- |
| | | SV40 (AA) |
| 424 | PAAKRVKLD | NLS- |
| | | cMyc (AA) |
| 425 | RQRRNELKRSP | NLS- |
| | | cMyc (AA) |
| 426 | NQSSNFGPMKGGNFGGRSSGPYGGGGQYFAKPRNQGGY | NLS- |
| | | hRNPA1M9 |
| | | (AA) |
| 427 | RMRIZFKNKGKDTAELRRRRVEVSVELRKAKKDEQILKRRNV | NLS-IBB |
| | | domain |
| | | importin- |
| | | alpha (AA) |
| 428 | VSRKRPRP | NLS- |
| | | myoma T |
| | | protein |
| | | (AA) |
| 429 | PPKKARED | NLS- |
| | | myoma T |
| | | protein |
| | | (AA) |
| 430 | PQPKKKPL | NLS- |
| | | human p53 |
| | | (AA) |
| 431 | SALIKKKKKMAP | NLS- |
| | | mouse c- |
| | | abl IV |
| | | (AA) |
| 432 | DRLRR | NLS- |
| | | influenza |
| | | NS1 (AA) |
| 433 | PKQKKRK | NLS- |
| | | influenza |
| | | NS1 (AA) |
| 434 | RKLKKKIKKL | NLS- |
| | | hepatitis |
| | | delta |
| | | antigen |
| | | (AA) |
| 435 | REKKKFLKRR | NLS- |
| | | mouse Mx1 |
| | | (AA) |
| 436 | KRKGDEVDGVDEVAKKKSKK | NLS- |
| | | human |
| | | poly(ADP- |
| | | ribose) |
| | | polymerase |
| | | (AA) |
| 437 | RKCLQAGMNLEARKTKK | NLS- |
| | | gluco- |
| | | corticoid |
| | | (AA) |
| 438 | LLPKNYHLENEVARLKKLVGER | SunTag |
| | | GCN4 |
| | | peptide |
| | | (AA) |
| 439 | GGSGG | GGSGG |
| | | linker (AA) |
| 440 | MLSGKKAAAAAAAAAAAATGTEAGPGTAGGSENGSEVAAQPAGLSGPAEVGPGAV | LSD1 (AA) |
| | GERTPRKKEPPRASPPGGLAEPPGSAGPQAGPTVVPGSATPMETGIAETPEGRRTSRRK | |
| | RAKVEYREMDESLANLSEDEYYSEEERNAKAEKEKKLPPPPPQAPPEEENESEPEEPSG | |
| | VEGAAFQSRLPHDRMTSQEAACFPDIISGPQQTQKVFLFIRNRTLQLWLDNPKIQLTFE | |
| | ATLQQLEAPYNSDTVLVHRVHSYLERHGLINFGIYKRIKPLPTKKTGKVIIIGSGVSGLA | |
| | AARQLQSFGMDVTLLEARDRVGGRVATFRKGNYVADLGAMVVTGLGGNPMAVVS | |
| | KQVNMELAKIKQKCPLYEANGQAVPKEKDEMVEQEFNRLLEATSYLSHQLDFNVLN | |
| | NKPVSLGQALEVVIQLQEKHVKDEQIEHWKKIVKTQEELKELLNKMVNLKEKIKELH | |
| | QQYKEASEVKPPRDITAEFLVKSKHRDLTALCKEYDELAETQGKLEEKLQELEANPPS | |
| | DVYLSSRDRQILDWHFANLEFANATPLSTLSLKHWDQDDDFEFTGSHLTVRNGYSCV | |
| | PVALAEGLDIKLNTAVRQVRYTASGCEVIAVNTRSTSQTFIYKCDAVLCTLPLGVLKQ | |
| | QPPAVQFVPPLPEWKTSAVQRMGFGNLNKVVLCFDRVFWDPSVNLFGHVGSTTASR | |
| | GELFLFWNLYKAPILLALVAGEAAGIMENISDDVIVGRCLAILKGIFGSSAVPQPKETV | |
| | VSRWRADPWARGSYSYVAAGSSGNDYDLMAQPITPGPSIPGAPQPIPRLFFAGEHTIR | |
| | NYPATVHGALLSGLREAGRIADQFLGAMYTLPRQATPGVPAQQSPSM | |
| 441 | NNNNGATT | PAM (N. |
| | | meningitidis) |
| 442 | NNNNRYAC | PAM |
| | | (C. jejuni) |
| 443 | NNAGAAW | PAM (S. |
| | | thermophilus) |
| 444 | NAAAAC | PAM (T. |
| | | denticola) |
| 445 | TTTV | PAM |
| | | (Cpf1) |
| 446 | NGAN | PAM |
| 447 | NGNG | PAM |
| 448 | NGAG | PAM |
| 449 | NGCG | PAM |

1. An epigenetic-modifying DNA-targeting system,
said DNA-targeting system comprising a fusion protein comprising:
(a) a DNA-targeting domain capable of being targeted to a target site in a gene or regulatory DNA element thereof in a T cell; and
(b) at least one effector domain capable of increasing transcription of the gene;
wherein increased transcription of the gene promotes a stem cell-like memory T-cell phenotype.

2. The epigenetic-modifying DNA-targeting system of claim 1, wherein the DNA-targeting system is not able to introduce a genetic disruption or a DNA break at or near the target site.

3. The epigenetic-modifying DNA-targeting system of claim 1 or claim 2, wherein the DNA-targeting domain comprises a Clustered Regularly Interspaced Short Palindromic Repeats associated (Cas)-guide RNA (gRNA) combination comprising (a) a Cas protein or a variant thereof and (b) at least one gRNA; a zinc finger protein (ZFP); a transcription activator-like effector (TALE); a meganuclease; a homing endonuclease; or an I-SceI enzyme or a variant thereof, optionally wherein the DNA-targeting domain comprises a catalytically inactive variant of any of the foregoing.

4. The epigenetic-modifying DNA-targeting system of any of claims 1-3, wherein the DNA-targeting domain comprises a Cas-gRNA combination comprising (a) a Cas protein or a variant thereof and (b) at least one gRNA.

5. An epigenetic-modifying DNA-targeting system,
said DNA-targeting system comprising:
(a) a fusion protein comprising a Clustered Regularly Interspaced Short Palindromic Repeats associated (Cas) protein or variant thereof and at least one effector domain capable of increasing transcription of a gene is a T cell; and
(b) at least one gRNA that targets the Cas protein or variant thereof of the fusion protein to a target site in the gene or regulatory DNA element thereof,
wherein increased transcription of the gene promotes a stem cell-like memory T-cell phenotype.

6. The epigenetic-modifying DNA-targeting system of any of claims 1-5, wherein the stem cell-like memory T cell phenotype comprises one or more cell-surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−, or combinations thereof.

7. The epigenetic-modifying DNA-targeting system of any of claims 1-6, wherein the stem cell-like memory T cell phenotype comprises expression of CCR7 and/or CD27.

8. The epigenetic-modifying DNA-targeting system of any of claims 1-7, wherein the stem cell-like memory T cell phenotype comprises expression of CCR7 and CD27.

9. The epigenetic-modifying DNA-targeting system of any of claims 1-8, wherein the stem cell-like memory T cell phenotype is characterized by polyfunctional activity of the T cell to produce two or more cytokines following stimulation of the T cell with a stimulatory agent, optionally wherein the two or more cytokines are selected from among interferon-gamma (IFN-gamma), interleukin 2 (IL-2), and TNF-alpha.

10. The epigenetic-modifying DNA-targeting system of any of claims 3-9, wherein at least one gRNA is capable of complexing with the Cas protein or variant thereof, and targeting the Cas protein or the variant thereof to the target site.

11. The epigenetic-modifying DNA-targeting system of any of claims 3-10, wherein the at least one gRNA comprises a gRNA spacer sequence that is capable of hybridizing to the target site or is complementary to the target site.

12. The epigenetic-modifying DNA-targeting system of any of claims 3-11, wherein the Cas protein or a variant thereof is a Cas9 protein or a variant thereof.

13. The epigenetic-modifying DNA-targeting system of any of claims 3-11, wherein the Cas protein or a variant thereof is a Cas12 protein or a variant thereof.

14. The epigenetic-modifying DNA-targeting system of any of claims 3-13, wherein the Cas protein or a variant thereof is a variant Cas protein, wherein the variant Cas protein lacks nuclease activity or is a deactivated Cas (dCas) protein.

15. The epigenetic-modifying DNA-targeting system of claim 14, wherein the variant Cas protein is a variant Cas9 protein that lacks nuclease activity or that is a deactivated Cas9 (dCas9) protein.

16. The epigenetic-modifying DNA-targeting system of claim 12, wherein the Cas9 protein or a variant thereof is a Staphylococcus aureus Cas9 (SaCas9) protein or a variant thereof.

17. The epigenetic-modifying DNA-targeting system of claim 15, wherein the variant Cas9 is a Staphylococcus aureus dCas9 protein (dSaCas9) that comprises at least one amino acid mutation selected from D10A and N580A, with reference to numbering of positions of SEQ ID NO: 405.

18. The epigenetic-modifying DNA-targeting system of claim 15 or claim 17, wherein the variant Cas9 protein comprises the sequence set forth in SEQ ID NO: 406, or an amino acid sequence that has at least 90%, 91%, 92%, 93%, 94%, 95%, 96%, 97%, 98%, or 99% sequence identity thereto.

19. The epigenetic-modifying DNA-targeting system of claim 12, wherein the Cas9 protein or variant thereof is a Streptococcus pyogenes Cas9 (SpCas9) protein or a variant thereof.

20. The epigenetic-modifying DNA-targeting system of claim 15, wherein the variant Cas9 is a Streptococcus pyogenes dCas9 (dSpCas9) protein that comprises at least one amino acid mutation selected from D10A and H840A, with reference to numbering of positions of SEQ ID NO: 407.

21. The epigenetic-modifying DNA-targeting system of claim 15 or claim 20, wherein the variant Cas9 protein comprises the sequence set forth in SEQ ID NO: 408, or an amino acid sequence that has at least 90%, 91%, 92%, 93%, 94%, 95%, 96%, 97%, 98%, or 99% sequence identity thereto.

22. The epigenetic-modifying DNA-targeting system of any of claims 1-21, wherein the regulatory DNA element is an enhancer or a promoter.

23. The epigenetic-modifying DNA-targeting system of any of claims 1-22, wherein the gene is a DNA-binding gene.

24. The DNA-targeting system of any of claims 1-23, wherein the gene is selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10.

25. The epigenetic-modifying DNA-targeting system of any of claims 1-24, wherein the target site comprises the sequence set forth in any one of SEQ ID NOS: 1-132, a contiguous portion thereof of at least 14 nucleotides (nt), or a complementary sequence of any of the foregoing.

26. The epigenetic-modifying DNA-targeting system of any of claims 3-25, wherein the at least one gRNA comprises a gRNA spacer sequence comprising the sequence set forth in SEQ ID NO:133-264, or a contiguous portion thereof of at least 14 nt.

27. The epigenetic-modifying DNA-targeting system of claim 26, wherein the at least one gRNA further comprises the sequence set forth in SEQ ID NO:398.

28. The epigenetic-modifying DNA-targeting system of any of claims 3-27, wherein the at least one gRNA comprises a gRNA that comprises the sequence set forth in any one of SEQ ID NOS: 265-396, optionally wherein the at least one gRNA is the gRNA set forth in any one of SEQ ID NOS: 265-396.

29. The epigenetic-modifying DNA-targeting system of any of claims 1-24, wherein the gene is selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23.

30. The epigenetic-modifying DNA-targeting system of any of claims 1-24 and 29, wherein the target site comprises the sequence set forth in any one of SEQ ID NOS: 1-28, a contiguous portion thereof of at least 14 nucleotides (nt), or a complementary sequence of any of the foregoing.

31. The epigenetic-modifying DNA-targeting system of any of claims 3-24, 29, and 30, wherein the at least one gRNA comprises a gRNA spacer sequence comprising the sequence set forth in SEQ ID NO:133-160, or a contiguous portion thereof of at least 14 nt.

32. The epigenetic-modifying DNA-targeting system of claim 31, wherein the at least one gRNA further comprises the sequence set forth in SEQ ID NO:398.

33. The epigenetic-modifying DNA-targeting system of any of claims 3-24 and 29-32, wherein the at least one gRNA comprises a gRNA that comprises the sequence set forth in any one of SEQ ID NOS: 265-292, optionally wherein the at least one gRNA is the gRNA set forth in any one of SEQ ID NOS: 265-292.

34. The epigenetic-modifying DNA-targeting system of any of claims 1-24, wherein the gene is selected from the list consisting of: DDIT3, HES2, HKR1, PLAG1, PRDM4, TEF, TSHZ1, ZNF141, ZNF562, ZNF691, ZNF692, and ZSCAN5A.

35. The DNA-targeting system of any of claims 1-24 and 34, wherein the target site comprises the sequence set forth in any one of SEQ ID NOS: 1-12, a contiguous portion thereof of at least 14 nucleotides (nt), or a complementary sequence of any of the foregoing.

36. The DNA-targeting system of any of claims 3-24, 34, and 35, wherein the at least one gRNA comprises a gRNA spacer sequence comprising the sequence set forth in SEQ ID NO: 133-144, or a contiguous portion thereof of at least 14 nt.

37. The DNA-targeting system of claim 36, wherein the at least one gRNA further comprises the sequence set forth in SEQ ID NO:398.

38. The DNA-targeting system of any of claims 3-24 and 34-37, wherein the at least one gRNA comprises a gRNA that comprises the sequence set forth in any one of SEQ ID NOS: 265-276, optionally wherein the at least one gRNA is the gRNA set forth in any one of SEQ ID NOS: 265-276.

39. The DNA-targeting system of any of claims 3-38, wherein the gRNA spacer sequence is between 14 nt and 24 nt, or between 16 nt and 22 nt in length.

40. The DNA-targeting system of any of claims 3-39, wherein the gRNA spacer sequence is 18 nt, 19 nt, 20 nt, 21 nt or 22 nt in length.

41. The DNA-targeting system of any of claims 3-40, wherein the gRNA comprises modified nucleotides for increased stability.

42. The DNA-targeting system of any of claims 1-41, wherein the at least one effector domain induces, catalyzes, or leads to transcription activation, transcription co-activation, transcription elongation, or increased transcription of the gene.

43. The DNA-targeting system of any of claims 1-42, wherein the at least one effector domain induces transcription activation.

44. The DNA-targeting system of any of claims 1-43, wherein the at least one effector domain comprises at least one VP16 domain, or a VP16 tetramer (“VP64”) or a variant thereof.

45. The DNA-targeting system of any of claims 1-44, wherein the at least one effector domain comprises the sequence set forth in SEQ ID NO: 409, a portion thereof, or an amino acid sequence that has at least 90%, 91%, 92%, 93%, 94%, 95%, 96%, 97%, 98%, or 99% sequence identity to any of the foregoing.

46. The DNA-targeting system of any of claims 1-43, wherein at least one effector domain is selected from a p65 activation domain, a p300 domain, Rta, CBP, VPR, VPH, HSF1, a TET protein, optionally wherein the TET protein is TET1, an ERF protein, optionally wherein the ERF protein is ERF1 or ERF3, LSD1, SunTag, or a domain, portion, or variant of any of the foregoing.

47. The DNA-targeting system of any of claims 1-43 and 46, wherein at least one effector domain comprises a sequence selected from any one of SEQ ID NOS: 411-417, 438, or 440, or a domain thereof, a portion thereof, or an amino acid sequence that has at least 90%, 91%, 92%, 93%, 94%, 95%, 96%, 97%, 98%, or 99% sequence identity to any of the foregoing.

48. The DNA-targeting system of any of claims 1-47, wherein the at least one effector domain is fused to the N-terminus, the C-terminus, or both the N-terminus and the C-terminus, of the DNA-targeting domain or a component thereof.

49. The DNA-targeting system of any of claims 1-48, further comprising one or more nuclear localization signals (NLS).

50. The DNA-targeting system of claim 49, further comprising one or more linkers connecting two or more of: the DNA-targeting domain, the at least one effector domain, and the one or more nuclear localization signals.

51. The DNA-targeting system of any of claims 1-50, wherein the fusion protein comprises the sequence set forth in SEQ ID NO:400, or an amino acid sequence that has at least 90%, 91%, 92%, 93%, 94%, 95%, 96%, 97%, 98%, or 99% sequence identity thereto.

52. The DNA-targeting system of any of claims 1-51, wherein increased transcription of the gene further promotes increased production of IL-2 by the T cell.

53. The DNA-targeting system of any of claims 1-52, wherein the epigenetic-modifying DNA-targeting system increases expression of the gene in a T cell by a log 2 fold-change of at or greater than 1.0.

54. The DNA-targeting system of any of claims 1-53, wherein the epigenetic-modifying DNA-targeting system reduces surface expression of a T cell exhaustion marker selected from the group consisting of PD-1, CTLA-4, TIM-3, TOX, LAG-3, BTLA, 2B4, CD160, CD39, VISTA, and TIGIT.

55. A guide RNA (gRNA) that binds a target site in a gene or regulatory DNA element thereof in a T cell, wherein increased transcription of the gene, when targeted by an epigenetic-modifying DNA-targeting system comprising the gRNA, promotes a stem cell-like memory T cell phenotype.

56. The gRNA of claim 55, wherein the stem cell-like memory T cell phenotype comprises one or more cell-surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−.

57. The gRNA of claim 55 or claim 56, wherein the stem cell-like memory T cell phenotype comprises expression of CCR7 and/or CD27.

58. The gRNA of claim 55 or claim 56, wherein the stem cell-like memory T cell phenotype comprises expression of CCR7 and CD27.

59. The gRNA of any of claims 55-58, wherein the stem cell-like memory T cell phenotype is characterized by polyfunctional activity of the T cell to produce two or more cytokines following stimulation of the T cell with a stimulatory agent, optionally wherein the two or more cytokines are selected from among interferon-gamma (IFN-gamma), interleukin 2 (IL-2), and TNF-alpha.

60. The gRNA of any of claims 55-59, wherein the gene is selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10.

61. A guide RNA (gRNA) that binds a target site in a gene or regulatory DNA element thereof in a T cell, wherein increased transcription of the gene, when targeted by an epigenetic-modifying DNA-targeting system comprising the gRNA, promotes a stem cell-like memory T cell phenotype, and wherein the gene is selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10.

62. The gRNA of any of claims 55-61, wherein the target site is in a regulatory DNA element and the regulatory DNA element is an enhancer or a promoter.

63. The gRNA of any of claims 55-62, wherein the target site comprises the sequence set forth in any one of SEQ ID NOS: 1-132, a contiguous portion thereof of at least 14 nucleotides (nt), or a complementary sequence of any of the foregoing.

64. The gRNA of any of claims 55-63, wherein the gRNA comprises a gRNA spacer sequence comprising the sequence set forth in SEQ ID NO:133-264, or a contiguous portion thereof of at least 14 nt.

65. The gRNA of claim 64, wherein the gRNA further comprises the sequence set forth in SEQ ID NO:398.

66. The gRNA of any of claims 55-65, wherein the gRNA comprises a gRNA that comprises the sequence set forth in any one of SEQ ID NOS: 265-396, optionally wherein the gRNA is the gRNA set forth in any one of SEQ ID NOS: 265-396.

67. The gRNA of claim 60 or claim 61, wherein the gene is selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23.

68. The gRNA of claim 60, claim 61, or claim 67, wherein the target site comprises the sequence set forth in any one of SEQ ID NOS: 1-28, a contiguous portion thereof of at least 14 nucleotides (nt), or a complementary sequence of any of the foregoing.

69. The gRNA of claim 60, 61, 67 or 68, wherein the gRNA comprises a gRNA spacer sequence comprising the sequence set forth in SEQ ID NO:133-160, or a contiguous portion thereof of at least 14 nt.

70. The gRNA of claim 69, wherein the gRNA further comprises the sequence set forth in SEQ ID NO:398.

71. The gRNA of claim 60, claim 61 or any of claims 67-70, wherein the gRNA comprises a gRNA that comprises the sequence set forth in any one of SEQ ID NOS: 265-292, optionally wherein the gRNA is the gRNA set forth in any one of SEQ ID NOS: 265-292.

72. The gRNA of claim 60 or claim 61, wherein the gene is selected from the list consisting of: DDIT3, HES2, HKR1, PLAG1, PRDM4, TEF, TSHZ1, ZNF141, ZNF562, ZNF691, ZNF692, and ZSCAN5A.

73. The gRNA of claim 60, claim 61 or claim 72, wherein the target site comprises the sequence set forth in any one of SEQ ID NOS: 1-12, a contiguous portion thereof of at least 14 nucleotides (nt), or a complementary sequence of any of the foregoing.

74. The gRNA of claim 60, 61, 72 or 73, wherein the gRNA comprises a gRNA spacer sequence comprising the sequence set forth in SEQ ID NO:133-144, or a contiguous portion thereof of at least 14 nt.

75. The gRNA of claim 74, wherein the gRNA further comprises the sequence set forth in SEQ ID NO:398.

76. The gRNA of any of claims 60, 61, and 72-75, wherein the gRNA comprises a gRNA that comprises the sequence set forth in any one of SEQ ID NOS: 265-276, optionally wherein the gRNA is the gRNA set forth in any one of SEQ ID NOS: 265-276.

77. The gRNA of any of claims 55-76, wherein the gRNA spacer sequence is between 14 nt and 24 nt, or between 16 nt and 22 nt in length.

78. The gRNA of any of claims 55-77, wherein the gRNA spacer sequence is 18 nt, 19 nt, 20 nt, 21 nt or 22 nt in length.

79. The gRNA of any of claims 55-78, wherein the gRNA comprises modified nucleotides for increased stability.

80. The gRNA of any of claims 55-79, wherein the gRNA is capable of complexing with a Cas protein or variant thereof.

81. The gRNA of any of claims 55-80, wherein the gRNA is capable of hybridizing to the target site or is complementary to the target site.

82. A CRISPR Cas-guide RNA (gRNA) combination comprising:
(a) a Clustered Regularly Interspaced Short Palindromic Repeats associated (Cas) protein or variant thereof; and
(b) at least one gRNA of any of claims 55-81 that targets the Cas protein or variant thereof to a target site in a gene or regulatory DNA element thereof in a T cell.

83. The CRISPR Cas-gRNA combination of claim 82, wherein the Cas protein or a variant thereof is a Cas9 protein or a variant thereof.

84. The CRISPR Cas-gRNA combination of claim 82 or claim 83, wherein the Cas protein or a variant thereof is a variant Cas protein, wherein the variant Cas protein lacks nuclease activity or is a deactivated Cas (dCas) protein.

85. The CRISPR Cas-gRNA combination of claim 83 or claim 84, wherein the variant Cas protein is a variant Cas9 protein that lacks nuclease activity or that is a deactivated Cas9 (dCas9) protein.

86. The CRISPR Cas-gRNA combination of claim 83, wherein the Cas9 protein or a variant thereof is a Staphylococcus aureus Cas9 (SaCas9) protein or a variant thereof.

87. The CRISPR Cas-gRNA combination of claim 83 or claim 84, wherein the variant Cas9 is a Staphylococcus aureus dCas9 protein (dSaCas9) that comprises at least one amino acid mutation selected from D10A and N580A, with reference to numbering of positions of SEQ ID NO: 405.

88. The CRISPR Cas-gRNA combination of claim 83, 84 or 87, wherein the variant Cas9 protein comprises the sequence set forth in SEQ ID NO: 406, or an amino acid sequence that has at least 90%, 91%, 92%, 93%, 94%, 95%, 96%, 97%, 98%, or 99% sequence identity thereto.

89. The CRISPR Cas-gRNA combination of claim 83, wherein the Cas9 protein or variant thereof is a Streptococcus pyogenes Cas9 (SpCas9) protein or a variant thereof.

90. The CRISPR Cas-gRNA combination of any of claim 83 or claim 84, wherein the variant Cas9 is a Streptococcus pyogenes dCas9 (dSpCas9) protein that comprises at least one amino acid mutation selected from D10A and H840A, with reference to numbering of positions of SEQ ID NO: 407.

91. The CRISPR Cas-gRNA combination of claim 83, claim 84 or claim 90, wherein the variant Cas9 protein comprises the sequence set forth in SEQ ID NO: 408, or an amino acid sequence that has at least 90%, 91%, 92%, 93%, 94%, 95%, 96%, 97%, 98%, or 99% sequence identity thereto.

92. A polynucleotide encoding the DNA-targeting system of any of claims 1-54, the fusion protein of the DNA-targeting system of any of claims 1-54, the gRNA of any of claims 55-81, the CRISPR Cas-gRNA combination of any of claims 82-91, or a portion or a component of any of the foregoing.

93. A plurality of polynucleotides encoding the DNA-targeting system of any of claims 1-54, the fusion protein of the DNA-targeting system of any of claims 1-54, the gRNA of any of claims 55-81, the CRISPR Cas-gRNA combination of any of claims 82-91, or a portion or a component of any of the foregoing.

94. A vector comprising the polynucleotide of claim 92.

95. A vector comprising the plurality of polynucleotides of claim 93.

96. The vector of claim 94 or claim 95, wherein the vector is a viral vector.

97. The vector of claim 96, wherein the vector is an adeno-associated virus (AAV) vector.

98. The vector of claim 97, wherein the vector is selected from among AAV1, AAV2, AAV3, AAV4, AAV5, AAV6, AAV7, AAV8, and AAV9.

99. The vector of claim 96, wherein the vector is a lentiviral vector.

100. The vector of claim 94 or claim 95, wherein the vector is a non-viral vector.

101. The vector of claim 100, wherein the non-viral vector is selected from: a lipid nanoparticle, a liposome, an exosome, or a cell penetrating peptide.

102. The vector of any of claims 94-101, wherein the vector exhibits immune cell or T-cell tropism.

103. The vector of any of claims 94-102, wherein the vector comprises one vector, or two or more vectors.

104. A modified T cell comprising the DNA-targeting system of any of claims 1-54, the gRNA of any of claims 55-81, the CRISPR Cas-gRNA combination of any of claims 82-91, the polynucleotide of claim 92, the plurality of polynucleotides of claim 93, the vector of any of claims 94-103, or a portion or a component of any of the foregoing.

105. A modified T cell comprising an epigenetic or phenotypic modification resulting from being contacted by the DNA-targeting system of any of claims 1-54, the gRNA of any of claims 55-81, the CRISPR Cas-gRNA combination of any of claims 82-91, the polynucleotide of claim 92, the plurality of polynucleotides of claim 93, the vector of any of claims 94-103, or a portion or a component of any of the foregoing.

106. The modified T cell of claim 104 or claim 105, wherein the modified T cell exhibits increased transcription of one or more genes that promote a stem cell-like memory T-cell phenotype, in comparison to a comparable unmodified T cell.

107. The modified T cell of claim 106, wherein the one or more genes are selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10.

108. The modified T cell of claim 106, wherein the one or more genes are selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23.

109. The modified T cell of claim 106, wherein the one or more genes are selected from the list consisting of: DDIT3, HES2, HKR1, PLAG1, PRDM4, TEF, TSHZ1, ZNF141, ZNF562, ZNF691, ZNF692, and ZSCAN5A.

110. The modified T cell of any of claims 106-109, wherein the transcription is increased by at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold.

111. The modified T cell of any of claims 104-110, wherein the modified T cell exhibits a stem cell-like memory T-cell phenotype.

112. The modified T cell of claim 111, wherein the stem cell-like memory T cell phenotype comprises expression of CCR7 and/or CD27.

113. The modified T cell of claim 111, wherein the stem cell-like memory T cell phenotype comprises expression of CCR7 and CD27.

114. The modified T cell of any of claims 111-113, wherein the stem cell-like memory T cell phenotype comprises one or more cell-surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−.

115. The modified T cell of any of claims 104-114, wherein the modified T cell is capable of a stronger and/or more persistent immune response, in comparison to a comparable unmodified T cell.

116. The modified T cell of any of claims 104-115, wherein the modified T cell is characterized by polyfunctional activity of the T cell to produce two or more cytokines following stimulation of the T cell with a stimulatory agent, optionally wherein the two or more cytokines are selected from among interferon-gamma (IFN-gamma), interleukin 2 (IL-2), and TNF-alpha.

117. The modified T cell of any of claims 104-116, wherein the modified T cell is derived from a cell from a subject.

118. The modified T cell of any of claims 104-117, wherein the modified T cell is derived from a primary T cell.

119. The modified T cell of any of claims 104-117, wherein the modified T cell is derived from a T cell progenitor, a pluripotent stem cell, or an induced pluripotent stem cell.

120. The modified T cell of any of claims 104-119, wherein the modified T cell further comprises an engineered T cell receptor (eTCR) or chimeric antigen receptor (CAR).

121. A method of increasing the transcription of one or more genes in a T cell, the method comprising introducing into a T cell the DNA-targeting system of any of claims 1-54, the gRNA of any of claims 55-81, the CRISPR Cas-gRNA combination of any of claims 82-91, the polynucleotide of claim 92, the plurality of polynucleotides of claim 93, the vector of any of claims 94-103, or a portion or a component of any of the foregoing.

122. The method of claim 121, wherein the one or more genes is a gene epigenetically modified by the DNA-targeting system.

123. The method of claim 121 or claim 122, wherein the transcription of the one or more genes is increased in comparison to a comparable T cell not subjected to the method.

124. The method of any of claims 121-123, wherein the transcription of the one or more genes is increased by at least about 1.2-fold, 1.25-fold, 1.3-fold, 1.4-fold, 1.5-fold, 1.6-fold, 1.7-fold, 1.75-fold, 1.8-fold, 1.9-fold, 2-fold, 2.5-fold, 3-fold, 4-fold, or 5-fold.

125. The method of any of claims 121-124, wherein the increased transcription of the one or more genes promotes a stem cell-like memory T cell phenotype in the T cell.

126. A method of promoting a stem cell-like memory T cell phenotype in a T cell, the method comprising introducing into the T cell the DNA-targeting system of any of claims 1-54, the gRNA of any of claims 55-81, the CRISPR Cas-gRNA combination of any of claims 82-91, the polynucleotide of claim 92, the plurality of polynucleotides of claim 93, the vector of any of claims 94-103, or a portion or a component of any of the foregoing.

127. The method of claim 125 or claim 126, wherein the stem cell-like memory T cell phenotype comprises one or more cell-surface markers selected from CCR7+, CD27+, CD45RA+, CD45RO−, CCR7+, CD62L+, CD28+, CD27+, IL-7Rα+, CXCR3+, CD95+, CD11a+, IL-2Rβ+, CD58+, and CD57−.

128. The method of any of claims 125-127, wherein the stem cell-like memory T cell phenotype comprises expression of CCR7 and/or CD27.

129. The method of any of claims 125-128, wherein the stem cell-like memory T cell phenotype is characterized by polyfunctional activity of the T cell to produce two or more cytokines following stimulation of the T cell with a stimulatory agent, optionally wherein the two or more cytokines are selected from among interferon-gamma (IFN-gamma), interleukin 2 (IL-2), and TNF-alpha.

130. The method of any of claims 121-129, wherein the T cell is a T cell in a subject and the method is carried out in vivo.

131. The method of any of claims 121-129, wherein the T cell is a T cell from a subject, or derived from a cell from the subject, and the method is carried out ex vivo.

132. The method of claim 131, wherein the T cell is a primary T cell.

133. The method of claim 131, wherein the T cell is derived from a T cell progenitor, a pluripotent stem cell, or an induced pluripotent stem cell.

134. A modified T cell produced by the method of any of claims 121-133.

135. A method of cell therapy for treating a disease in a subject in need thereof, comprising administering to the subject a cellular composition that comprises the modified T cell of any of claims 104-120 and 134.

136. The method of claim 135, wherein the modified T cell is obtained from or derived from a cell from said subject in need thereof.

137. The method of claim 135, wherein the subject is a first subject, and the modified T cell is obtained from or derived from a cell from a second subject.

138. The method of any of claims 135-137, wherein the subject in need thereof is a human.

139. The method of any of claims 135-138, wherein the administered modified T cell exhibits a stronger and/or more persistent immune response in the subject, in comparison to a comparable unmodified T cell.

140. The method of any of claims 135-139, wherein the subject has or is suspected of having a disease, condition, or disorder, optionally wherein the disease, condition, or disorder is cancer, viral infection, autoimmune disease, or graft-versus-host disease, or the subject has undergone or is expected to undergo organ transplantation.

141. The method of any of claims 135-140, wherein the subject has or is suspected of having cancer.

142. A pharmaceutical composition comprising the modified T cell of any of claims 104-120 and 134.

143. A pharmaceutical composition comprising the DNA-targeting system of any of claims 1-54, the gRNA of any of claims 55-81, the CRISPR Cas-gRNA combination of any of claims 82-91, the polynucleotide of claim 92, the plurality of polynucleotides of claim 93, the vector of any of claims 94-103, or a portion or a component of any of the foregoing.

144. The pharmaceutical composition of claim 142 or claim 143, for use in treating a disease, condition, or disorder in a subject.

145. The pharmaceutical composition of claim 142 or claim 143, for use in the manufacture of a medicament for treating a disease, condition, or disorder in a subject.

146. The pharmaceutical composition of claim 144 or claim 145, wherein the subject has or is suspected of having a disease, condition, or disorder, optionally wherein the disease, condition, or disorder is cancer, viral infection, autoimmune disease, or graft-versus-host disease, or the subject has undergone or is expected to undergo organ transplantation.

147. The pharmaceutical composition of any of claims 144-146, wherein the subject has or is suspected of having cancer.

148. The pharmaceutical composition of any of claims 144-147, wherein the pharmaceutical composition is to be administered to the subject in vivo.

149. The pharmaceutical composition of any of claims 144-147, wherein the subject is a first subject, and the pharmaceutical composition is to be administered ex vivo to T cells from the first subject, or to T cells from a second subject.

150. The pharmaceutical composition of claim 149, wherein following administration to T cells from the first subject or second subject, the T cells are administered to the first subject.

151. The pharmaceutical composition of any of claims 143-148, wherein following administration of the pharmaceutical composition, the expression of one or more genes is increased in T cells of the subject.

152. The pharmaceutical composition of claim 149 or claim 150, wherein following administration of the pharmaceutical composition to the T cells from the first or second subject, the expression of one or more genes is increased in the T cells.

153. The pharmaceutical composition of claim 151 or claim 152, wherein the one or more genes are selected from the list consisting of: DDIT3, HES2, PATZ1, ZBED5, ZNF319, HELT, PLAG1, SOX21, ZNF141, ZNF470, EBF4, HKR1, ZBTB7A, ZNF691, ZNF692, FOXD3, HMGN3, PRDM4, TSHZ1, ZSCAN5A, PRDM8, ZNF219, ZNF562, ZNF816, ZSCAN23, AEBP1, ARID3A, ARNTL2, BACH1, BATF2, BRD4, CDX4, CENPB, DMRT1, E2F2, EMX1, ESRRB, ETS1, FERD3L, FEV, FLYWCH1, FOXJ3, FOXO3, GLIS3, HDAC9, HIC2, HIVEP1, HOXA2, HOXC9, HSFY1, ISL2, KAT2A, KDM5D, KLF13, KLF6, MEOX2, MLXIPL, MNT, MYCN, MYT1L, NEUROG3, NFE2, NR2F1, NR2F2, NR3C1, PHF20, PITX2, POU2AF1, POU3F2, POU3F3, PRDM11, PRMT3, PROP1, PURA, RELB, RFX3, SAFB, SETBP1, SMAD2, SMAD6, SNAPC2, SNAPC4, SON, SOX11, SOX12, SOX14, SOX30, SPIC, STAT1, TBX15, TEF, THAP7, TOX2, TRERF1, TRIM3, VDR, YBX2, ZBTB14, ZBTB4, ZBTB46, ZFHX2, ZFP57, ZFP62, ZFP69B, ZIC1, ZIC4, ZNF136, ZNF16, ZNF174, ZNF253, ZNF26, ZNF263, ZNF285, ZNF317, ZNF331, ZNF345, ZNF420, ZNF423, ZNF48, ZNF496, ZNF501, ZNF524, ZNF541, ZNF557, ZNF619, ZNF735, ZNF747, ZNF783, ZNF805, and ZSCAN10.

154. The pharmaceutical composition of claim 151 or claim 152, wherein the one or more genes are selected from the list consisting of: DDIT3, HES2, HKR1, PLAG1, PRDM4, TEF, TSHZ1, ZNF141, ZNF562, ZNF691, ZNF692, ZSCAN5A, EBF4, ETS1, FOXD3, HELT, HMGN3, PATZ1, PRDM8, SOX21, TBX15, ZBED5, ZBTB7A, ZNF219, ZNF319, ZNF470, ZNF816, and ZSCAN23.

155. The pharmaceutical composition of claim 151 or claim 152, wherein the one or more genes are selected from the list consisting of: DDIT3, HES2, HKR1, PLAG1, PRDM4, TEF, TSHZ1, ZNF141, ZNF562, ZNF691, ZNF692, and ZSCAN5A.

156. A method for treating a disease in a subject in need thereof, comprising administering to the subject the DNA-targeting system of any of claims 1-54, the gRNA of any of claims 55-81, the CRISPR Cas-gRNA combination of any of claims 82-91, the polynucleotide of claim 92, the plurality of polynucleotides of claim 93, the vector of any of claims 94-103, the modified T cell of any of claims 104-120 and 134, the pharmaceutical composition of any of claims 142-155, or a portion or a component of any of the foregoing.

157. Use of the DNA-targeting system of any of claims 1-54, the gRNA of any of claims 55-81, the CRISPR Cas-gRNA combination of any of claims 82-91, the polynucleotide of claim 92, the plurality of polynucleotides of claim 93, the vector of any of claims 94-103, the modified T cell of any of claims 104-120 and 134, the pharmaceutical composition of any of claims 142-155, or a portion or a component of any of the foregoing, for the treatment of a disease or disorder.

158. Use of the DNA-targeting system of any of claims 1-54, the gRNA of any of claims 55-81, the CRISPR Cas-gRNA combination of any of claims 82-91, the polynucleotide of claim 92, the plurality of polynucleotides of claim 93, the vector of any of claims 94-103, the modified T cell of any of claims 104-120 and 134, the pharmaceutical composition of any of claims 142-155, or a portion or a component of any of the foregoing, in the manufacture of a medicament for treating a disorder.

159. A composition comprising the DNA-targeting system of any of claims 1-54, the gRNA of any of claims 55-81, the CRISPR Cas-gRNA combination of any of claims 82-91, the polynucleotide of claim 92, the plurality of polynucleotides of claim 93, the vector of any of claims 94-103, the modified T cell of any of claims 104-120 and 134, the pharmaceutical composition of any of claims 142-155, or a portion or a component of any of the foregoing, for the treatment of a disease or a disorder.
